(19) United States
(12) Patent Application Publication
(10) Pub. No.: US 2025/0113829 A1
(43) Pub. Date: Apr. 10, 2025

(54) HIGH MOLECULAR WEIGHT MODIFIED RNA COMPOSITIONS

(71) Applicant: NanoSUR LLC, Miami, FL (US)

(72) Inventors: Darren H. Wakefield, Madison, WI (US); Grace B. Arhancet, Miami, FL (US); Vladimir Trubetskoy, Madison, WI (US); Juan P. Arhancet, Miami, FL (US); Jason Klein, Verona, WI (US)

(21) Appl. No.: 18/948,365

(22) Filed: Nov. 14, 2024

Related U.S. Application Data
(63) Continuation-in-part of application No. 18/687,470, filed on Jan. 1, 0001.
(60) Provisional application No. 63/239,165, filed on Aug. 31, 2021.
(60) Provisional application No. 63/598,701, filed on Nov. 14, 2023.

(51) Int. Cl. A01N 63/60; A01P 7/04; C12N 15/113

(52) U.S. Cl. CPC A01N 63/60; A01P 7/04; C12N 15/113; C12N 2310/14; C12N 2330/30

(57) ABSTRACT

Post-transcriptionally chemically modified double strand RNAs (MdsRNAs) and post-transcriptionally chemically modified RNAs (MRNAs) having a high molecular weight polyalkyloxy modification at the 2′-OH position are described. This modification allows for greater bioavailability of the compound, better stability of the compound, and allows for greater stability against nucleases. The MdsRNAs and MRNAs can be economically produced in a readily scalable manner. Compositions of MdsRNAs and MRNAs having chemically modified nucleotides are described, such that the MdsRNAs and MRNAs are modified to contain high molecular weight polyalkyloxy polymers. Synthetic methods for efficiently making these MdsRNAs and MRNAs and methods of using MdsRNAs and MRNAs are described.

### RELATED APPLICATIONS

This application is a continuation-in part of U.S. application Ser. No. 18/687,470, filed on Feb. 28, 2024, which is the U.S. National Stage of International Application No. PCT/US2022/075740, filed Aug. 31, 2022, which designates the U.S., published in English, and claims the benefit of U.S. Provisional Application No. 63/239,165, filed on Aug. 31, 2021. This application also claims the benefit of U.S. Provisional Application No. 63/598,701, filed on Nov. 14, 2023. The entire teachings of the above application(s) are incorporated herein by reference.

### GOVERNMENT SUPPORT

This invention was made with government support under Award Number 1853008 from the National Science Foundation. The government has certain rights in the invention.

### INCORPORATION BY REFERENCE OF MATERIAL IN XML

This application incorporates by reference the Sequence Listing contained in the following extensible Markup Language (XML) file being submitted concurrently herewith:
a) File name: 55811003011.xml; created Nov. 14, 2024, 339,824 Bytes in size.

### BACKGROUND

Because they dominate all terrestrial environments that support human life, insects are usually humanity's most important competitors for food, fiber, and other natural resources. Insects have a direct impact on agricultural food production by chewing the leaves of crop plants, sucking out plant juices, boring within the roots, stems or leaves, and spreading plant pathogens. Insects feed on natural fibers, destroy wooden building materials, ruin stored grain, and accelerate the process of decay.

The economic impact of insects is measured not only by the market value of products they destroy and the cost of damage they inflict but also by the money and resources expended on prevention and control of pest outbreaks. Although dollar values for these losses are nearly impossible to calculate, especially when they affect human health and welfare, economists generally agree that insects consume or destroy around 10% of gross national product in large, industrialized nations and up to 25% of gross national product in some developing countries.

One possible method for controlling insect populations is the use of RNA interference (RNAi), which is a naturally occurring biological process by which double-stranded ribonucleic acid (dsRNA) silences (knocks down) target gene expression in a sequence specific manner. Cellular enzymes use dsRNA to target and cleave single stranded RNA (ssRNA), including messenger RNA (mRNA) and non-coding RNA. RNAi is known to occur in many eukaryotes, including plants, insects, acari, fungi and animals, and offers great potential for selective and efficient regulation of gene expression.

The dsRNA has an antisense strand containing sequence complementary to a sequence in the mRNA or non-coding RNA and a sense strand containing sequence complementary to the guide strand sequence and substantially identical to the sequence in the mRNA or non-coding RNA. The sense and antisense sequences can be present on separate RNA strands or on a single strand. When present on a single strand, the complementary sequences are connected by a non-hybridizing hairpin or loop sequence.

RNAi-mediated gene suppression on targeted plants, insects, acari, and fungi affecting crops described in the prior art has been achieved using exogenously supplied unmodified dsRNA (UdsRNA) (U.S. Pat. No. 9,121,022; Ivashuta et al. 2015; U.S. Publication No. 20160215290; Koch et al. 2016). It has been found, that when dsRNAs are used to induce RNAi in insects and are supplied in the insects' diet, 60 base pair (bp) or longer dsRNAs are sometimes required for efficient uptake and processing (Bolognesi et al. 2012).

Preparation of unmodified ssRNA capable of folding into UdsRNAs between 25 and 1000 base pairs (bp), such ssRNA having a first sense section followed by a ssRNA linker followed by a second sense section followed by a ssRNA linker followed by a third sense section followed by a ssRNA linker followed by a section antisense to the first sense section followed by a ssRNA linker followed by another section antisense to the second sense section followed by a ssRNA linker followed by another section antisense to the third sense section has been described (Arhancet, US20180265868 A1).

Preparation of unmodified ssRNA capable of folding into UdsRNAs between 25 and 1000 base pairs (bp), such ssRNA having a first sense section followed by a ssRNA linker followed by a second sense section followed by a ssRNA linker followed by a section antisense to the second sense section followed by a ssRNA linker followed by another section antisense to the first sense section has been described (Arhancet, US20160177299 A1).

Preparation of UdsRNA longer than about 30 base pairs (bp) has been achieved by in vitro transcription (Timmons 2006) and by fermentation (Fire et al. 1998). Commercially feasible large-scale methods for preparation and purification of the UdsRNA, including those in which the sense and antisense strands are linked by ssRNA in a hairpin, has been described (Arhancet et al., U.S. Pat. No. 9,822,361B2, Baum et al. U.S. Pat. No. 9,445,603B2). However, UdsRNAs are sensitive to degradation by nucleases in the environment and the host, reducing efficacy of inhibition of gene expression (Baum 2016).

Merino et al. (JACS vol. 127, No. 12, 2005 pp 4223-4231) invented an analytical technique (SHAPE) describing the chemical modification of RNA constructs containing short ssRNA sections and very short dsRNA stems by reacting them with small molecules (e.g. NMIA). Because they determined the position of the chemically modified nucleotide using a reverse transcription reaction that stops at the first modification found by the reverse transcriptase enzyme, they used reaction conditions conducive to chemical modification extents of equal or less than 1 nucleotide per reacted RNA construct (“NMIA reacts with single-hit kinetics”). For example, the RNA construct described in FIG. 7 by Merino et al. comprises dsRNA stems not longer than 8 base pairs and 32 nucleotides in the ssRNA sections of the construct. 32=20 (tRNA^{Asp})+4 (3′ linker)+4 (RT primer binding site)+4 (5′ linker). Thus, of those RNA constructs that reacted with NMIA, just 1 in 32 RNA nucleotides in all the ssRNA sections were actually reacted, i.e., about 3.1% of the nucleotides in the ssRNA sections. Furthermore, Wilkinson et al. (Nature protocols 1.3 (2006): 1610-1616) showed in FIG. 4 that the transcript derived from the unreacted RNA construct was, by far, the most abundant of all transcripts. Consequently, significantly less than about 3.1% of the nucleotides in the ssRNA sections of the starting RNA construct were reacted with NMIA. This novel analytical technique (SHAPE) has been used by others describing products similar to the RNA construct in FIG. 7 by Merino et al., e.g., as described by Chen et al. (The EMBO Journal 25.13 (2006): 3156-3166), Badorrek et al. (Proceedings of the National Academy of Sciences 103.37 (2006): 13640-13645), Kierzek et al. (Biochemistry 45.2 (2006): 581-593), Costantino et al. (Nature structural & molecular biology 15.1 (2008): 57-64), Duncan et al. (Biochemistry 47.33 (2008): 8504-8513), Watts et al. (Nature 460.7256 (2009): 711-716), Deigan et al. (Proceedings of the National Academy of Sciences 106.1 (2009): 97-102), Nodin et al. (Bioorganic & Medicinal Chemistry Letters 25.3 (2015): 566-570), Weng et al. (Nature chemical biology 16.5 (2020): 489-492). Huston et al. (Proceedings of the National Academy of Sciences 121.29 (2024): €2312080121), etc. They all have in common the very low % of reacted nucleotides in the RNA construct tested. This is needed to avoid having RNA constructs reacted with more than one molecule of reactant since the transcription would stop at the first. Furthermore, for this reason the maximum allowable % of nucleotides reacted diminishes as the total number of reactive nucleotides increases. However, because unmodified (unreacted) single stranded nucleotides in an RNA construct are much more unstable (prone to hydrolysis) than modified (reacted) single stranded nucleotides, and because such instability results in the ablation of the efficacy obtained by using the intact (not hydrolyzed) construct, there remains a need for stable RNA constructs that trigger RNA interference (comprising dsRNA stems longer than 19 base pairs) comprising ssRNA sections, or other stable active ingredients of similar selectivity or mechanism of action comprising ssRNA sections and dsRNA stems.

Brown et al. (US20170305956) describe a dsRNA construct in which the sense section and the antisense section are in different RNA strands, wherein the shortest of the two strands, i.e., the antisense strand, is shorter than 35 nucleotides, wherein the sense strand comprises a tetraloop and the tetraloop comprises at least one nucleotide that has been reacted (conjugated) with a reactant. Other similar constructs have been described in which a sense section and an antisense section are in different strands wherein the shortest strand has less than 35 nucleotides, and wherein some nucleotides in either strand have been reacted, e.g., by Monahan et al. (WO 2004/076630), Inberg et al. (WO 2015/066681). However, because there is a direct relationship between the efficacy of the transcription of DNA into RNA and the length of the transcript, there remains a need for transcripts longer than 65 nucleotides that can be post transcriptionally modified. Furthermore, because the hybridization of sense and antisense strands is favored when they are in the same strand, there is a need for transcripts longer than 65 nucleotides that have sense and antisense strands in the same transcript that can be post transcriptionally modified.

Goldsborough (WO 00/66605) described naturally occurring RNA materials, e.g., messenger RNA, ribosomal RNA, transfer RNA, heterogenous nuclear RNA, viral RNA, etc., post transcriptionally modified that can be used to infer the concentration of the unmodified RNA material in a given sample to be analyzed by increasing the concentration of such modified RNA material using a polymerase capable of its replication. Thus, there remains a need for modified RNA materials having sense and antisense strands flanked by linker strands wherein such sense and antisense strands are complementary to an RNA expressed in a target organism and are specifically designed to decrease the concentration of such an expressed RNA.

DsRNA degradation has been addressed in in vitro and in vivo research and for human therapeutics (Ku et al. 2015) by using chemical synthesis of small (<30 bp) interfering dsRNAs (siRNA) with nucleotides modified by chemical means. Preparation of siRNA with chemically modified nucleotides involves sequential protection-deprotection chemical reactions for each nucleotide added in the elongating single strand RNA (ssRNA) chain (Micura 2002). The complexity and expense of such processes are significantly increased for RNA molecules which trigger RNAi (RNAi triggers) that are longer than about 30 bp. While chemically synthesized siRNAs targeting insects using nucleotides chemically modified at the 2′-OH position of the ribose have been also described (Gong et al. 2013), the cost and synthetic complexity of modified siRNAs is neither economically feasible or sufficiently scalable, for preparation of amounts larger than a few grams, or of chemically modified dsRNA longer than about 30 bp.

Post-transcriptional chemical modification of ssRNA for analytical purposes was also described by Merino (2005). Merino reacted ssRNA in aqueous media containing 10% DMSO with N-methylisatoic anhydride (NMIA) to produce the 2′aO-esters of N-methyl-anthranilic acid at single stranded nucleotides. Derivatization by this method was inefficient. Less than 15% of ssRNA chains in a reaction vessel were modified and those that were modified had, on average, a single 2′nO-ester of NMIA per ssRNA chain. Under these conditions, dsRNA reacted more than 80 times less efficiently, with less than 0.18% of nucleotides in a stem region being modified and only within one (1) nucleotide of the end of a stem (e.g. within one nucleotide of a single strand region) Similar results have been observed for reaction of RNA with other reactants (Nodin 2015). 1-methyl-7-nitroisatoic anhydride (1M7), benzoyl cyanide (BzCN), 2-methyl-3-furoic acid imidazolide (FAI), and 2-methylnicotinic acid imidazolide (NAI) have been used to post-transcriptionally produce 2′-ribose esters of RNA, but have a similarly low percentage of the modification, with modification primarily occurring at riboses of unpaired nucleotides or their immediately adjacent paired nucleotides.

Unmodified poly-ribonucleic acid is an exceptionally unstable molecule. Unlike DNA, it contains a hydroxyl group at the 2′ position of the ribose which renders the RNA polymer sensitive to hydrolysis. Deprotonation of 2′-OH and, consequently, nucleophilic attack of 2′ oxygen on the backbone phosphorus is the primary molecular mechanism of cleavage of phosphodiester bonds by various nucleolytic enzymes, including RNase A (Elliot and Ladomery, 2011). Stabilization of the nucleic acid backbone against nucleolysis has become a success-defining issue, much the same as for the evolution of siRNA-based therapeutics (Nair et al., 2014). The issue of RNA stability is especially important in intestinal milieu rich in nucleolytic activity with various endonucleases and phosphodiesterases responsible for digestion of nucleic acids (Whitt and Savage, 1988; Liu et al., 2015). A highly effective strategy for stabilizing RNA molecules is the derivatization of 2′—O group. Various modifications at this position have been demonstrated to improve enzymatic stability of various RNA molecules, including nanoassemblies (Liu et al. 2010) and RNAi silencing molecules (Khvorova and Watts, 2017). These modifications are generally applied during chemical synthesis of small interfering RNAs (siRNAs).

While many advances have been made in the use of dsRNA, especially those that have chemical modifications (specifically polyalkyloxy polymers) at the 2′-OH position, there is the need for chemically modified dsRNA compositions that are more efficient, more soluble, and more bioavailable than have been available in the past. There is also a need for synthetic methods of making these chemically modified dsRNA compositions in an efficient manner to allow for mass-scale production.

### SUMMARY

Described herein are post-transcriptionally chemically modified double strand RNAs (MdsRNAs) having a high molecular weight polyalkyloxy modification at the 2′-OH position. This modification allows for greater bioavailability of the compound, better stability of the compound, and allows for greater stability against nucleases. As described herein, these MdsRNAs can be economically produced in a readily scalable manner.

Also described herein are post-transcriptionally modified RNA constructs comprising ssRNA sections linking two different dsRNA stems, having a high molecular weight polyalkyloxy modification at the 2′-OH position in some of the ribose rings in the ssRNA sections and/or the dsRNA stem.

This disclosure describes compositions of modified double strand RNA (MdsRNA) having chemically modified nucleotides, such that the MdsRNAs are modified to contain high molecular weight polyalkyloxy polymers, optionally further comprising 2′-O chemically modified nucleotides to contain low molecular weight (LMW) moieties (LMW modified nucleotides). This disclosure also describes synthetic methods for efficiently making these modified MdsRNAs.

This disclosure describes methods to efficiently produce modified double strand RNA (MdsRNA) having chemically modified nucleotides, such that the MdsRNAs are modified to contain high molecular weight polyalkyloxy polymers, optionally further comprising 2′-O chemically modified nucleotides to contain low molecular weight (LMW) moieties (LMW modified nucleotides).

Thus, in one aspect, the present disclosure provides compositions comprising a post-transcriptionally chemically modified double strand RNA (MdsRNA) wherein the MdsRNA comprises a double strand RNA wherein no more than about 30% of all the nucleotides independently comprise Formula (I)

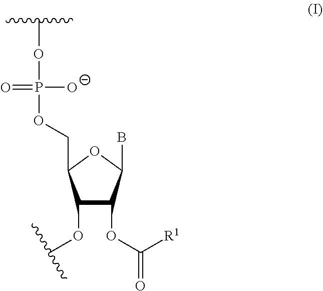

or an acceptable salt thereof, wherein B and R¹ are defined herein.

In an embodiment of this aspect, the post-transcriptional chemical modification of the double strand RNA comprises no more than about 30% of all the nucleotides being modified with high molecular weight polyalkyloxy polymers. In another embodiment, the post-transcriptional chemical modification of the double strand RNA further comprises at least about 2% to about 50% of all the nucleotides being modified with LMW moieties. In one embodiment, the post-transcriptional chemical modification of the double strand RNA further comprises from about 2% to about 50% of all the nucleotides being modified with LMW moieties. In an embodiment, the post-transcriptional chemical modification of the double strand RNA comprises about a 3:5:2 ratio of high molecular weight modified nucleotides to LMW modified nucleotides to unmodified nucleotides. In another embodiment, the post-transcriptional chemical modification of the double strand RNA comprises about a 3:6.5:0.5 ratio of high molecular weight modified nucleotides to LMW modified nucleotides to unmodified nucleotides. In still another embodiment, the post-transcriptional chemical modification of the double strand RNA comprises about a 1:7:2 ratio of high molecular weight modified nucleotides to LMW modified nucleotides to unmodified nucleotides. In still another embodiment, the post-transcriptional chemical modification of the double strand RNA comprises about a 0.5:9:0.5 ratio of high molecular weight modified nucleotides to LMW modified nucleotides to unmodified nucleotides. In still another embodiment, the post-transcriptional chemical modification of the double strand RNA comprises about a 0.3:4:5.7 ratio of high molecular weight modified nucleotides to LMW modified nucleotides to unmodified nucleotides. In still another embodiment, the post-transcriptional chemical modification of the double strand RNA comprises about a 0.01:4:5.99 ratio of high molecular weight modified nucleotides to LMW modified nucleotides to unmodified nucleotides. In still another embodiment, the post-transcriptional chemical modification of the double strand RNA comprises about a 0.06:0:96.4 ratio of high molecular weight modified nucleotides to LMW modified nucleotides to unmodified nucleotides.

In another aspect, the disclosure provides a method of preparing a composition comprising a post-transcriptionally chemically modified double strand RNA (MdsRNA) wherein the MdsRNA comprises a double strand RNA wherein no more than about 30% of all the nucleotides independently comprise Formula (I)

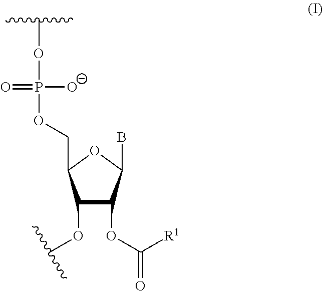

or an acceptable salt thereof, wherein B and R¹ are defined herein.

In another aspect, the disclosure provides a method of preparing a composition comprising a post-transcriptionally chemically modified RNA (MRNA) wherein the MRNA comprises single strand RNA sections linking two different dsRNA stems or a sense section and an antisense section in the same dsRNA stem wherein no more than about 50% (e.g., about 2% to about 50%, or about 5% to 35%, or about 10% to 30%, or about 15% to 20%) of all the nucleotides in the ssRNA independently comprise Formula (I):

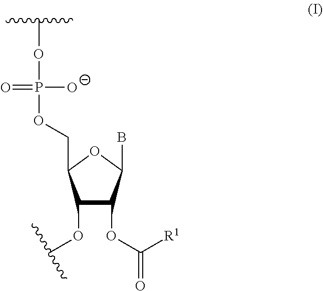

or an acceptable salt thereof, wherein B and R¹ are defined herein.

Methods for making embodiments of the compositions described herein are disclosed. In an embodiment of the methods of preparing compositions, the post-transcriptional chemical modification of the double strand RNA comprises no more than about 30% of all the nucleotides being modified with high molecular weight polyalkyloxy polymers. In another embodiment of the methods of preparing compositions, the post-transcriptional chemical modification of the double strand RNA further comprises at least about 2% of all the nucleotides being modified with LMW moieties. In one embodiment, the post-transcriptional chemical modification of the double strand RNA further comprises from about 2% to about 50% of all the nucleotides being modified with LMW moieties. In an embodiment of the methods of preparing compositions, the post-transcriptional chemical modification of the double strand RNA comprises about a 3:5:2 ratio of high molecular weight modified nucleotides to LMW modified nucleotides to unmodified nucleotides. In another embodiment of the methods of preparing compositions, the post-transcriptional chemical modification of the double strand RNA comprises about a 3:6.5:0.5 ratio of high molecular weight modified nucleotides to LMW modified nucleotides to unmodified nucleotides. In still another embodiment of the methods of preparing compositions, the post-transcriptional chemical modification of the double strand RNA comprises about a 1:7:2 ratio of high molecular weight modified nucleotides to LMW modified nucleotides to unmodified nucleotides. In still another embodiment of the methods of preparing compositions, the post-transcriptional chemical modification of the double strand RNA comprises about a 0.5:9:0.5 ratio of high molecular weight modified nucleotides to LMW modified nucleotides to unmodified nucleotides. In still another embodiment of the methods of preparing compositions, the post-transcriptional chemical modification of the double strand RNA comprises about a 0.3:4:5.7 ratio of high molecular weight modified nucleotides to LMW modified nucleotides to unmodified nucleotides. In still another embodiment of the methods of preparing compositions, the post-transcriptional chemical modification of the double strand RNA comprises about a 0.01:4:5.99 ratio of high molecular weight modified nucleotides to LMW modified nucleotides to unmodified nucleotides. In still another embodiment of the methods of preparing compositions, the post-transcriptional chemical modification of the double strand RNA comprises about a 0.06:0:96.4 ratio of high molecular weight modified nucleotides to LMW modified nucleotides to unmodified nucleotides.

Thus, in one aspect, the present disclosure describes methods for producing MdsRNA deliverable to pests comprising:
a) transcribing genomic DNA belonging to a pest into dsRNA formed from a ssRNA strand comprising from about 1 to about 21, or from about 2 to about 15, or from about 2 to about 10, or from about 3 to about 7 sense sections followed by from about 1 to about 21, or from about 2 to about 15, or from about 2 to about 10, or from about 3 to about 7 sections antisense to the sense sections;
b) adding a salt comprising a quaternary ammonium, e.g., or a quaternary phosphonium cation or a pyrrolidinium or a pyridium or a piperidimium and a neutralizing anion to produce an isolate comprising more than half of the dsRNA produced in (a); and
c) conducting a chemical reaction between suitable reagents and at least about 2% to about 30%, or about 5% to 35%, or about 10% to 30%, or about 15% to 20% of the 2′OH groups in the dsRNA resulting from (b) in a blend comprising at least about 10% to about 100%, or about 20% to 100%, or about 30% to 100%, or about 60% to 100%, or about 70% to 95% of the salt used in (b), resulting in a post-transcriptionally chemically modified double strand RNA (MdsRNA) wherein (a), (b), and (c) are defined herein.

In further aspects, the disclosure provides methods of modifying the expression of polynucleotides of interest in an insect, a fungus, a weed or an acari, using any of the compositions, target insects, fungi, weeds or acari, and sequences set forth herein.

### BRIEF DESCRIPTION OF THE DRAWINGS

FIG. 1 shows cumulative mortality for Diamondback moth larvae after administration of select compounds of Formula (I).

FIG. 2 shows decrease of dsRNA content on cabbage leaves in the field for treatment C2, unmodified dsSNF7, and modified dsSNF7 treatments NS2 (PEG-dsSNF7) and NS5 (NMIA-dsSNF7).

FIG. 3 shows the mortality rate of Diamondback moth field larvae after administration of select compounds of Formula (I) after 3-days (3 DA-A) and 4-days (4 DA-A).

FIG. 4 shows Agarose gel electrophoresis analysis of 300 bp dsRNA modified with activated 10k PEG. Lines: 1—DNA ladder; 2—original dsRNA; 3—10k PEG-MdsRNA reaction mixture.

FIGS. 5A-5C show schematic representations of A) transcribed ssRNA wherein sections i* (* denotes antisense section of RNA base pairs), j*, and k* are antisense to sections i′-i, j and k, respectively; PAC (ssRNA strand with high affinity or high binding constant with capsid protein) sites link sections k and i* as well as sections k* and i′; and short ssRNA sections/link section i and j, j and k, i* and j*, and j* and k*; B) transcribed ssRNA shown in (A) folded into Type A MRNAs and C) a transcribed type (A) RNA with modifying moieties of high or low molecular weight.

FIG. 6 shows a transcribed type (B) RNA.

FIG. 7 shows a transcribed type (C) RNA.

FIG. 8 shows a comparison of MdsRNA to the three different types of MRNA ((A), (B), and (C)) with the squiggly lines representing modifying moieties of high or low molecular weight.

### DESCRIPTION

### Definitions

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of ±20 percent, ±15%, ±10%, ±5%, ±1%. Further, any numerical range presented herein is inclusive of the numbers stated in the range. Any numerical range that is presented herein that does not state “about” is intended to include the term “about” for the numerical values of the range boundaries presented. For example, the term “about” when used in reference to a number of nucleotides or base pairs may refer to ±10 nucleotides, ±9, ±8, ±7, ±6, ±5, ±4, ±3, ±2, ±1 nucleotides when referring to ssRNA; ±10 base pairs, ±9, ±8, ±7, ±6, ±5, ±4, ±3, ±2, ±1 base pairs when referring to dsRNA or dsDNA.

As used herein, the term “sequence” or “nucleotide sequence” refers to a succession or order of nucleobases, nucleotides, and/or nucleosides, described with a succession of letters using the standard nucleotide nomenclature and the key for modified nucleotides described herein. The sequences as described herein are listed from the 5′ terminus to the 3′ terminus.

As used herein, the term “linker” refers to a ssRNA string of from about 1 to about 15 nucleotides of a substantially non-hybridizing region of an RNA transcript.

A sense RNA strand is defined as a single-stranded RNA molecule designed to hybridize with a complementary antisense RNA strand, forming a double-stranded RNA (dsRNA) structure. The sense strand must have a minimum length of 15 nucleotides and must form at least 12 base pairs with the antisense strand. Base pairing follows the standard Watson-Crick rules (A-U, C-G) and may also include G-U wobble pairing.

An antisense RNA strand is defined as a single-stranded RNA molecule designed to hybridize with a complementary sense RNA strand, forming a double-stranded RNA (dsRNA) structure. The antisense strand must have a minimum length of 15 nucleotides and must form at least 12 base pairs with the sense strand. Base pairing follows the standard Watson-Crick rules (A-U, C-G) and may also include G-U wobble pairing.

The skilled artisan would understand that the reverse complementary strand (i.e., described from the 3′ terminus to the 5′ terminus) can be the full reverse complementary strand or a strand comprising stretches of contiguous nucleotides of 11 nucleotides and 60 nucleotides long, wherein each of such stretches of 11 to 60 contiguous nucleotides are the reverse complementary strand of a stretch of equal length in the sequences as described herein. In some embodiments, a MdsRNA is at least 40, at least 30, at least 50, at least 70, at least 80, at least 90, or at least 100 base pairs in length. A MdsRNA sense strand contains a sense sequence and a MdsRNA antisense strand contains an antisense sequence. The antisense sequence is 100% (perfectly) complementary or at least about 90% (substantially) complementary or at least about 80% (partial) complementary to a nucleotide sequence present in a target gene transcribed mRNA or non-coding RNA (i.e., expressed RNA). The sense sequence is 100% (perfectly) complementary or at least about 90% (substantially) complementary or at least about 80% (partially) complementary the antisense sequence. A sense sequence may also be 100% identical, at least about 90% identical, or at least about 80% identical to a nucleotide sequence (target sequence) present in a target gene mRNA or non-coding RNA. The sense sequence and a corresponding antisense sequence are partially (at least about 80%), substantially (about 90%), or fully (100%) complementary to each other. In some embodiments, the region of complementarity (antisense sequence) or identity (sense sequence) between the MdsRNA and a corresponding sequence in the target gene transcribed mRNA or non-coding RNA sequence is greater than 19, at least 20, at least 30, at least 40, at least 50, at least 60, at least 70, at least 80, at least 90, or at least 100 nucleotides in length. In some embodiments, the antisense sequence contains a contiguous sequence from about 19 to about 100, or from about 19 to about 80, or from about 19 to about 60, or from about 19 to about 40, or from about 19 to about 30, or from about 20 to about 30 nucleotides in length that is 100% complementary or from about 80% to about 100%, or from about 85% to about 100%, or from about 90% to about 100% complementary to a corresponding contiguous sequence in the target gene transcribed mRNA or non-coding RNA.

In some embodiments, the region of complementarity (antisense sequence) or identity (sense sequence) between the MdsRNA and a corresponding sequence in the target gene transcribed mRNA or non-coding RNA sequence is from about 19 to about 60, or from about 20 to about 35, or from about 50 to about 60 nucleotides in length. In some embodiments, the antisense sequence contains a contiguous sequence from about 19 to about 60, or from about 20 to about 35, or from about 50 to about 60 nucleotides in length that is 100% complementary or at least about 80%, at least about 85%, at least about 90%, at least about 95%, at least about 96%, at least about 97%, at least about 98%, at least about 99%, complementary to a corresponding contiguous sequence in the target gene transcribed mRNA or non-coding RNA. MdsRNA sense and antisense sequences can be either the same length or they can be of different lengths. MdsRNA sense and antisense sequences can be either the same length or they can be 60 different lengths. Suitable sense and antisense sequences are identified using known methods readily available in the art.

As used herein, the term “nucleotide” refers to one base pairing unit (e.g., a unit of Formula I or an unmodified ribose) comprising a purine or pyrimidine base pairing moiety. The term may refer to the nucleotide unit with or without the attached intersubunit linkage, although, when referring to a “charged subunit”, the charge typically resides within the intersubunit linkage.

The purine or pyrimidine base pairing moiety, also referred to herein simply as a “nucleobases,” “base,” or “bases,” may be adenine, cytosine, guanine, uracil, thymine or inosine. Also included are bases such as pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, quesosine, 2-thiouridine, 4-thiouridine, wybutosine, wybutoxosine, 4-acetyltidine, 5-(carboxyhydroxymethyl) uridine, 5′-carboxymethylaminomethyl-2-thiouridine, 5-carboxymethylaminomethyluridine, β-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, 13-D-mannosylqueosine, uridine-5-oxyacetic acid, 2-thiocytidine, threonine derivatives and others (Burgin et al., 1996, Biochemistry, 35:14090; Uhlman & Peyman, supra). By “modified bases” in this aspect is meant nucleotide bases other than adenine (A), guanine (G), cytosine (C), thymine (T), and uracil (U), as illustrated above; such bases can be used at any position in the antisense molecule. Persons skilled in the art will appreciate that depending on the uses of the oligomers, Ts and Us are interchangeable. For instance, with other antisense chemistries such as 2′-O-methyl antisense oligonucleotides that are more RNA-like, the T bases may be shown as U.

As used herein, a “modified nucleotide” is a nucleotide other than a ribonucleotide (2′-hydroxyl nucleotide). In some embodiments, at least about 2%, at least about 5%, at least about 10%, at least about 20%, at least about 30%, at least about 40%, at least about 50%, at least about 60%, at least about 70%, at least about 80%, or at least about 90% of the nucleotides in a MdsRNA are post-transcriptionally chemically modified. In some embodiments, at least about 2%, at least about 5%, at least about 10%, at least about 20%, at least about 30%, at least about 40%, at least about 50%, at least about 60%, at least about 70%, at least about 80%, at least about 85%, at least about 90%, at least about 95%, at least about 96%, at least about 97%, at least about 98%, at least about 99% of the nucleotides in a ssRNA strand linking two different dsRNA stems or a sense section and an antisense section in the same dsRNA stem are post-transcriptionally chemically modified. Modified nucleotides include, but are not limited to, nucleotides having a ribose 2′-OH substitution.

“Independently” means that the modified nucleotides can each have the same modification, or they can have different modifications from each other, selected from high molecular weight polyalkyloxy polymers, and if present, low molecular weight moieties.

An “unmodified” dsRNA is an RNA molecule that has not been chemically modified.

As used herein, the term “polyalkoxy” or “polyalkyloxy” as used interchangeably herein refers to a suitable water-soluble polymer characterized by repeating alkoxy units. Select, but non-limiting, examples of such polymers are polyethylene glycol (PEG), polypropylene glycol (PPG), poloxamers, hyaluronic acid, polyvinyl alcohols, polyoxazolines, polyanhydrides, poly(ortho esters), polycarbonates, polyurethanes, polyacrylic acids, polyacrylamides, polyacrylates, polymethacrylates, polyorganophosphazenes, polysiloxanes, polyvinylpyrrolidone, polycyanoacrylates, polyesters, or any derivatives of the foregoing.

In an embodiment, the term “polyalkoxy” or “polyalkyloxy” used herein refers to a suitable linear or branched polyethylene glycol (PEG) polymer. In some embodiments, the polyalkoxy is a linear or branched polypropylene glycol (PPG) polymer. In some embodiments, the polyalkyloxy is a poloxamer. In some embodiments the polymer is an ethylene glycol-propylene glycol block copolymer poloxamer. In another embodiments the polymer is a poly(ethylene glycol)-block-poly(propylene glycol)-block-poly(ethylene glycol), PEG-PPG-PEG poloxamer. In yet another embodiment the polymer is an ethylene oxide-propylene oxide triblock copolymer. In some embodiments, the polyalkyloxy has a molecular weight of at least about 400 Da, at least about 1 kDa, at least about 5 kDa, at least about 10 kDa, with an upper molecular weight of about 40 kDa. In another embodiment, the molecular weight of the polyalkyloxy is from about 400 Da and about 40 kDa or from about 5 kDa and about 40 kDa.

As used herein, the term “poloxamers” refers to nonionic triblock copolymers composed of a central hydrophobic chain of polyoxypropylene flanked by two hydrophilic chains of polyoxyethylene. Select, but non-limiting, examples of poloxamers are poloxamers 407, 338, 188, 184, and 401 (i.e., F127, F108, L68, L64 and L121 Pluronic®, BASF). The first two digits multiplied by 100 give the approximate molecular mass of the polyoxypropylene core, and the last digit multiplied by 10 gives the percentage polyoxyethylene content. “L” stands for liquid and “F” stands for flake solid.

The amphiphilic character, that is the presence of both hydrophilic and lipophilic groups, of the resulting polymer modified dsRNAs of the disclosure modulate their physical and chemical properties such as solubility, absorption and permeability through plant tissue and target cell membranes and resistance to nucleases. The polymers that produced the most efficacious MdsRNAs have Hydrophilic-Lipohilic Balance (HLB) numbers ranging from 8 to 27. In one embodiment, the HLB is from about 2 to about 30. In another embodiment, the HLB is from about 15 to about 27.

As used herein, the term “high molecular weight polyalkyloxy” refers to a compound, substituent, or other chemical moiety comprising a polyalkyloxy group having a molecular weight of at least about 400 Da, with an upper molecular weight being about 40 kDa. In some embodiments, the molecular weight of the polyalkyloxy is at least about 1 kDa. In some embodiments, the molecular weight of the polyalkyloxy is at least about 5 kDa. In another embodiment, the molecular weight of the polyalkyloxy is at least about 10 kDa. In another embodiment, the molecular weight of the polyalkyloxy is from about 400 Da and about 40 kDa. In yet another embodiment, the molecular weight of the polyalkyloxy is from about 5 kDa and about 40 kDa.

As used herein, a “low molecular weight (LMW) modified nucleotide” is a nucleotide having a 2′-OH low molecular weight modification. Low molecular weight modifications are moieties that are from about 43 daltons to about 1,000 daltons. Examples of modifications, for example as identified herein as R² in Formula III include, but are not limited to, C₁-C₂₅ alkyl, C₁-C₂₅ alkenyl, C₁-C₂₅ alkynyl, C₅-C₁₂ aryl or C₅-C₁₂ heteroaryl, wherein any of these is optionally substituted with one or more substituents selected from halo, C₁₋₁₂ alkyl, C₁-C₁₂ aminoalkyl, or C₁-C₁₂ alkoxy (i.e., R² as described herein). In some embodiments, at least about 2%, at least about 5%, at least about 10%, at least about 20%, at least about 30%, at least about 40%, at least about 50%, at least about 60%, at least about 70%, at least about 80%, at least about 90%, or at least about 100% of the nucleotides in a MdsRNA are post-transcriptionally chemically modified as LMW modified nucleotides. In some embodiments, the chemical modifications are esters of N-methyl-anthranilic acid (from modification with N-methylisatoic anhydride (NMIA)), esters of N-benzyl-anthranilic acid (from modifications with N-benzylisatoic anhydride (NBIA)), dimethyl furoyl, esters of a fatty acid (e.g., C1-C18, such as but not limited to lauryl, oleic, linoleic), acetic acid, propionic acid, esters of amino acids (e.g., tyrosine, tryptophan, leucine), low molecular weight PEG, nitrogen containing moieties.

Without being limited by theory, it is believed that the presence of the LMW moieties allows the composition to better dissolve in organic solvents, for delivery of the compositions to plants and ultimately to the target insects. The compositions can be made more concentrated, in amounts that are economical for the end-user. Further, the decomposition rate in the field for the compositions of the application has been shown to decrease by filling in the spaces in the MdsRNA that are not modified with the high molecular polyalkyloxy polymers. In other words, the nucleotides that are not modified with high molecular weight polyalkyloxy polymer can be modified with low molecular weight moieties, as described herein. The degree of modification of the nucleotides with low molecular weight moieties will depend on the desired properties of the compositions with respect to decomposition rate, solubility and concentration, among other benefits.

The term “alkyl” or “alkyl group” as used herein describes a univalent group derived from alkanes by removal of a hydrogen atom from any carbon atom —CₙH₂ₙ₊₁. An alkyl group can be straight or branched chain or cyclic and include methyl, ethyl, propyl, isopropyl, butyl, hexyl and the like. As used herein, a lower alkyl group contains from 1 to 25 carbon atoms in the principal chain.

The term “alkenyl” as used herein are acyclic branched or unbranched hydrocarbons having one carbon-carbon double bond and the general formula —CₙH₂ₙ₋₁. One or more of the hydrogen atoms can be substituted. An alkyl group can be straight or branched chain and include ethenyl, propenyl, isopropenyl, butenyl, isobutenyl, hexenyl, and the like. As used herein, an alkenyl contains from 2 to 25 carbon atoms in the principal chain.

The term “alkoxide” or “alkoxy” as used herein is the conjugate base of an alcohol. The alcohol can be straight chain, branched, cyclic, and includes aryloxy compounds and include methoxy, ethoxy, isoproyloxy, butoxy, and the like.

The term “alkynyl” as used herein are acyclic branched or unbranched hydrocarbons having a carbon-carbon triple bond and the general formula —CₙH₂ₙ₋₃. They can be straight or branched chain and include ethynyl, propynyl, butynyl, isobutynyl, hexynyl, and the like. As used herein a lower alkynyl containing from 2 to 25 carbon atoms in the principal chain.

The terms “aryl” or “Ar” as used herein alone or as part of another group denote optionally substituted homocyclic aromatic groups. Aryl groups can be monocyclic or bicyclic groups containing from 5 to 12 carbons in the ring portion, such as phenyl, biphenyl, naphthyl, substituted phenyl, substituted biphenyl, or substituted naphthyl.

The term “heteroaryl” as used herein alone or as part of another group denotes optionally substituted aromatic groups having at least one heteroatom in at least one ring. In some embodiments, heteroaromatic group contains 5 or 6 atoms in each ring. In some embodiments, a heteroaromatic group contains 1 or 2 oxygen atoms and/or 1 to 4 nitrogen atoms in the ring and is bonded to the remainder of the molecule through a carbon. Exemplary groups include furyl, benzofuryl, oxazolyl, isoxazolyl, oxadiazolyl, benzoxazolyl, benzoxadiazolyl, pyrrolyl, pyrazolyl, imidazolyl, triazolyl, tetrazolyl, pyridyl, pyrimidyl, pyrazinyl, pyridazinyl, indolyl, isoindolyl, indolizinyl, benzimidazolyl, indazolyl, benzotriazolyl, tetrazolopyridazinyl, carbazolyl, purinyl, quinolinyl, isoquinolinyl, imidazopyridyl, and the like. Exemplary substituents include one or more of the following groups: hydrocarbyl, substituted hydrocarbyl, alkyl, alkoxy, acyl, acyloxy, alkenyl, alkenoxy, aryl, aryloxy, amino, amido, acetal, carbamyl, carbocyclo, cyano, ester, ether, halogen, heterocyclo, hydroxyl, keto, ketal, phosphor, nitro, and thio.

The term “heteroatom” refers to atoms other than carbon and hydrogen.

The term “halogen” or “halo” refers to as used herein alone or as part of another group refer to chlorine, bromine, fluorine or iodine.

As used herein, the term “greater stability” refers to the compounds of the instant application in comparison to unmodified dsRNAs or MdsRNAs modified with low molecular weight polyalkyloxy. In some embodiments, greater stability means increased persistence in agricultural fields, in plant tissue, on plant leaves, etc. In some embodiments, greater stability means increased in vivo half-life. In some embodiments, greater stability means enhanced resistance to physiological conditions. In other embodiments, greater stability means enhanced resistance to nucleases.

As used herein, “acceptable salts” refers to salts derived from suitable inorganic and organic acids and inorganic and organic bases that are, within the scope of sound judgment, suitable for use in contact with the tissues of humans, lower animals, and plants without undue toxicity, irritation, allergic response and the like, and are commensurate with a reasonable benefit/risk ratio.

A salt of a compound of the present disclosure can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two.

As used herein, the term “scalable” refers to the processes ability to produce quantities of the target chemical in commercially relevant amounts. For example, the process of the instant application may allow for the production of compounds in the hundreds of milligram scale. In some examples, the process of the instant application may allow for the production of compounds in the gram scale. In some examples, the process of the instant application may allow for the production in the kilogram scale. In some examples, the process of the instant application may allow for the production in the metric ton scale.

As used herein, the term “activation agent” or “activating agent” refers to a compound that increases the nucleophilicity or electrophilicity of target moiety. The change in electron density can be the result of an ionic or covalent bond from the activating group. In some embodiments, the activation agents described herein increase the electrophilicity of a target carbonyl. Non-limiting examples of activation agents are lewis acids, protons, and coupling reagents. Non-limiting examples of coupling reagents are benzotriazol-1-yloxytris(dimethylamino)phosphonium hexafluorophosphate (BOP), benzotriazol-1-yloxytripyrrolidinophosphonium hexafluorophosphate (PyBOP), (2-(1H-benzotriazol-1-yl)-1,1,3,3-tetramethyluronium hexafluorophosphate, Hexafluorophosphate Benzotriazole Tetramethyl Uronium (HBTU), N,N′-Dicyclohexylcarbodiimide (DCC), carbonyldiimidazole (CDI), and 1-[Bis(dimethylamino)methylene]-1H-1,2,3-triazolo[4,5-b]pyridinium 3-oxide hexafluorophosphate, Hexafluorophosphate Azabenzotriazole Tetramethyl Uronium (HATU).

As used herein, the term “suitable leaving group” refers to a molecular fragment that departs with a pair of electrons in heterolytic bond cleavage. The leaving group helps facilitate the reaction by lowering the energy of activation. Non-limiting examples of suitable leaving groups are halides, tosylated or mesylated alcohols, pseudo-halides, amines, heterocycles, and the like. In a particular embodiment, the suitable leaving group is imidazole.

As used herein, the term “anhydrous solvent” refers to a solvent that has been physically or chemically treated to reduce the water content of the solvent. Anhydrous solvents may contain less than about 1% by weight water, less than about 0.3% by weight water, or less than about 0.1% by weight water.

As used herein, the term “sequence identity”, “sequence similarity” or “homology” is used to describe sequence relationships between two or more nucleotide sequences. The percentage of “sequence identity” between two sequences is determined by comparing two optimally aligned sequences over a comparison window, such that the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. A sequence that is identical at every position in comparison to a reference sequence is said to be identical to the reference sequence and vice-versa.

As used herein, a “comparison window” refers to a conceptual segment of at least 6 contiguous positions, usually about 50 to about 100, more usually about 100 to about 150, in which a sequence is compared to a reference sequence of the same number of contiguous positions after the two sequences are optimally aligned. The comparison window may comprise additions or deletions (i.e. gaps) of about 20% or less as compared to the reference sequence (which does not comprise additions or deletions) for optimal alignment of the two sequences. Those skilled in the art should refer to the detailed methods used for sequence alignment in the Wisconsin Genetics Software Package Release 7.0, Genetics Computer Group, 575 Science Drive Madison, Wis., USA) or refer to Ausubel et al. (1998) for a detailed discussion of sequence analysis.

As used herein, the variations of the terms including “RNA” refers to a ribonucleic acid complementary to an expressed RNA in a target pest (e.g., insect, weed, invasive species) as described herein. “RNA” may also refer to a ribonucleic acid that is complementary to a target gene sequence.

As used herein, the term “RNA transcript” refers to the product resulting from RNA polymerase-catalyzed transcription of a DNA sequence. When the RNA transcript is a perfect complementary copy of the DNA sequence, it is referred to as the primary transcript or it may be an RNA sequence derived from post-transcriptional processing of the primary transcript and is referred to as the mature RNA.

As used herein, the term “sense RNA” refers to an RNA transcript corresponding to a sequence or segment that, when produced by the target pest, is in the form of a mRNA that is capable of being translated into protein by the target pest cell.

As used herein, the term “anti-sense RNA” refers to an RNA transcript that is complementary to all or a part of a mRNA that is normally produced in a cell of a target pest. The complementarity of an anti-sense RNA may be with any part of the specific gene transcript, i.e., at the 5′ non-coding sequence, 3′ non-translated sequence, introns, or the coding sequence.

As used herein, the term “dsRNA stem” refers to a first ssRNA strand hybridized to a second ssRNA wherein such second ssRNA strand is the reverse complement of the first ssRNA strand. A first nucleotide sequence when observed in the 5′ to 3′ direction is said to be a “complement of”, or “complementary to”, a second reference nucleotide sequence observed in the 3′ to 5′ direction if the sequence of the first nucleotide is the reverse complement of the reference nucleotide sequence. For illustration, the nucleotide sequence “CATTAG” corresponds to a reference sequence “CATTAG” and is complementary to a reference sequence “GTAATC.” Nucleic acid sequence molecules are said to exhibit “complete complementarity” when every nucleotide of one of the sequences read 5′ to 3′ is complementary to every nucleotide of the other sequence when read 3′ to 5′. dsRNA stems can have complete or incomplete complementarity. Incomplete complementarity can be from about 50% to about 99% complementarity, preferably from about 60% to about 99% complementarity, more preferably from about 70% to about 99% complementarity, and most preferably from about 80% to about 99% complementarity.

As used herein, “loop” refers to a structure formed by a single strand of a nucleic acid, in which complementary regions that flank a particular single stranded nucleotide region hybridize in a way that the single stranded nucleotide region between the complementary regions is excluded from duplex formation or Watson-Crick base pairing. A loop is a single stranded nucleotide region of any length.

As used herein, “link” or “linker” or “ssRNA link” or “ssRNA linker” refers to a single strand of a nucleic acid, flanked by two strands in different dsRNA stems or in the same dsRNA stem. When it is flanked by two strands in the same dsRNA, it is also referred to as a “loop”. A “link” or “linker” or “ssRNA link” or “ssRNA linker” is a single stranded nucleotide region of any length.

As used herein a “hairpin” refers to a structure comprising a dsRNA stem and a loop.

As used herein, “section,” “RNA section,” or “sense/anti-sense section” refers to a portion of a ssRNA, dsRNA, MdsRNA, or MRNA that contains RNA nucleobases, in either the sense or antisense direction, that is connected to a link or linker on at least the 5′ or 3′ end of the RNA, but can also be connected at both the 5′ and 3′ end of the RNA.

As used herein, “PAC” or “PAC site” refers to a ssRNA strand with high affinity or high binding constant with a capsid protein, for example the capsid protein of the bacteriophages MS2 or Qβ. For example, the wildtype 19 nt hairpin that binds to the wildtype MS2 capsid protein.

As used herein “ssRNA” refers to single strand RNA.

As used herein “dsRNA” refers to double strand RNA.

As used herein, “MdsRNA” refers to dsRNA wherein some of the 2′OH groups in its dsRNA nucleotides have been post-transcriptionally chemically modified.

As used herein, “MRNA” refers to RNA comprising ssRNA sections and dsRNA stems wherein from about 10% and 100% of the 2′OH groups in its ssRNA riboses have been post-transcriptionally chemically modified and between about 0% and 30% of its dsRNA nucleotides have been post-transcriptionally chemically modified.

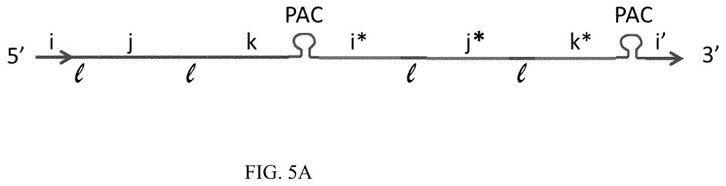

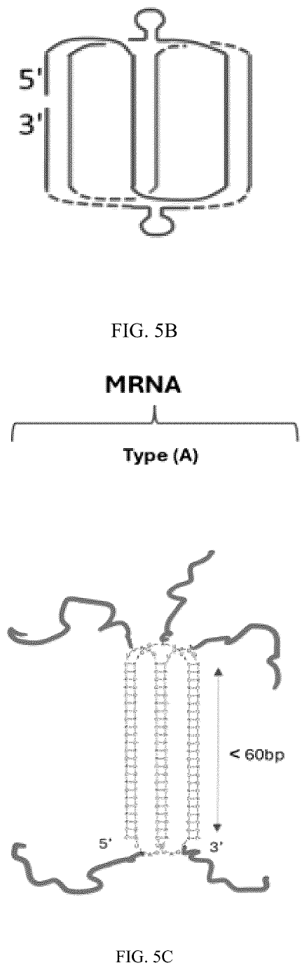

As used herein, “type (A) RNA” may refer to an RNA molecule, RNA compound, RNA strand, a single strand RNA (ssRNA), or RNA construct that comprises N or N+1 sense sections, 2N−1 or 2N, respectively, linker sections, and N antisense sections, with the sense sections called “Ax”, the antisense sections to “Ax” called “Ay*”, wherein x is an integer and varies from 1 to N+1 and y is an integer and varies from 1 to N, and with the linker sections called “Lz” wherein z is an integer and varies from 1 to 2N. Further, “N” is an integer from about 2 to about 51, meaning that if N=3 then there are 3 or 4 sense sections, 5 or 6 linker sections, respectively, and 3 antisense sections. In type (A) RNA when N=3, sections are arranged in the following order: 5′-A1-L1-A2-L2-A3-L3-A1*-L4-A2*-L5-A3*-3′ or 5′-A1-L1-A2-L2-A3-L3-A1*-L4-A2*-L5-A3*-L6-A4-3′, noting that “Ax” refers to the x^{th} sense section, “Ay*” as the y^{th} antisense section (reverse complementary) to the Ax^{th} section, and “Lz” as the z^{th} linker section. Examples of type (A) RNA are shown in FIGS. 5A-5C, in which 5A and 5B depict the unfolded and folded, respectively, structures of a type (A) RNA with 4 sense sections, 3 antisense sections, 2 linker sections each comprising a PAC sequence, and 4 additional linker sections, and in which FIG. 5C depicts the folded structure of a type (A) RNA with 3 sense sections, 3 antisense sections, and 5 linker sections. In preferred embodiments, “N” is an odd integer for type (A) RNA.

An example of type (A) RNA is shown in FIG. 5A, in which N=3. A1 sense section is depicted as i, A4 sense section is represented as i′, which, when folded as shown in FIG. 5B, i and i′ are adjacent to each other and where i is located 5′ of i′ and i′ is located 3′ of i, A2 is depicted as sense section j, A3 is depicted as section k, A1* is depicted as section i*, A2* is depicted as section j*, A3 is depicted as section k*, linker sections L1, L2, L4, and L5 are depicted as sections [symbol] , and linker sections L3 and L6 each comprise a PAC sequence.

As used herein, “type (B) RNA” may refer to an RNA molecule, RNA compound, RNA strand, a single strand RNA (ssRNA), or RNA construct that comprises N sense sections, 2N−1 linker sections, and N antisense sections, with the sense sections called “By”, the antisense sections to “By” called “By*”, wherein y is an integer and varies from 1 to N, and with the linker sections called “Jz” wherein z is an integer and varies from 1 to 2N−1. Further, “N” is an integer from about 2 to about 50, meaning that if N=3 then there are 3 sense sections, 5 linker sections, and 3 antisense sections. In type (B) RNA when N=3, sections are arranged in the following order: 5′-B1-J1-B2-J2-B3-J3-B3*-J4-B2*-J5-B1*-3′, noting that “By” refers to the y^{th} sense section, “By*” as the y^{th} antisense section (reverse complementary) to the By^{th} section, and “Jz” as the z^{th} linker section.

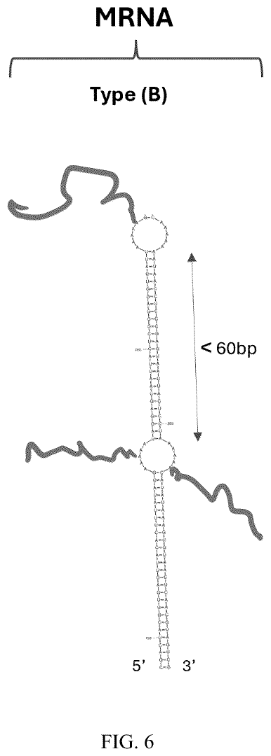

An example of type (B) RNA is shown in FIG. 6, wherein N=2, therefore there are 2 stems formed by the hybridization of reverse complementary sequences, wherein the first stem is formed by the hybridization of the first sense section, located 5′ of the rest of the sections, and the section antisense to the first sense section, located 3′ of the rest of the sections, the second stem is formed by the hybridization of the sense section located 3′ of the first sense section and the antisense section located 5′ of section antisense of the first section, the first linker section located between the first two sense sections, the second linker section located 3′ of the second sense section and the third linker section located between the two antisense sections.

As used herein, “type (C) RNA” may refer to an RNA molecule, RNA compound, RNA strand, a single strand RNA (ssRNA), or RNA construct that comprises N sense sections, 2N−1 linker sections, and N antisense sections, with the sense sections called “Cy”, the antisense sections to “Cy” called “Cy*”, wherein y is an integer and varies from 1 to N and with the linker sections called “Kz” wherein z is an integer and varies from 1 to 2N−1. Further, “N” is an integer from about 2 to about 50, meaning that if N=3 then there are 3 sense sections, 5 linker sections, and 3 antisense sections. In type (C) RNA, sections are arranged in the following order: 5′-C1-K1-C1*-K2-C2-K3-C2*-K4-C3-K5-C3*-3′, noting that “Cy” refers to the y^{th} sense section, “Cy*” as the y^{th} antisense section (reverse complementary) to the Cy^{th} section, and “Kz” as the z^{th} linker section.

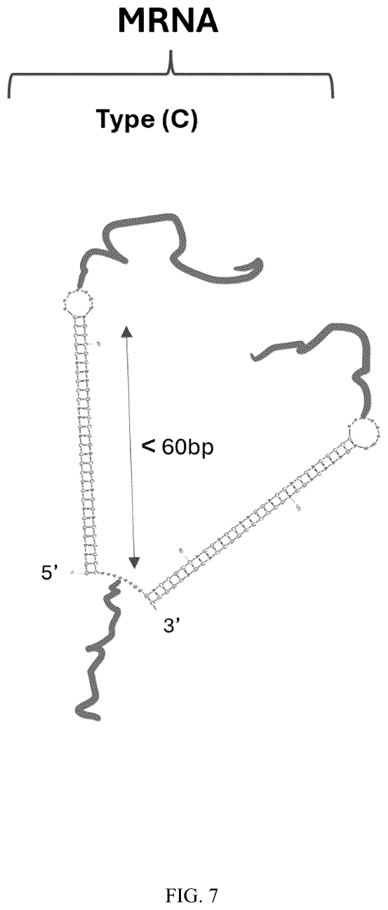

An example of type (C) RNA is shown in FIG. 7, wherein N=2; therefore there are 2 stems formed by the hybridization of reverse complementary sequences, wherein the first stem is formed by the hybridization of the first sense section, located 5′ of the rest of the sections, and the first antisense section, located 3′ of the first sense section, the second stem is formed by the hybridization of the second sense section, located 3′ of the first antisense section, and the second antisense section, located 3′ of the second sense section, the first linker section, located between the first sense and antisense sections, the second linker section, located between the two stems, and the third linker section, located between the second sense and antisense sections.

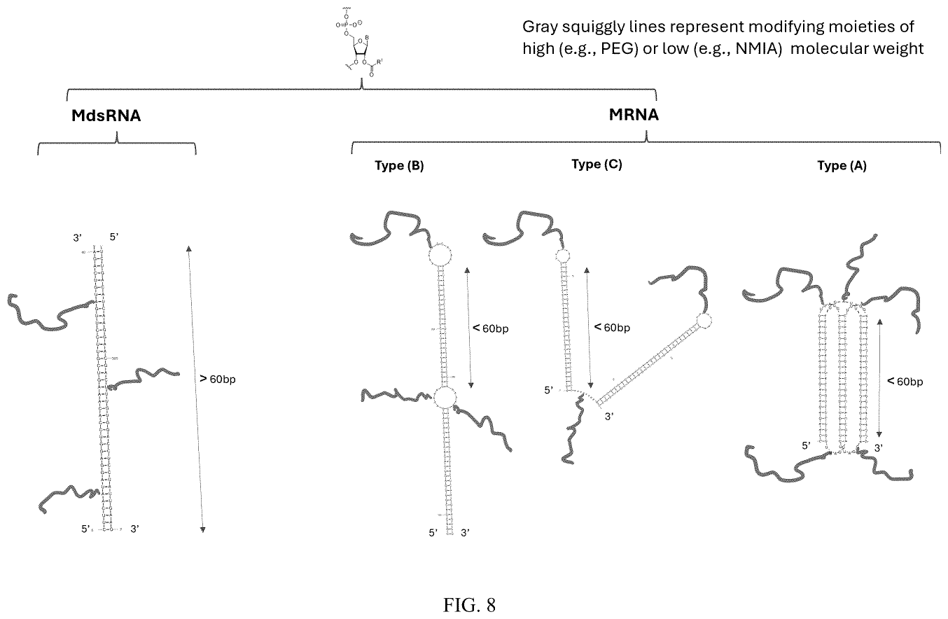

As described herein, there are two types of type (A) RNA, wherein one type has N sense sections, 2N−1 linker sections, and N antisense sections, and the other type has N+1 sense sections, 2N linker sections, and N antisense sections. Both types (B) and (C) RNA have N sense sections, 2N−1 linker sections, and N antisense sections. Each type of RNA is differentiated by the ordering the sense and antisense sections, and further differentiated by the three-dimensional structure once folded, as shown in FIG. 8.

The RNA for any of the three types of constructs, type (A), type (B) and type (C), can be transcribed as reverse complementary pairs, i.e., as reverse complementary ssRNA strands, each transcribed from each of the two reverse complementary strands of the same DNA template used in the transcription. During transcription the two reverse complementary ssRNA strands assemble as a long dsRNA stem. After transcription, a refolding step that may comprise, for example, heating (to break the Watson & Crick base pairs, unfold dsRNA stem, and separate the two strands that form the stem) and cooling (to refold into itself each of the separated reverse complementary ssRNA strands) in an appropriate blend of solvents, can be conducted resulting in the formation of pairs of any of the constructs type (A), type (B) or type (C).

As used herein, a “pest” refers to an organism that is harmful to plants, crops, livestock, forestry, humans, or human concerns. Some non-limiting examples of pests include insects, plants in unwanted locations (e.g., weeds), and/or fungi.

As used herein, “cation” refers to a positively charged ion present in a molecule or metal ion. When used in combination with an anion, a salt is formed, or when in solution, typically water, a solution of cations and anions forms, which can be referred to as an “aqueous solution” or a “buffer.” Some non-limiting examples of cations include: imidazolium, 1-alkylimidazolium, 1-alkyl-3-methylimidazolium, 1-alkylpyridinium, 1-alkyl-1-methylpyrrolidinium, 1-butyl-2,3-dimethylimidazolium, 1-propylamine-3-methylimidazolium, 1-(4-sulfonylbutyl)-3-methylimidazolium, 1-cyclohexyl-3-methylimidazolium, 1-benzyl-3-methylimidazolium, N,N-dimethyl(cyanoethyl)ammonium, 2-(dodecyloxy)-N,N,N-trimethyl-2-oxocthanaminium, N,N-dimethylammonium, tetraalkylammonium, cholinium, N,N-dimethyl-N-(2-hydroxyethoxyethyl) ammonium, N,N-dimethyl(2-methoxyethyl)ammonium, 1,12-di-(2,3-dimethylimidazolium)dodecane, 1-nonyl-3-vinylimidazolium, 1,9-di-(3-vinylimidazolium)nonane, 1,12-di-(tripropylphosphonium)dodecane, benzyltributyl ammonium, benzyltrimethyl ammonium, tetramethyl ammonium, tetraethyl ammonium, and cetyltrimethyl.

As used herein, “anion” refers to a negatively charged ion present in a molecule or metal ion. Some non-limiting examples of anions include: bromide, chloride, iodide, hydroxide, acesulfamate, tetrafluoroborate, nitrate, bis(trifluoromethylsulfonyl)imide, hexafluorophosphate, saccharinate, dimethylcarbamate, hydrogenosulfate, dihydrogenophosphate, alkylsulfate, and trifluoromethanesulfonate.

In some preferred embodiments, a combination of cations and anions include: benzyltributyl ammonium chloride, benzyltrimethyl ammonium chloride, benzyltrimethyl ammonium bromide, tetramethyl ammonium chloride, tetraethyl ammonium chloride, cetyltrimethyl ammonium bromide, or cetyltrimethyl ammonium chloride.

A promoter is a region (sequence) of DNA that initiates transcription of a gene. A promoter can be a bacterial promoter, archaea promoter, eukaryotic promoter, or a Pol I, Pol II, or Poll III promoter. In some embodiments, a bacterial promoter comprises the sequence 5′-TTGACA-3′ about 35 units upstream from the transcription start site and the sequence 5′-TATAAT-3′ about 10 bp upstream from the transcription start site. Other promoters, suitable for use with different expression systems are well known in the art.

### Compositions

MdsRNAs are described having no more than about 30% (e.g., from about 2% to about 30%) of all the ribose rings chemically modified at the 2′-OH position with high molecular weight polyalkyloxy polymers, and optionally at least about 10% to about 60% of all the nucleotides being chemically modified with LMW modified moieties at the 2′-OH position. The MdsRNAs are capable of inhibiting gene expression in a sequence specific manner, such as through RNA interference or antisense mechanisms. Modified RNA constructs having both dsRNA and ssRNA are also described having from about 10% to about 60% of all the ribose rings in the ssRNA sections linking two different dsRNA stems or a sense section and an antisense section in the same dsRNA stem that are chemically modified at the 2′-OH position with high molecular weight polyalkyloxy polymers, and optionally at least about 2% to about 30% of all the nucleotides in such ssRNA sections are chemically modified with LMW modified moieties. Such RNA compositions (MRNAs) are capable of inhibiting gene expression in a sequence specific manner, such as through RNA interference or antisense mechanisms.

Thus, in an aspect, provided herein is a composition comprising a post-transcriptionally chemically modified double strand RNA (MdsRNA) wherein the MdsRNA comprises a double strand RNA wherein no more than about 30% (e.g., from about 2% to about 30%) of all the nucleotides independently comprise Formula (I)

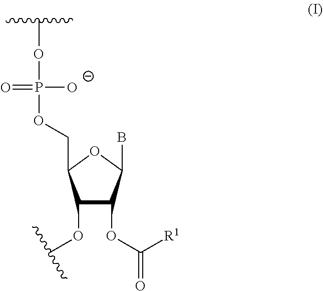

or an acceptable salt thereof, wherein:
B is a nucleobase;
R¹ is selected from:

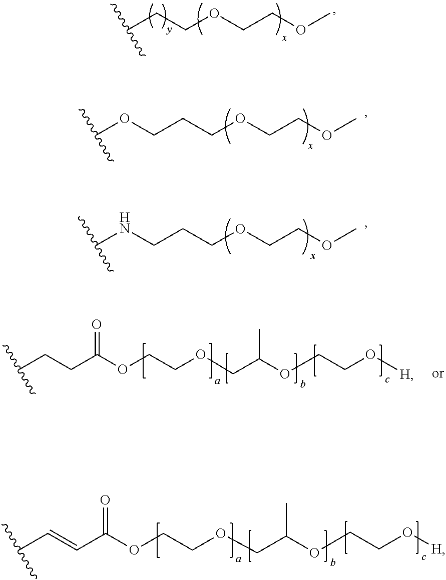

wherein y is an integer from 1-8, x is an integer from 12-1000, a is an integer from 12-1000, b is an integer from 12-1000, and c is an integer from 12-1000.

In another aspect, provided herein is a composition comprising a post-transcriptionally chemically modified single strand RNA (MRNA) comprising molecules longer than 55 nucleotides (e.g., from about 55 nucleotides to about 900 nucleotides), wherein the ssRNA is of type (A) or (B) or (C), wherein about 10% to about 95% of all the nucleotides in the linker strands L1 through L2N, J1 through J(2N−1) or K1 through K(2N−1) independently comprise Formula (I) above.

In another aspect, provided herein are compositions comprising a post-transcriptionally chemically modified double strand RNA (MdsRNA) wherein the MdsRNA comprises a double strand RNA wherein no more than about 30% (e.g., from about 2% to about 30%) of all the nucleotides independently comprise Formula (I):

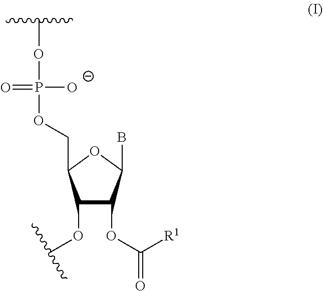

or an acceptable salt thereof, wherein:
B is a nucleobase;
R¹ is selected from:

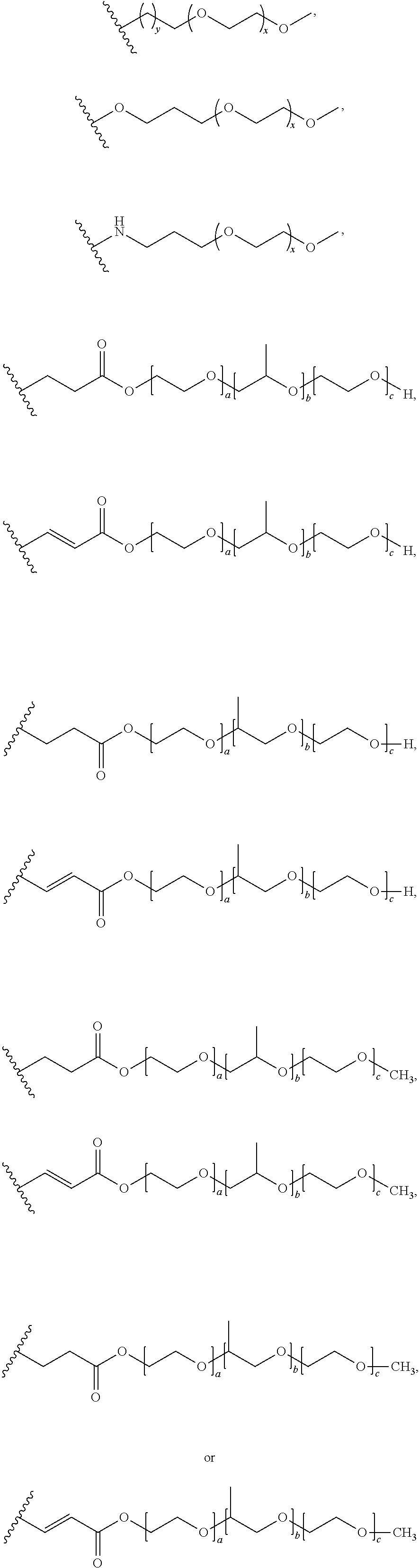

wherein y is an integer from 1-8, x is an integer from 12-1000, a is an integer from 12-1000, b is an integer from 12-1000, and c is an integer from 12-1000; and
optionally wherein at least about 2% (e.g., from about 2% to about 50% or even up to 85%) of all the nucleotides independently comprise LMW modified nucleotides of Formula (III):

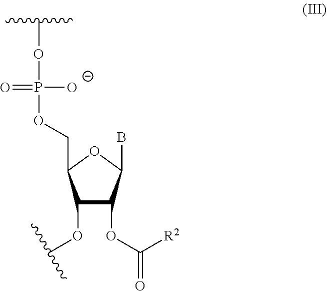

or an acceptable salt thereof, wherein:
B is a nucleobase;
R² is selected from C₁-C₂₅ alkyl, C₁-C₂₅ alkenyl, C₁-C₂₅ alkynyl, C₅-C₁₂ aryl or C₅-C₁₂ heteroaryl, wherein R² is optionally substituted with one or more substituents selected from halo, C₁₋₁₂ alkyl, C₁-C₁₂ aminoalkyl, or C₁-C₁₂ alkoxy.

In another aspect, provided herein is a composition comprising a post-transcriptionally chemically modified single strand RNA wherein the ssRNA is of type (A) or (B) or (C), wherein strands AN or BN or CN or AN* or BN* or CN* have about 19 nucleotides to about 600 nucleotides, wherein about 10% to about 95% of all the nucleotides in the linker strands L1 through L2N or L1 to L(2N−1), J1 through J(2N−1) or K1 through K(2N−1) independently comprise Formula (I)

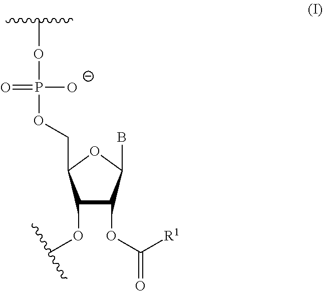

or an acceptable salt thereof, wherein:
B is a nucleobase;
R¹ is selected from:

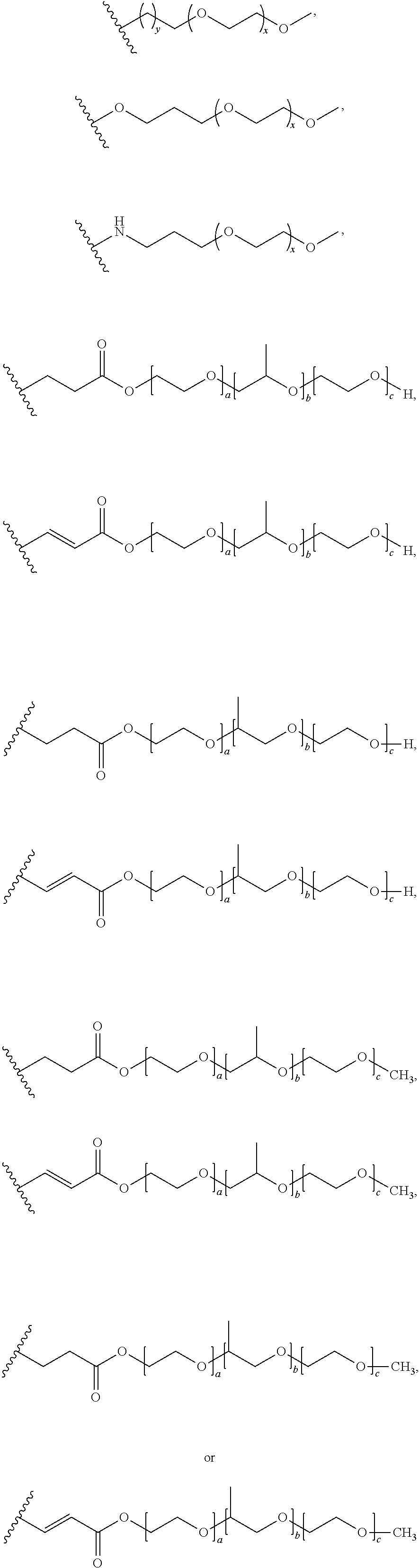

wherein y is an integer from 1-8, x is an integer from 12-1000, a is an integer from 12-1000, b is an integer from 12-1000, and c is an integer from 12-1000; and optionally wherein at least about 2% to about 50% of all the nucleotides in the linker L1 through L2N or L1 through L(2N−1), J1 through J(2N−1) or K1 through K(2N−1) independently comprise LMW modified nucleotides of Formula (III):

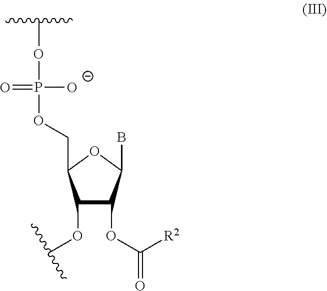

or an acceptable salt thereof, wherein:
B is a nucleobase;
R² is selected from C₁-C₂₅ alkyl, C₁-C₂₅ alkenyl, C₁-C₂₅ alkynyl, C₅-C₁₂ aryl or C₅-C₁₂ heteroaryl, wherein R² is optionally substituted with one or more substituents selected from halo, C₁₋₁₂ alkyl, C₁-C₁₂ aminoalkyl, or C₁-C₁₂ alkoxy.

Embodiments of this aspect of the composition follow.

In an embodiment, R¹ is selected from:

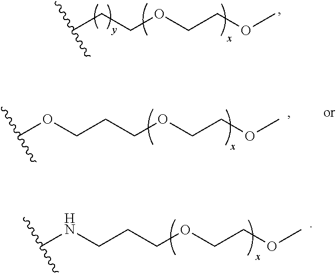

In another embodiment, x is an integer from 80-1000.

In another embodiment, x is an integer from 50-900.

In another embodiment, a is an integer from 80-1000.

In yet another embodiment, b is an integer from 80-1000.

In a further embodiment, c is an integer from 80-1000.

In an embodiment, R¹ has a molecular weight from about 5,000 Da to about 15,000 Da.

In an embodiment, R¹ has a molecular weight from about 5,000 Da to about 40,000 Da.

In an embodiment, R¹ is polyethylene glycol (PEG).

In an embodiment, R¹ is poloxamer 407, 338, 188, 184, 401 or any combination thereof.

In an embodiment, a is 101, b is 56, and c is 101.

In an embodiment, R¹ is a HMW polymer with an HLB ranging from about 2 to about 30. In another embodiment, R¹ is a HMW polymer with an HLB ranging from about 15 to about 27.

In an embodiment, R² is N-methyl anthranoyl (NMA), N-benzyl anthranoyl (NBA), dimethyl furoyl, -Tyr, -Trp, -Leu, octanoyl, lauroyl, linoleyl, oleyl, nicotinoyl or benzoyl.

In an embodiment, R¹ is

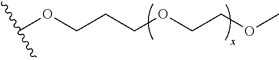

and R² is N-methyl anthranoyl.

In an embodiment, R¹ is

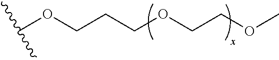

and R² is lauroyl.

In an embodiment, R¹ is

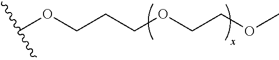

and R² is linoleyl.

In an embodiment, R¹ is

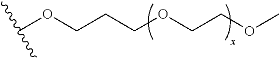

and R² is oleyl.

In embodiments, the ratio of Formula (I) to Formula (III) to unmodified nucleotides in the MdsRNA is about 3:5:2; 3:6.5:0.5; 1:7:2; 0.05:9:0.5; 1.3:4:5.7; 0.01:4:5.99; 0.06:0:96.4.

In embodiments, the ratio of Formula (I) to Formula (III) to unmodified nucleotides in the linker strands L1 through L2N or L1 through L(2N−1), J1 through J(2N−1) or K1 through K(2N−1) is about 3:5:2; 3:6.5:0.5; 1:7:2; 0.05:9:0.5; 1.3:4:5.7; 0.01:4:5.99; 0.06:0:96.4.

In embodiments, from about 2% to about 50% of all nucleotides in the MdsRNA or the MRNA sequence are modified with LMW moieties.

In embodiments, from about 2% to about 50% of all nucleotides in the linker strands L1 through L2N or L1 through L(2N−1), J1 through J(2N−1) or K1 through K(2N−1) are modified with LMW moieties.

In another embodiment, the MdsRNA or the MRNA comprises a sequence complementary to an expressed RNA in a target pest (e.g., insect, weed, invasive species). For example, an efficacious sequence or target gene useful in the products and methods of the disclosure will inhibit the expression of the target gene, act on the mid gut of an insect and increase mortality or induce growth stunting or stop instar development.

In another embodiment, the MdsRNA will inhibit the expression of a regulatory element of one or more target genes, increase mortality or induce growth stunting, or stop instar development. Examples of regulatory elements of target genes include but are not limited to long non-coding RNAs, circular RNAs and micro RNAs.

In an embodiment, the target insect is Diamondback moth (Plutella xylostella), Gypsy moth, Red imported fire ant (Solenopsis invicta), Fall armyworm, Colorado potato beetle, Canola flea beetle, Aedes aegypti, or Western corn root worm (Diabrotica virgifera virgifera). In another embodiment, the target insect is Pea aphid (Acyrthosiphon pisum), Soybean aphid, Piezodorus guildinii. In another embodiment the target is an acari, such as Verroa mite. In another embodiment the target is a weed, such as Palmer amaranth. In yet another embodiment, the target is a fungus such as Palmer amaranth, Fusarium graminearum (Gibberella zeae) or Botrytis. Accordingly, the MdsRNA or the MRNA used in the compositions of the disclosure will be a nucleotide sequence that can inhibit the expression of target genes or regions in these pests. For example, the MdsRNA comprises a sequence complementary to a target region in Diamondback moth, such as but not limited to AChE2, P450, P450 CYP6BF1v1, Cytokine receptor DOMELESS, DOUX, Protein MESH transcript variant X1, Venom carboylesterase-6 and VPASE-E. In another embodiment, the MdsRNA comprises a sequence complementary to a target region in Fall armyworm, such as but not limited to P450 CYP9A58, Cytokine receptor DOMELESS, Dredd, VPASE, Protein MESH transcript variant X1, P450 CYP321A8, P450 CYP6B2-like. In another embodiment, the MdsRNA used in the compositions comprises microRNA sequences.

In another embodiment, the MdsRNA or the MRNA comprises a sequence complementary to a target region in Western corn root worm, such as but not limited to SNF7.

In an embodiment, the MdsRNA or the MRNA comprises a sequence selected from one of SEQ ID Nos. 1-298 and its reverse complementary strand. In a particular embodiment, the sequence is selected from one of SEQ ID Nos. 13 or 248-251 and its reverse complementary strand. In an embodiment, the target insect is a Lepidopteran and can be targeted using any one of SEQ ID Nos. 1-298. In an embodiment, the Lepidopteran is targeted using any one of SEQ ID Nos. 13 or 248-275.

In another aspect, provided herein is a composition comprising a post-transcriptionally modified double strand RNA (MdsRNA) wherein the MdsRNA comprises a double strand RNA wherein no more than about 30% (e.g., from about 2% to about 30%) of all the nucleotides independently comprise Formula (I):

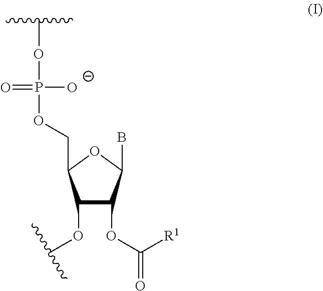

or an acceptable salt thereof, wherein:
B is a nucleobase;
R¹ is a linear or branched polyalkyloxy having a molecular weight between about 400 Da and about 15 kDa;
optionally wherein at least about 2% to about 50% of all the nucleotides independently comprise LMW modified nucleotides of Formula (III):

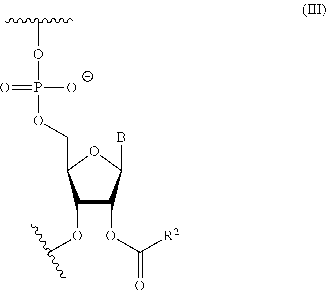

or an acceptable salt thereof, wherein:
B is a nucleobase; and
R² is selected from C₁-C₂₅ alkyl, C₁-C₂₅ alkenyl, C₁-C₂₅ alkynyl, C₅-C₁₂ aryl or C₅-C₁₂ heteroaryl, wherein R² is optionally substituted with one or more substituents selected from halo, C₁₋₁₂ alkyl, C₁-C₁₂ aminoalkyl, or C₁-C₁₂ alkoxy.

In still another aspect, provided herein is a composition comprising a post-transcriptionally chemically modified single strand RNA comprising molecules longer than 55 nucleotides to about 900 nucleotides, wherein the ssRNA is of type (A) or (B) or (C), wherein about 10% to about 95% of all the nucleotides in the linker strands L1 through L2N or L1 through L(2N−1), J1 through J(2N−1) or K1 through K(2N−1) independently comprise Formula (I):
Formula (I):

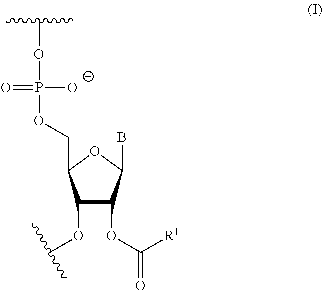

or an acceptable salt thereof, wherein:
B is a nucleobase;
R¹ is a linear or branched polyalkyloxy having a molecular weight of about 400 Da to about 15 kDa;
optionally wherein at least about 2% to about 50% of all the nucleotides in the linker strands L1 through L2N or L1 through L(2N−1), J1 through J(2N−1) or K1 through K(2N−1) independently comprise LMW modified nucleotides of Formula (III):

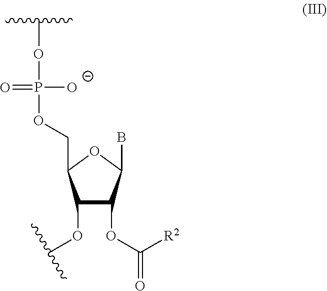

or an acceptable salt thereof, wherein:
B is a nucleobase; and
R² is selected from C₁-C₂₅ alkyl, C₁-C₂₅ alkenyl, C₁-C₂₅ alkynyl, C₅-C₁₂ aryl or C₅-C₁₂ heteroaryl, wherein R² is optionally substituted with one or more substituents selected from halo, C₁₋₁₂ alkyl, C₁-C₁₂ aminoalkyl, or C₁-C₁₂ alkoxy.

Embodiments of this aspect of the composition follow.

In an embodiment, R¹ is a linear or branched polyalkyloxy or a poloxamer having a molecular weight of about 1 kDa to about 15 kDa.

In another embodiment, R¹ is a linear or branched polyalkyloxy or a poloxamer having a molecular weight of about 5 kDa to about 15 kDa.

In another embodiment, R¹ is a linear or branched polyalkyloxy or a poloxamer having a molecular weight of about 5 kDa to about 10 kDa.

In an embodiment, R¹ is a polyethylene glycol polymer.

In an embodiment, R¹ is a poloxamer. In an embodiment, the poloxamer is a triblock polymer comprising poly(ethylene glycol)-block-poly(propylene glycol)-block-poly(ethylene glycol).

In an embodiment, R¹ is a HMW polymer with an HLB ranging from about 2 to about 30. In another embodiment, R¹ is a HMW polymer with an HLB ranging from about 15 to about 27.

In an embodiment, R² is C₁-C₂₅ alkyl. In an embodiment, the C₁-C₂₅ alkyl is substituted with one or more substituents selected from halo, C₁₋₁₂ alkyl, C₁-C₁₂ aminoalkyl, or C₁-C₁₂ alkoxy. In an embodiment, the C₁-C₂₅ alkyl is substituted with one, two, or three substituents selected from halo, C₁₋₁₂ alkyl, C₁-C₁₂ aminoalkyl, or C₁-C₁₂ alkoxy.

In an embodiment, R² is N-methyl anthranoyl (NMA), N-benzyl anthranoyl (NBA), dimethyl furoyl, -Tyr, -Trp, -Leu, octanoyl, lauroyl, linoleyl, oleyl, nicotinoyl or benzoyl.

In embodiments, the ratio of Formula (I) to Formula (III) to unmodified nucleotides is about 3:5:2; 3:6.5:0.5; 1:7:2; 0.05:9:0.5; 1.3:4:5.7; 0.01:4:5.99; 0.06:0:96.4.

In embodiments, from about 2% to about 50% of all nucleotides are modified with LMW moieties.

In another embodiment, the MdsRNA and the MRNA comprises a sequence complementary to an expressed RNA in target insects, weeds, fungi or acari, as discussed herein, the sequence listing and examples of this disclosure.

In an embodiment, R¹ is selected from:

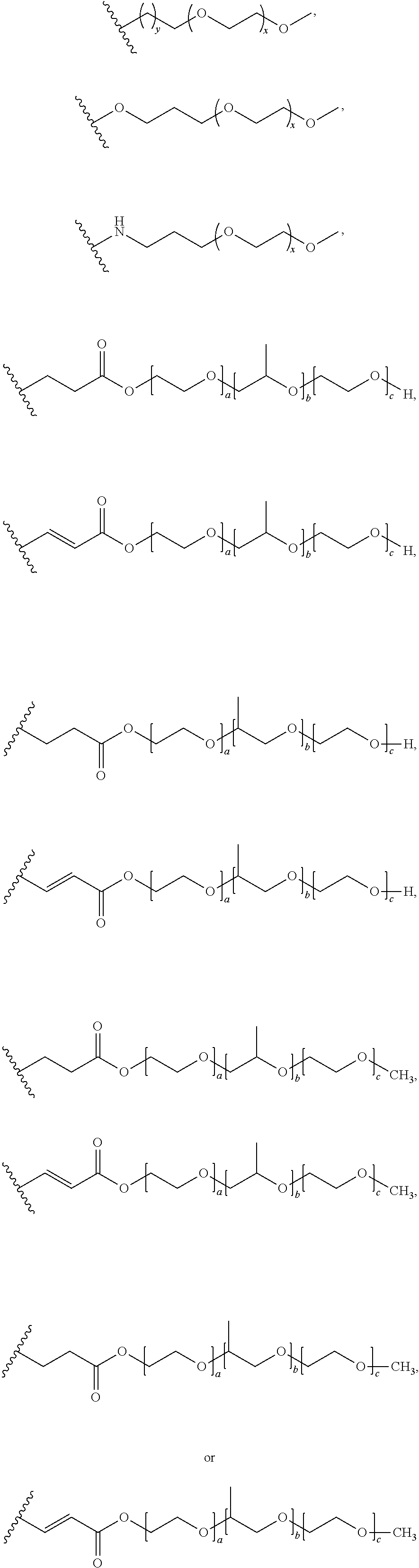

wherein y is an integer from 1-8, x is an integer from 12-1000, a is an integer from 12-1000, b is an integer from 12-1000, and c is an integer from 12-1000.

In an embodiment, R¹ is selected from:

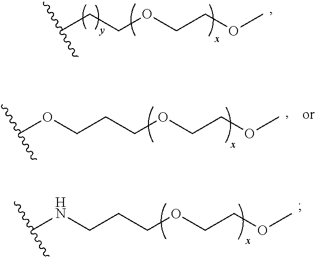

and
wherein at least 2% to about 50% of all the nucleotides in the linker strands L1 through L2N or L1 through L(2N−1), J1 through J(2N−1) or K1 through K(2N−1) independently comprise LMW modified nucleotides of Formula (III) as defined above.

In another embodiment, x is an integer from 80-1000.

In another embodiment, x is an integer from 50-900.

In another embodiment, a is an integer from 80-1000.

In yet another embodiment, b is an integer from 80-1000.

In a further embodiment, c is an integer from 80-1000.

In an embodiment, R¹ has a molecular weight from about 5,000 Da to about 15,000 Da.

In an embodiment, R¹ has a molecular weight from about 5,000 Da and to about 40,000 Da.

In an embodiment, R¹ is polyethylene glycol (PEG).

In an embodiment, R¹ is poloxamer 407, 338, 188, 184, 401, or any combination thereof.

In an embodiment, a is 101, b is 56, and c is 101.

In an embodiment, R¹ is a HMW polymer with an HLB ranging from about 2 to about 30. In another embodiment, R¹ is a HMW polymer with an HLB ranging from about 15 to about 27.

In an embodiment, R² is N-methyl anthranoyl (NMA), N-benzyl anthranoyl (NBA), dimethyl furoyl, -Tyr, -Trp, -Leu, octanoyl, lauroyl, linoleyl, oleyl, nicotinoyl or benzoyl.

In embodiments, the ratio of Formula (I) to Formula (III) to unmodified nucleotides is about 3:5:2; 3:6.5:0.5; 1:7:2; 0.05:9:0.5; 1.3:4:5.7; 0.01:4:5.99; 0.06:0:96.4.

In embodiments, from about 2% to about 50% of all nucleotides in the linker strands L1 through L2N or L1 through L(2N−1), J1 through J(2N−1) or K1 through K(2N−1) are modified with LMW moieties.

In an embodiment, the sequence of the MdsRNA or the MRNA is selected from one of SEQ ID NOs: 1-298, or 13 or 248-251.

In another embodiment, the MdsRNA and the MRNA comprises a sequence complementary to an expressed RNA in a target insect, weed, fungi, acari or in any of the targets as discussed herein and the sequence listing and examples of this disclosure.

In an embodiment, the compound of Formula (I) is

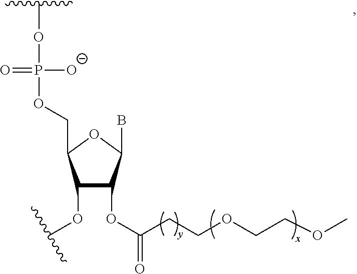

or an acceptable salt thereof, wherein each variable is defined above.

In another embodiment, the compound of Formula (I) is:

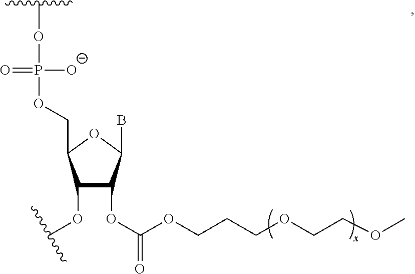

or an acceptable salt thereof, wherein each variable is defined above.

In yet another embodiment, the compound of Formula (I) is:

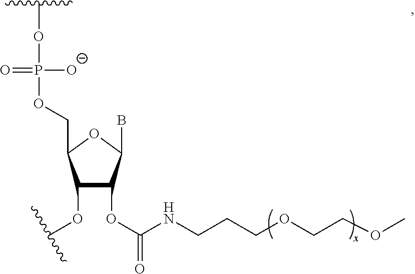

or an acceptable salt thereof, wherein each variable is defined above.

In another embodiment, the compound of Formula (I) is:

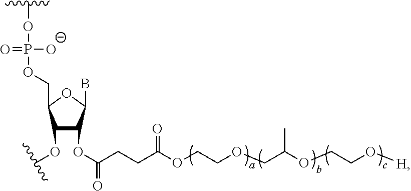

or an acceptable salt thereof, wherein each variable is defined above. In another embodiment, the terminal-H can be substituted with —CH₃.

In a further embodiment, the compound of Formula (I) is:

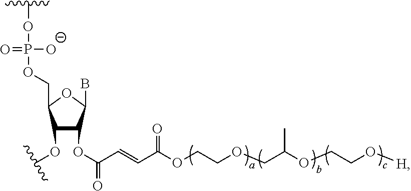

or an acceptable salt thereof, wherein each variable is defined above. In another embodiment, the terminal-H can be substituted with —CH₃.

In another embodiment, the compound of Formula (I) is:

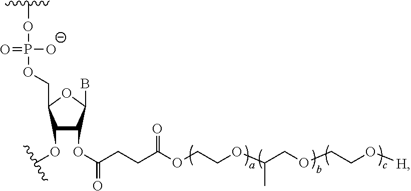

or an acceptable salt thereof, wherein each variable is defined above. In another embodiment, the terminal-H can be substituted with —CH₃.

In a further embodiment, the compound of Formula (I) is:

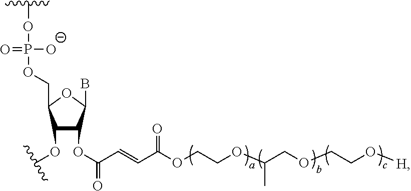

or an acceptable salt thereof, wherein each variable is defined above. In another embodiment, the terminal-H can be substituted with —CH₃. In some embodiment, each B of Formula I is the same.

In another embodiment, each B of Formula I is different.

In some embodiment, each R¹ of Formula I is the same.

In some embodiment, each R¹ of Formula I is different.

In a further embodiment, R¹ is prepared with thiol-polyethylene glycol (PEG) and acrylate or methacrylate via Michael Addition-Click reaction.

In some embodiments, the base pairs (bp) in the MdsRNA is at least about 40, at least about 50, at least about 60, at least about 70, at least about 80, at least about 90, at least about 100, at least about 200, at least about 350, at least about 400, at least about 500, at least about 600, at least about 700, at least about 800, at least about 900, or at least about 1,000 in length (e.g., from about 40 bp to about 1000 bp and increments within this range). In an embodiment, the base pairs in the MdsRNA is from about 40 base pairs to about 1,000 base pairs. In another embodiment, the MdsRNA is from about 50 base pairs to about 900 base pairs. In an embodiment, the MdsRNA is from about 70 base pairs to about 800 base pairs. In another embodiment, the MdsRNA is from about 80 base pairs to about 700 base pairs. In an embodiment, the MdsRNA is from about 90 base pairs to about 600 base pairs. In an embodiment, the MdsRNA is from about 100 nucleotides to about 500 nucleotides. In another embodiment, the MdsRNA is from about 200 base pairs to about 400 base pairs.

In some embodiments, the base pairs in each of the dsRNA stems comprised in the MRNA is from about 25 base pairs to about 60 base pairs long. In an embodiment, the base pairs in each of the dsRNA stems comprised in the MRNA is from about 30 base pairs to about 60 base pairs long. In another embodiment, the base pairs in each of the dsRNA stems comprised in the MRNA is from about 35 base pairs to about 60 base pairs. In another embodiment, the base pairs in each of the dsRNA stems comprised in the MRNA is from about 40 base pairs to about 60 base pairs long. In another embodiment, the base pairs in each of the dsRNA stems comprised in the MRNA is from about 45 base pairs to about 60 base pairs. In another embodiment, the base pairs in each of the dsRNA stems comprised in the MRNA is from about 50 base pairs to about 60 base pairs long. In another embodiment, the base pairs in each of the dsRNA stems comprised in the MRNA is from about 55 base pairs to about 60 base pairs long. In another embodiment, the base pairs in each of the dsRNA stems comprised in the MRNA is from about 25 base pairs to about 55 base pairs. In another embodiment, the base pairs in each of the dsRNA stems comprised in the MRNA is from about 25 base pairs to about 50 base pairs. In another embodiment, the base pairs in each of the dsRNA stems comprised in the MRNA is from about 25 base pairs to about 45 base pairs. In another embodiment, the base pairs in each of the dsRNA stems comprised in the MRNA is from about 25 base pairs to about 40 base pairs. In another embodiment, the base pairs in each of the dsRNA stems comprised in the MRNA is from about 25 base pairs to about 35 base pairs. In another embodiment, the base pairs in each of the dsRNA stems comprised in the MRNA is from about 25 base pairs to about 30 base pairs.

A target sequence useful in the products and methods of this disclosure will be selected to target the lowest guanine-cytosine (GC) content, such as no more than about 60% and preferably about 40% or less of the GC content (e.g., from about 30% to about 60%) of the target insect, fungus or weed, to allow for the highest level of modification of dsRNA under mild reaction conditions. In another embodiment, the MdsRNA targets a sequence with a low guanine-cytosine content. In an embodiment, the guanine-cytosine content is no more than about 50%. In an embodiment, the guanine-cytosine content is about 40% or less. In an embodiment, the guanine-cytosine content is between about 30% and about 40%. In an embodiment, guanine-cytosine content is about 30%, about 35%, about 40%, about 50%, about 55%, or about 60%. The degree of modification of the nucleotides will depend upon the GC content of the dsRNA. The degree of modification (e.g., the percentage of nucleotides that can be modified) can range from about 2% to about 90%, with from about 2% to about 30% being high molecular weight polyalkyloxy polymers, and with from about 2% to about 90% being low molecular weight filler moieties.

Without being bound by theory, it is believed that when the reaction conditions are such that the sense and antisense strands in the dsRNA are only partially dissociated (i.e., only partially unzipped), for example at temperatures below 60° C., or at low concentrations of a solvent or co-solvent capable of dissociating the sense and antisense strands, e.g., at DMSO concentrations below 70%, or at temperatures below 60° C. and at lower than 70% DMSO, the riboses in the nucleotides at positions located less than 10 contiguous nucleotides away from the beginning or the end of a perfectly paired stretch of dsRNA, may be preferentially modified, i.e., they may be modified to a larger extent than the riboses in nucleotides located more than 10 contiguous nucleotides away. For example, under such partially unzipping reaction conditions, the riboses in the nucleotides at positions 1 through 10 and 290 through 300 in a 300 bp dsRNA may be modified to a larger extent than those at positions 11 through 289.

Post transcriptionally modified double strand RNA (MdsRNA) and MRNA compounds are significantly less susceptible to degradation by nucleases in the environment and the host. MdsRNA and MRNA down-regulate expression of polynucleotides present in the target host by the RNAi mechanism. MdsRNA and MRNA compounds consist of unmodified dsRNA and ssRNA, respectively, in which some of the H atoms of its 2′OH groups have been replaced with different chemical moieties, for example with, benzoyl, lauroyl, oleyl, linoleyl, N-methyl anthranoyl, nicotinoyl, or furoyl (U.S. Pat. No. 10,131,911). These chemical moieties are generally non-toxic and, in any case, susceptible to eventual degradation in the environment, thereby reducing negative environmental impact. MdsRNAs and MRNAs are expected to be similarly non-toxic as unmodified dsRNA and ssRNA.

In some embodiments, a MdsRNA sense strand is connected to the antisense strand. A sense strand may be connected to an antisense strand via a non-hybridizing hairpin or loop sequence. A loop sequence can be about 3 nucleotides to about 100 or more nucleotides in length (e.g., from about 4 nucleotides to about 150 nucleotides). In some embodiments, a loop is 150 or more nucleotides in length (e.g., from about 150 nucleotides to about 300 nucleotides) (Hauge et al. 2009). In some embodiments, from about 10% to about 60% of the H atoms of the 2′OH groups in the linker strands L1 through L2N or L1 through L(2N−1), J1 through J(2N−1) or K1 through K(2N−1) have been replaced with chemical moieties and between about 2% and 30% of the H atoms of the 2′OH groups in the sense and antisense strands have been replaced with different chemical moieties. In some non-limiting embodiments, the different chemical moieties that have been replaced in the sense strands comprise C1-C25 alkyl, C1-C25 alkenyl, C1-C25 alkynyl, C5-C12 aryl or C5-C12 heteroaryl, wherein each moiety is optionally substituted with one or more substituents selected from halo, C1-12 alkyl, C1-C12 aminoalkyl, or C1-C12 alkoxy. In some embodiments, a MdsRNA and MRNA further comprises one or more additional sequences including, but not limited to: promoter sequences, 5′ sequences, 3′ sequences, terminator sequences, and polyA sequences.

In one embodiment, two dsRNA stems are linked by a ssRNA section (linker) having less than 200 nucleotides long (e.g., from about 1 nucleotide to about 200 nucleotides). In another embodiment, two dsRNA stems are linked by a ssRNA section having less than 40 nucleotides long (e.g., from about 1 nucleotide to about 40 nucleotides). In another embodiment, two dsRNA stems are linked by a ssRNA section having less than 10 nucleotides long (e.g., from about 1 nucleotide to about 10 nucleotides).

In aspect, two dsRNA stems are linked by a ssRNA section comprising a hairpin having from about 15 nucleotides to about 25 nucleotides long, such hairpin (PAC) having a large binding constant with a protein, for example, having a dissociation constant between 5 nM and 100 nM. In one embodiment two dsRNA stems are linked by a ssRNA section comprising a hairpin having from about 15 nucleotides to about 25 nucleotides long, such hairpin having a large binding constant with a capsid protein. In another embodiment two dsRNA stems are linked by a ssRNA section comprising a hairpin having from about 15 nucleotides to about 25 nucleotides long, such hairpin having a large binding constant with a capsid protein of a levividirae virus. In another embodiment two dsRNA stems are linked by a ssRNA section comprising a hairpin having from about 15 nucleotides and 25 long, such hairpin having a large binding constant with a capsid protein of the bacteriophage MS2. In another embodiment two dsRNA stems are linked by a ssRNA section comprising a hairpin having from about 15 nucleotides to about 25 nucleotides long, such hairpin having a large binding constant with a capsid protein of the bacteriophage Qβ. In another embodiment, two dsRNA stems are linked by a ssRNA section comprising a hairpin having about 19 nucleotides, such hairpin having a large binding constant with a capsid protein of the bacteriophage MS2. In another embodiment, two dsRNA stems are linked by a ssRNA section comprising the sequence 5′-acatgaggattacccatgt-3′ (SEQ ID NO: 299). In another embodiment two dsRNA stems are linked by a ssRNA section comprising the sequence 5′-acatgaggatcacccatgt-3′ (SEQ ID NO: 300). In another embodiment two dsRNA stems are linked by a ssRNA section comprising the sequence 5′-attcct-3′. In another embodiment two dsRNA stems are linked by a ssRNA section comprising the sequence 5′-atccct-3′.

In some embodiments, the transcribed RNA strand to be chemically modified comprises ssRNA sections and dsRNA stems. In some embodiments, the transcribed RNA strand to be chemically modified to comprise several different sense sections followed by the corresponding antisense sections wherein the sense and the antisense sections are connected by non-hybridizing RNA sections hereby called RNA linkers or linkers. In some embodiments, the transcribed RNA strand to be chemically modified comprises a post-transcriptionally chemically modified single strand RNA of type (A) or (B) or (C). In some embodiments, between about 10% and 100% of the H atoms of the 2′OH groups in the RNA linker strands L1 through L2N or L1 through L(2N−1), J1 through J(2N−1) or K1 through K(2N−1) have been replaced with chemical moieties, as disclosed herein, and between about 0% and 30% of the H atoms of the 2′OH groups in the sense and antisense strands have been replaced with different chemical moieties.

A promoter is a region (sequence) of DNA that initiates transcription of a gene. A promoter can be a bacterial promoter, archaea promoter, eukaryotic promoter, or a Pol I, Pol II, or Poll III promoter. In some embodiments, a bacterial promoter comprises the sequence 5′-TTGACA-3′ about 35 units upstream from the transcription start site and the sequence 5′-TATAAT-3′ about 10 bp upstream from the transcription start site. Other promoters, suitable for use with different expression systems are well known in the art.

Particular embodiments of the current disclosure call for higher molecular polyalkyloxy polymers to be covalently bound to the 2′-OH position of the intersubunit linkages. Preferred embodiments of the disclosure call for a low percentage of the overall subunits to be substituted with such a polymer (e.g., less than about 30%; from about 1% to about 30%). In some embodiments, the percentage of the overall subunits to be substituted with such a polymer is about 1%. In some embodiments, the percentage of substituted subunits is about 1% to about 30%. In other embodiments, the percentage of substituted subunits is about 1% to about 5%. In yet other embodiments, the percentage of substituted subunits is less than about 1%. In another embodiment, the percentage of substituted subunits is between about 0.5% and about 1%. The compounds of the disclosure show equal or superior properties to compounds having lower weight polyalkyloxy polymers at the 2′-OH position.

Without being bound by any theory, the compounds of the disclosure show equal or superior properties to compounds having lower weight polyalkyloxy polymer compounds because the instant compounds have better solubility, enhanced bioavailability, and are resistant to nucleases. Unexpectedly, while PEG and similar groups are known to prevent crossing of membranes, the instant compounds readily cross the cell-membrane. Also, unexpectedly, the low percentage levels (e.g., about 1% to about 30%) of the polyalkyloxy polymers of the disclosure still impart the beneficial stability properties of other compounds with significantly higher percentages of PEG or PEG-type substitutions (e.g., the instant compounds are resistant to exonucleases at about 1% to about 30%).

Without being bound by any theory, the compounds of the disclosure (i.e., compounds having high molecular weight polyalkyloxy polymers) are resistant to hydrolysis by carboxylate esterase. This may be due to the polyalkyloxy polymers being too sterically large for the catalytic pocket of the esterase. The larger polyalkyloxy polymers may further hydrogen bond with the 2′OH groups of the unmodified nucleotides, thus creating further steric hinderance, even at the low levels of modification as described herein. Finally, the modifications of the MdsRNA strands, as described herein, may preferentially occur at the ends of the MdsRNA strands due to the steric hinderance of the molecules. Thus, the MdsRNA strands are resistant to exonucleases while still being available to endonucleases, allowing the compounds of the disclosure to enter into the RNAi process easier.

Embodiments and aspects in this and the other sections of the disclosure regarding the composition, methods of making the compositions, nucleotide sequences and uses for target insects, fungi, weeds or acari are intended to be covered herein in this disclosure.

### Selected Sequences

DNA Sequences that Encode MS2 PAC Sites, from which the dsRNA is Transcribed

In an embodiment, the sequence is SEQ ID NO: 2

In another embodiment, the sequence is SEQ ID NO: 3.

In another embodiment, the sequence is SEQ ID NO: 4.

In another embodiment, the sequence is SEQ ID NO: 5.

DNA sequences that do not encode MS2 PAC sites, from which the dsRNA is transcribed

In another embodiment, the sequence is SEQ ID NO: 6.

In another embodiment, the sequence is SEQ ID NO: 7.

In an embodiment, the sequence is Plutella xylostella acetylcholinesterase 2 mRNA, GenBank AY061975.1 nucleotide #s: 512-811;

(SEQ ID NO: 1)
catatcgga ggattgcctc tatttgaaca tatgggtgcc
gcagcacttg cgcgtccgtc accatcagga caagccatta
accgagcgac cgaaggttcc aatactagtg tggatttacg
gcgggggtta catgagtggc acggcgacac ttgatctata
taaagccgac ataatggcgt cttcgagtga tgtgatcgta
gcctcgatgc agtatagggt tggcgcgttt ggatttttgt
accttaacaa atatttttcc cctggtagcg aggaagcggc
aggaaatatg ggcttgtggg a.

In another embodiment, the sequence is Plutella xylostella acetylcholinesterase 1 mRNA, GenBank: AY970293.1 nucleotide #s: 889-1188:

(SEQ ID NO: 2)
tc acaatgtcac attgtttgga gaatcgtccg gtgcagtttc
cgtgtcatta cacttactgt ctccgctgtc aagaaacatg
ttttctcaag ctattatgca atctgcagcc gcatctgcac
cttgggccat catttccaga gaggagagtg tgataagggg
catccgcctg gccgaggccg tccactgc.

In another embodiment, the sequence is Plutella xylostella tyrosine hydroxylase mRNA, GenBank: JN410829.1 nucleotide #s: 301-600;

(SEQ ID NO: 3)
gctgaggtcg gtggaataga cggaaatgca gatgatgatt
acaccttgac cgaggaggag gtgatcttgc agaactccgc
cagcgagtcc ccggaggccg agcaggcgct gcaacaagcg
gctttgcttc tgcgcctgcg cgacggcatg ggctcgctcg
cgcgcatcct caagaccatc gacaactaca agggatgcgt
tcaacacctc gagactcgcc cctccaacgc caacgacatc
caattcgatg ctctcatcaa agtgagcatg tcccgtggca
acctgctcca actcatccga.

In another embodiment, the sequence is Plutella xylostella integrin beta 1 mRNA, GenBank: GQ178290.1 nucleotide #s: 531-830;

(SEQ ID NO: 4)
tgcaggtcaa gccgcagagg gtcaagctgc agctgcgcat
gaaccagatg cagaaactag acgtcgccta ttcccaagcc
caagactacc cggtggacct gtactacttg atggacctga
gtcgttccat gaagaacgac aaggagaagc tcagtacatt
gggcagtctg ctgtccagca ctatgaggaa tatcacgccc
aacttccgtc ttggcttcgg ctccttcgtg gacaagctcg
tcatgcccta cgtgtctact gtgcctaaga atttgatatc
cccttgtgat ggctgcgcgg.

In another embodiment, the sequence is Plutella xylostella charged multivesicular body protein 4b-like mRNA (XM_011555904.1) nucleotide #s: 321-620;

(SEQ ID NO: 5)
ccaggaaaca tggcactaag aacaaaagag cggccatcgc
tgcacttaaa cgcaagaagc gttacgagaa gcaactcaca
cagattgacg gcacgctcag ccagatagag atgcagagag
aggcattgga gggcgccaac actaacactc aagtactgaa
cacgatgcga gaggccgccg cggctatgaa gctcgctcac
aaggatattg acgtagacaa agtgcatgat atcatggacg
acatcgctga acaacatgat gtggctcgcg aaatcacgga
tgccatcagc aacaatgtgg.

In another embodiment, the sequence is Plutella xylostella peptidoglycan recognition protein mRNA, GenBank: EU399240.1 nucleotide #s: 31-330;

(SEQ ID NO: 6)
tcagtgt tttgttgttg tgctcatgca gggtgtggcg
tggtgaccag acagcagtgg gatgggctgg acccgataca
gttggagtac ctgccccggc ccctggggct ggtggtggtc
cagcacaccg ccacccccgc gtgtgacact gacgccgcgt
gtgtggagct ggtgcagaa atacagacca atcatatgga
tgtgctgaag ttttgggata ttggaccgaa cttcctgatt ggt.

In another embodiment, the sequence is Plutella xylostella mRNA for vacuolar ATP syntethase subunit E, GenBank: AB189032.1 (identical to NM_001305532.1) nucleotide #s: 64-363;

(SEQ ID NO: 7)
gcgctca gcgatgcaga tgtccaaaaa cagatcaagc
atatgatggc cttcatcgag caagaggcaa atgaaaaggc
cgaagaaatc gatgctaagg ctgaggagga gttcaacatc
gagaaggggc gtctggtgca gcagcagcgc ctcaagatca
tggagtacta cgagaagaag gagaagcagg tggaactcca
gaagaagatc caatcctcca acatgctgaa ccaggcccgt
ctgaaggtgc tgaaggtgcg cgaggaccac gtgggccacg
tgttggacga gacgcgccgc cgc.

In another embodiment, the sequence is Diabrotica virgifera virgifera charged multivesicular body protein 4b (LOC114337301), mRNA Sequence ID: XM_028287710.1;

(SEQ ID NO: 8)
cacaactgac agataacgtc agtagttgtc tattttcact
ggtgactaat ttttgagaat tagtaattgg tttcgtattt
ttttcttaac aaaagggcaa aatgagcttt tttggaaaat
tgttcggggg gaaaaaggaa gagatagccc ctagtcctgg
ggaggctatt caaaaactca gagagactga agaaatgtta
ataaaaaaac aggatttttt agaaaagaag atagaagaat
ttaccatggt agcaaagaaa aatgcgtcga aaaataaaag
agttgcactc caagccctca aaaagaagaa acgattggaa
aagacccaac tacaaataga tggaaccctt acaactattg
aaatgcagag ggaagccctc gaaggagcta gcacaaatac
tgctgtatta gattctatga aaaatgctgc agatgccctt
aagaaagctc ataagaattt gaatgtagat gatgttcacg
atatcatgga tgacatagcc gaacaacacg acatagccaa
cgaaatcaca aacgctatta gcaatcctgt cggattcacc
gacgatctgg atgacgatga attagaaaaa gaattagaag
agctcgaaca agaaggattg gaagaagacc tgctccaagt
gccaggtcca actcaactgc cggctgtgcc tgctgatgca
gttgctacta aaccaatcaa accagcagct aaaaaagttg
aagatgatga cgatatgaaa gaattggaag cctgggcctc
gtaaaattcc tgaaatctga atatttgtaa acgaaatcac
ccatctaaga tgaaaacatt ataaatatat aggtaataac
agctaaaaac gtttcaatgt agaacaagct tttgctgaaa
aggcgtcttg caaaaaatgt tgataattta gaattctcta
tatattatat atttgccctt taaggaaaga tttcttttat
agtcatagtt caaccaaacg tttcataaat tagaatacag
gggtgtcaga attctacgtg aatttattga aaaaaaagaa
ctttccttaa agttggcata atgatatttt tgaaattatg
taaataggta tatgtatgct cttactaacg gttttaatat
tgggtttaga agcaccttaa ttttattttt atacaaggta
gtaacatttt ttcattgtaa tttgttaaaa aatattgtat
aacgcaaaaa atggtgtgat aaagcaaaat attattgagt
gcttattttc ttattttatt aaaagatctt atttacgtta
aatctaaata tattgggtat ctcagaatat gttaacaaag
gtttcttgta tcaacagaaa aatacaaact tattcattat
tgtggttcat ttgattatgc ttgtgtatat ttaatctgcc
ataaacaagt tttgataaat gtcactgctc tgta.

In another embodiment, the sequence is Acyrthosiphon pisum V-type proton ATPase subunit E Genbank #: XM_008185078.2 nucleotide #s: 540-724;

(SEQ ID NO: 9)
t tagccaacactggaataaac gtcaaaataa acattgataa
aagtattaaa ttaccgactc aagaaatagg aggcgtcgtg
gtcacgtcca aagatcgaag ggtacatgtt gaaaatacgc
ttgtagtgag attgctctat ctcacccaac aagcaatacc
aataatatgc actggactgt ttgg.

In another embodiment, the sequence is Solenopsis invicta's (RIFA) actin muscle (LOC105205816, GenBank: XM_011175337.1 nucleotides #465-763;

(SEQ ID NO: 10)
gatctc tctccctcga ctctaacacc agcgaaagta
acagccaatc aagatgtgtg acgatgatgt tgcggcatta
gtcgtggaca atgggtccgg tatgtgcaag gctggattcg
cgggggatga tgcaccacgc gctgtgtttc ccagcatcgt
cggtcgtcct cgtcatcagg gtgtgatggt cggtatgggt
caaaaagaca gttatgttgg cgacgaggcg caaagtaaga
gaggtatatt gacactaaag tatcctatag aacatg.

In yet another embodiment, the sequence is Gibberella zeae isolate NX3 cytochrome P450 51B gene GenBank: FJ216402.1 nucleotide positions 804-1023;

(SEQ ID NO: 11)
cagcaag tttgacgagt ccctggccgc tctctaccac
gacctcgata tgggcttcac ccccatcaac ttcatgcttc
actgggcccc tctcccctgg aaccgtaagc gcgaccacgc
ccagcgcact gttgccaaga tctacatgga cactatcaag
gagcgccgcg ccaagggcaa caacgaatcc gagcatgaca
tgatgaagca ccttatgaac tct.

In another embodiment, the sequence is Fusarium graminearum PH-1 cytochrome P450 51 NCBI Reference Sequence: XM_011327038.1 nucleotide positions 163-400;

(SEQ ID NO: 12)
attggaag caccgtacaa tatggcatcg acccgtacgc
ttttttcttc gactgcagag ataaatacgg cgactgcttt
acctttattc tccttggcaa atcaacgact gtctttcttg
gtcccaaggg caatgacttt atcctcaacg gcaaacacgc
cgatctcaac gccgaggacg tttatgggaa acttaccacg
cccgtgtttg gtgaggaggt tgtttatgac tgctccaatg.

### Method of Preparation of High-Molecular Weight Polyalkyloxy Polymers by Ionic Solvation

In another aspect, provided herein are methods of preparing a composition comprising a post-transcriptionally chemically modified double strand RNA (MdsRNA) wherein the MdsRNA comprises a double strand RNA wherein no more than about 30% (e.g., from about 2% to about 30%) of all the nucleotides independently comprise Formula (I):

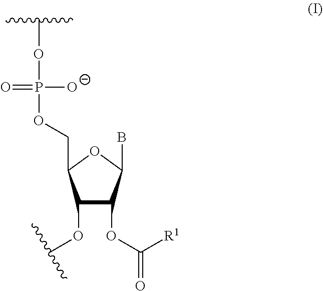

or an acceptable salt thereof, wherein:
B is a nucleobase;
R¹ is selected from:

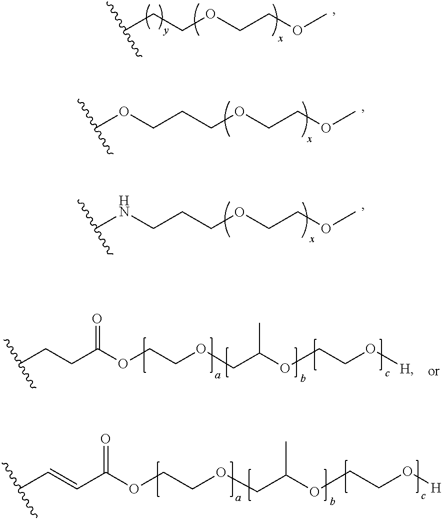

wherein y is an integer from 1-8, x is an integer from 12-1000, a is an integer from 12-1000, b is an integer from 12-1000, and c is an integer from 12-1000; and
the method comprising:
(a) contacting a compound of Formula (II):

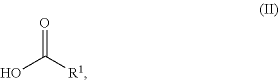

with an activation agent to form a compound of Formula (IIA):

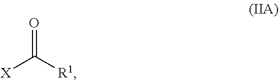

wherein X is a suitable leaving group; and
(b) contacting a compound of Formula (IA):

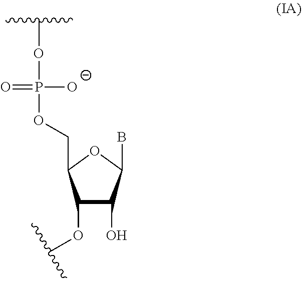

with a compound of Formula (IIA) to form a compound of Formula (I).

In another aspect, provided herein are methods of preparing a composition comprising a post-transcriptionally chemically modified double strand RNA (MdsRNA) wherein the MdsRNA comprises a double strand RNA wherein no more than about 30% (e.g., from about 2% to about 30%) of all the nucleotides independently comprise Formula (I):

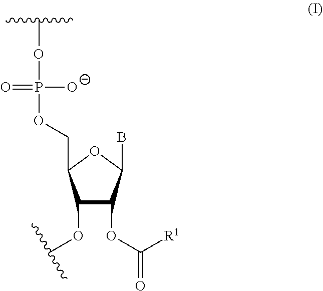

or an acceptable salt thereof, wherein:
B is a nucleobase;
R¹ is selected from:

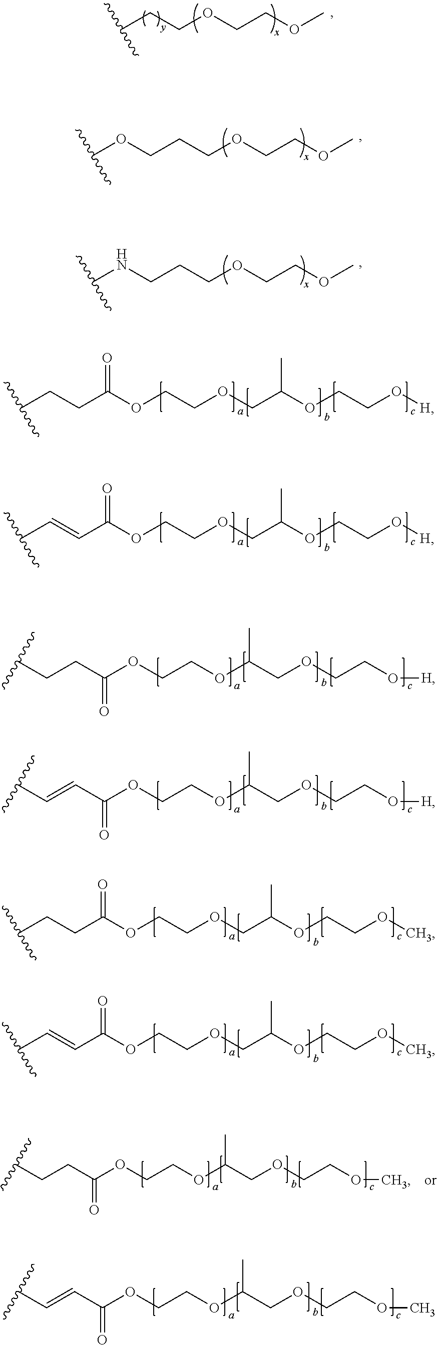

wherein y is an integer from 1-8, x is an integer from 12-1000, a is an integer from 12-1000, b is an integer from 12-1000, and c is an integer from 12-1000; and
optionally wherein at least about 2% (e.g., from about 10% to about 60%) of all the nucleotides independently comprise LMW modified nucleotides of Formula (III):

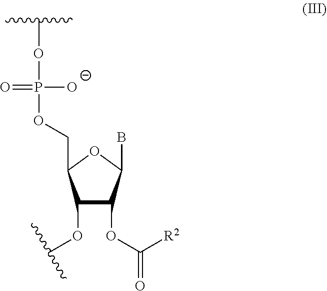

or an acceptable salt thereof, wherein:
B is a nucleobase;
R² is selected from C₁-C₂₅ alkyl, C₁-C₂₅ alkenyl, C₁-C₂₅ alkynyl, C₅-C₁₂ aryl or C₅-C₁₂ heteroaryl, wherein R² is optionally substituted with one or more substituents selected from halo, C₁₋₁₂ alkyl, C₁-C₁₂ aminoalkyl, or C₁-C₁₂ alkoxy;
the method comprising:

(a) contacting a compound of Formula (II):

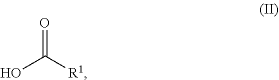

with an activation agent to form a compound of Formula (IIA):

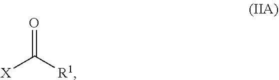

wherein X is a suitable leaving group;
(b) contacting a compound of Formula (IA):

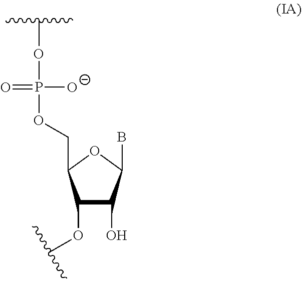

with a compound of Formula (IIA) to form a compound of Formula (I);
(c) optionally contacting a compound of Formula (V):

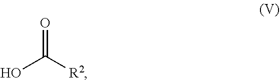

with an activation agent to form a compound of Formula (VA):

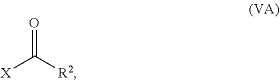

wherein X is a suitable leaving group;
(d) optionally contacting a compound of Formula (IA):

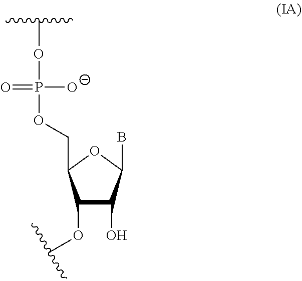

with a compound of Formula (VA) to form a compound of Formula (III).

In an embodiment, (a) and (b) are carried out in an anhydrous solvent.

In an embodiment, (c) and (d) are carried out in an anhydrous solvent.

In an embodiment, the anhydrous solvent is selected from DMSO or DCM.

In another embodiment, (a) and (b) are carried out without intervening purification.

In another embodiment, (c) and (d) are carried out without intervening purification.

In yet another embodiment, there is a purification step between (a) and (b).

In another embodiment, there is a purification step between (c) and (d).

In an embodiment, an ionic solvent is added after (a).

In an embodiment, an ionic solvent is added after (c).

In an embodiment, the ionic solvent is selected from benzyltributyl ammonium chloride or benzyltrimethyl ammonium chloride or benzyltrimethyl ammonium bromide.

In an embodiment, the ionic solvent is selected from tetramethyl ammonium chloride, tetraethyl ammonium chloride cetyltrimethyl ammonium bromide, or cetyltrimethyl ammonium chloride.

In another embodiment, the activation agent is carbonyldiimidazole.

In yet another embodiment, the suitable leaving group is:

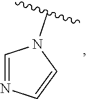

wherein [symbol] represents the covalent point of attachment to carbonyl of Formula (IA).

In yet another embodiment, the suitable leaving group is:

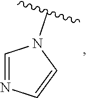

wherein [symbol] represents the covalent point of attachment to carbonyl of Formula (VA).

In a further embodiment, (b) has a ratio of less than ten equivalents of the compound of Formula (IIA) per nucleotide of the dsRNA.

In a further embodiment, (d) has a ratio of between about two equivalents and about fifty equivalents of the compound of Formula (VA) per nucleotide of the dsRNA.

In another embodiment, (b) has a ratio of less than two equivalents of the compound of Formula (IIA) per nucleotide of the dsRNA.

In another embodiment, (d) has a ratio of between about four equivalents and about twenty-five equivalents of the compound of Formula (VA) per nucleotide of the dsRNA.

In another embodiment, the compound of Formula (II) is the anhydride.

In another embodiment, the compound of Formula (V) is the anhydride.

In yet another embodiment, R¹ is selected from:

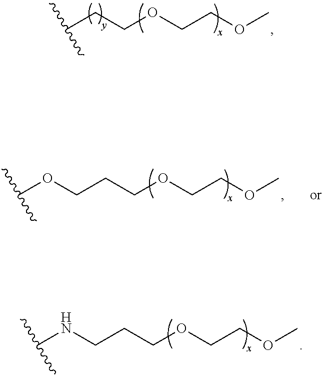

In another embodiment, x is an integer from 80-1000.

In another embodiment, x is an integer from 50-900.

In another embodiment, a is an integer from 180-1000.

In yet another embodiment, b is an integer from 80-1000.

In a further embodiment, c is an integer from 80-1000.

In an embodiment, R¹ has a molecular weight from about 5,000 Da to about 10,000 Da.

In an embodiment, R¹ has a molecular weight from about 5,000 Da to about 40,000 Da.

In an embodiment, R¹ is polyethylene glycol (PEG).

In an embodiment, R¹ is poloxamer 407, 338, 188, 184, 401, or any combination thereof.

In an embodiment, a is 101, b is 56, and c is 101.

In an embodiment, R¹ is a HMW polymer with an HLB ranging from about 2 to about 30. In another embodiment, R¹ is a HMW polymer with an HLB ranging from about 8 to about 27.

In an embodiment, steps (c) and (d) are present.

In an embodiment, steps (b) and (d) are performed sequentially.

In an embodiment, steps (b) and (d) are performed simultaneously.

In another embodiment, the MdsRNA comprises a sequence complementary to an expressed RNA in a target insect, fungus, weed or acari, as discussed in detail in this disclosure, such as but not limited to target (insect fungus, weed or acari), target sequences and target regions.

In another embodiment, R² is selected from N-methyl anthranoyl (NMA), N-benzyl anthranoyl (NBA), dimethyl furoyl, -Tyr, -Trp, -Leu, octanoyl, lauroyl, linoleyl, oleyl, nicotinoyl or benzoyl.

Embodiments in other sections of the disclosure regarding the composition, methods of making the compositions, nucleotide sequences and uses for target insects, fungi, weeds or acari are intended to be covered herein this disclosure.

In some embodiments, the disclosed method of preparation is superior to prior methods in both cost and scalability. Prior methods of synthesis required water or another suitable polar protic solvent to reduce the need for excess solvent dilution. However, this led to significant degradation of the necessary polyalkyloxy polymer anhydrides. Prior methods sometimes employed as much as 200 equivalents of modifying groups per one bp of dsRNA. The addition of ionic solvent has provided the superior and unexpected benefit of reducing the amount of solvent used, reducing the degradation of starting material, and allowing efficient synthesis of the desired product. In some embodiments of the method, (b) has a ratio of less than 0.05 equivalents of the compound of Formula (IIA) (e.g., the polyalkyloxy polymer) per one bp of the dsRNA. In another embodiment of the method, (b) has a ratio of less than 0.1 equivalents of the compound of Formula (IIA). In particular embodiments of the method, (b) has a ratio of about 0.2, about 0.3, about 0.4, about 0.5, about 0.6, about 0.7, about 0.8, about 0.9, or about 1.0 equivalents of the compound of Formula (IIA) per one bp of the dsRNA.

Without wishing to be held to any particular theory, the ionic solvent is not acting as a phase transfer catalyst. Rather, the ionic solvent is acting, unexpectedly, as a superior solvating agent. The ionic solvent also serves to help “unzip” the dsRNA by shielding the charges on the molecules. This use of such an ionic solvent is unreported in the prior art.

Thus, in an embodiment, the ionic solvent improves dsRNA dissociation by shielding the resulting charge on the single strand RNA.

### Method of Preparation of Low-Molecular Weight Modifications by Ionic Solvation

In another aspect, provided herein are methods of preparing a composition comprising a post-transcriptionally modified double strand RNA (MdsRNA) wherein the MdsRNA comprises a double strand RNA wherein no more than about 55% (e.g., from about 20% to about 55% but could be up to about 85%) of all the nucleotides independently comprise Formula (VI):

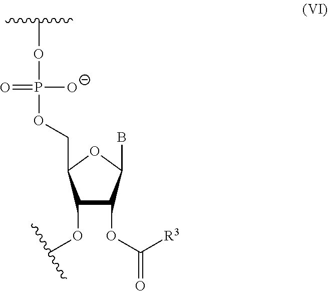

or an acceptable salt thereof, wherein:
B is a nucleobase;
R³ is selected from: amino acids, fatty acids, alkyl; substituted alkyl; alkenyl; substituted alkenyl; alkynyl; substituted alkynyl; aryl; substituted aryl; C1-C10 alkyl, C1-C10 alkenyl, or C1-C10 alkynyl wherein alkyl and alkenyl can be linear, branched or cyclic; hydrogen; methyl; ethyl; propyl; isopropyl; butyl; isobutyl; tert-butyl; pentyl; hexyl; cyclohexyl; heptyl; octyl; nonyl; decyl; vinyl; allyl; ethynyl; benzyl; cinnamyl; C6-C14 aryl; C6-C14 substituted aryl; heterocyclyl; C5-C14 heterocyclyl; phenyl; mono or disubstituted phenyl wherein the substituents are selected from C1-C10 alkyl, C1-C10 alkenyl, C1-C6 alkoxy, halogen, nitro, methylsulfonyl, and trifluoromethyl; 2-nitrophenyl; 4-nitrophenyl; 2;4-dinitrophenyl; 2-trifluoromethylphenyl; 4-trifluoromethylphenyl; styryl; C8-C16 substituted styryl; 2-aminophenyl; mono or disubstituted 2-aminophenyl wherein the substituents are selected from C1-C10 alkyl, C1-C10 alkenyl, C1-C6 alkoxy, halogen, nitro, methylsulfonyl, and trifluoromethyl; N-alkyl-2-aminophenyl or N-aryl-2-aminophenyl wherein alkyl has the formula —CₘH₂ₘ₊₁ (wherein m is an integer less than or equal to 12) and aryl is an aromatic moiety; 2-amino-3-methyl-phenyl; 2-amino-5-chlorophenyl; 2-methyl-5-chlorophenyl; N-methyl-2-aminophenyl; N-ethyl-2-aminophenyl; N-propyl-2-aminophenyl; N-butyl-2-aminophenyl; N-pentyl-2-aminophenyl; N-methyl-2-amino-4-nitrophenyl; 2-methyl-3-furyl; 2-methylnicotyl or N-trifluoromethyl-2-aminophenyl; silanyl; substituted silanyl; C1-C10 alkylsilanyl; C3-C12 trialkylsilanyl; C2-C12 alkoxyalkyl; C2-C12 alkoxyalkenyl; C2-C12 alkylthioalkyl; alkylsulfonyl; C1-C10 alkylsulfonyl; C1-C10 haloalkyl; C1-C10 haloalkenyl or C1-C10 aminoalkyl; —(CH₂CH₂O)ₚCH₃, —(CH₂CH₂O)ₚH, or —(CH₂CH₂O)ₚCOOR₄ wherein p is an integer from 2 to 8 and R⁴ is H, alkyl, substituted alkyl, aryl, or substituted aryl; —(CH₂CH₂O)₈COOH; —CH₂CH₂OH; —(CH₂CH₂O)₄OH; —(CH₂CH₂O)₆OH; —(CH₂CH₂O)₈OH; —(CH₂CH₂O)₈COOMe; —(CH₂CH₂O)₄OMe; —(CH₂CH₂O)₆OMe; —(CH₂CH₂O)₈OMe; —CH₂OCH₃; —CH₂OCH₂CH₃; or —CH₂OCH₂CH₂OCH₃; and
the method comprising:
(a) contacting a compound of Formula (IV):

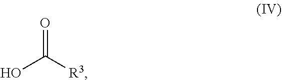

with an activation agent to form a compound of Formula (IVA):

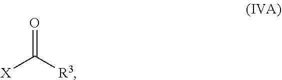

wherein X is a suitable leaving group; and
(b) contacting a compound of Formula (VIA):

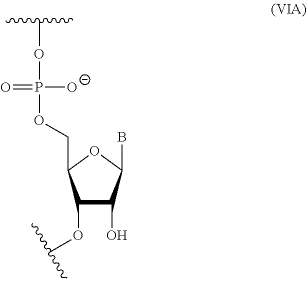

with a compound of Formula (IVA) to form a compound of Formula (VI).

In an embodiment, (a) and (b) are carried out in an anhydrous solvent.

In an embodiment, the anhydrous solvent is selected from DMSO or DCM.

In another embodiment, (a) and (b) are carried out without intervening purification.

In yet another embodiment, there is a purification step between (a) and (b).

In an embodiment, an ionic solvent is added after (a).

In an embodiment, the ionic solvent is selected from benzyltributyl ammonium chloride or benzyltrimethyl ammonium chloride.

In another embodiment, the activation agent is carbonyldiimidazole.

In yet another embodiment, the suitable leaving group is:

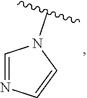

wherein [symbol] represents the covalent point of attachment to carbonyl of Formula (IVA).

In a further embodiment, (b) has a ratio of between about two equivalents and about fifty equivalents of the compound of Formula (IVA) per nucleotide of the dsRNA.

In another embodiment, (b) has a ratio of between about four equivalents and about twenty-five equivalents of the compound of Formula (IVA) per nucleotide of the dsRNA. In another embodiment, the compound of Formula (IV) is the anhydride.

In another embodiment, R³ is selected from N-methyl anthranoyl (NMA), N-benzyl anthranoyl (NBA), dimethyl furoyl, -Tyr, -Trp, -Leu, octanoyl, lauroyl, linoleyl, oleyl, nicotinoyl or benzoyl.

In another embodiment, the MdsRNA comprises a sequence complementary to an expressed RNA in a target insect, fungus, weed or acari, as discussed in detail in this disclosure, such as but not limited to target (insect fungus, weed or acari), target sequences and target regions.

Embodiments in other sections of the disclosure regarding the composition, methods of making the compositions, nucleotide sequences and uses for target insects, fungi, weeds or acari are intended to be covered in this disclosure.

In some embodiments, the disclosed method of preparation is superior to prior methods in both cost and scalability. Prior methods of synthesis required water or another suitable polar protic solvent to reduce the need for excess solvent dilution. However, this led to significant degradation of the necessary anhydrides. Prior methods sometimes employed as much as 200 equivalents of modifying groups per one bp of dsRNA. The addition of ionic solvent has provided the superior and unexpected benefit of reducing the amount of solvent used, reducing the degradation of starting material, and allowing efficient synthesis of the desired product. In some embodiments of the method, (b) has a ratio of less than 0.05 equivalents of the compound of Formula (IVA) per one bp of the dsRNA. In another embodiment of the method, (b) has a ratio of less than 0.1 equivalents of the compound of Formula (IVA). In particular embodiments of the method, (b) has a ratio of 0.2 equivalents of the compound of Formula (IVA) per one bp of the dsRNA.

Without wishing to be held to any particular theory, the ionic solvent is not acting as a phase transfer catalyst. Rather, the ionic solvent is acting, unexpectedly, as a superior solvating agent. The ionic solvent also serves to help “unzip” the dsRNA by shielding the charges on the molecules. This use of such an ionic solvent is unreported in the prior art.

Low molecular weight compositions have been described in U.S. Pat. Nos. 10,131,911, 10,640,769, and 11,174,480, the entire teachings of which are incorporated by reference.

Thus, in an aspect, provided herein is a method to produce a post transcriptionally chemically modified double strand RNA (MdsRNA) deliverable to pests, comprising:
a) transcribing DNA of a target pest into dsRNA formed from a ssRNA strand comprising from about one to about twenty sense sections followed by one or more sections antisense to the sense sections;
b) adding a salt comprising a quaternary ammonium or a quaternary phosphonium cation or a pyrrolidinium or a pyridium or a piperidimium and a neutralizing anion (e.g., from about 0.25 equivalents to about 2 molar equivalents of nucleotide) to produce an isolate comprising more than half (e.g., from about 50% to about 99%) of the dsRNA produced in step (a); and
c) conducting a chemical reaction between an activated compound IIA or IVA and from about 2% to about 50% of the 2′OH groups in the dsRNA resulting from step (b) in a liquid comprising at least 1 wt % (e.g., from about 1 wt % to about 80 wt % of the salt used in step (b), resulting in a post-transcriptionally chemically modified double strand RNA (MdsRNA).

In one embodiment, the step (a) comprises transcribing DNA into dsRNA formed from a ssRNA strand wherein the ssRNA comprises a sense section i, a sense section j, a sense section k, a section i* antisense to i, a section j* antisense to j, and a section k* antisense to k, wherein each section is from about 20 nucleotides to about 60 nucleotides long, wherein such sections are located in order 5′-i-j-k-i*-j*-k*-3′.

In one embodiment, the step (a) comprises transcribing two reverse complementary strands of DNA in which the first of the DNA strands comprises a promoter; a dsRNA encoding region positioned transcriptionally downstream of the promoter, wherein said dsRNA encoding region comprises a) one sense-oriented nucleotide sequence, which substantially corresponds to a target sequence, b) one anti-sense-oriented nucleotide sequence which is substantially complementary to the target sequence, and c) one nucleotide sequence separating the sense and antisense nucleotide sequences and which encodes from about 1 to about 200 nucleotides of a non-hybridizing region of an RNA transcript; and one transcription terminator sequence, positioned 3′ to the dsRNA encoding region wherein the dsRNA encoding region and transcription terminators are operably linked to the promoter. The second of the DNA strands comprises a promoter; a dsRNA encoding region positioned transcriptionally downstream of the promoter, wherein the dsRNA encoding region is reverse complementary to the dsRNA encoding region of the first DNA strand, and one transcription terminator sequence, positioned 3′ to the ssRNA encoding region wherein the dsRNA encoding region and transcription terminator are operably linked to the promoter.

In another embodiment, the step (a) is conducted by a DNA-dependent RNA polymerase in an aqueous phase substantially devoid of microorganisms.

In another embodiment, the step (a) is conducted by a microorganism chosen from a bacterium and a yeast.

In another embodiment, the step (a) is conducted by a microorganism that also expresses a coat protein of the capsid substantially identical (e.g., from about 90% to about 100% identity) to that of a levividirae virus.

In another embodiment, the step (a) is conducted by a microorganism that also expresses a coat protein of the capsid substantially identical (e.g., from about 90% to about 100% identity) to that of bacteriophage MS2.

In another embodiment, the step (a) is conducted by Escherichia coli.

In another embodiment, the step (a) is conducted by the strain HT115 (D3) of E. coli.

In another embodiment, the step (a) is conducted by the strain M-JM109lacY of E. coli.

In another embodiment, the step (a) is conducted by the rnc-deficient strain of E. coli BL21 (DE3).

In another embodiment, the step (a) is conducted by Ensifer meliloti.

In another embodiment, the step (a) is conducted by strain 1021 of Ensifer meliloti.

In another embodiment, the step (a) is conducted by Pseudomonas syringae.

In another embodiment, the step (a) is conducted by strain LM2691 of Pseudomonas syringae.

In another embodiment, the step (a) is conducted by Corynebacterium glutamicum.

In another embodiment, the step (a) is conducted by the rnc-deficient strain 2256LΔrnc of Corynebacterium glutamicum.

In another embodiment, the step (a) is conducted by Chlamydomonas reinhardtii.

In another embodiment, the step (a) is conducted by Lactobacillus plantarum.

In another embodiment, the step (a) is conducted by Yarrowia lipolytica.

In another embodiment, the step (a) is conducted by Rhodococcus rhodnii.

In another embodiment, the step (a) is conducted by strain LMG5362 of Rhodococcus rhodnii.

In another embodiment, the step (a) is conducted by Saccharomyces cerevisiae.

In another embodiment, the step (a) is conducted by the strain HF7c of Saccharomyces cerevisiae.

In another embodiment, the step (a) is conducted by the strain CEN.PK of Saccharomyces cerevisiae.

In another embodiment, the step (a) is conducted by Pichia pastoris.

In another embodiment, the step (b) comprises:
i. precipitation of the dsRNA from an aqueous phase by addition of a salt comprising a quaternary ammonium or a quaternary phosphonium cation or a pyrrolidinium or a pyridium or a piperidimium;
ii. isolation of the insoluble precipitate obtained in step (i) by centrifugation or filtration; and
iii. removal of substantially all the water from the insoluble precipitate obtained in step (ii).

In another embodiment, the salt added in step (b) comprises a quaternary ammonium cation chosen from trimethyloctyl ammonium, trimethyldecyl ammonium, trimethyldodecyl ammonium, trimethyltetradecyl ammonium, trimethylhexadecyl ammonium, trimethyloctadecyl ammonium, trimethylbenzyl ammonium, tributylbenzyl ammonium, choline, amyltriethylammonium, butyltrimethylammonium, benzylethyldimethylammonium, cyclohexyltrimethylammonium, diethyl(methyl)-propylammonium, diethyl(2-methoxyethyl)-methylammonium, ethyl(2-methoxyethyl)-dimethylammonium, ethyl(3-methoxypropyl)dimethylammonium, ethyl(dimethyl)(2-phenylethyl)-ammonium, methyltri-n-octylammonium, tetrabutylammonium, tetrahexylammonium, tetraamylammonium, tetra-n-octylammonium, tetraheptylammonium, tetraamylammonium, tetrapropylammonium, tributylmethylammonium, trimethylpropylammonium, tributyl(methyl)-ammonium, 1-allyl-3-methylimidazolium, 1-butyl-3-methylimidazolium, 1-butyl-2,3-dimethylimidazolium, 3,3′-(butane-1,4-diyl)-bis(1-vinyl-3-imidazolium), 1,2-dimethyl-3-propylimidazolium, 1-decyl-3-methylimidazolium, 1,3-dimethylimidazolium, 1-dodecyl 3-methylimidazolium, 1-ethyl-3-methylimidazolium, 1-ethyl-2,3-dimethylimidazolium, 1-hexyl-3-methylimidazolium, 1-(2-hydroxyethyl)-3-methylimidazolium, 1-methyl-3-noctylimidazolium, 1-methyl-3-pentylimidazolium, 1-benzyl-3-methylimidazolium, 4-ethyl-4-methylmorpholinium, tributylhexylphosphonium, tributylhexadecylphosphonium, tributylmethylphosphonium, tributyl-noctylphosphonium, tetrabutylphosphonium, tetra-n-octylphosphonium, tributyl(2-methoxyethyl)-phosphonium, tributylmethylphosphonium, trihexyl(tetradecyl)-phosphonium, trihexyl(tetradecyl)-phosphonium, tributyl(ethyl)phosphonium, tributyl(methyl)phosphonium, 1-allyl-1-methylpyrrolidinium, 1-butyl-1-methylpyrrolidinium, 1-ethyl-1-methylpyrrolidinium, 1-methyl-1-propylpyrrolidinium, 1-(2-methoxyethyl)-1-methylpyrrolidinium, 1-methyl-1-n-octylpyrrolidinium, 1-methyl-1-pentylpyrrolidinium, tributylsulfonium, tricthylsulfonium.

In another embodiment, the salt added in step (b) comprises a neutralizing anion chosen from chloride, bromide, fluoride, iodide, acetate, propionate, butyrate, hexanoate, octanoate, decanoate, laurate, myristate, palmitate, palmitoleate, stearate, oleate, oxalate, succinate, bis(trifluoromethanesulfonyl)-imide, tetrafluoroborate, hexafluorophosphate, p-toluenesulfonate, trifluoromethanesulfonate, tetrachloroferrate, methanesulfonate, tribromide, hydrogen sulfate, thiocyanate, triflate, hexafluoroantimonate, dimethyl phosphate, methyl sulfate, dicyanamide, nitrate.

In some embodiments, the base pairs in the MdsRNA resulting from performing step (c) is at least about 40 bp, at least about 50 bp, at least about 60 bp, at least about 70 bp, at least about 80 bp, at least about 90 bp, at least about 100 bp, at least about 200 bp, at least about 350 bp, at least about 400 bp, at least about 500 bp, at least about 600 bp, at least about 700 bp, at least about 800 bp, at least about 900 bp, or at least about 1,000 bp in length (e.g., from about 40 bp to about 1,000 bp in length, or increment of this range). In an embodiment, the base pairs in the MdsRNA is from about 40 base pairs to about 1,000 base pairs. In another embodiment, the MdsRNA is from about 50 base pairs to about 900 base pairs. In an embodiment, the MdsRNA is from about 70 base pairs to about 800 base pairs. In another embodiment, the MdsRNA is from about 80 base pairs to about 700 base pairs. In an embodiment, the MdsRNA is from about 90 base pairs to about 600 base pairs. In an embodiment, the MdsRNA is from about 100 base pairs to about 500 base pairs. In another embodiment, the MdsRNA is from about 200 base pairs to about 400 base pairs, from about 25 to about 60, from about 25 to about 35, or from about 50 to about 60 base pairs in length. In some embodiments, the dsRNA in the MdsRNA is from about 25 bp to about 60 bp, from about 25 bp to about 35 bp, or from about 50 bp to about 60 base pairs in length.

In some embodiments, a MdsRNA sense strand is connected to the antisense strand. A sense strand may be connected to an antisense strand via a non-hybridizing hairpin or loop sequence. A loop sequence can be about 4 nucleotides to about 200 nucleotides or about 300 nucleotides (e.g., from about 4 nucleotides to about 200 nucleotides) nucleotides in length. In some embodiments, a loop is about 100 to about 200 nucleotides in length (Hauge et al. 2009). In some embodiments, a MdsRNA further comprises one or more additional sequences including, but not limited to, promoter sequences, 5′ sequences, 3′ sequences, terminator sequences, and polyA sequences.

Particular embodiments of the current disclosure call for methods to produce higher molecular polyalkyloxy polymers to be covalently bound to the 2′-OH position of the intersubunit linkages. Preferred embodiments of the disclosure call for methods to produce MdsRNAs in which a low percentage of the overall subunits are substituted with such a polymer (e.g., less than about 30%). In some embodiments, the percentage of the overall subunits are substituted with such a polymer is about 1%. In some embodiments, the percentage of substituted subunits is about 1% to about 30%. In other embodiments, the percentage of substituted subunits is about 1% to about 5%. In yet other embodiments, the percentage of substituted subunits is less than about 1%. In another embodiment, the percentage of substituted subunits is between about 0.1% and about 1%. The compounds of the disclosure show equal or superior properties to compounds having lower weight polyalkyloxy polymers at the 2′-OH position.

Embodiments and aspects in this and the other sections of the disclosure regarding the methods of making the compositions, nucleotide sequences and uses for target insects, fungi, weeds or acari are intended to be covered herein in this disclosure.

### Methods of Use

In another aspect of the disclosure, provided herein is a method of modifying the expression of a polynucleotide of interest in an insect comprising administering a composition of the disclosure.

In some embodiments, a target gene is selected such that inhibiting expression of the target gene kills, inhibits growth or appetite of, or slows reproduction of an animal, fungus, or weed. Inhibiting expression of the target gene can control, kill, inhibit growth or appetite of, or slow reproduction of the animal, fungus, or weed. In some embodiments, the insect, fungus, or plant is of agricultural significance. In some embodiments, an agriculturally significant animal, fungus, or plant is an insect, fungus, or weed. In an embodiment, the modified expression reduces the fertility rate of the target insect. In some embodiments, the described MdsRNAs can be used to control, kill, inhibit growth, appetite, or feeding of, or slow reproduction of an animal, fungus, or plant in an agricultural or urban setting. In some embodiments, a plant target gene is selected such that inhibiting expression of the gene in the plant increases plant growth, viability, quality, or yield.

In another embodiment, the MdsRNA comprises a sequence complementary to an expressed RNA in a target insect. For example, an efficacious sequence or target gene useful in the products and methods of the disclosure will inhibit the expression of the target gene, act on the mid gut of the insect and increase mortality or induce growth stunting, or stop instar development.

In an embodiment, the target insect is Diamondback moth (Plutella xylostella), Gypsy moth, Red imported fire ant (Solenopsis invicta), Fall armyworm, Colorado potato beetle, Canola flea beetle, Aedes aegypti, or Western corn root worm (Diabrotica virgifera virgifera). In another embodiment, the target insect is Pea aphid (Acyrthosiphon pisum), Soybean aphid, Piezodorus guildinii. In another embodiment the target is an acari, such as Verroa mite. In another embodiment the target is a weed, such as Palmer amaranth. In yet another embodiment, the target is a fungus such as Palmer amaranth, Fusarium graminearum (Gibberella zeae) or Botrytis. Accordingly, the MdsRNA used in the compositions of the disclosure will be a nucleotide sequence that can inhibit the expression of target genes or regions in these insects. For example, the MdsRNA comprises a sequence complementary to a target region in Diamondback moth, such as but not limited to AChE2, P450, CYP6BF1v1, DOUX, Cytokine receptor DOMELESS, Protein MESH transcript variant X1, Venom carboylesterase-6 and VPASE-E. In another embodiment, the MdsRNA comprises a sequence complementary to a target region in Fall armyworm, such as but not limited to P450, Cytokine receptor DOMELESS, VPASE, Dredd, Protein MESH transcript variant X1, P450 CYP9A58, P450 CYP321A8, P450 CYP6B2-like.

In another embodiment, the MdsRNA comprises a sequence complementary to a target region in Western corn root worm, such as but not limited to SNF7.

In an embodiment, the MdsRNA comprises a sequence selected from one of SEQ ID Nos: 1-298 and its perfect or imperfect reverse complementary strand. In a particular embodiment, the sequence is selected from one of SEQ ID Nos: 13 or 248-251 and its perfect or imperfect reverse complementary strand. In an embodiment, the target insect is a Lepidopteran and can be targeted using any one of SEQ ID Nos: 1-298 and its perfect or imperfect reverse complementary strand thereof for each sequence specified. In an embodiment, the Lepidopteran is targeted using any one of SEQ ID Nos: 13 or 248-251.

In an embodiment, the target insect is a Lepidopteran. In an embodiment, the Lepidopteran is an army worm, corn ear worm, cabbage butterfly, or cotton boll worm.

In another embodiment, the MdsRNA comprises a sequence complementary to the P450, CYP6FB1v1, MESH, AChE2, VPASE, DOMELESS, DOUX or Venom target region in a Lepidopteran.

In an embodiment, the treatment of at least about 30% control of the target insect. In an embodiment, the treatment of at least about 50% control of the target insect. In an embodiment, the treatment shows greater than about 50% control of the target insect. In an embodiment, the treatment shows between about 50% to about 90% control of the target insect. In an embodiment, the treatment shows between about 50% to about 75% control of the target insect. In an embodiment, the treatment shows between about 30% to about 50% control of the target insect.

The compositions and methods herein described are further illustrated in the following examples, which are provided by way of illustration and are not intended to be limiting. It will be appreciated that variations in proportions and alternatives in elements of the components shown will be apparent to those skilled in the art and are within the scope of embodiments of the present disclosure. Theoretical aspects are presented with the understanding that Applicant does not seek to be bound by the theory presented. All parts or amounts, unless otherwise specified, are by weight.

### Agricultural/Agrochemical Compositions

In some embodiments, compositions containing the described MdsRNAs are described. In some embodiments, the MdsRNA-containing compositions are formulated for agricultural application (agrochemical compositions).

As used herein, an agrochemical composition comprises an effective amount of at least one MdsRNA and optionally one or more acceptable carriers or excipients. Carriers and excipients are substances other than the MdsRNA that have been appropriately evaluated for safety and are intentionally included in a composition. Excipients may act to a) aid in processing of the MdsRNA during manufacture, b) protect, support or enhance stability or bioavailability of the MdsRNA, c) assist in product identification, and/or d) enhance any other attribute of the overall safety, effectiveness, of delivery of the MdsRNA during storage or use. An acceptable carrier or excipient may or may not be an inert substance. As used herein, “effective amount,” refers to that amount of a MdsRNA to produce the intended result.

Carrier and excipients include, but are not limited to, stability enhancers, absorption enhancers, anti-adherents, anti-foaming agents, anti-oxidants, binders, buffering agents (pH regulating agents), chelating agents, coating agents, colors, delivery enhancers, dextran, dextrose, diluents, disintegrates, dispersants, emulsifiers, extenders, fillers, foam control agents, glidants, humectants, lubricants, oils, pigments, polymers, preservatives, saline, salts, solvents, sugars, surfactants, suspending agents, sustained release matrices, sweeteners, thickening agents, tonicity agents, vehicles, water-repelling agents, and wetting agents.

In some embodiments, an agrochemical composition comprises one or more adjuvants or surfactants. In some embodiments, the one or more adjuvant or surfactants are independently selected from anionic surfactants, cationic surfactants, amphoteric surfactants, nonionic surfactants, anti-condensates, thickeners, emulsifiers, spreaders, stickers, organosilanes, fatty esters and oils. In a particular embodiment, the one or more adjuvants or surfactants are optionally selected from non-ionic, organo silicone surfactants (e.g., KINETIC® from the Helena company; nonionic organosilicone-based wetter/spreader/penetrant spray adjuvant), DYNE-AMIC® (from the Helena company; blend of highly refined methylated seed oils in combination with specialized organosilicone-based nonionic surfactants) and SILWET® (from Momentive; nonionic surfactant).

In some embodiments, the stability enhancer comprises cetyltrimethylammonium bromide (deca hexyl trimethyl ammonium bromide) or cetyltrimethylammonium chloride (deca hexyl trimethyl ammonium chloride).

In some embodiments, an agrochemical composition comprises one or more agents selected from an herbicide, a fungicide or biofungicide, insecticide or bioinsecticide, acaricide or bioacaricide, and fertilizer.

The described MdsRNAs and compositions containing MdsRNAs can be processed in a number of different ways known to those skilled in the art to facilitate application of such material onto plants or into baits and for use in the field or in urban environments. The described MdsRNAs and compositions comprising MdsRNAs disclosed herein can be packaged or included in a kit, container, pack, or dispenser.

In some embodiments, an agrochemical composition contains two or more different MdsRNAs. The MdsRNAs may have different antisense sequences complementary to the same target gene, different antisense sequences complementary to different target genes in the same or different hosts, different or similar lengths, or different or similar post transcriptional modification.

In some embodiments, an agrochemical composition is an emulsifiable agricultural concentrate. In some embodiments, an emulsifiable agricultural concentrate further contains a least one agent that can be, but is not limited to, carrier, or organic solvent, surfactant, excipient, herbicide, fungicide, insecticide, fertilizer, or combinations thereof.

In some embodiments, an agrochemical composition contains one or more herbicide. Non-limiting examples of suitable herbicides include, but are not limited to, imidazolinone, acetochlor, acifluorfen, aclonifen, acrolein, AKH-7088, alachlor, alloxydim, ametryn, amidosulfuron, amitrole, ammonium sulfamate, anilofos, asulam, atrazine, azafenidin, azimsulfuron, BAS 620H, BAS 654 00H, BAY FOE 5043, benazolin, benfluralin, benfuresate, bensulfuron-methyl, bensulide, bentazone, benzofenap, bifenox, bilanafos, bispyribac-sodium, bromacil, bromobutide, bromofenoxim, bromoxynil, butachlor, butamifos, butralin, butroxydim, butylate, cafenstrole, carbetamide, carfentrazone-ethyl, chlormethoxyfen, chloramben, chlorbromuron, chloridazon, chlorimuron-ethyl, chloroacetic acid, chlorotoluron, chlorpropham, chlorsulfuron, chlorothal-dimethyl, chlorthiamid, cinmethylin, cinosulfuron, clethodim, clodinafop-propargyl, clomazone, clomeprop, clopyralid, cloransulam-methyl, cyanazine, cycloate, cyclosulfamuron, cycloxydim, cyhalofop-butyl, 2,4-D, daimuron, dalapon, dazomet, 2,4 DB, desmedipham, desmetryn, dicamba, dichlobenil, dichlorprop, dichlorprop-P, diclofop-methyl, difenzoquat metilsulfate, diflufenican, dimefuron, dimepiperate, dimethachlor, dimethametryn, dimethenamid, dimethipin, dimethylarsinic acid, dinitramine, dinocap, dinoterb, diphenamid, diquat dibromide, dithiopyr, diuron, DNOC, EPTC, esprocarb, ethalfluralin, ethametsulfuron-methyl, ethofumesate, ethoxysulfuron, etobenzanid, fenoxaprop-P-ethyl, fenuron, ferrous sulfate, flamprop-M, flazasulfuron, fluazifop-butyl, fluazifop-P-butyl, fluchloralin, flumetsulam, flumiclorac-pentyl, flumioxazin, fluometuron, fluoroglycofen-ethyl, flupoxam, flupropanate, flupyrsulfuron-methyl-sodium, flurenol, fluridone, flurochloridone, fluroxypyr, flurtamone, fluthiacet-methyl, fomesafen, fosamine, glufosinate-ammonium, glyphosate, glyphosinate, halosulfuron-methyl, haloxyfop, HC-252, hexazinone, imazamethabenz-methyl, imazamox, imazapyr, imazaquin, imazethapyr, imazosuluron, imidazilinone, indanofan, ioxynil, isoproturon, isouron, isoxaben, isoxaflutole, lactofen, lenacil, linuron, MCPA, MCPA-thioethyl, MCPB, mecoprop, mecoprop-P, mefenacet, metamitron, metazachlor, methabenzthiazuron, methylarsonic acid, methyldymron, methyl isothiocyanate, metobenzuron, metobromuron, metolachlor, metosulam, metoxuron, metribuzin, metsulfuron-methyl, molinate, monolinuron, naproanilide, napropamide, naptalam, neburon, nicosulfuron, nonanoic acid, norflurazon, oleic acid (fatty acids), orbencarb, oryzalin, oxadiargyl, oxadiazon, oxasulfuron, oxyfluorfen, paraquat dichloride, pebulate, pendimethalin, pentachlorophenol, pentanochlor, pentoxazone, petroleum oils, phenmedipham, picloram, piperophos, pretilachlor, primisulfuron-methyl, prodiamine, prometon, prometryn, propachlor, propanil, propaquizafop, propazine, propham, propisochlor, propyzamide, prosulfocarb, prosulfuron, pyraflufen-ethyl, pyrazolynate, pyrazosulfuron-ethyl, pyrazoxyfen, pyributicarb, pyridate, pyriminobac-methyl, pyrithiobac-sodium, quinclorac, quinmerac, quinoclamine, quizalofop, quizalofop-P, rimsulfuron, sethoxydim, siduron, simazine, simetryn, sodium chlorate, STS system (sulfonylurea), sulcotrione, sulfentrazone, sulfometuron-methyl, sulfosulfuron, sulfuric acid, tar oils, 2,3,6-TBA, TCA-sodium, tebutam, tebuthiuron, terbacil, terbumeton, terbuthylazine, terbutryn, thenylchlor, thiazopyr, thifensulfuron-methyl, thiobencarb, tiocarbazil, tralkoxydim, tri-allate, triasulfuron, triaziflam, tribenuron-methyl, triclopyr, trietazine, trifluralin, triflusulfuron-methyl, vernolate, and combinations thereof.

In some embodiments, an agrochemical composition contains one or more fungicides.

Suitable biofungicides include, but are not limited to, Streptomyces lydicus, Bacillus amyloliquefaciens, Bacillus subtillis GB03, Ulocladium oudemansii U3, Streptomyces griseoviridis, and Gliocladium catenulatum JII446.

Suitable fungicides include, but are not limited to, carbamate fungicides such as 3,3′-ethylenebis(tetrahydro-4,6-dimethyl-2H-1,3,5-thiadiazine-2-thione), zinc or manganese ethylenebis(dithiocarbamate), bis(dimethyldithiocarbamoyl)disulfide, zinc propylene-bis-(dithiocarbamate), bis(dimethyldithiocarbamoyl)ethylenediamine, nickel dimethyl-dithiocarbamate, methyl 1-(butylcarbamoyl)-2-benzimidazolecarbamate, 1,2bis(3-methoxycarbonyl-2-thioureido)benzene, 1-isopropylcarbamoyl-3-(3,5-dichlorophenyl)-hydantoin, potassium N-hydroxymethyl-N-methyldithiocarbamate, and 5methyl-10-butoxycarbonylamino-10,11-dehydrodibenzo (b,f)azepine; pyridine fungicides such as zinc bis(1-hydroxy-2(1H)pyridinethionate) and 2-pyridinethiol-1-oxide sodium salt; phosphorus fungicides such as O,O-diisopropyl S-benzylphosphorothioate and O-ethyl S,S-diphenyldithiophosphate; phthalimide fungicides such as N(2,6diethylphenyl)phthalimide and N-(2,6-diethylphenyl)-4-methylphthalimide; dicarboxyimide fungicides such as N-trichloromethylthio-4-cyclohexene-1,2-dicarboxyimide and N-tetrachloroethylthio-4-cyclohexene-1,2-dicarboxyimide; oxathine fungicides such as 5,6-dihydro-2-methyl-1,4-oxathinc-3-carboxanilido-4,4-dioxide and 5,6-dihydro-2-methyl-1,4-oxathine-3-carboxanilide; naphthoquinone fungicides such as 2,3-dichloro-1,4-naphthoquinone, 2-oxy-3-chloro-1,4-naphthoquinone copper sulfate; pentachloronitrobenzene; 1,4-dichloro-2,5-dimethoxybenzene; 5-methyl-s-triazol (3,4b)benzthiazole; 2-(thiocyanomethylthio) benzothiazole; 3-hydroxy-5-methylisooxazole; N2,3-dichlorophenyltetrachlorophthalamic acid; 5-ethoxy-3-trichloromethyl-1-2,4-thiadiazole; 2,4-dichloro-6-(O-chloroanilino)-1,3,5-triazine; 2,3dicyano-1,4-dithio-anthraquinone; copper 8-quinolinate, polyoxine; validamycin; cycloheximide; iron methanearsonate; diisopropyl-1,3-dithiolane-2-iridene malonate; 3allyloxy-1,2-benzoisothiazol-1,1-dioxide; kasugamycin; Blasticidin S; 4,5,6,7tetra-chlorophthalide; 3-(3,5-dichlorophenyl)-5-ethenyl-5-methyloxazolizine-2,4-dione; N-(3,5-dichlorophenyl)-1,2-dimethylcyclopropane-1,2-dicarboxyimide; S-n-butyl-5′-para-t-butylbenzyl-N-3-pyridyldithiocarbonylimidate; 4-chlorophenoxy-3,3-dimethyl-1-(1H,1,3,4-triazol-1-yl)-2-butanone; methyl-D,L-N-(2,6-dimethylphenyl)-N-(2′-methoxyacetyl) alaninate; N-propyl-N-[2-(2,4,6-trichlorophenoxy)ethyl]phosphor-1-carboxamide; N-(3,5-dichlorophenyl)-succinimide; tetrachloroisophthalonitrile; 2dimethylamino-4-methyl-5-n-butyl-6-hydroxypyrimidine; 2,6-dichloro-4-nitroaniline; 3methyl-4-chlorobenzthiazol-2-one; 1,2,5,6-tetrahydro-4H-pyrrolo[3,2,1-I,j]phosphor-2-one; 3′-isopropoxy-2-methyl-benzanilide; 1-[2-(2,4-dichlorophenyl)-4-ethyl-1,3-dioxorane-2-ylmethyl]-1H,1,2,4-triaz ol; 1,2-benzisothiazoline-3-one; basic copper chloride; basic copper sulfate; N′-dichlorofluoromethylthio-N,N-dimethyl-N-phenylsulfamide; ethyl-N-(3-dimethylamino-propyl)thiocarbamate hydrochloride; piomycin; S,S-6-methylquinoxaline-2,3-diyldithio-carbonate; complex of zinc and manneb; di-zinc bis(dimethyldithiocarbamate)ethylenebis(dithiocarbamate) and glyphosate; chlorothalonil-based fungicides, strobilurin-based fungicides such as azoxystrobin, pyraclostrobin, and trifloxystrobin; and triazole-based fungicide such as myclobutanil, propiconazole, tebuconazol, tetraconazole, and combinations thereof.

In some embodiments, an agrochemical composition contains one or more insecticides.

Suitable insecticides include but are not limited to biological insecticides containing different strains of Bacillus thuringiensis; pyrethrins and pyrethroids such as permethrin, cypermethrin and deltamethrin; spinosyns such as Spinosad and spinetoram; diamides such as chlorantraniliprole and cyantraniliprole; avermectins such as abamectin.

Suitable insecticides include, but are not limited to, phosphoric insecticides such as O,O-diethyl O-(2-isopropyl-4-methyl-6-pyrimidinyl)phosphorothioate, O,O-dimethyl S-2-[(ethylthio)ethyl]phosphorodithioate, O,O-dimethyl O-(3-methyl-4-nitrophenyl)-thiophosphate, O,O-dimethyl S—(N-methylcarbamoylmethyl)-phosphorodithioate, O,O-dimethyl S—(N-methyl-N-formylcarbamoylmethyl)phosphoro-dithioate, O,O-dimethyl S-2-[(ethylthio)ethyl]phosphorodithioate, O,O-diethyl S-2-[(ethylthio)ethyl]phosphorodithioate, O,O-dimethyl-1-hydroxy-2,2,2-trichloroethylphophonate, O,O-diethyl-O-(5-phenyl-3-isooxazolyl)phosphorothioate, O,O-dimethyl O-(2,5-dichloro-4-bromophenyl)phosphorothioate, O,O-dimethyl O-(3-methyl-4-methylmercaptophenyl)-thiophosphate, O-ethyl O-p-cyanophenyl phenyl-phosphorothioate, O,O-dimethyl-S-(1,2-dicarbocthoxyethyl)phosphorodithioate, 2-chloro-(2,4,5-trichlorophenyl)vinyldimethyl phosphate, 2-chloro-1-(2,4-dichlorophenyl)-vinyldimethyl phosphate, O,O-dimethyl O-p-cyanophenyl phosphorothioate, 2,2-dichlorovinyl dimethyl phosphate, O,O-diethyl O-2,4-dichlorophenyl phosphorothioate, ethyl mercaptophenylacetate O,O-dimethyl phosphoro-dithioate, S-[(6-chloro-2-oxo-3-benzooxazolinyl)methyl] O,O-diethyl phosphorodithioate, 2-chloro-1-(2,4-dichlorophenyl) vinyl diethylphosphate, O,O-diethyl O-(3-oxo-2-phenyl-2H-pyridazine-6-yl)phosphorothioate, O,O-dimethyl S-(1-methyl-2-ethylsulfinyl)-ethyl phosphorothiolate, O,O-dimethyl S-phthalimidomethyl phosphorodithioate, O,O-diethyl S-(N-cthoxycarbonyl-N-methylcarbamoylmethyl)phosphorodithioate, O,O-dimethyl S-[2-methoxy-1,3,4-thiadiazol-5-(4H)-I-(4)-methyl] dithiophosphate, 2-methoxy-4H-1,3,2-benzooxaphosphorine 2-sulfide, O,O-diethyl O-(3,5,6-trichloro-2-pyridyl)phosphorothiate, O-ethyl O-2,4-dichlorophenyl thionobenzene phosphonate, S-[4,6-diamino-s-triazine-2-yl-methyl] O,O-dimethyl phosphorodithioate, O-ethyl O-p-nitrophenyl phenyl phosphorothioate, O,S-dimethyl N-acetyl phosphoroamidothioate, 2-diethylamino-6-methylpyrimidine-4-yl-diethylphosphorothionate, 2-diethylamino-6-methylpyrimidine-4-yl-dimethylphosphorothionate, O,O-diethyl O—N-(methylsulfinyl)phenyl phosphoro-thioate, O-ethyl S-propyl O-2,4-dichlorophenyl phosphorodithioate and cis-3-(dimethoxyphosphinoxy)N-methyl-cis-crotone amide; carbamate insecticides such as 1-naphthyl N-methylcarbamate, S-methyl N-[methylcarbamoyloxy]thioacetoimidate, m-tolyl methylcarbamate, 3,4-xylyl methylcarbamate, 3,5-xylyl methylcarbamate, 2-sec-butylphenyl N-methylcarbamate, 2,3-dihydro-2,2-dimethyl-7-benzofuranylmethyl-carbamate, 2-isopropoxyphenyl N-methylcarbamate, 1,3-bis(carbamoylthio)-2-(N,N-dimethylamino) propane hydrochloride and 2-diethylamino-6-methylpyrimidine-4-yl-dimethylcarbamate; and other insecticides such as N,N-dimethyl N′-(2-methyl-4-chlorophenyl) formamidine hydrochloride, nicotine sulfate, milbemycin, 6-methyl-2,3-quinoxalinedithiocyclic S,S-dithiocarbonate, 2,4-dinitro-6-sec-butylphenyl dimethyl-acrylate, 1,1-bis(p-chlorophenyl) 2,2,2-trichloroethanol, 2-(p-tert-butylphenoxy)isopropyl-2′-chloroethylsulfite, azoxybenzene, di-(p-chlorophenyl)-cyclopropyl carbinol, di[tri(2,2-dimethyl-2-phenylethyl)tin]oxide, 1-(4-chlorophenyl)-3-(2,6-difluorobenzoyl)urea, S-tricyclohexyltin O,O-diisopropylphosphorodithioate, and combinations thereof.

In some embodiments, an agrochemical composition contains one or more fertilizers. A variety of fertilizers are suitable for inclusion in the compositions. The fertilizer can be a single nutrient fertilizer (N, P, or K), binary fertilizer (e.g., NP, NK, or PK), a NPK fertilizer, or a multinutrient fertilizer (e.g., may provide one or more of calcium, magnesium, sulfur, copper, iron, manganese, molybdenum, zinc, boron, silicon, cobalt, or vanadium). The fertilizer can be of natural origin or synthetic origin. The fertilizer can be liquid or solid, and may provide slow or controlled release.

In some embodiments, the MdsRNAs comprise less than 50% by weight of a composition (e.g., from about 0.001% by weight to about 50% by weight of a composition). In some embodiments, the amount of MdsRNA in an agriculture composition is less than 5% by weight of the composition. In some embodiments, the MdsRNA is present in the composition in an amount less than about 1% by weight, less than about 0.9% by weight, less than about 0.8% by weight, less than about 0.7% by weight, less than about 0.6% by weight, less than about 0.5% by weight, less than about 0.4% by weight, less than about 0.3% by weight, less than about 0.2% by weight, less than about 0.1% by weight, less than about 0.05% by weight, less than about 0.01% by weight, or less than about 0.001% by weight of the composition.

In some embodiments, the agrochemical composition is formulated as a liquid. Liquid formulations can be prepared by mixing the MdsRNA and other agents in a liquid until dissolution of all the components is achieved in the weight percentages described below. The liquid can be an aqueous, ionic, or organic liquid. Suitable liquids include, but are not limited to, water, alcohols (e.g. methanol and ethanol), ketones (e.g. acetone, methyl ethyl ketone and cyclohexanone), aromatic hydrocarbons (e.g. benzene, toluene, xylene, ethylbenzene and methylnaphthalene), aliphatic hydrocarbons (e.g. hexane and kerosene), esters (e.g. ethyl acetate and butyl acetate), nitriles (e.g. acetonitrile and isobutyronitrile), ethers (e.g. dioxane and diisopropyl ether), acid amides (e.g. dimethylformamide and dimethylacetamide), and halogenated hydrocarbons (e.g. dichloroethane, trichloroethylene and carbon tetrachloride).

In some embodiments, the liquid formulation is an aqueous formulation. In some embodiments, an aqueous formulation contains only water, the MdsRNA and other agents. In some embodiments, additional compounds, solvents, or adjuvants are provided with the aqueous formulation.

In some embodiments, the agrochemical composition is formulated as a powder or dust. The powder or dust can be granulated to be suitable for applying the powder or dust directly to a crop (i.e., by dusting the crop), or it can be granulated for eventual dissolution in a solvent such as water. In some embodiments, the composition is a lyophilisate. Typically, the MdsRNA and the other agents are lyophilized together. In some embodiments, one or more MdsRNAs and the other agents can be lyophilized separately.

A variety of suitable solid and gaseous carriers can be utilized in the compositions. Suitable solid carriers include, but are not limited to, fine powders or granules of clays (e.g. kaolin clay, diatomaceous earth, synthetic hydrated silicon dioxide, attapulgite clay, bentonite and acid clay), talcs, bulking agents, inorganic minerals (e.g., sericite, powdered quartz, powdered sulfur, activated carbon, calcium carbonate and hydrated silica), and salts for chemical fertilizers (e.g. ammonium sulfate, ammonium phosphate, ammonium nitrate, urea and ammonium chloride). Suitable gaseous carriers include, for example, butane gas, carbon dioxide, and fluorocarbon gas.

In some embodiments, an agrochemical composition includes a dispersant. Examples of dispersants include, but are not limited to, methyl cellulose, polyvinyl alcohol, sodium lignin sulfonates, polymeric alkyl naphthalene sulfonates, sodium naphthalene sulfonate, polymethylene bisnaphthalene sulfonate, neutralized polyoxyethylated derivatives, and ring-substituted alkyl phenol phosphates. Stabilizers may also be used to produce stable emulsions. Exemplary stabilizers include, but are not limited to magnesium, aluminum silicate, and xanthan gum.

In some embodiments, an agrochemical composition is formulated as a spray in the form of an aerosol. When formulated as an aerosol spray, the composition is generally charged in a container under pressure together with a propellant. Examples of suitable propellants include fluorotrichloromethane and dichlorodifluoromethane.

In some embodiments, an agrochemical composition includes a seed. In some embodiments, an agrochemical composition comprises an antifungal MdsRNA and a seed. In some embodiments, an agrochemical composition comprises a MdsRNA, a seed, and further comprises a fungicide.

In some embodiments, the amount of the MdsRNA in a fungicidal composition (agrochemical composition containing a fungicide) is less than about 5% by weight, less than about 1% by weight, less than about 0.9% by weight, less than about 0.8% by weight, less than about 0.7% by weight, less than about 0.6% by weight, less than about 0.5% by weight, less than about 0.4% by weight, less than about 0.3% by weight, less than about 0.2% by weight, less than about 0.1% by weight, less than about 0.05% by weight, less than about 0.01% by weight, or less than about 0.001% by weight of the fungicidal composition (e.g., from about 0.001% by weight to about 5% by weight of the fungicidal composition). The weight of the fungicidal composition does not include the weight of the seed.

In some embodiments, the fungicidal composition is present inside the seed coat, or internal to the seed. In some embodiments, the fungicidal composition is formed over the seed such that it covers the exterior of the seed, either fully or partially. Methods for coating a seed include those known in the art.

### Methods for Controlling Agricultural Pests

In some embodiments, MdsRNAs or compositions containing MdsRNAs are used to control agricultural pests or treat agricultural pest infestation. The MdsRNAs can be administered to the pest, to an area occupied by the pest, or to a food source of the pest.

In some embodiments, methods are provided for treating for or controlling pests. In some embodiments, the pest is an insect, fungus, acari or weed. The methods comprise applying a composition comprising one or more described MdsRNAs to an area to be treated. In some embodiments, the MdsRNA is present in the composition in an amount of less than 5% by weight. In some embodiments, the composition is applied directly to a surface. In some embodiments, the surface is a plant surface upon which the targeted insect or fungal pest feeds.

In some embodiments, the gene expression level and/or mRNA level of a target gene in a target host is reduced by at least about 5%, 10%, 20%, 30%, 40%, 50%, 60%, 70%, 80%, or 90%, following application of MdsRNAs or MdsRNA-containing composition. In some embodiments, mortality of the agricultural pest in increased at least 5%, 10%, 20%, 30%, 40%, 50%, 60%, 70%, 80%, or 90% following application of MdsRNAs or MdsRNA-containing composition.

As used herein, controlling a pest means to reduce crop damage or decreased yield caused by the pest, or to increase morbidity, inhibit growth or appetite or feeding of, or slow reproduction of the pest compared with the damage, decreased yield morbidity, growth, appetite, feeding, or reproduction as measured in the absence of treatment with MdsRNAs.

### Crop Application

In some embodiments, methods of reducing expression of a target gene in a target plant other than a weed are described. The methods comprise applying a composition containing one or more of the described MdsRNAs to the plant. In some embodiments, the plant is a crop plant. A crop plant is a plant that can be grown and harvested for profit or subsistence. A crop plant can be, but is not limited to, a food plant, horticultural plant, floriculture plant, or industrial plant. In some embodiments, the plant is a cultivated plant. The plant can be in a laboratory, greenhouse, nursery, field, orchard or other agricultural setting, garden, or another natural or urban setting. In some embodiments, a target plant is a plant considered desirable in a particular situation or location.

### Insect Infestation

In some embodiments, the animal is an insect. In some embodiments, the insect is a Coleopteran (such as a beetle). A Coleopteran can be, but are not limited to, bark beetle, elm leaf beetle, Asian longhorn beetle, death watch beetle, mountain pine beetle, coconut hispine beetle or the Colorado potato beetle. In some embodiments, the insect is a Lepidopteran (such as a butterfly or moth). A Lepidopteran can be, but is not limited to, army worm, corn ear worm, cabbage butterfly, or cotton boll worm. In some embodiments, the insect is a Hymenopteran (such as sawflies, wasps, bees, ants). A Hymenopteran can be, but is not limited to, fire ant, Argentine ant, carpenter ant, leafcutter ant, army ant, wheat stem sawfly, larch sawfly, spruce sawfly, or bed bug. In some embodiments, the insect is a Dipteran (such as a fly). A Dipteran can be, but is limited to, fly, mosquito, gnat, or leafminer. In some embodiments, the insect is a Hemipteran (such as a true bug). A Hemipteran can be, but is not limited to, aphid, hopper, bug, whiteflies, mealybug, or flea. In some embodiments, the insect is a Western corn root worm.

In some embodiments, the insect is an insect having resistance to one or more conventional known insecticides. In some embodiments, the insect, such as a Red imported fire ant has the potential to have a negative impact on biodiversity (Wojcik et al. 2001 and/or resistance to insecticides (Zhang et al. 2016). In some embodiments, the insect, such as a mosquito, has the potential to impact human health as a vector for disease, such as, but not limited to, Malaria, Dengue, Zika and Chikungunya (Hemingway et al. 2004). In some embodiments, the insect, such as Asian citrus psyllid, is a vector of the citrus greening disease (Tiwari et al. 2011).

Coleopteran, Lepidopteran, Hymenopteran, Dipteran, and Hemipteran insect pests are known to be susceptible to RNAi introduced either by direct injection or by feeding on plant matter treated with siRNA precursors. Field application of naked RNAs is generally impractical due to the sensitivity of RNA to environmental specific and non-specific degradation (Baum 2016). Furthermore, RNA is highly susceptible to degradation during the course of feeding and in transit through the insect gut. For example, in general, the Lepidoptera seem to degrade RNA much more aggressively than the Coleoptera, which may account for their relatively poor susceptibility to RNAi mediated control methods. The stability of the described MdsRNAs serves to protect the MdsRNA from host nucleases before delivery to the RNAi pathway, and limits non-specific environmental degradation. The described MdsRNAs are nevertheless sufficiently biodegradable to be considered environmentally safe.

A composition comprising one or more MdsRNAs or MRNAs can be applied to a plant prior to infection to prevent an insect infection. The composition may also be applied after the appearance of signs of infection to treat an insect infection. The composition can be applied by a variety of methods depending on the plant part to be treated. By way of example, the composition can be applied to a plant seed prior to planting to prevent insect infection of the seed. The composition can be applied to the soil at the time of planting or just before planting to prevent insect infestation of the newly planted seed (i.e., as a pre-emergent). In some embodiments, the composition can be applied to a plant after its germination or to the foliage of the plant after emergence to either treat or prevent insect infestation (i.e., as a post-emergent). In an exemplary embodiment, the application occurs during the stages of germination, seedling growth, vegetative growth, and reproductive growth. In some embodiments, application occurs during vegetative and reproductive growth stages.

Applying the composition to a pre-emergent seed may involve various seed coating techniques such as film coating, pelleting, encapsulation, drum coating, and fluidized bed coating. Applying to a post-emergent plant may involve spraying or crop dusting techniques.

An effective amount of the composition can be applied to a plant or seed by several methods generally known in the art. As will be appreciated by a skilled artisan, the amount of composition comprising an “an effective amount” can and will vary depending upon the plant and its stage of production, the fungal target, and environmental conditions. Generally speaking, for a typical application, the plant or its progeny is treated with an amount of the composition sufficient to provide a concentration of active ingredients from about 0.01 mg/kg to about 10% by weight. It is envisioned that the method may involve more than one application of the composition to the plant or its progeny. For example, the number of applications may range from about 1 to about 5 or more. The applications, as detailed herein, can be applied at the same or different stages of the plant's life cycle.

### Fungal Infection

In some embodiments, the MdsRNAs or MRNAs are used to treat or prevent fungal infection. In some embodiments, the fungus can be, but is not limited to, a Hypocrealesan, Venturia, Podosphaera, Erysiphe, Monolinia, Mycosphaerella, Uncinula; Basidiomycete, Hemileia, Rhizoctonia, Puccinia, Fungi imperfecti, Botrytis, Helminthosporium, Rhynchosporium, Fusarium, Septoria, Cercospora, Alternaria, Pyricularia, Pseudocercosporella, Oomycete fungi, Phytophthora, Peronospora, Bremia, Pythium, Plasmopara, Phakopsora Pachyrhizi, Phakopsora meibomiac, Scleropthora macrospora, Sclerophthora rayissiae, Sclerospora graminicola, Peronosclerospora sorghi, Peronosclerospora philippinensis, Peronosclerospora sacchari Peronosclerospora maydis, Physopella zeae, Cercospora zeae-maydis, Colletotrichum graminicola, Hypocreale, Gibberella zeae, Exserohilum turcicum, Kabatiellu zeae, Bipolaris maydis, Gibberella avenacea, Fusarium culmorum, Fusarium oxysporum, Fusarium sporotrichioides, or Fusarium graminearum. In some embodiments, treatment of Fusarium graminearum can reduce the production of mycotoxins, the risk of emergence of resistance to fungicides based on demethylation inhibitors (DMI), orcarcinogenicity concerns about conventional DMI like Tebuconazole.

In some embodiments, the described agrochemical compositions can be applied to a plant prior to infection to prevent a fungal infection. In some embodiments, the described agrochemical compositions can be applied to a plant after the appearance of signs of infection to treat a fungal infection. The composition can be applied by a variety of methods depending on the plant part to be treated. By way of example, the composition can be applied to a plant seed prior to planting to prevent fungal infection of the seed. The composition can be applied to the soil at the time of planting or just before planting to prevent microbial infestation of the newly planted seed (i.e., as a pre-emergent). In some embodiments, the composition can be applied to a plant after its germination or to the foliage of the plant after emergence to either treat or prevent microbial infestation (i.e., as a post emergent). In an exemplary embodiment, the application occurs during the stages of germination, seedling growth, vegetative growth, and reproductive growth. More typically, applications occur during vegetative and reproductive growth stages.

Applying the composition to a pre-emergent seed may involve various seed coating techniques such as film coating, pelleting, encapsulation, drum coating, and fluidized bed coating. Applying to a post-emergent plant may involve spraying or crop dusting techniques.

An effective amount of the composition can be applied to a plant or seed by several methods generally known in the art. As will be appreciated by a skilled artisan, the amount of composition comprising an “an effective amount” can and will vary depending upon the plant and its stage of production, the fungal target, and environmental conditions. Generally speaking, for a typical application, the plant or its progeny is treated with an amount of the composition sufficient to provide a concentration of active ingredients from about 0.01 mg/kg to about 5,000 mg/kg. It is envisioned that the method may involve more than one application of the composition to the plant or its progeny. For example, the number of applications may range from about 1 to about 5 or more. The applications, as detailed herein, can be made at the same or different stages of the plant's life cycle.

### Weeds

The compostions of the disclosure can be used to control, prevent, eliminate, slow the growth of weeds. A weed is a plant considered undesirable in a particular situation or location. A weed can be, but is not limited to, Palmer Amaranth, Common Lambsquarters, Horseweed, Morning Glory, Waterhemp, Nutsedge, Kochia, Common Ragweed, Giant Ragweed, or Nightshade.

### EXAMPLES

### Section 1: Preparation, Formulation, and Efficacy Targeting Agricultural Pests of MdsRNAs and MRNAs Modified with Polymers or Combinations of Polymers/Small Molecules

### Example 1. Preparation of High Molecular Weight Polyalkyloxy Polymer dsRNA in DMSO

### Activation of PEG

One equivalent PEG-acetic acid was dissolved in dimethyl sulfoxide (DMSO) at 50 mg/ml and to this solution was added 1.05 equivalent of carbonyldiimidazole (CDI). The reaction mixture was stirred for at least 2 hours, preferably overnight under argon. This solution was then added directly to dsRNA following the ionic solvation.

Reaction of dsRNA with Activated PEG

dsRNA obtained using production methods known in the art, e.g., as described by Timmons, L. et al., Nature 395.6705 (1998): 854-854, was isolated using methods known in the art, e.g., as described by Meyerink, B. et al., The Journal of Undergraduate Research 7.1 (2009): 6. Benzyl triethyl ammonium chloride (Benzyl-TriBA-Cl) was dissolved in DMSO at 200 mM. An aliquot of this solution was added to freeze-dried dsRNA in a reaction tube. The tube was warmed to 65° C. in a shaker for 1 hr. Then the temperature was reduced to 55° C. and catalytic amount of pyridine was added followed by a 200 μg/μL solution of DMAP. A 200 μg/μL solution of activated PEG was added in 1-3 portions. The mixture was kept at 55° C. for 90 min and then was quenched with 500 mM citrate pH 4.5, and diluted to 40 mL with water. The resulting mixture containing the crude PEG-dsRNA was purified using tangential flow filtration, TFF, to yield MdsRNA with >90% purity.

The following MdsRNAs were prepared according to the procedure described above:

TABLE 1
Modified MdsRNA Compounds Synthesized
by Procedure of Example 1.

| MdsRNA | Sequence ID No. | Modifier | Input |
|---|---|---|---|
| 1 | dsP450 (115) | PEG 10K | 0.025 |
| 2 | dsP450 (115) | PEG 10K | 0.05 |
| 3 | dsP450 (115) | PEG 10K | 0.1 |
| 4 | dsP450 (115) | PEG 10K | 0.2 |
| 5 | dsP450 (115) | PEG 5K | 0.1 |
| 6 | dsP450 (115) | PEG 5K | 0.2 |
| 7 | dsP450 (115) | PEG 5K | 0.3 |
| 8 | dsVPASE (7) | PEG 10K | 0.025 |
| 9 | dsVPASE (7) | PEG 10K | 0.05 |
| 10 | dsVPASE (7) | PEG 10K | 0.1 |
| 11 | dsVPASE (7) | PEG 10K | 0.2 |
| 12 | dsVPASE (7) | PEG 10K | 0.3 |
| 13 | dsVPASE (7) | PEG 10K | 0.4 |
| 14 | dsVPASE (7) | PEG 10K | 0.5 |

^{a}Input is moles of PEG/mole of RNA nucleotide.
^{b}Modifier is the —OC(O)R¹ modification as defined in Formula I.
^{c}Sequence is the sequence of nucelobases of the RNA strand backbone.

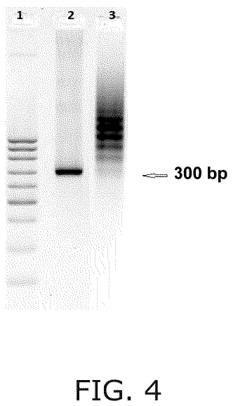

As can be seen by FIG. 4, the agarose gel (2%) analysis of the activated 10k PEG/dsRNA reaction mixture (line 3) demonstrates the covalent modification of the 300 bp molecule with 1, 2, 3, 4 and so on chains of 10k PEG polymer. The original unmodified dsRNA molecule is all consumed during the reaction.

### Example 2. Preparation of High Molecular Weight Polyalkyloxy Polymer dsRNA Using DCM for the Activation Reaction

One equivalent PEG-acetic acid was dissolved in dichloromethane (DCM) at 300 mg/ml and to this solution was added 1.05 equivalent of CDI. The reaction mixture was stirred for at least 2 hours, preferably overnight under argon. The reaction mixture was then diluted to 5 mg/ml with fresh DCM and extracted with 0.5 volumes of IN HCL followed by 0.5 volumes saturated NaCl. The aqueous layer was then washed with Ethyl acetate followed by DCM. The combined organic layers were dried over sodium sulfate and solvent was evaporated under vacuum to yield a viscous liquid. The viscous liquid was triturated with 2 volumes of acetonitrile (CAN) and stripped under a stream of argon to remover water and then dried under high vac and P₂O₅ for several hours. The resulting solid was reacted with dsRNA following the ionic solvation method. The following MdsRNAs were prepared according to this procedure:

TABLE 2
Modified MdsRNA Compounds Synthesized
by Procedure of Example 2.

| MdsRNA | Sequence ID No. | Modifier | Input |
|---|---|---|---|
| 15 | dsP450 (115) | PEG 5K | 0.1 |
| 16 | dsP450 (115) | PEG 5K | 0.2 |
| 17 | dsP450 (115) | PEG 5K | 0.3 |

^{a}Input is moles of PEG/mole of RNA nucleotide.
^{b}Modifier is the —OC(O)R¹ modification as defined in Formula I.
^{c}Sequence is the sequence of nucelobases of the RNA strand backbone.

### Example 3. Preparation of High Molecular Weight Polyalkyloxy Polymer dsRNA in DMSO

### Activation of Polyalkyloxy Polymer PEG

The polyaloxy polymer was reacted with a linker to introduce a reactive COOH at one end of the polymer chain. One eq. of polyalkyloxy polymer was dissolved in 1,4-dioxane. DMAP (0.5 eq.), DIEA (3 eq.), and the corresponding anhydride (1.5.eq.) were added and the reaction mixture was stirred overnight at room temperature. Then the mixture was diluted with DCM and extracted with sodium bicarbonate, brine. The organic layer was dried over sodium sulfate, and concentrated at reduced pressure. The resulting gum was triturated with fresh DCM and the solid polyalkyloxy-COOH was collected via filtration.

One eq. of poyalkyloxy-COOH was dissolved in dimethyl sulfoxide (DMSO) at 50 mg/ml and to this solution was added 1.05 equivalent of carbonyldiimidazole (CDI). The reaction mixture was stirred for at least 2 hours, preferably overnight under argon. This solution was then added directly to dsRNA.

The preparation method described above was used to prepare the following activated polyalkyloxy polymers which were used to yield the desired MdsRNAs: Methoxy PEG acetic acid (MPEGA) 1K, 2K, 5K, and 10K; Methoxy PEG acetic acid (MPEGA-Y) 40K, Y-shaped; Methoxy PEG succinic acid (MPEGS) 5K; Methoxy PEG glutamic acid (MPEGG) 5K; Methoxy PEG 3,3-methylglutamic acid (MPEGM) 5K; Carbamoyl PEG 5K (CPEG), Poloxalene succinic L64, 2,9K; Poloxalene succinic L68; Poloxalene succinic L121; Poloxalene succinic F108, 14K; Poloxalene succinic F127, 12.5K.

Reaction of dsRNA with Activated Polyalkyloxy Polymer PEG

Benzyl-TriButyl Ammonium-Chloride was dissolved in DMSO at 200 mM. An aliquot of this solution was added to freeze-dried dsRNA in a reaction tube. The tube was warmed to 65° C. in a shaker for 1 hr. Then the temperature was reduced to 55° C. and catalytic amount of pyridine was added followed by a 200 μg/μL solution of DMAP. A 200 μg/μL solution of activated polyalkyloxy polymer was added in portions. The mixture was kept at 55° C. for 90 min and then was quenched with 500 mM citrate pH 4.5 and diluted to 40 mL with water. The resulting mixture containing the crude M-dsRNA was purified using tangential flow filtration (TFF), to yield MdsRNA with >90% purity. The MdsRNAs were characterized by gel electrophoresis with non-denaturing agarose gel and denaturing polyacrylamide gel.

The extent of modification (% Modification), i.e. the ratio of number of bases esterified with polymer to the total number of bases in the dsRNA was determined by a combination hydrolysis/HPLC-ELSD method. Hydrolysis of the MdsRNA samples was obtained upon heating at 99° C. a mixture of an aqueous solution of the purified product and 0.5M NaOH solution. The resulting polyalkyloxy polymer and RNA nucleotides were quantified using a Shimadzu LC-2030C HPLC fitted with a C18 100 A LC Column 250×4.6 mm and an ELSD. A calibration curve was built using dsRNA starting material and polyalkyloxy polymer as standards.

The insecticidal activity of the MdsRNAs was screened using a leaf disc assay. MdsRNAs targeting Diamondback moth (DBM) (P. xylostella) were tested on DBM larvae using cabbage leaf discs. The MdsRNA treatments were dissolved in water with an adjuvant and diluted to obtain the desired concentration. The desired treatment solution was sprayed on both sides of fresh cabbage leaf discs (3.5 cm diameter). Each treatment was applied with 3 repetitions and a water only control. The discs were placed on a wet paper towel in a container. P. xylostella eggs (8-12) were transferred to each treated disc and the containers were incubated at 26° C. and 72% relative humidity (RH). On days 2, 3, and 4 fresh treated discs were provided, and untreated discs were provided daily after that. On days 5 and 8 larvae mortality and stunting were recorded. The % efficacy and the std dev. were calculated for each treatment and used to rate the treatments on a scale of 1 to 3 as follows: % efficacy 0-30% Rate=1; % efficacy 30-50% Rate=2, % efficacy>50% Rate=3. Treatments with rating of 3 were advanced to further optimization studies.

MdsRNAs targeting Fall armyworm (FAW) (S. frugiperda) were tested on DBM larvae using corn leaf squares (9 cm2). One egg of FAW was placed on each corn leaf and the containers were incubated as described above. A minimum of 10 repetitions per treatment and a negative control were run. On days 4, 5, and 6 fresh treated leaf squares were provided, and untreated discs were provided daily after that. On days 8 and 11 larvae mortality and stunting were recorded. The % efficacy was calculated for each treatment and used to rate the treatments on a scale of 1 to 3 as follows: % efficacy 0-30% Rate=1; % efficacy 30-50% Rate=2, % efficacy>50% Rate=3. Treatments with rating of 3 were advanced to further optimization studies.

The following MdsRNAs were prepared according to the procedures described above:

TABLE 3
Modified dsRNA Triggers Synthesized
Using Procedure in Example 3.

| | | | | Effi- |
|---|---|---|---|---|
| | | | % Modifi- | cacy |
| MdsRNA | Sequence ID No. | Modifier | cation | rate |
| 18 | ABCH-1 (172) | MPEGA 5K | 1.7 | 2 |
| 19 | ABCH-1 (172) | MPEGA 5K | 3.1 | 2 |
| 20 | ABCH-1 (172) | MPEGA 5K | 4.1 | |
| 21 | ABCH-1 short (256) | MPEGA 5K | 1.0a | 2 |
| 22 | ABCH-1 short (256) | MPEGA 5K | 2.0a | 2 |
| 23 | ABCH-1 short (256) | MPEGA 5K | 3.0a | 2 |
| 24 | AChE-2 (1) | MPEGA 10K | 1.0a | 2 |
| 25 | AChE-2 (1) | MPEGA | 1.0a/20 | 2 |
| | | 10K/NMA | | |
| 26 | B1 (4) | MPEGA 5K | 1.0a | |
| 27 | B1 (4) | MPEGA 5K | 2.0a | |
| 28 | B1 (4) | MPEGA 5K | 3.0a | |
| 29 | Chit5 (269) | MPEGA 5K | 2.0a | 2 |
| 30 | DOMELESS (265) | MPEGA 5K | 2.0 | 3 |
| 31 | DOMELESS FAW (187) | MPEGA 5K | 2.4 | 3 |
| 32 | DREDD (266) | MPEGA 5K | 2.4 | 3 |
| 33 | DREDD FAW (204) | MPEGA 5K | 2.7 | 2 |
| 34 | DOUX (268) | MPEGA 5K | 2.4 | 3 |
| 35 | GSS1 (267) | MPEGA 5K | 2.4 | 2 |
| 36 | GST1 FAW (226) | MPEGA 5K | 2.4 | 1 |
| 37 | JHEH (257) | MPEGA 5K | 2.0 | 2 |
| 38 | MESH (259) | MPEGA 5K | 2.0 | 3 |
| 39 | MESH (259) | MPEGA 5K | 4.9 | 3 |
| 40 | MESH (259) | MPEGA 5K | 5.7 | 3 |
| 41 | MESH (259) | MPEGA 5K | 6.1 | 3 |
| 42 | MESH (259) | MPEGA 5K | 7.0 | 3 |
| 43 | MESH FAW (236) | MPEGA 5K | 2.1 | 3 |
| 44 | PBAN (81) | MPEGA 5K | 2.0a | 3 |
| 45 | PPO (253) | MPEGA 5K | 3.0a | 2 |
| 46 | PPO-1 (63) | MPEGA 5K | 2.0 | 3 |
| 47 | P450 (115) | MPEGAY 40K | 1.0a | 1 |
| 48 | P450 (115) | MPEGAY 40K | 2.0a | 2 |
| 49 | P450 (115) | MPEGAY 40K | 3.0a | 3 |
| 50 | P450 (115) | MPEGA 10K | 0.025a | |
| 51 | P450 (115) | MPEGA 10K | 0.05a | 2 |
| 52 | P450 (115) | MPEGA 10K | 0.1a | 3 |
| 53 | P450 (115) | MPEGA 10K | 2.2 | 3 |
| 54 | P450 (115) | MPEGA 10K | 2.9 | 3 |
| 55 | P450 (115) | MPEGA 10K | 3.7 | 3 |
| 56 | P450 (115) | MPEGA 5K | 0.8 | 2 |
| 57 | P450 (115) | MPEGA 5K | 2.0 | 3 |
| 58 | P450 (115) | MPEGA 5K | 3.4 | 3 |
| 59 | P450 (115) | MPEGA 5K | 4.8 | 3 |
| 60 | P450 (115) | MPEGA 5K | 5.8 | 3 |
| 61 | P450 (115) | MPEGS 5K | 5.0a | |
| 62 | P450 (115) | MPEGG 5K | 5.0a | |
| 63 | P450 (115) | MPEGM 5K | 5.0a | |
| 64 | P450 (115) | MPEGA 2K | 1.0a | 1 |
| 65 | P450 (115) | MPEGA 2K | 2.0a | 1 |
| 66 | P450 (115) | MPEGA 2K | 3.0a | 1 |
| 67 | P450 (115) | MPEGA 1K | 1.0a | |
| 68 | P450 (115) | MPEGA 1K | 2.0a | |
| 69 | P450 (115) | MPEGA 1K | 3.0a | |
| 70 | P450 (115) | L64 | 5.0a | 3 |
| 71 | P450 (115) | L121 | 5.0a | 2 |
| 72 | P450 (115) | L68 | 5.0a | 2 |
| 73 | P450 (115) | L108 | 5.0a | 3 |
| 74 | P450 (115) | L108 | 8.0a | 3 |
| 75 | P450 (115) | L127 | 5.0a | 2 |
| 76 | P450 (115) | L127 | 8.0a | 2 |
| 77 | P450 (115) | CPEG | 5.0a | |
| 78 | P450 (115) | CPEG | 10.0a | |
| 79 | BI-P450 (254) | MPEGA 5K | 2.0a | 3 |
| 80 | P450 short (127) | MPEGA 5K | 1.0 | |
| 81 | P450 short (127) | MPEGA 5K | 1.8 | |
| 82 | P450 short (127) | MPEGA 5K | 2.5 | |
| 83 | P450 FAW (255) | MPEGA 5K | 3.0 | 2 |
| 84 | CYP321A8 FAW (152) | MPEGA 5K | 2.4 | 1 |
| 85 | CYP6B2 FAW (168) | MPEGA 5K | 2.0 | 2 |
| 86 | CYP9A58 FAW (160) | MPEGA 5K | 2.1 | 2 |
| 87 | Snf7 (5) | MPEGA 5K | 1.2 | |
| 88 | Snf7 (5) | MPEGA 5K | 2.2 | |
| 89 | Snf7 (5) | MPEGA 5K | 2.9 | |
| 90 | Snf7CRW (264) | MPEGA | 2.0a/35 | |
| | | 10K/NMA | | |
| 91 | SSK FAW (263) | MPEGA 5K | 2.0 | 1 |
| 92 | VPASE A FAW (247) | MPEGA 5K | 2.0 | 2 |
| 93 | VPASE A/E (28) | MPEGA 5K | 2.0 | 3 |
| 94 | VPASE (7) | MPEGAY 40K | 1.0a | 2 |
| 95 | VPASE (7) | MPEGAY 40K | 2.0a | 2 |
| 96 | VPASE (7) | MPEGAY 40K | 3.0a | 2 |
| 97 | VPASE (7) | MPEGA 10K | 0.025a | |
| 98 | VPASE (7) | MPEGA 10K | 0.05a | |
| 99 | VPASE (7) | MPEGA 10K | 0.7 | 2 |
| 100 | VPASE (7) | MPEGA 10K | 1.0 | 3 |
| 101 | VPASE (7) | MPEGA 10K | 1.2 | 2 |
| 102 | VPASE (7) | MPEGA 10K | 1.3 | 3 |
| 103 | VPASE (7) | MPEGA 10K | 1.4 | 2 |
| 104 | VPASE (7) | MPEGA 5K | 0.9 | |
| 105 | VPASE (7) | MPEGA 5K | 2.1 | 3 |
| 106 | VPASE (7) | MPEGA 5K | 3.3 | 3 |
| 107 | VPASE (7) | MPEGA 5K | 4.2 | 3 |
| 108 | VPASE (7) | MPEGA 5K | 5.5 | 3 |
| 109 | VPASE (7) | MPEGA 5K | 6.9 | 3 |
| 110 | VPASE (7) | MPEGA 5K | 8.6 | 3 |
| 111 | VPASE (7) | MPEGA 2K | 1.0a | |
| 112 | VPASE (7) | MPEG 2K | 2.0a | |
| 113 | VPASE (7) | MPEGA 2K | 3.0a | |
| 114 | VPASE (7) | MPEGA 1K | 1.0a | |
| 115 | VPASE (7) | MPEGA 1K | 2.0a | |
| 116 | VPASE (7) | MPEGA 1K | 3.0a | |
| 117 | BI-VPASE (262) | MPEGA 5K | 2.0 | 3 |
| 118 | VPASE short (27) | MPEGA 5K | 1.0a | 3 |
| 119 | VPASE short (27) | MPEGA 5K | 2.0a | 3 |
| 120 | VPASE short (27) | MPEGA 5K | 3.0a | 3 |
| 121 | VPASE short (27) | MPEGA 5K | 5.2 | 3 |
| 122 | VPASE short (27) | MPEGA 5K | 6.9 | 3 |
| 123 | VPASE short (27) | MPEGA 5K | 8.6 | 3 |
| 124 | VPASE FAW (261) | MPEGA 5K | 0.8 | |
| 125 | VPASE FAW (261) | MPEGA 5K | 3.4 | |
| 126 | Venom (260) | MPEGA 5K | 2.0 | 3 |
| 127 | TH (3) | MPEGA 5K | 1.0a | |
| 128 | TH (3) | MPEGA 5K | 2.0a | 3 |
| 129 | TH (3) | MPEGA 5K | 3.0a | |
| 130 | P450 (115) | CPEG 5K | 0.5i | 3 |
| 131 | P450 (115) | CPEG 5K | 1.0i | 3 |
| 132 | CYP9A58 FAW (160) | MPEGA 5K | 3.0a | 1 |
| 133 | CYP9A58 FAW (160) | MPEGA 5K | 9.0a | 3 |
| 134 | MESH FAW (236) | MPEGA 5K | 3.0a | 3 |
| 135 | MESH FAW (236) | MPEGA 5K | 9.0a | 3 |
| 136 | CYP9A58 FAW (160) | MPEGA 10K | 6.0a | 3 |
| 137 | CYP9A58 FAW (160) | L108 | 10.0a | 2 |
| 138 | MESH FAW (236) | L108 | 10.0a | 3 |
| 139 | MESH FAW (236) | MPEGA 10K | 6.0a | 3 |
| 140 | MI FAW (275) | MPEGA 10K | 24 | 3 |

aEstimated from moles of PEG/moles of RNA nucleotide in the reacting mixture.
^{b}Modifier is the —OC(O)R¹ modification as defined in Formula I.
^{c}Sequence is the sequence of nucleobases of the RNA strand backbone.

### Example 4a. Preparation of High Molecular Weight Polyalkyloxy Polymer-NMIA dsRNA

MPEGA-dsAChE2 was prepared and purified according to the procedure described in Example 3 using Methoxy PEG acetic acid 10K at input 0.1. Benzyl-TriBA-Cl was dissolved in DMSO at 200 mM. An aliquot (3.4 mL) of this solution was added to freeze-dried MPEGA-dsAChE (34 mg) in a reaction tube. The tube was warmed to 65° C. in a shaker for 1 hr. Then the temperature was reduced to 55° C. and 110 μL pyridine are added followed by 500 μL of a 200 μg/μL solution of DMAP and 442 μL of a 200 μg/μL solution of NMIA. After 5 min, another 442 μL of NMIA solution were added (Input=10, moles of NMIA/mole of RNA nucleotide). The mixture was kept at 55° C. for 90 min and then is quenched with 500 mM citrate, and diluted to 40 mL with water. The resulting mixture containing the crude PEG-NMA-dsAChE2 was purified using tangential flow filtration, TFF, to yield MdsRNA with >90% purity. The PEG-NMA-dsAChE2 was characterized by gel electrophoresis with non-denaturing agarose gel and denaturing polyacrylamide gel. % Modification by HPLC: PEG 1%, NMIA 37%.

### Example 4b. Preparation of High Molecular Weight Polyalkyloxy Polymer-(R)-2 ((tert-Butoxycarbonyl)amino)-3-(tert-butyldisulfanyl)propanoic acid (TBDS) dsRNA

MPEGA-EPSPS was prepared and purified according to the procedure described in Example 3 using Methoxy PEG acetic acid 5K at input 0.6. Benzyl-TriBA-Cl was dissolved in DMSO at 200 mM. An aliquot (1.0 mL) of this solution was added to freeze-dried MPEGA-EPSPS (10 mg) in a reaction tube. The tube was warmed to 65° C. in a shaker for 1 hr. Then the temperature was reduced to 55° C. and 8.5 L pyridine are added followed by 29 μL of a 200 μg/μL solution of DMAP and 230 μL of a 200 μg/μL solution of TBDS. After 5 min, another 442 μL of NMIA solution were added (Input=9, moles of TBDS/mole of RNA nucleotide). The mixture was kept at 55° C. for 240 min and then is quenched with 500 mM citrate, and diluted to 40 mL with water. The resulting mixture containing the crude PEG-TBDS-dsEPSPS was purified using tangential flow filtration, TFF, to yield MdsRNA with >90% purity. The PEG-TBDS-dsEPSPS was characterized by gel electrophoresis with non-denaturing agarose gel and denaturing polyacrylamide gel. % Modification by HPLC: PEG 2.2%, TBDS 10.9%.

### Example 5. Preparation of High Molecular Weight Polyalkyloxy Polymer-FA dsRNAs

Following similar procedures as described in Examples 4a and 4b. MPEGA-P450 5K (115) was reacted with activated lauryl (LAU), oleic (OLE) and linoleic (LIN) fatty acids to yield the MPEGA-FA-P450 materials described in Table 4.

TABLE 4
Modified dsRNA Triggers Synthesized
Using Procedure in Example 4.

| | | | | Moles of | |
|---|---|---|---|---|---|
| | | | | Mod 2/Moles | |
| | | | | of nucleo- | |
| | | | | tide in | Effi- |
| | Modifier | % Mod | Modifier | reaction | cacy |
| MdsRNA | 1 | 1 | 2 | mixture | Rate |
| 130 | MPEGA 5K | 0.6 | LIN | 10 | 2 |
| 131 | MPEGA 5K | 0.6 | LIN | 15 | 2 |
| 132 | MPEGA 5K | 0.9 | LIN | 20 | 2 |
| 133 | MPEGA 5K | 1.0 | LAU | 10 | |
| 134 | MPEGA 5K | 0.7 | LAU | 15 | |
| 135 | MPEGA 5K | 0.6 | LAU | 20 | 2 |
| 136 | MPEGA 10K | 0.6 | OLE | 15 | |
| 137 | MPEGA 10K | 1.3 | OLE | 15 | |
| 138 | MPEGA 10K | 0.6 | LIN | 15 | |
| 139 | MPEGA 10K | 1.3 | LIN | 15 | |

### Example 6. Cabbage Disc Assay

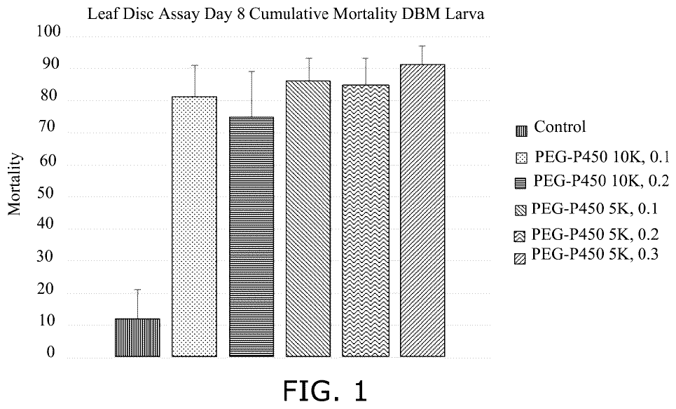

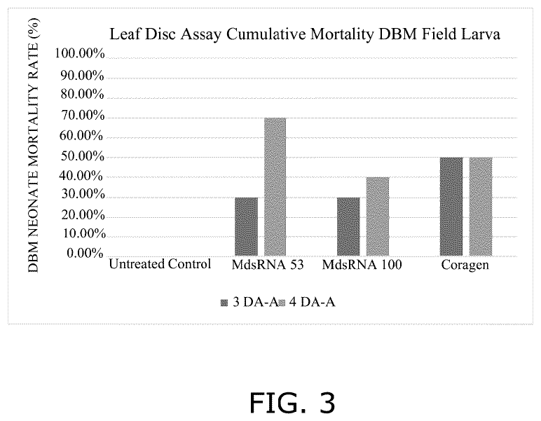

The insecticidal activity of modified dsRNA materials was tested on DBM larvae using a cabbage leaf disc bioassay and DBM eggs collected from the field. Cabbage leaves were collected at stage 4-5. Leaves were prepared by washing with tap water using a nozzle to remove insects, dust etc. The leaves were then wiped with paper towels. Leaf discs of 3.5 cm diameter were punched from the cabbage leaves using a metal cutter. An aqueous solution of the desired treatment solution was prepared at 500 ppm or 150 ppm concentration and 60 μL were sprayed on both sides of the disc. After the treatment was applied to both sides of the leaf disc, four 1 cm diameter discs were then punched from the treated 3.5 cm. The resulting treated 1 cm diameter discs were used in the assay. Three P. xylostella egg were placed on each treated disc placed in a petri dish. The plates were incubated at 26° C. and 72% room humidity (RH). After 48 hours only one neonate was transferred to a new (2^{nd}) treated disc. A third treated disc was provided after 24 hours of the 2^{nd} treated disc. The larvae were then provided with an untreated disc every day for up to 10 days. Mortality was recorded daily starting on the 2^{nd} day after incubation. Mortality rate was calculated for each test during the duration of the experiment and cumulative mortality after 5 days and 7 or 9 days was calculated. After 7 days of incubation the number of 2^{nd} or 1^{st} instar larva was added to the dead larva count. The efficacy of some PEG-P450 (115) examples on DBM larva is shown in FIG. 1. The efficacy of two PEG-dsRNA examples on field collected DBM eggs vs. efficacy of a leading commercial product is shown in FIG. 3 and Table 5.

TABLE 5
DBM Larva % Control (Calculated using Schneider-Orelli)*

| | MdsRNA | | | | | | | | |
|---|---|---|---|---|---|---|---|---|---|
| Name | Description | Rate | | 3DA-A | | 4DA-A | | 5DA-A | |
| Untreated | | | | 0.00% | c | 0.00% | c | 0.00% | c |
| control | | | | | | | | | |
| MdsRNA #53 | PEG-P450 | 60 | uL/seed | 33.33% | b | 75% | a | 44.44% | ab |
| | (115) | | | | | | | | |
| MdsRNA #100 | PEG-VPASE | 60 | uL/seed | 22.22% | bc | 37.50% | b | 33.33% | b |
| | (7) | | | | | | | | |
| Coragen | | 0.01% | v/v | 66.67% | a | 66.67% | ab | 66.67% | a |

DA-A: days after infestation, infestation.
*Means followed by the same letter do not significantly differ (P = 0.05, LSD)

### Example 7. Preparation of NMA dsRNAs Using Ionic Solvation Method

Benzyl-TriButyl Ammonium-Chloride is dissolved in DMSO at 200 mM. An aliquot of this solution is added to freeze-dried dsRNA in a reaction tube. The tube is warmed to 65° C. in a shaker for 1 hr. Then the temperature is reduced to 55° C. and 110 μL pyridine are added followed by a 200 μg/μL solution of DMAP and two additions of a 200 μg/μL solution of N-methyl isatoic anhydride (NMIA). The mixture is kept at 55° C. for 90 min and then is quenched with 500 mM citrate, and diluted with water. The resulting mixture containing the crude NMA-dsRNA is purified using tangential flow filtration, TFF, to yield MdsRNA with >90% purity. The following NMA-dsRNA were prepared according to the procedure described above: NMA-P450 (115) (37% NMA), NMA-VPASE (7) (81% NMA), NMIA-AChE (1) (40%, 80% NMA), NMA-B1 (4), NMA-TH (3). This procedure was also used to prepare dsRNA analogs for NBA, dimethyl furoyl, -Tyr, -Trp, -Leu, and octanoyl. This procedure can be used for other modifications such as, but not limited to, lauroyl, linoleyl, and the like.

### Example 8. Persistence of PEG-dsRNA in Field Cabbage Plants

Longevity of an RNAi active ingredient in a field has always been of primary importance in development of advanced pesticides. RNA is known to be a very unstable molecule vulnerable to enzymatic degradation. Estimations of half-life for dsRNA in the field range between 0.5 and 0.7 days after foliar application on soybean plants (Bachman et al., 2020). The time decay of dsRNA sequences sprayed on cabbage leaves for native dsRNAs and 2′-O modified dsRNAs was studied in a small plot field trial.

Cabbage leaves samples exposed to sprays were collected using a leaf puncher at these time points after spraying: 0 days (1 hr), 1, 2, 3, and 5 days. Per each data point, 12 leaf circles from 12 individual plants from each plot were collected and immediately frozen on dry ice. Then the bags should be stored in dry ice or in −80° C. freezer until ready to be analyzed.

The dsRNA used in this trial is a dsSNF7 sequence that has been previously reported in field dissipation study (Bachman et al., 2020). QuantGene, a nucleic acid detection platform marketed by Invitrogen, was the preferred analytical method used to quantify of dsRNA sequences on the cabbage plant leaves after spray. The sequence and corresponding Quantigene probe sets were published previously (Armstrong et al., 2013). The sequence is not directed against Diamondback Moth (DBM) and used here only for analytical purposes. Naïve (unmodified) dsSNF7, treatment C2, was used as a comparison.

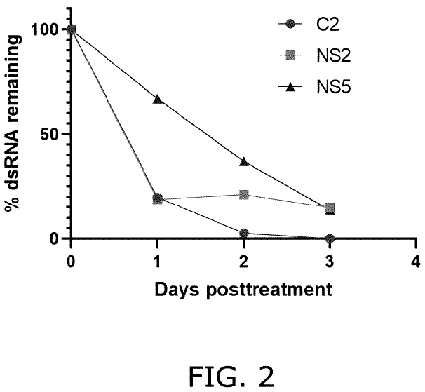

Two modifying groups were used: N-methyl anthranoyl and PEGA. The dsRNAs modified with these two groups demonstrated exceptional biological activities against DBM in previous studies (see, for example, Section 1, Example 3, Table 3). Treatment NS2 (PEGA-NMA-dsSNF7, MdsRNA 90, Table 3 in Section 1) showed a different profile from all others dsRNAs including NS5 (NMA-dsSNF7, NMA˜100%), hinting at existence of a highly stabilized MdsRNA fraction which exhibited almost no decay in the tested timeframe (Sec FIG. 2).

### Example 9. Control of Glyphosate-Resistant Palmer Amaranth with MdsRNAs

The herbicidal activity of modified dsRNAs materials was tested on glyphosate-resistant Palmer amaranth in the greenhouse. Plants from seeds of P. amaranth producing 10 to >100 copies of the EPSPS gene collected in a field in AZ were used in this example. Treatment solutions were topically applied on the leaves of plants at V4-6 stage, plant height 5-8 cm. 30 μL of solution was applied to each plant, enough to cover 1 or 2 leaves. Control plants were tested with a Roundup solution (1 μg/μL). For treated plants, a solution containing a mixture of MdsRNA (9 μg/plant)+Roundup (30 μg/plant) with no additional adjuvant was used. Table 6 shows the treatment descriptions.

TABLE 6
Treatments used to control Palmer amaranth.

| | Glyphosate rate | MdsRNA description | MdsRNA rate |
|---|---|---|---|
| Control | 30 ug/plant | — | — |
| Treatment 1 | 30 ug/plant | PEG5K-TBDS-EPSPS | 9 ug/plant |
| Treatment 2 | 30 ug/plant | PEG5K-EPSPS | 9 ug/plant |

The plants were observed at 14 and 20 days after treatment. At 20 days, the control plants, treated only with glyphosate, survived and some grew very tall. The plants treated with MdsRNAs formulations 1 and 2 were dead or severely stunted.

### Example 10. Preparation of Emulsions with MdsRNAs

The modification of dsRNA described above can change its physical properties, thus opening new ways for delivery as well as new applications. For example, an emulsifiable concentrate or EC is a common pesticide formulation strategy for organic-solvent soluble molecules consisting of an organic solvent concentrate of the active ingredient, often containing 15% detergent, that is diluted with water in the field before the application.

Vegetable oil and methyl esters of fatty acids derived from corn oil and sugar-like detergents such as polysorbates (Tween), sorbitan and galactose, Xylose and Rhammose glycolipids were found to produce stable EC.

A mixture of soybean oil (85%): Tween-80 (15%) was used to mixture solubilized or formed fine suspensions of MdsRNAs up to 5% weight with MPEGA-5K-SEQ1, LIN-dsSEQ1 and PEG-LIN-dsSEQ1.

Another organic solvent-based approach to formulate MdsRNA as oil in water or water in oil emulsions (W/O). W/O emulsions would have the following advantages over oil-in-water (O/W) emulsions. RNA-containing aqueous phase would be more protected from enzymatic degradation if surrounded by the oil phase. Also, the aqueous phase of W/O emulsions is less prone to dehydration which might be important to preserve the stability of the emulsion in field applications.

The surfactants needed to achieve stable emulsions are usually classified according to their hydrophile-lipophile balance numbers. HLB range suitable for generation of stable

W/O emulsions is usually 3 to 6 (Kruglyakov, 2000). A pair of surfactants with different HLB values were chosen to fall in this range (Liu et al., 2022). Soybean oil, almond oil, and olive oil were successfully used as the oil component in the emulsion. Table A shows different examples of stable W/O and O/W formulations with MPEGA5K-P450.

TABLE A

| | | Water/ | Veg. | | | |
|---|---|---|---|---|---|---|
| Sample | Water, | Glycerol | Oil | Adjuvant | Adjuvant | Emulsion |
| # | μL | (1:1), μL | 2, μL | 1, μL | 2, μL | type |
| 1 | 300 | 0 | 600 | 20 | 80 | W/O |
| 2 | 0 | 300 | 600 | 20 | 80 | W/O |
| 3 | 800 | 0 | 100 | 80 | 20 | O/W |
| 4 | 0 | 800 | 100 | 80 | 20 | O/W |

### Section 2: dsRNAs Constructs Targeting Agricultural Pests

### Example 1. The Following DNA Cassette, Flanked by the SphI and the NruI Restriction Sites, Comprising the Code for a Viral Coat Protein was Prepared

SEQ ID NO: 276. YP_009640125.1 Coat protein
[Escherichia phage MS2]
GCATGCAGATCTCGATCCCGCGAAATTAATACGACTCACTATAGGGAGA
CCACAACGGTTTCCCTCTAGATCCCTACATTAGAGAAGAGAGCTAATAA
AGAAGGAGATATACATATGGCGTCTAACTTTACCCAATTCGTTCTGGTT
GATAACGGCGGTACGGGTGACGTTACCGTAGCTCCGTCCAACTTCGCCA
ACGGTGTTGCGGAATGGATTAGCTCTAACAGCCGCTCTCAGGCCTACAA
AGTCACGTGCTCCGTTCGTCAGTCTAGCGCGCAGAATCGCAAATACACC
ATCAAAGTTGAAGTACCGAAAGTCGCAACGCAGACCGTAGGCGGCGTAG
AACTCCCAGTTGCGGCCTGGCGCTCTTACCTCAACATGGAACTGACTAT
TCCGATTTTTGCGACGAACTCCGACTGCGAACTGATTGTTAAGGCAATG
CAGGGCCTGCTGAAAGACGGTAATCCGATCCCATCTGCAATCGCTGCTA
ACTCTGGCATTTACTAATAAGCGGACGCGCTGCCACCGCTGAGCAATAA
CTAGCATAACCCCTTGGGGCCTCTAAACGGGTCTTGAGGGGTTTTTTGC
TGAAAGGAGGAACTATATCCGTCGCGA

### Example 2. The Following DNA Cassettes Coding for MS2 PAC Sites and ssRNA Linkers Linking Different dsRNA Stems, Flanked by the HindIII and the SphI Restriction Sites, Comprising Codes for dsRNAs Targeting Agricultural Pests were Prepared

SEQ ID NO: 277. DQ088989.1. Plutella xylostella cytochrome P450
(CYP6BF1v2) 3x58
AAGCTTAGATCTCGATCCCGCGAAATTAATACGACTCACTATAGGGAGACCAC
AACGGTTTCCCTCTAGATCACAAGTTTGTACAAAAAAGCAGGCTAAGAAGGA
GATATACATACGCCGGCCATTCAAACATGAGGATTACCCATGTATTTAAATAC
CCATGTCCAGGCGCGCTCCGCGATCGCGGCAATAAAGCGGTTACAAGCCCGC
AAAAATAGCAGAGTAATGTCGCGATAGCGCGGCATTAATGCAGCTTTATTGattc
ctaGTCCTCAGATACGGCTACCCTTCCTTCTTCTACAGCGTGGGATTGGAGCTCT
ATTCCAattcctaGCAAGTAGGTATACGGGACAATACTTTTCCGTCCTTCTTCAGAA
AACCGCTCCGGACGatccctAcatgaggattacccatgtatccctCAATAAAGCTGCATTttaccgtttc
ttccgatctgttatacttgacgttataaacagtattcctaTGGAATAGAGCTCCAATCCCACGCTGTAGA
AGAAGGAAGGGTAGCCGTATCTGAGGACattcctaCgtccggagcggttttctgaagaaggacggaa
aagtattgtcccgtatacctacttgcatccctAcatgaggatcacccatgtatccctACTGTTTATAACGTCAAGT
ATAACAGATCGGAAGAAACGGTAAggccggcatggtcccagcctcctcgctggcgccggctgggcaac
attccgaggggaccgtcccctcggtaatggcgaatgggacccCGGACCGAATACCCGGTCTGAACGAG
GGCGGCCGCGGTACCCAAGAAGTACTTAGAGTTAATTAAGGAGTTCAAACAT
GAGGATCACCCATGTCGAAGCTCCCACACCCTAGCATAACCCCTTGGGGCCTC
TAAACGGGTCTTGAGGGGTTTTTTGCTGAAAGGAGGAACTATATCCGGAATTC
CGCAGGACTGGGCGGCGGGCTAGCATCGTCCATTCCGACAGCATCGCCAGTCA
CTATGGCGTGCTGCTAGCGCTATATGCGTTGATGCAATTTCTATGCGCACCCGT
TCTCGGAGCACTGTCCGACCGCTTTGGCCGCCGCCCAGTCCTGCGCATGC
SEQ ID NO: 278 DQ088989.1. Plutella xylostella cytochrome P450
(CYP6BF1v2) 5x58
AAGCTTAGATCTCGATCCCGCGAAATTAATACGACTCACTATAGGGAGACCAC
AACGGTTTCCCTCTAGATCACAAGTTTGTACAAAAAAGCAGGCTAAGAAGGA
GATATACATACGCCGGCCATTCAAACATGAGGATTACCCATGTATTTAAATAC
CCATGTCCAGGCGCGCTCCGCGATCGCGGCAATAAAGCGGTTACAAGCCCGC
AAAAATAGCAGAGTAATGTCGCGATAGCGCGGCATTAATGCAGCTTTATTGattc
ctaGTCCTCAGATACGGCTACCCTTCCTTCTTCTACAGCGTGGGATTGGAGCTCT
ATTCCAattcctaACTGTTTATAACGTCAAGTATAACAGATCGGAAGAAACGGTAA
ATTTTGCTGGAATAGattcctaCCGTCTATGCCATTATCAATACTGTCTCCCGTTAT
GTATTTGTTCTTCTTCCAATCGGattcctaGCAAGTAGGTATACGGGACAATACTTT
TCCGTCCTTCTTCAGAAAACCGCTCCGGACGatccctAcatgaggattacccatgtatccctCAA
TAAAGCTGCATTctatggactgtataacttcgtgtgcattcggcgtcgactctggattcctaTGGAATAGAGC
TCCAATCCCACGCTGTAGAAGAAGGAAGGGTAGCCGTATCTGAGGACattcctaCt
attccagcaaaatttaccgtttcttccgatctgttatacttgacgttataaacagtattcctaCcgattggaagaagaacaaatacataa
cgggagacagtattgataatggcatagacggattcctaCgtccggagcggttttctgaagaaggacggaaaagtattgtcccgtat
acctacttgcatccctAcatgaggatcacccatgtatccctCCAGAGTCGACGCCGAATGCACACGAA
GTTATACAGTCCATAGggccggcatggtcccagcctcctcgctggcgccggctgggcaacattccgaggggac
cgtcccctcggtaatggcgaatgggacccCGGACCGAATACCCGGTCTGAACGAGGGCGGCCG
CGGTACCCAAGAAGTACTTAGAGTTAATTAAGGAGTTCAAACATGAGGATCA
CCCATGTCGAAGCTCCCACACCCTAGCATAACCCCTTGGGGCCTCTAAACGGG
TCTTGAGGGGTTTTTTGCTGAAAGGAGGAACTATATCCGGAATTCCGCAGGAC
TGGGCGGCGGGCTAGCATCGTCCATTCCGACAGCATCGCCAGTCACTATGGCG
TGCTGCTAGCGCTATATGCGTTGATGCAATTTCTATGCGCACCCGTTCTCGGAG
CACTGTCCGACCGCTTTGGCCGCCGCCCAGTCCTGCGCATGC
SEQ ID NO: 279 NM_001305532.1. Plutella xylostella V-type proton ATPase
subunit E
(LOC105389010) 3x58
AAGCTTAGATCTCGATCCCGCGAAATTAATACGACTCACTATAGGGAGACCAC
AACGGTTTCCCTCTAGATCACAAGTTTGTACAAAAAAGCAGGCTAAGAAGGA
GATATACATACGCCGGCCATTCAAACATGAGGATTACCCATGTATTTAAATAC
CCATGTCCAGGCGCGCTCCGCGATCGCGGCAATAAAGCGGTTACAAGCCCGC
AAAAATAGCAGAGTAATGTCGCGATAGCGCGGCATTAATGCAGCTTTATTGattc
ctaAcgcgccccccgcgcaaatattcaactttcttattattattatgtagaactaaaatgtattcctaAttaaatgcgtacaacattattaaca
tgtatgaaagaatccgtattaagtcagaaaatatccctAcatgaggattacccatgtatccctCAATAAAGCTGCA
TTGTTCTTGATCTTCTCCTTGTACTGCGCCTGCGCGCGCTCGAGCattcctaACATT
TTAGTTCTACATAATAATAATAAGAAAGTTGAATATTTGCGCGGGGGGCGCGT
attcctaATTTTCTGACTTAATACGGATTCTTTCATACATGTTAATAATGTTGTACG
CATTTAATatccctAcatgaggatcacccatgtatccctGctcgagcgcgcgcaggcgcagtacaaggagaagatcaa
gaacGgccggcatggtcccagcctcctcgctggcgccggctgggcaacattccgaggggaccgtcccctcggtaatggcgaa
tgggacccCGGACCGAATACCCGGTCTGAACGAGGGCGGCCGCGGTACCCAAGAA
GTACTTAGAGTTAATTAAGGAGTTCAAACATGAGGATCACCCATGTCGAAGCT
CCCACACCCTAGCATAACCCCTTGGGGCCTCTAAACGGGTCTTGAGGGGTTTT
TTGCTGAAAGGAGGAACTATATCCGGAATTCCGCAGGACTGGGCGGCGGGCT
AGCATCGTCCATTCCGACAGCATCGCCAGTCACTATGGCGTGCTGCTAGCGCT
ATATGCGTTGATGCAATTTCTATGCGCACCCGTTCTCGGAGCACTGTCCGACC
GCTTTGGCCGCCGCCCAGTCCTGCGCATGC
SEQ ID NO: 280. NM_001305532.1. Plutella xylostella V-type proton ATPase
subunit E (LOC105389010) 5x58
AAGCTTAGATCTCGATCCCGCGAAATTAATACGACTCACTATAGGGAGACCAC
AACGGTTTCCCTCTAGATCACAAGTTTGTACAAAAAAGCAGGCTAAGAAGGA
GATATACATACGCCGGCCATTCAAACATGAGGATTACCCATGTATTTAAATAC
CCATGTCCAGGCGCGCTCCGCGATCGCGGCAATAAAGCGGTTACAAGCCCGC
AAAAATAGCAGAGTAATGTCGCGATAGCGCGGCATTAATGCAGCTTTATTGattccta
AcgcgccccccgcgcaaatattcaactttcttattattattatgtagaactaaaatgtattcctaAttaaatgcgtacaacattattaacatgt
atgaaagaatccgtattaagtcagaaaatattcctaTtagcttagctgtagcttttaggagacacagttaaattgaaatgttat
accgatgcttattcctaTgtaagcgtaagcattttattgatataattctggattgttgccataacaattattacaatccctAcatgaggat
tacccatgtatccctCAATAAAGCTGCATTGTTCTTGATCTTCTCCTTGTACTGCGCCTG
CGCGCGCTCGAGCattcctaACATTTTAGTTCTACATAATAATAATAAGAAAGTTG
AATATTTGCGCGGGGGGCGCGTattcctaATTTTCTGACTTAATACGGATTCTTTCA
TACATGTTAATAATGTTGTACGCATTTAATattcctaAAGCATCGGTATAACATTTC
AATTTAACTGTGTCTCCTAAAAGCTACAGCTAAGCTAAattcctaTGTAATAATTGT
TATGGCAACAATCCAGAATTATATCAATAAAATGCTTACGCTTACAatccctAcatg
aggatcacccatgtatccctGctcgagcgcgcgcaggcgcagtacaaggagaagatcaagaacGgccggcatggtcccagc
ctcctcgctggcgccggctgggcaacattccgaggggaccgtcccctcggtaatggcgaatgggacccCGGACCGAA
TACCCGGTCTGAACGAGGGCGGCCGCGGTACCCAAGAAGTACTTAGAGTTAA
TTAAGGAGTTCAAACATGAGGATCACCCATGTCGAAGCTCCCACACCCTAGCATA
ACCCCTTGGGGCCTCTAAACGGGTCTTGAGGGGTTTTTTGCTGAAAGGAGG
AACTATATCCGGAATTCCGCAGGACTGGGCGGCGGGCTAGCATCGTCCATTCC
GACAGCATCGCCAGTCACTATGGCGTGCTGCTAGCGCTATATGCGTTGATGCA
ATTTCTATGCGCACCCGTTCTCGGAGCACTGTCCGACCGCTTTGGCCGCCGCCC
AGTCCTGCGCATGC

### Example 3. The Following DNA Cassettes Coding for MS2 PAC Sites and ssRNA Linkers Linking Different dsRNA Stems and Linking the Sense and Antisense Strands within the Same dsRNA, Flanked by the HindIII and the SphI Restriction Sites, Comprising Codes for dsRNAs Targeting Agricultural Pests are Prepared

DQ088989.1. Plutella xylostella cytochrome P450
(CYP6BF1v2) 3x58-NOPAC
SEQ ID NO: 281
AAGCTTAGATCTCGATCCCGCGAAATTAATACGACTCACTATAGGGAGACCAC
AACGGTTTCCCTCTAGATCACAAGTTTGTACAAAAAAGCAGGCTAAGAAGGA
GATATACATACGCCGGCCATTCAAATTTAAATACCCATGTCCAGGCGCGCTCC
GCGATCGCGGCAATAAAGCGGTTACAAGCCCGCAAAAATAGCAGAGTAATGT
CGCGATAGCGCGGCATTAATGCAGCTTTATTGattcctaGTCCTCAGATACGGCTA
CCCTTCCTTCTTCTACAGCGTGGGATTGGAGCTCTATTCCAattcctaGCAAGTAGG
TATACGGGACAATACTTTTCCGTCCTTCTTCAGAAAACCGCTCCGGACGatccctC
AATAAAGCTGCATTttaccgtttcttccgatctgttatacttgacgttataaacagtattcctaTGGAATAGAGC
TCCAATCCCACGCTGTAGAAGAAGGAAGGGTAGCCGTATCTGAGGACattcctaCg
tccggagcggttttctgaagaaggacggaaaagtattgtcccgtatacctacttgcatccctACTGTTTATAACGTCA
AGTATAACAGATCGGAAGAAACGGTAAggccggcatggtcccagcctcctcgctggcgccggctgg
gcaacattccgaggggaccgtcccctcggtaatggcgaatgggacccCGGACCGAATACCCGGTCTGAAC
GAGGGCGGCCGCGGTACCCAAGAAGTACTTAGAGTTAATTAAGGAGTTCAAC
GAAGCTCCCACACCCTAGCATAACCCCTTGGGGCCTCTAAACGGGTCTTGAGG
GGTTTTTTGCTGAAAGGAGGAACTATATCCGGAATTCCGCAGGACTGGGCGGC
GGGCTAGCATCGTCCATTCCGACAGCATCGCCAGTCACTATGGCGTGCTGCTA
GCGCTATATGCGTTGATGCAATTTCTATGCGCACCCGTTCTCGGAGCACTGTCC
GACCGCTTTGGCCGCCGCCCAGTCCTGCTCGCGA
NM_001305532.1. Plutella xylostella V-type proton ATPase
subunit E (LOC105389010) 1x300 DQ088989.1. Plutella xylostella cytochrome P450
(CYP6BF1v2) 3x58-
SEQ ID NO: 282
NOPACAAGCTTAGATCTCGATCCCGCGAAATTAATACGACTCACTATAGGGAGAC
CAC
AACGGTTTCCCTCTAGATCACAAGTTTGTACAAAAAAGCAGGCTAAGAAGGA
GATATACATACGCCGGCCATTCAAATTTAAATACCCATGTCCAGGCGCGCTCC
GCGATCGCACCAATTGCAGGCGTGAAGAATGTCCTCAGATACGGCTACCCTTC
CTTCTTCTACAGCGTGGGATTGGAGCTCTATTCCAGCAAAATTTACCGTTTCTT
CCGATCTGTTATACTTGACGTTATAAACAGTCGTAACGGCGCCAAATCTTCGA
GGAATGACATGGTGGATCTTATTTCCGATTGGAAGAAGAACAAATACATAAC
GGGAGACAGTATTGATAATGGCATAGACGGTGGAAACAAGAAGGTGCGTATC
GAAGTCGACGACGAACTTTTGGTGAGCCAATGTGTGCTGTTCTTGTTTAAACC
CTCTAGCTGCTTTACAAAGTACTGGTTCCCTTTCCAGCGGGATGCTTTATCTAA
ACGCAATGAGAGAGGTATTCCTCAGGCCACATCGCTTCCTAGTTCCGCTGGGA
TCCATCGTTGGCGGCCGAAGCCGCCATTCCATAGTGAGTTCTGGCGCGCCAAG
AACAGCACACATTGGCTCACCAAAAGTTCGTCGTCGACTTCGATACGCACCTT
CTTGTTTCCACCGTCTATGCCATTATCAATACTGTCTCCCGTTATGTATTTGTTC
TTCTTCCAATCGGAAATAAGATCCACCATGTCATTCCTCGAAGATTTGGCGCC
GTTACGACTGTTTATAACGTCAAGTATAACAGATCGGAAGAAACGGTAAATTT
TGCTGGAATAGAGCTCCAATCCCACGCTGTAGAAGAAGGAAGGGTAGCCGTA
TCTGAGGACATTCTTCACGCCTGCAATTGGTCGGACCGAATACCCGGTCTGAA
CGAGGGCGGCCGCGGTACCCAAGAAGTACTTAGAGTTAATTAAGGAGTTCAA
CGAAGCTCCCACACCCTAGCATAACCCCTTGGGGCCTCTAAACGGGTCTTGAG
GGGTTTTTTGCTGAAAGGAGGAACTATATCCGGAATTCCGCAGGACTGGGCGG
CGGGCTAGCATCGTCCATTCCGACAGCATCGCCAGTCACTATGGCGTGCTGCT
AGCGCTATATGCGTTGATGCAATTTCTATGCGCACCCGTTCTCGGAGCACTGTC
CGACCGCTTTGGCCGCCGCCCAGTCCTGCGGCAATAAAGCGGTTACAAGCCCG
CAAAAATAGCAGAGTAATGTCGCGATAGCGCGGCATTAATGCAGCTTTATTGat
tcctaAcgcgccccccgcgcaaatattcaactttcttattattattatgtagaactaaaatgtattcctaAttaaatgcgtacaacattat
taacatgtatgaaagaatccgtattaagtcagaaaatatccctCAATAAAGCTGCATTGTTCTTGATCTTC
TCCTTGTACTGCGCCTGCGCGCGCTCGAGCattcctaACATTTTAGTTCTACATAAT
AATAATAAGAAAGTTGAATATTTGCGCGGGGGGCGCGTattcctaATTTTCTGACT
TAATACGGATTCTTTCATACATGTTAATAATGTTGTACGCATTTAATatccctGctcg
agcgcgcgcaggcgcagtacaaggagaagatcaagaacGgccggcatggtcccagcctcctcgctggcgccggctgggca
acattccgaggggaccgtcccctcggtaatggcgaatgggacccCGGACCGAATACCCGGTCTGAACG
AGGGCGGCCGCGGTACCCAAGAAGTACTTAGAGTTAATTAAGGAGTTCAACGAA
GCTCCCACACCCTAGCATAACCCCTTGGGGCCTCTAAACGGGTCTTGAGGG
GTTTTTTGCTGAAAGGAGGAACTATATCCGTCGCGA
SEQ ID NO: 283 1x300-6 DQ088989.1. Plutella xylostella cytochrome P450
(CYP6BF1v2)
AAGCTTAGATCTCGATCCCGCGAAATtaatacgactcactatagggACCAATTGCAGGCGT
GAAGAATGTCCTCAGATACGGCTACCCTTCCTTCTTCTACAGCGTGGGATTGG
AGCTCTATTCCAGCAAAATTTACCGTTTCTTCCGATCTGTTATACTTGACGTTA
TAAACAGTCGTAACGGCGCCAAATCTTCGAGGAATGACATGGTGGATCTTATT
TCCGATTGGAAGAAGAACAAATACATAACGGGAGACAGTATTGATAATGGCA
TAGACGGTGGAAACAAGAAGGTGCGTATCGAAGTCGACGACGAACTTTTGGT
GAGCCAATGTGTGCTGTTCTTaagtactgcgatcgcgttaacgctttatcacgataccttctaccacatatcacta
acaacatcaacactcatcactctcgacgacatccactcgatcactactctcacacgaccgattaactcctcatccacgcggccgcct
gcaggagcAAGAACAGCACACATTGGCTCACCAAAAGTTCGTCGTCGACTTCGAT
ACGCACCTTCTTGTTTCCACCGTCTATGCCATTATCAATACTGTCTCCCGTTAT
GTATTTGTTCTTCTTCCAATCGGAAATAAGATCCACCATGTCATTCCTCGAAGA
TTTGGCGCCGTTACGACTGTTTATAACGTCAAGTATAACAGATCGGAAGAAAC
GGTAAATTTTGCTGGAATAGAGCTCCAATCCCACGCTGTAGAAGAAGGAAGG
GTAGCCGTATCTGAGGACATTCTTCACGCCTGCAATTGGTaagcttgccatctgttttcttgca
agatcagctgagcaataactagcatAGATAACAGATACttcgGtatctgttatctgttTTTTTTgctgaaaggagg
aactatatccggaTCGCGA
SEQ ID NO: 284 1x300-10 DQ088989.1. Plutella xylostella cytochrome P450
(CYP6BF1v2)
AAGCTTAGATCTCGATCCCGCGAAATtaatacgactcactatagggACCAATTGCAGGCGT
GAAGAATGTCCTCAGATACGGCTACCCTTCCTTCTTCTACAGCGTGGGATTGG
AGCTCTATTCCAGCAAAATTTACCGTTTCTTCCGATCTGTTATACTTGACGTTA
TAAACAGTCGTAACGGCGCCAAATCTTCGAGGAATGACATGGTGGATCTTATT
TCCGATTGGAAGAAGAACAAATACATAACGGGAGACAGTATTGATAATGGCA
TAGACGGTGGAAACAAGAAGGTGCGTATCGAAGTCGACGACGAACTTTTGGT
GAGCCAATGTGTGCTGTTCTTaagtactgcgatcgcgttaacgctttatcacgataccttctaccacatatcacta
acaacatcaacactcatcactctcgacgacatccactcgatcactactctcacacgaccgattaactcctcatccacgcggccgcct
gcaggagcAAGAACAGCACACATTGGCTCACCAAAAGTTCGTCGTCGACTTCGAT
ACGCACCTTCTTGTTTCCACCGTCTATGCCATTATCAATACTGTCTCCCGTTATGT
ATTTGTTCTTCTTCCAATCGGAAATAAGATCCACCATGTCATTCCTCGAAGA
TTTGGCGCCGTTACGACTGTTTATAACGTCAAGTATAACAGATCGGAAGAAAC
GGTAAATTTTGCTGGAATAGAGCTCCAATCCCACGCTGTAGAAGAAGGAAGG
GTAGCCGTATCTGAGGACATTCTTCACGCCTGCAATTGGTaagcttgccatctgttttcttgca
agatcagctgagcaataactagcatAGATAACAGATACttcgGtatctgttatctgttTTTTTTcAACAGAT
AGCCGCGttcgCGCGGCtatctgttTTTTTTgctgaaaggaggaactatatccggaTCGCGA

### Example 4. Kanamycin-Resistant High Copy Number pUC Vector (SEQUENCE ID NO: 285), with Restriction Sites HindIII and NruI, was Used for Cloning

Kanamycin-resistant pUC vector (high copy number)
SEQ ID NO: 285
TTAGAAAAACTCATCGAGCATCAAATGAAACTGCAATTTATTCATATCAG
GATTATCAATACCATATTTTTGAAAAAGCCGTTTCTGTAATGAAGGAGAA
AACTCACCGAGGCAGTTCCATAGGATGGCAAGATCCTGGTATCGGTCTGC
GATTCCGACTCGTCCAACATCAATACAACCTATTAATTTCCCCTCGTCAA
AAATAAGGTTATCAAGTGAGAAATCACCATGAGTGACGACTGAATCCGGT
GAGAATGGCAAAAGTTTATGCATTTCTTTCCAGACTTGTTCAACAGGCCA
GCCATTACGCTCGTCATCAAAATCACTCGCATCAACCAAACCGTTATTCA
TTCGTGATTGCGCCTGAGCGAGGCGAAATACGCGATCGCTGTTAAAAGGA
CAATTACAAACAGGAATCGAGTGCAACCGGCGCAGGAACACTGCCAGCGC
ATCAACAATATTTTCACCTGAATCAGGATATTCTTCTAATACCTGGAACG
CTGTTTTTCCGGGGATCGCAGTGGTGAGTAACCATGCATCATCAGGAGTA
CGGATAAAATGCTTGATGGTCGGAAGTGGCATAAATTCCGTCAGCCAGTT
TAGTCTGACCATCTCATCTGTAACATCATTGGCAACGCTACCTTTGCCAT
GTTTCAGAAACAACTCTGGCGCATCGGGCTTCCCATACAAGCGATAGATT
GTCGCACCTGATTGCCCGACATTATCGCGAGCCCATTTATACCCATATAA
ATCAGCATCCATGTTGGAATTTAATCGCGGCCTCGACGTTTCCCGTTGAA
TATGGCTCATATTCTTCCTTTTTCAATATTATTGAAGCATTTATCAGGGT
TATTGTCTCATGAGCGGATACATATTTGAATGTATTTAGAAAAATAAACA
AATAGGGGTCAGTGTTACAACCAATTAACCAATTCTGAACATTATCGCGA
GCCCATTTATACCTGAATATGGCTCATAACACCCCTTGTTTGCCTGGCGG
CAGTAGCGCGGTGGTCCCACCTGACCCCATGCCGAACTCAGAAGTGAAAC
GCCGTAGCGCCGATGGTAGTGTGGGGACTCCCCATGCGAGAGTAGGGAAC
TGCCAGGCATCAAATAAAACGAAAGGCTCAGTCGAAAGACTGGGCCTTTC
GCCCGGGCTAATTAGGGGGTGTCGCCCTTATTCGACTCTATAGTGAAGTT
CCTATTCTCTAGAAAGTATAGGAACTTCTGAAGTGGGGAAGCTTAAATTT
TCGCGAAAAATGAAGTGAAGTTCCTATACTTTCTAGAGAATAGGAACTTC
TATAGTGAGTCGAATAAGGGCGACACAAAATTTATTCTAAATGCATAATA
AATACTGATAACATCTTATAGTTTGTATTATATTTTGTATTATCGTTGAC
ATGTATAATTTTGATATCAAAAACTGATTTTCCCTTTATTATTTTCGAGA
TTTATTTTCTTAATTCTCTTTAACAAACTAGAAATATTGTATATACAAAA
AATCATAAATAATAGATGAATAGTTTAATTATAGGTGTTCATCAATCGAA
AAAGCAACGTATCTTATTTAAAGTGCGTTGCTTTTTTCTCATTTATAAGG
TTAAATAATTCTCATATATCAAGCAAAGTGACAGGCGCCCTTAAATATTC
TGACAAATGCTCTTTCCCTAAACTCCCCCCATAAAAAAACCCGCCGAAGC
GGGTTTTTACGTTATTTGCGGATTAACGATTACTCGTTATCAGAACCGCC
CAGGGGGCCCGAGCTTAAGACTGGCCGTCGTTTTACAACACAGAAAGAGT
TTGTAGAAACGCAAAAAGGCCATCCGTCAGGGGCCTTCTGCTTAGTTTGA
TGCCTGGCAGTTCCCTACTCTCGCCTTCCGCTTCCTCGCTCACTGACTCG
CTGCGCTCGGTCGTTCGGCTGCGGCGAGCGGTATCAGCTCACTCAAAGGC
GGTAATACGGTTATCCACAGAATCAGGGGATAACGCAGGAAAGAACATGT
GAGCAAAAGGCCAGCAAAAGGCCAGGAACCGTAAAAAGGCCGCGTTGCTG
GCGTTTTTCCATAGGCTCCGCCCCCCTGACGAGCATCACAAAAATCGACG
CTCAAGTCAGAGGTGGCGAAACCCGACAGGACTATAAAGATACCAGGCGT
TTCCCCCTGGAAGCTCCCTCGTGCGCTCTCCTGTTCCGACCCTGCCGCTT
ACCGGATACCTGTCCGCCTTTCTCCCTTCGGGAAGCGTGGCGCTTTCTCA
TAGCTCACGCTGTAGGTATCTCAGTTCGGTGTAGGTCGTTCGCTCCAAGC
TGGGCTGTGTGCACGAACCCCCCGTTCAGCCCGACCGCTGCGCCTTATCC
GGTAACTATCGTCTTGAGTCCAACCCGGTAAGACACGACTTATCGCCACT
GGCAGCAGCCACTGGTAACAGGATTAGCAGAGCGAGGTATGTAGGCGGTG
CTACAGAGTTCTTGAAGTGGTGGGCTAACTACGGCTACACTAGAAGAACA
GTATTTGGTATCTGCGCTCTGCTGAAGCCAGTTACCTTCGGAAAAAGAGT
TGGTAGCTCTTGATCCGGCAAACAAACCACCGCTGGTAGCGGTGGTTTTT
TTGTTTGCAAGCAGCAGATTACGCGCAGAAAAAAAGGATCTCAAGAAGAT
CCTTTGATCTTTTCTACGGGGTCTGACGCTCAGTGGAACGACGCGCGCGT
AACTCACGTTAAGGGATTTTGGTCATGAGCTTGCGCCGTCCCGTCAAGTC
AGCGTAATGCTCTGCTT

### Example 5. Cloning of Plasmid DNAs Coding for a Coat Protein and dsRNA Between 20 and 60 bp Long into the Vector Prepared in Example 4

Plasmid DNAs comprising sequences coding for dsRNAs are obtained by cloning one DNA sequence from those prepared in Section 2, Example 2 followed by one DNA sequence from those obtained in Section 2, Example 1 into a kanamycin-resistant high copy number pUC vector with SEQ ID NO: 285 between restriction sites HindIII and NruI. For example, SEQ ID NO: 277, followed by SEQ ID NO: 276 was cloned into SEQ ID NO: 285 and the plasmid DNA with SEQ ID NO: 286 was produced.

pNANOSURPxP450BI3X58C
SEQ ID NO: 286
TTAGAAAAACTCATCGAGCATCAAATGAAACTGCAATTTATTCATATCAGGAT
TATCAATACCATATTTTTGAAAAAGCCGTTTCTGTAATGAAGGAGAAAACTCA
CCGAGGCAGTTCCATAGGATGGCAAGATCCTGGTATCGGTCTGCGATTCCGAC
TCGTCCAACATCAATACAACCTATTAATTTCCCCTCGTCAAAAATAAGGTTATC
AAGTGAGAAATCACCATGAGTGACGACTGAATCCGGTGAGAATGGCAAAAGT
TTATGCATTTCTTTCCAGACTTGTTCAACAGGCCAGCCATTACGCTCGTCATCA
AAATCACTCGCATCAACCAAACCGTTATTCATTCGTGATTGCGCCTGAGCGAG
GCGAAATACGCGATCGCTGTTAAAAGGACAATTACAAACAGGAATCGAGTGC
AACCGGCGCAGGAACACTGCCAGCGCATCAACAATATTTTCACCTGAATCAGG
ATATTCTTCTAATACCTGGAACGCTGTTTTTCCGGGGATCGCAGTGGTGAGTA
ACCATGCATCATCAGGAGTACGGATAAAATGCTTGATGGTCGGAAGTGGCAT
AAATTCCGTCAGCCAGTTTAGTCTGACCATCTCATCTGTAACATCATTGGCAAC
GCTACCTTTGCCATGTTTCAGAAACAACTCTGGCGCATCGGGCTTCCCATACA
AGCGATAGATTGTCGCACCTGATTGCCCGACATTATCGCGAGCCCATTTATAC
CCATATAAATCAGCATCCATGTTGGAATTTAATCGCGGCCTCGACGTTTCCCGT
TGAATATGGCTCATATTCTTCCTTTTTCAATATTATTGAAGCATTTATCAGGGT
TATTGTCTCATGAGCGGATACATATTTGAATGTATTTAGAAAAATAAACAAAT
AGGGGTCAGTGTTACAACCAATTAACCAATTCTGAACATTATCGCGAGCCCAT
TTATACCTGAATATGGCTCATAACACCCCTTGTTTGCCTGGCGGCAGTAGCGC
GGTGGTCCCACCTGACCCCATGCCGAACTCAGAAGTGAAACGCCGTAGCGCC
GATGGTAGTGTGGGGACTCCCCATGCGAGAGTAGGGAACTGCCAGGCATCAA
ATAAAACGAAAGGCTCAGTCGAAAGACTGGGCCTTTCGCCCGGGCTAATTAG
GGGGTGTCGCCCTTATTCGACTCTATAGTGAAGTTCCTATTCTCTAGAAAGTAT
AGGAACTTCTGAAGTGGGGAAGCTTAGATCTCGATCCCGCGAAATTAATACGA
CTCACTATAGGGAGACCACAACGGTTTCCCTCTAGATCACAAGTTTGTACAAA
AAAGCAGGCTAAGAAGGAGATATACATACGCCGGCCATTCAAACATGAGGAT
TACCCATGTATTTAAATACCCATGTCCAGGCGCGCTCCGCGATCGCGGCAATA
AAGCGGTTACAAGCCCGCAAAAATAGCAGAGTAATGTCGCGATAGCGCGGCATT
AATGCAGCTTTATTGattcctaGTCCTCAGATACGGCTACCCTTCCTTCTTCTAC
AGCGTGGGATTGGAGCTCTATTCCAattcctaGCAAGTAGGTATACGGGACAATAC
TTTTCCGTCCTTCTTCAGAAAACCGCTCCGGACGatccctAcatgaggattacccatgtatccctC
AATAAAGCTGCATTttaccgtttcttccgatctgttatacttgacgttataaacagtattcctaTGGAATAGAGC
TCCAATCCCACGCTGTAGAAGAAGGAAGGGTAGCCGTATCTGAGGACattcctaCg
tccggagcggttttctgaagaaggacggaaaagtattgtcccgtatacctacttgcatccctAcatgaggatcacccatgtatccct
ACTGTTTATAACGTCAAGTATAACAGATCGGAAGAAACGGTAAggccggcatggtccc
agcctcctcgctggcgccggctgggcaacattccgaggggaccgtcccctcggtaatggcgaatgggacccCGGACCG
AATACCCGGTCTGAACGAGGGCGGCCGCGGTACCCAAGAAGTACTTAGAGTT
AATTAAGGAGTTCAAACATGAGGATCACCCATGTCGAAGCTCCCACACCCTAG
CATAACCCCTTGGGGCCTCTAAACGGGTCTTGAGGGGTTTTTTGCTGAAAGGA
GGAACTATATCCGGAATTCCGCAGGACTGGGCGGCGGGCTAGCATCGTCCATT
CCGACAGCATCGCCAGTCACTATGGCGTGCTGCTAGCGCTATATGCGTTGATG
CAATTTCTATGCGCACCCGTTCTCGGAGCACTGTCCGACCGCTTTGGCCGCCGC
CCAGTCCTGCGCATGCAGATCTCGATCCCGCGAAATTAATACGACTCACTATA
GGGAGACCACAACGGTTTCCCTCTAGATCCCTACATTAGAGAAGAGAGCTAAT
AAAGAAGGAGATATACATATGGCGTCTAACTTTACCCAATTCGTTCTGGTTGA
TAACGGCGGTACGGGTGACGTTACCGTAGCTCCGTCCAACTTCGCCAACGGTG
TTGCGGAATGGATTAGCTCTAACAGCCGCTCTCAGGCCTACAAAGTCACGTGC
TCCGTTCGTCAGTCTAGCGCGCAGAATCGCAAATACACCATCAAAGTTGAAGT
ACCGAAAGTCGCAACGCAGACCGTAGGCGGCGTAGAACTCCCAGTTGCGGCC
TGGCGCTCTTACCTCAACATGGAACTGACTATTCCGATTTTTGCGACGAACTCC
GACTGCGAACTGATTGTTAAGGCAATGCAGGGCCTGCTGAAAGACGGTAATC
CGATCCCATCTGCAATCGCTGCTAACTCTGGCATTTACTAATAAGCGGACGCG
CTGCCACCGCTGAGCAATAACTAGCATAACCCCTTGGGGCCTCTAAACGGGTC
TTGAGGGGTTTTTTGCTGAAAGGAGGAACTATATCCGTCGCGAAAAATGAAGT
GAAGTTCCTATACTTTCTAGAGAATAGGAACTTCTATAGTGAGTCGAATAAGG
GCGACACAAAATTTATTCTAAATGCATAATAAATACTGATAACATCTTATAGT
TTGTATTATATTTTGTATTATCGTTGACATGTATAATTTTGATATCAAAAACTG
ATTTTCCCTTTATTATTTTCGAGATTTATTTTCTTAATTCTCTTTAACAAACTAG
AAATATTGTATATACAAAAAATCATAAATAATAGATGAATAGTTTAATTATAG
GTGTTCATCAATCGAAAAAGCAACGTATCTTATTTAAAGTGCGTTGCTTTTTTC
TCATTTATAAGGTTAAATAATTCTCATATATCAAGCAAAGTGACAGGCGCCCT
TAAATATTCTGACAAATGCTCTTTCCCTAAACTCCCCCCATAAAAAAACCCGCCG
AAGCGGGTTTTTACGTTATTTGCGGATTAACGATTACTCGTTATCAGAACC
GCCCAGGGGGCCCGAGCTTAAGACTGGCCGTCGTTTTACAACACAGAAAGAG
TTTGTAGAAACGCAAAAAGGCCATCCGTCAGGGGCCTTCTGCTTAGTTTGATG
CCTGGCAGTTCCCTACTCTCGCCTTCCGCTTCCTCGCTCACTGACTCGCTGCGC
TCGGTCGTTCGGCTGCGGCGAGCGGTATCAGCTCACTCAAAGGCGGTAATACG
GTTATCCACAGAATCAGGGGATAACGCAGGAAAGAACATGTGAGCAAAAGGC
CAGCAAAAGGCCAGGAACCGTAAAAAGGCCGCGTTGCTGGCGTTTTTCCATA
GGCTCCGCCCCCCTGACGAGCATCACAAAAATCGACGCTCAAGTCAGAGGTG
GCGAAACCCGACAGGACTATAAAGATACCAGGCGTTTCCCCCTGGAAGCTCCC
TCGTGCGCTCTCCTGTTCCGACCCTGCCGCTTACCGGATACCTGTCCGCCTTTC
TCCCTTCGGGAAGCGTGGCGCTTTCTCATAGCTCACGCTGTAGGTATCTCAGTT
CGGTGTAGGTCGTTCGCTCCAAGCTGGGCTGTGTGCACGAACCCCCCGTTCAG
CCCGACCGCTGCGCCTTATCCGGTAACTATCGTCTTGAGTCCAACCCGGTAAG
ACACGACTTATCGCCACTGGCAGCAGCCACTGGTAACAGGATTAGCAGAGCG
AGGTATGTAGGCGGTGCTACAGAGTTCTTGAAGTGGTGGGCTAACTACGGCTA
CACTAGAAGAACAGTATTTGGTATCTGCGCTCTGCTGAAGCCAGTTACCTTCG
GAAAAAGAGTTGGTAGCTCTTGATCCGGCAAACAAACCACCGCTGGTAGCGG
TGGTTTTTTTGTTTGCAAGCAGCAGATTACGCGCAGAAAAAAAGGATCTCAAG
AAGATCCTTTGATCTTTTCTACGGGGTCTGACGCTCAGTGGAACGACGCGCGC
GTAACTCACGTTAAGGGATTTTGGTCATGAGCTTGCGCCGTCCCGTCAAGTCA
GCGTAATGCTCTGCTT

In another example, SEQ ID NO: 278, followed by SEQ ID NO: 276 were cloned into SEQ ID NO: 281 and the plasmid DNA with SEQ ID NO: 287 was produced.

pNANOSURPxP450BI5X58C
SEQ ID NO: 287
TTAGAAAAACTCATCGAGCATCAAATGAAACTGCAATTTATTCATATCAGGAT
TATCAATACCATATTTTTGAAAAAGCCGTTTCTGTAATGAAGGAGAAAACTCA
CCGAGGCAGTTCCATAGGATGGCAAGATCCTGGTATCGGTCTGCGATTCCGAC
TCGTCCAACATCAATACAACCTATTAATTTCCCCTCGTCAAAAATAAGGTTATC
AAGTGAGAAATCACCATGAGTGACGACTGAATCCGGTGAGAATGGCAAAAGT
TTATGCATTTCTTTCCAGACTTGTTCAACAGGCCAGCCATTACGCTCGTCATCA
AAATCACTCGCATCAACCAAACCGTTATTCATTCGTGATTGCGCCTGAGCGAG
GCGAAATACGCGATCGCTGTTAAAAGGACAATTACAAACAGGAATCGAGTGCAA
CCGGCGCAGGAACACTGCCAGCGCATCAACAATATTTTCACCTGAATCAGG
ATATTCTTCTAATACCTGGAACGCTGTTTTTCCGGGGATCGCAGTGGTGAGTA
ACCATGCATCATCAGGAGTACGGATAAAATGCTTGATGGTCGGAAGTGGCAT
AAATTCCGTCAGCCAGTTTAGTCTGACCATCTCATCTGTAACATCATTGGCAAC
GCTACCTTTGCCATGTTTCAGAAACAACTCTGGCGCATCGGGCTTCCCATACA
AGCGATAGATTGTCGCACCTGATTGCCCGACATTATCGCGAGCCCATTTATAC
CCATATAAATCAGCATCCATGTTGGAATTTAATCGCGGCCTCGACGTTTCCCGT
TGAATATGGCTCATATTCTTCCTTTTTCAATATTATTGAAGCATTTATCAGGGT
TATTGTCTCATGAGCGGATACATATTTGAATGTATTTAGAAAAATAAACAAAT
AGGGGTCAGTGTTACAACCAATTAACCAATTCTGAACATTATCGCGAGCCCAT
TTATACCTGAATATGGCTCATAACACCCCTTGTTTGCCTGGCGGCAGTAGCGC
GGTGGTCCCACCTGACCCCATGCCGAACTCAGAAGTGAAACGCCGTAGCGCC
GATGGTAGTGTGGGGACTCCCCATGCGAGAGTAGGGAACTGCCAGGCATCAA
ATAAAACGAAAGGCTCAGTCGAAAGACTGGGCCTTTCGCCCGGGCTAATTAG
GGGGTGTCGCCCTTATTCGACTCTATAGTGAAGTTCCTATTCTCTAGAAAGTAT
AGGAACTTCTGAAGTGGGGAAGCTTAGATCTCGATCCCGCGAAATTAATACGA
CTCACTATAGGGAGACCACAACGGTTTCCCTCTAGATCACAAGTTTGTACAAA
AAAGCAGGCTAAGAAGGAGATATACATACGCCGGCCATTCAAACATGAGGAT
TACCCATGTATTTAAATACCCATGTCCAGGCGCGCTCCGCGATCGCGGCAATA
AAGCGGTTACAAGCCCGCAAAAATAGCAGAGTAATGTCGCGATAGCGCGGCA
TTAATGCAGCTTTATTGattcctaGTCCTCAGATACGGCTACCCTTCCTTCTTCTAC
AGCGTGGGATTGGAGCTCTATTCCAattcctaACTGTTTATAACGTCAAGTATAAC
AGATCGGAAGAAACGGTAAATTTTGCTGGAATAGattcctaCCGTCTATGCCATTA
TCAATACTGTCTCCCGTTATGTATTTGTTCTTCTTCCAATCGGattcctaGCAAGTA
GGTATACGGGACAATACTTTTCCGTCCTTCTTCAGAAAACCGCTCCGGACGatcc
ctAcatgaggattacccatgtatccctCAATAAAGCTGCATTctatggactgtataacttcgtgtgcattcggcgtcga
ctctggattcctaTGGAATAGAGCTCCAATCCCACGCTGTAGAAGAAGGAAGGGTAGC
CGTATCTGAGGACattcctaCtattccagcaaaatttaccgtttcttccgatctgttatacttgacgttataaacagtattcct
aCcgattggaagaagaacaaatacataacgggagacagtattgataatggcatagacggattcctaCgtccggagcggttttctg
aagaaggacggaaaagtattgtcccgtatacctacttgcatccctAcatgaggatcacccatgtatccctCCAGAGTCGA
CGCCGAATGCACACGAAGTTATACAGTCCATAGggccggcatggtcccagcctcctcgctggcg
ccggctgggcaacattccgaggggaccgtcccctcggtaatggcgaatgggacccCGGACCGAATACCCGGT
CTGAACGAGGGCGGCCGCGGTACCCAAGAAGTACTTAGAGTTAATTAAGGAG
TTCAAACATGAGGATCACCCATGTCGAAGCTCCCACACCCTAGCATAACCCCTTG
GGGCCTCTAAACGGGTCTTGAGGGGTTTTTTGCTGAAAGGAGGAACTATAT
CCGGAATTCCGCAGGACTGGGCGGCGGGCTAGCATCGTCCATTCCGACAGCAT
CGCCAGTCACTATGGCGTGCTGCTAGCGCTATATGCGTTGATGCAATTTCTATG
CGCACCCGTTCTCGGAGCACTGTCCGACCGCTTTGGCCGCCGCCCAGTCCTGC
GCATGCAGATCTCGATCCCGCGAAATTAATACGACTCACTATAGGGAGACCAC
AACGGTTTCCCTCTAGATCCCTACATTAGAGAAGAGAGCTAATAAAGAAGGA
GATATACATATGGCGTCTAACTTTACCCAATTCGTTCTGGTTGATAACGGCGGT
ACGGGTGACGTTACCGTAGCTCCGTCCAACTTCGCCAACGGTGTTGCGGAATG
GATTAGCTCTAACAGCCGCTCTCAGGCCTACAAAGTCACGTGCTCCGTTCGTC
AGTCTAGCGCGCAGAATCGCAAATACACCATCAAAGTTGAAGTACCGAAAGT
CGCAACGCAGACCGTAGGCGGCGTAGAACTCCCAGTTGCGGCCTGGCGCTCTT
ACCTCAACATGGAACTGACTATTCCGATTTTTGCGACGAACTCCGACTGCGAA
CTGATTGTTAAGGCAATGCAGGGCCTGCTGAAAGACGGTAATCCGATCCCATC
TGCAATCGCTGCTAACTCTGGCATTTACTAATAAGCGGACGCGCTGCCACCGC
TGAGCAATAACTAGCATAACCCCTTGGGGCCTCTAAACGGGTCTTGAGGGGTT
TTTTGCTGAAAGGAGGAACTATATCCGTCGCGAAAAATGAAGTGAAGTTCCTA
TACTTTCTAGAGAATAGGAACTTCTATAGTGAGTCGAATAAGGGCGACACAAA
ATTTATTCTAAATGCATAATAAATACTGATAACATCTTATAGTTTGTATTATAT
TTTGTATTATCGTTGACATGTATAATTTTGATATCAAAAACTGATTTTCCCTTT
ATTATTTTCGAGATTTATTTTCTTAATTCTCTTTAACAAACTAGAAATATTGTAT
ATACAAAAAATCATAAATAATAGATGAATAGTTTAATTATAGGTGTTCATCAA
TCGAAAAAGCAACGTATCTTATTTAAAGTGCGTTGCTTTTTTCTCATTTATAAG
GTTAAATAATTCTCATATATCAAGCAAAGTGACAGGCGCCCTTAAATATTCTG
ACAAATGCTCTTTCCCTAAACTCCCCCCATAAAAAAACCCGCCGAAGCGGGTT
TTTACGTTATTTGCGGATTAACGATTACTCGTTATCAGAACCGCCCAGGGGGC
CCGAGCTTAAGACTGGCCGTCGTTTTACAACACAGAAAGAGTTTGTAGAAACG
CAAAAAGGCCATCCGTCAGGGGCCTTCTGCTTAGTTTGATGCCTGGCAGTTCC
CTACTCTCGCCTTCCGCTTCCTCGCTCACTGACTCGCTGCGCTCGGTCGTTCGG
CTGCGGCGAGCGGTATCAGCTCACTCAAAGGCGGTAATACGGTTATCCACAGA
ATCAGGGGATAACGCAGGAAAGAACATGTGAGCAAAAGGCCAGCAAAAGGC
CAGGAACCGTAAAAAGGCCGCGTTGCTGGCGTTTTTCCATAGGCTCCGCCCCC
CTGACGAGCATCACAAAAATCGACGCTCAAGTCAGAGGTGGCGAAACCCGAC
AGGACTATAAAGATACCAGGCGTTTCCCCCTGGAAGCTCCCTCGTGCGCTCTC
CTGTTCCGACCCTGCCGCTTACCGGATACCTGTCCGCCTTTCTCCCTTCGGGAAGC
GTGGCGCTTTCTCATAGCTCACGCTGTAGGTATCTCAGTTCGGTGTAGGTCG
TTCGCTCCAAGCTGGGCTGTGTGCACGAACCCCCCGTTCAGCCCGACCGCTGC
GCCTTATCCGGTAACTATCGTCTTGAGTCCAACCCGGTAAGACACGACTTATC
GCCACTGGCAGCAGCCACTGGTAACAGGATTAGCAGAGCGAGGTATGTAGGC
GGTGCTACAGAGTTCTTGAAGTGGTGGGCTAACTACGGCTACACTAGAAGAAC
AGTATTTGGTATCTGCGCTCTGCTGAAGCCAGTTACCTTCGGAAAAAGAGTTG
GTAGCTCTTGATCCGGCAAACAAACCACCGCTGGTAGCGGTGGTTTTTTTGTTT
GCAAGCAGCAGATTACGCGCAGAAAAAAAGGATCTCAAGAAGATCCTTTGAT
CTTTTCTACGGGGTCTGACGCTCAGTGGAACGACGCGCGCGTAACTCACGTTA
AGGGATTTTGGTCATGAGCTTGCGCCGTCCCGTCAAGTCAGCGTAATGCTCTG
CTT

### Example 6. Cloning of Plasmid DNAs Coding for dsRNA (No Coat Protein) into the Vector Prepared in Example 4

Plasmid DNAs comprising sequences coding for dsRNAs are obtained by cloning one DNA sequence from those prepared in example 3 followed by one DNA sequence from those obtained in example 1 of this section into a kanamycin-resistant high copy number pUC vector with SEQ ID NO:285 between restriction sites HindIII and NruI. For example, SEQ ID NO: 281 is cloned into SEQ ID NO: 285 and the plasmid DNA with SEQ ID NO: 288 is produced.

pNANOSURPxP450BI3X58E
SEQ ID NO: 288
TTAGAAAAACTCATCGAGCATCAAATGAAACTGCAATTTATTCATATCAGGAT
TATCAATACCATATTTTTGAAAAAGCCGTTTCTGTAATGAAGGAGAAAACTCA
CCGAGGCAGTTCCATAGGATGGCAAGATCCTGGTATCGGTCTGCGATTCCGAC
TCGTCCAACATCAATACAACCTATTAATTTCCCCTCGTCAAAAATAAGGTTATC
AAGTGAGAAATCACCATGAGTGACGACTGAATCCGGTGAGAATGGCAAAAGT
TTATGCATTTCTTTCCAGACTTGTTCAACAGGCCAGCCATTACGCTCGTCATCA
AAATCACTCGCATCAACCAAACCGTTATTCATTCGTGATTGCGCCTGAGCGAG
GCGAAATACGCGATCGCTGTTAAAAGGACAATTACAAACAGGAATCGAGTGC
AACCGGCGCAGGAACACTGCCAGCGCATCAACAATATTTTCACCTGAATCAGG
ATATTCTTCTAATACCTGGAACGCTGTTTTTCCGGGGATCGCAGTGGTGAGTA
ACCATGCATCATCAGGAGTACGGATAAAATGCTTGATGGTCGGAAGTGGCAT
AAATTCCGTCAGCCAGTTTAGTCTGACCATCTCATCTGTAACATCATTGGCAACGC
TACCTTTGCCATGTTTCAGAAACAACTCTGGCGCATCGGGCTTCCCATACA
AGCGATAGATTGTCGCACCTGATTGCCCGACATTATCGCGAGCCCATTTATAC
CCATATAAATCAGCATCCATGTTGGAATTTAATCGCGGCCTCGACGTTTCCCGT
TGAATATGGCTCATATTCTTCCTTTTTCAATATTATTGAAGCATTTATCAGGGT
TATTGTCTCATGAGCGGATACATATTTGAATGTATTTAGAAAAATAAACAAAT
AGGGGTCAGTGTTACAACCAATTAACCAATTCTGAACATTATCGCGAGCCCAT
TTATACCTGAATATGGCTCATAACACCCCTTGTTTGCCTGGCGGCAGTAGCGC
GGTGGTCCCACCTGACCCCATGCCGAACTCAGAAGTGAAACGCCGTAGCGCC
GATGGTAGTGTGGGGACTCCCCATGCGAGAGTAGGGAACTGCCAGGCATCAA
ATAAAACGAAAGGCTCAGTCGAAAGACTGGGCCTTTCGCCCGGGCTAATTAG
GGGGTGTCGCCCTTATTCGACTCTATAGTGAAGTTCCTATTCTCTAGAAAGTAT
AGGAACTTCTGAAGTGGGGAAGCTTAGATCTCGATCCCGCGAAATTAATACGA
CTCACTATAGGGAGACCACAACGGTTTCCCTCTAGATCACAAGTTTGTACAAA
AAAGCAGGCTAAGAAGGAGATATACATACGCCGGCCATTCAAATTTAAATAC
CCATGTCCAGGCGCGCTCCGCGATCGCGGCAATAAAGCGGTTACAAGCCCGC
AAAAATAGCAGAGTAATGTCGCGATAGCGCGGCATTAATGCAGCTTTATTGattc
ctaGTCCTCAGATACGGCTACCCTTCCTTCTTCTACAGCGTGGGATTGGAGCTCT
ATTCCAattcctaGCAAGTAGGTATACGGGACAATACTTTTCCGTCCTTCTTCAGAA
AACCGCTCCGGACGatccctCAATAAAGCTGCATTttaccgtttcttccgatctgttatacttgacgttata
aacagtattcctaTGGAATAGAGCTCCAATCCCACGCTGTAGAAGAAGGAAGGGTAGC
CGTATCTGAGGACattcctaCgtccggagcggttttctgaagaaggacggaaaagtattgtcccgtatacctacttgca
tccctACTGTTTATAACGTCAAGTATAACAGATCGGAAGAAACGGTAAggccggcatg
gtcccagcctcctcgctggcgccggctgggcaacattccgaggggaccgtcccctcggtaatggcgaatgggacccCGGA
CCGAATACCCGGTCTGAACGAGGGCGGCCGCGGTACCCAAGAAGTACTTAGA
GTTAATTAAGGAGTTCAACGAAGCTCCCACACCCTAGCATAACCCCTTGGGGC
CTCTAAACGGGTCTTGAGGGGTTTTTTGCTGAAAGGAGGAACTATATCCGGAA
TTCCGCAGGACTGGGCGGCGGGCTAGCATCGTCCATTCCGACAGCATCGCCAG
TCACTATGGCGTGCTGCTAGCGCTATATGCGTTGATGCAATTTCTATGCGCACC
CGTTCTCGGAGCACTGTCCGACCGCTTTGGCCGCCGCCCAGTCCTGCTCGCGA
AAAATGAAGTGAAGTTCCTATACTTTCTAGAGAATAGGAACTTCTATAGTGAG
TCGAATAAGGGCGACACAAAATTTATTCTAAATGCATAATAAATACTGATAAC
ATCTTATAGTTTGTATTATATTTTGTATTATCGTTGACATGTATAATTTTGATAT
CAAAAACTGATTTTCCCTTTATTATTTTCGAGATTTATTTTCTTAATTCTCTTTA
ACAAACTAGAAATATTGTATATACAAAAAATCATAAATAATAGATGAATAGTTTA
ATTATAGGTGTTCATCAATCGAAAAAGCAACGTATCTTATTTAAAGTGCGT
TGCTTTTTTCTCATTTATAAGGTTAAATAATTCTCATATATCAAGCAAAGTGAC
AGGCGCCCTTAAATATTCTGACAAATGCTCTTTCCCTAAACTCCCCCCATAAA
AAAACCCGCCGAAGCGGGTTTTTACGTTATTTGCGGATTAACGATTACTCGTT
ATCAGAACCGCCCAGGGGGCCCGAGCTTAAGACTGGCCGTCGTTTTACAACAC
AGAAAGAGTTTGTAGAAACGCAAAAAGGCCATCCGTCAGGGGCCTTCTGCTT
AGTTTGATGCCTGGCAGTTCCCTACTCTCGCCTTCCGCTTCCTCGCTCACTGAC
TCGCTGCGCTCGGTCGTTCGGCTGCGGCGAGCGGTATCAGCTCACTCAAAGGC
GGTAATACGGTTATCCACAGAATCAGGGGATAACGCAGGAAAGAACATGTGA
GCAAAAGGCCAGCAAAAGGCCAGGAACCGTAAAAAGGCCGCGTTGCTGGCGT
TTTTCCATAGGCTCCGCCCCCCTGACGAGCATCACAAAAATCGACGCTCAAGT
CAGAGGTGGCGAAACCCGACAGGACTATAAAGATACCAGGCGTTTCCCCCTG
GAAGCTCCCTCGTGCGCTCTCCTGTTCCGACCCTGCCGCTTACCGGATACCTGT
CCGCCTTTCTCCCTTCGGGAAGCGTGGCGCTTTCTCATAGCTCACGCTGTAGGT
ATCTCAGTTCGGTGTAGGTCGTTCGCTCCAAGCTGGGCTGTGTGCACGAACCC
CCCGTTCAGCCCGACCGCTGCGCCTTATCCGGTAACTATCGTCTTGAGTCCAAC
CCGGTAAGACACGACTTATCGCCACTGGCAGCAGCCACTGGTAACAGGATTA
GCAGAGCGAGGTATGTAGGCGGTGCTACAGAGTTCTTGAAGTGGTGGGCTAA
CTACGGCTACACTAGAAGAACAGTATTTGGTATCTGCGCTCTGCTGAAGCCAG
TTACCTTCGGAAAAAGAGTTGGTAGCTCTTGATCCGGCAAACAAACCACCGCT
GGTAGCGGTGGTTTTTTTGTTTGCAAGCAGCAGATTACGCGCAGAAAAAAAGG
ATCTCAAGAAGATCCTTTGATCTTTTCTACGGGGTCTGACGCTCAGTGGAACG
ACGCGCGCGTAACTCACGTTAAGGGATTTTGGTCATGAGCTTGCGCCGTCCCG
TCAAGTCAGCGTAATGCTCTGCTT

In another example, SEQ ID NO: 282, is cloned into SEQ ID NO: 285 and the plasmid DNA with SEQ ID NO: 289 is produced.

pNANOSUR10317XVPASEBI3X58E
SEQ ID NO: 289
TTAGAAAAACTCATCGAGCATCAAATGAAACTGCAATTTATTCATATCAGGAT
TATCAATACCATATTTTTGAAAAAGCCGTTTCTGTAATGAAGGAGAAAACTCA
CCGAGGCAGTTCCATAGGATGGCAAGATCCTGGTATCGGTCTGCGATTCCGAC
TCGTCCAACATCAATACAACCTATTAATTTCCCCTCGTCAAAAATAAGGTTATC
AAGTGAGAAATCACCATGAGTGACGACTGAATCCGGTGAGAATGGCAAAAGTTT
ATGCATTTCTTTCCAGACTTGTTCAACAGGCCAGCCATTACGCTCGTCATCA
AAATCACTCGCATCAACCAAACCGTTATTCATTCGTGATTGCGCCTGAGCGAG
GCGAAATACGCGATCGCTGTTAAAAGGACAATTACAAACAGGAATCGAGTGC
AACCGGCGCAGGAACACTGCCAGCGCATCAACAATATTTTCACCTGAATCAGG
ATATTCTTCTAATACCTGGAACGCTGTTTTTCCGGGGATCGCAGTGGTGAGTA
ACCATGCATCATCAGGAGTACGGATAAAATGCTTGATGGTCGGAAGTGGCAT
AAATTCCGTCAGCCAGTTTAGTCTGACCATCTCATCTGTAACATCATTGGCAAC
GCTACCTTTGCCATGTTTCAGAAACAACTCTGGCGCATCGGGCTTCCCATACA
AGCGATAGATTGTCGCACCTGATTGCCCGACATTATCGCGAGCCCATTTATAC
CCATATAAATCAGCATCCATGTTGGAATTTAATCGCGGCCTCGACGTTTCCCGT
TGAATATGGCTCATATTCTTCCTTTTTCAATATTATTGAAGCATTTATCAGGGT
TATTGTCTCATGAGCGGATACATATTTGAATGTATTTAGAAAAATAAACAAAT
AGGGGTCAGTGTTACAACCAATTAACCAATTCTGAACATTATCGCGAGCCCAT
TTATACCTGAATATGGCTCATAACACCCCTTGTTTGCCTGGCGGCAGTAGCGC
GGTGGTCCCACCTGACCCCATGCCGAACTCAGAAGTGAAACGCCGTAGCGCC
GATGGTAGTGTGGGGACTCCCCATGCGAGAGTAGGGAACTGCCAGGCATCAA
ATAAAACGAAAGGCTCAGTCGAAAGACTGGGCCTTTCGCCCGGGCTAATTAG
GGGGTGTCGCCCTTATTCGACTCTATAGTGAAGTTCCTATTCTCTAGAAAGTAT
AGGAACTTCTGAAGTGGGGAAGCTTAGATCTCGATCCCGCGAAATTAATACGA
CTCACTATAGGGAGACCACAACGGTTTCCCTCTAGATCACAAGTTTGTACAAA
AAAGCAGGCTAAGAAGGAGATATACATACGCCGGCCATTCAAATTTAAATAC
CCATGTCCAGGCGCGCTCCGCGATCGCACCAATTGCAGGCGTGAAGAATGTCC
TCAGATACGGCTACCCTTCCTTCTTCTACAGCGTGGGATTGGAGCTCTATTCCA
GCAAAATTTACCGTTTCTTCCGATCTGTTATACTTGACGTTATAAACAGTCGTA
ACGGCGCCAAATCTTCGAGGAATGACATGGTGGATCTTATTTCCGATTGGAAG
AAGAACAAATACATAACGGGAGACAGTATTGATAATGGCATAGACGGTGGAA
ACAAGAAGGTGCGTATCGAAGTCGACGACGAACTTTTGGTGAGCCAATGTGT
GCTGTTCTTGTTTAAACCCTCTAGCTGCTTTACAAAGTACTGGTTCCCTTTCCA
GCGGGATGCTTTATCTAAACGCAATGAGAGAGGTATTCCTCAGGCCACATCGC
TTCCTAGTTCCGCTGGGATCCATCGTTGGCGGCCGAAGCCGCCATTCCATAGT
GAGTTCTGGCGCGCCAAGAACAGCACACATTGGCTCACCAAAAGTTCGTCGTC
GACTTCGATACGCACCTTCTTGTTTCCACCGTCTATGCCATTATCAATACTGTC
TCCCGTTATGTATTTGTTCTTCTTCCAATCGGAAATAAGATCCACCATGTCATT
CCTCGAAGATTTGGCGCCGTTACGACTGTTTATAACGTCAAGTATAACAGATCGG
AAGAAACGGTAAATTTTGCTGGAATAGAGCTCCAATCCCACGCTGTAGAA
GAAGGAAGGGTAGCCGTATCTGAGGACATTCTTCACGCCTGCAATTGGTCGGA
CCGAATACCCGGTCTGAACGAGGGCGGCCGCGGTACCCAAGAAGTACTTAGA
GTTAATTAAGGAGTTCAACGAAGCTCCCACACCCTAGCATAACCCCTTGGGGC
CTCTAAACGGGTCTTGAGGGGTTTTTTGCTGAAAGGAGGAACTATATCCGGAA
TTCCGCAGGACTGGGCGGCGGGCTAGCATCGTCCATTCCGACAGCATCGCCAG
TCACTATGGCGTGCTGCTAGCGCTATATGCGTTGATGCAATTTCTATGCGCACC
CGTTCTCGGAGCACTGTCCGACCGCTTTGGCCGCCGCCCAGTCCTGCGGCAAT
AAAGCGGTTACAAGCCCGCAAAAATAGCAGAGTAATGTCGCGATAGCGCGGC
ATTAATGCAGCTTTATTGattcctaAcgcgccccccgcgcaaatattcaactttcttattattattatgtagaactaaa
atgtattcctaAttaaatgcgtacaacattattaacatgtatgaaagaatccgtattaagtcagaaaatatccctCAATAAAGC
TGCATTGTTCTTGATCTTCTCCTTGTACTGCGCCTGCGCGCGCTCGAGCattcctaA
CATTTTAGTTCTACATAATAATAATAAGAAAGTTGAATATTTGCGCGGGGGGC
GCGTattcctaATTTTCTGACTTAATACGGATTCTTTCATACATGTTAATAATGTTG
TACGCATTTAATatccctGctcgagcgcgcgcaggcgcagtacaaggagaagatcaagaacGgccggcatggtc
ccagcctcctcgctggcgccggctgggcaacattccgaggggaccgtcccctcggtaatggcgaatgggacccCGGACC
GAATACCCGGTCTGAACGAGGGCGGCCGCGGTACCCAAGAAGTACTTAGAGT
TAATTAAGGAGTTCAACGAAGCTCCCACACCCTAGCATAACCCCTTGGGGCCT
CTAAACGGGTCTTGAGGGGTTTTTTGCTGAAAGGAGGAACTATATCCGTCGCG
AAAAATGAAGTGAAGTTCCTATACTTTCTAGAGAATAGGAACTTCTATAGTGA
GTCGAATAAGGGCGACACAAAATTTATTCTAAATGCATAATAAATACTGATAA
CATCTTATAGTTTGTATTATATTTTGTATTATCGTTGACATGTATAATTTTGATA
TCAAAAACTGATTTTCCCTTTATTATTTTCGAGATTTATTTTCTTAATTCTCTTT
AACAAACTAGAAATATTGTATATACAAAAAATCATAAATAATAGATGAATAG
TTTAATTATAGGTGTTCATCAATCGAAAAAGCAACGTATCTTATTTAAAGTGC
GTTGCTTTTTTCTCATTTATAAGGTTAAATAATTCTCATATATCAAGCAAAGTG
ACAGGCGCCCTTAAATATTCTGACAAATGCTCTTTCCCTAAACTCCCCCCATAA
AAAAACCCGCCGAAGCGGGTTTTTACGTTATTTGCGGATTAACGATTACTCGT
TATCAGAACCGCCCAGGGGGCCCGAGCTTAAGACTGGCCGTCGTTTTACAACA
CAGAAAGAGTTTGTAGAAACGCAAAAAGGCCATCCGTCAGGGGCCTTCTGCTT
AGTTTGATGCCTGGCAGTTCCCTACTCTCGCCTTCCGCTTCCTCGCTCACTGAC
TCGCTGCGCTCGGTCGTTCGGCTGCGGCGAGCGGTATCAGCTCACTCAAAGGC
GGTAATACGGTTATCCACAGAATCAGGGGATAACGCAGGAAAGAACATGTGA
GCAAAAGGCCAGCAAAAGGCCAGGAACCGTAAAAAGGCCGCGTTGCTGGCGTTT
TTCCATAGGCTCCGCCCCCCTGACGAGCATCACAAAAATCGACGCTCAAGT
CAGAGGTGGCGAAACCCGACAGGACTATAAAGATACCAGGCGTTTCCCCCTG
GAAGCTCCCTCGTGCGCTCTCCTGTTCCGACCCTGCCGCTTACCGGATACCTGT
CCGCCTTTCTCCCTTCGGGAAGCGTGGCGCTTTCTCATAGCTCACGCTGTAGGT
ATCTCAGTTCGGTGTAGGTCGTTCGCTCCAAGCTGGGCTGTGTGCACGAACCC
CCCGTTCAGCCCGACCGCTGCGCCTTATCCGGTAACTATCGTCTTGAGTCCAAC
CCGGTAAGACACGACTTATCGCCACTGGCAGCAGCCACTGGTAACAGGATTA
GCAGAGCGAGGTATGTAGGCGGTGCTACAGAGTTCTTGAAGTGGTGGGCTAA
CTACGGCTACACTAGAAGAACAGTATTTGGTATCTGCGCTCTGCTGAAGCCAG
TTACCTTCGGAAAAAGAGTTGGTAGCTCTTGATCCGGCAAACAAACCACCGCT
GGTAGCGGTGGTTTTTTTGTTTGCAAGCAGCAGATTACGCGCAGAAAAAAAGG
ATCTCAAGAAGATCCTTTGATCTTTTCTACGGGGTCTGACGCTCAGTGGAACG
ACGCGCGCGTAACTCACGTTAAGGGATTTTGGTCATGAGCTTGCGCCGTCCCG
TCAAGTCAGCGTAATGCTCTGCTT

### Example 7. Transformation of E. coli with Plasmid DNAs Coding for dsRNA

Each plasmid DNA comprising sequences coding for dsRNAs described in examples 5 and 6 of this section are separately electroporated into E. coli strain HT115 (DE3) with genotype F, mcrA, mcrB, IN (rrnD-rrnE) 1, rnc14::Tn.10 (Lambda DE3 lysogen: lacUV 5 promoter-T7 polymerase)) and the resulting recombinant transformant is selected on LB agar plates containing 50 μg/mL of kanamycin. Single colonies are isolated, and the presence of the intact plasmid DNA from examples 5 and 6 of this section are confirmed by restriction enzyme analysis.

### Example 8. Preparation of dsRNA

Each recombinant transformant E. coli colony isolated from example 7 is separately grown in shake flasks. Each flask containing 100 mL of Super Broth with kanamycin (25 μg/ml, or another antibiotic if the plasmid DNA confers resistance to it) is inoculated with 5 mL of overnight cultures obtained from each transformant E. coli colony and induced with IPTG at a start OD600 of 0.4. The shake flasks are incubated for 16 h at 37° C. The cells are pelleted by centrifugation, resuspended in PBS (phosphate buffered saline) lysis buffer (137 mM NaCl, 2.7 mM KCl, 8 mM Na₂HPO₄, and 2 mM KH₂PO₄, adjusted to pH 7.2 to 7.4), and lysed with a sonicator or a French press or by adding lysozyme, incubated at between 35° C. to 70° C. for between 30 minutes to 90 minutes, and the cell debris removed by centrifugation. Ammonium sulfate is added to the lysate to achieve a concentration between 20% and 40% or, alternatively, a salt containing a quaternary ammonium cation, for example cetyltrimethylammonium bromide (CTAB), to achieve a concentration between 0.1% and 1% and between 20 g/L to 50 g/L of diatomaceous earth as filtering aid. The solid is removed by centrifugation or filtration and discarded. When the solid is obtained by using ammonium sulfate instead of using a salt containing a quaternary ammonium cation, most of the excess ammonium sulfate in the supernatant is removed dializing against water or against a suitable buffer like 1 mM citrate buffer, pH 4, using a dialysis membrane permeable to ammonium sulfate and impermeable to the dsRNA, for example using a Slide-A-Lyzer having a 3.5 kDalton (ThermoFisher Scientific) molecular weight cut off, or using a 30 kDalton molecular weight cut off hollow fiber membrane (MICROKROS 20 CM 30K MPES 0.5 MM). To the clarified lysate is added a salt containing a quaternary ammonium or phosphonium cation, for example CTAB, to achieve a concentration between 0.2% and 2% and stirred for between 2 hours and 6 hours. The precipitated material comprising dsRNA and quaternary ammonium or phosphonium cation is isolated by centrifugation. The precipitate is washed by suspending it in 5% IPA with 50 mM NaCl and subsequent centrifugation and drying under vacuum. Most of the isolated dsRNA is modified as described in Section 1 and used in a subsequent chemical modification as described in example.

A small aliquot of the precipitate isolated by centrifugation is used to confirm the production of the desired dsRNA. The precipitate is dissolved in a solution of sodium chloride between 0.5M NaCl and 1M NaCl and the low molecular weight salts, including most of the ammonium or phosphonium cation, are removed by dialysis against water using a dialysis membrane permeable to the low molecular weight salts present and impermeable to the dsRNA, for example using a Slide-A-Lyzer having a 3.5 kDalton molecular weight cut off, or using a 30 kDalton molecular weight cut off hollow fiber membrane. The production of dsRNA is confirmed using gel electrophoresis by comparison with a dsRNA or dsDNA marker set of standards.

The production of dsRNA of the desired sequence is confirmed using a QuantiGene RNA Assay (thermofisher.com/us/en/home/life-science/gene-expressionanalysis-genotyping/quantigene-rna-assays) aimed at detecting and quantifying one or more of the dsRNA sequences expressed by the corresponding sequence from example 2 or 3 of this section. For example, the production of the dsRNA resulting from the use of the transformant clone obtained in example 5 of this section having the plasmid DNA pNANOSURPxP450BI3X58C (SEQ ID NO: 286) is confirmed by using a QuantiGene RNA Assay aimed at detecting SEQ ID NO: 290, which is identical to nucleotide numbers 269-327 of SEQ ID NO: 277. The singleplex QuantiGene assay was performed according to the published manual (QuantiGene RNA Assays for Gene Expression Profiling|Thermo Fisher Scientific—US).

Likewise, the production of the dsRNA resulting from the use of the transformant clone obtained in example 5 having the plasmid DNA pNANOSURPxP450BI5X58C (SEQ ID NO: 287) is confirmed by using a QuantiGene RNA Assay aimed at detecting SEQ ID NO: 290, which is identical to nucleotide numbers 269-327 of SEQ ID NO: 278:

SEQ ID NO: 290:
GTCCTCAGATACGGCTACCCTTCCTTCTTCTACAGCGTGGGATTGGAGCT
CTATTCCA.

The singleplex QuantiGene assay was performed according to the published manual (QuantiGene RNA Assays for Gene Expression Profiling|Thermo Fisher Scientific—US).

### Section 3: Plants with Male Fertility Modifications

### Example 1. Identification of Target Polynucleotides for Producing Maize Plants Exhibiting Reduced Male Fertility

A study was identified in which spikelets at the pollen maturation stage with a length of 0.7-0.8 cm were collected from a cytoplasmic male sterile (CMS) line and from its male fertility-restored line and relative concentrations of RNAs were assayed for each line (Liu et al. BMC Plant Biology (2018) 18:190). Liu et al. determined that some of the RNAs were expressed at lower concentrations in the male sterile soybean line than in the male fertile soybean line, for example RNAs related to GRMZM2G018566 and GRMZM2G151169 genes.

### Example 2. Preparation of dsRNA and MdsRNA that Result in Maize Plants Exhibiting Reduced Male Fertility after the Exogenous Supply of a Blend of Such dsRNA and MdsRNA to Maize Cells or Seeds

The following sequences, i.e. SEQ ID NO: 291 and SEQ ID NO: 292 (5′-3′), are used to target mitochondrial enzyme complexes 2-oxoglutarate dehydrogenase (OGDH) and isocitrate dehydrogenase (IDH), respectively:

SEQ ID NO: 291, for targeting GRMZM2G151169
CCTTGTTGTTCTGTTGCCTCATGGTTATGATGGCCAAGGTCCTGAGCATT
CTAGTGCCCGCTTGGAGCGCTTCCTTCAGATGAGTGATGATAATCCTTTT
GTCATACCTGAGATGGAACCAACACTTCGCAAGCAGATACAAGAGTGTAA
CTGGCAAGTTGTGAATGTGACAACTCCTGCAAATTATTTTCATCTGTTGC
GTCGGCAGATACATAGGGAGTTCCGGAAGCCGCTGATTGTAACAGCTCCT
AAGAACCTGCTTAGGCACAAGGATTGCAAGTCAAATCTTTCAGAGTTTGA
SEQ ID NO: 292, for targeting GRMZM2G018566
CATCAGGTTGTGAGAGGTGTTGTAGAAAGTTTGAAAATTATTACCCGCCA
AGCAAGTTTGAGAGTGGCAGAATATGCTTTTCATTATGCCAAGGCCAATG
GCCGGGAAAGGGTCTCTGCGATTCACAAGGCCAATATTATGAGGAAGACA
GATGGTCTTTTCCTCAAGTGTTGCCGTGAAGTGGCTGAGACGTACCCTGA
AATTCAATACGAGGAGGTCATCATTGACAATTGTTGTATGACGCTTGTGA
AGAATCCTGGTCTTTTTGATGTATTAGTGATGCCAAACCTCTATGGTGAC

UdsRNAs and MdsRNAs, each having one of the two strands complementary to GRMZM2G018566, are separately prepared following published methods (Arhancet et al. U.S. Pat. No. 10,131,911). Likewise, UdsRNAs and MdsRNAs, each having one of the two strands complementary to GRMZM2G151169 are also separately prepared. MdsRNAs are prepared by reaction of UdsRNAs with furoyl acylimidazole (FAI). 40%+20% of the 2′OH groups of the riboses of the corresponding UdsRNA are reacted with FAI (40%+20% derivatization extent). UdsRNA and MdsRNA materials targeting GRMZM2G018566 are labeled, respectively, RNA-018566 and FAI_RNA-018566. UdsRNA and MdsRNA materials targeting GRMZM2G151169 are labeled, respectively, RNA-151169 and FAI_RNA-151169.

### Example 3. Exogenous Supply of Blends of dsRNA and MdsRNA to Maize Plants to Cause Reduced Male Fertility

Maize plants are grown in pots at a greenhouse using published procedures (Ray et al. Journal of Experimental Botany 49.325 (1998): 1381-1386.). Each treatment consists of a test material (or water as control) sprayed once per day one time or two times or three times at a given stage of plant growth. Thus, spraying is done once or twice or three times just before the formation of male flowers (tassels) or during tasseling, at V5, or V6, or V7, or VT of plant growth (McWilliams et al. “Corn growth and management quick guide.” (1999)). Each test material is sprayed at four doses, namely 0.02 mg (at 6 mg of active material/L aqueous solution), 0.2 mg (at 60 or 6 mg/L), 2 mg (at 0.6 or 0.06 g/L), 20 mg (at 6 or 0.6 g/L) per plant. Three test materials are prepared, namely 100% RNA-circ2483, 100% NMIA_RNA-circ2483, and a blend of equal parts by weight of RNA-circ2483 and NMIA_RNA-circ2483. A cohort of five plants (5 replicates) is used for each test material and control (water) at each dose and at each stage of plant growth at which the cohort is sprayed.

### Example 4. Evaluation of Maize Plants Showing Reduced Male Fertility after Spraying with Blends of UdsRNA and MdsRNA

Male fertility of maize plants is evaluated based on anther exertion and pollen fertility using published methods (Liu, et al. Peer J 4 (2016): e2719). Summarily, anther exertion is recorded every other day when tiller tassels start to branch. In addition, pollen is collected from the upper, middle and bottom of non-dehiscent anthers on the main stalk of each plant and fertility was rated according to the pollen staining ability using 1% (w/v) potassium iodide and iodine (K1-12). Maize plants sprayed with UdsRNA, or MdsRNA or 1:1 blend of UdsRNA and MdsRNA show described phenotypes consistent with lower male fertility than maize plants sprayed with water.

### Example 5. Identification of Target Polynucleotides for Producing Sorghum Plants Exhibiting Reduced Male Fertility

A study was identified in which genes in corn plants (genealogically closely related to sorghum) from a CMS line were found to be expressed at different concentration than in its male fertility-restored line during pollen maturation (Liu et al. BMC Plant Biology (2018) 18:190). For example, the protein 2-oxoglutarate dehydrogenase E1 component (Zea mays) GenBank: AQK80631.1, is expressed at lower concentration in CMS plants. We determined that this protein has a high degree of similarity with a constituent of the 2-oxoglutarate dehydrogenase in Sorghum bicolor, GenBank XP_002446307.1.

### Example 6. Preparation of dsRNA and MdsRNA that Result in Sorghum Plants Exhibiting Reduced Male Fertility after the Exogenous Supply of a Blend of Such dsRNA and MdsRNA to Sorghum Cells or Seeds

Sequence SEQ ID NO: 293 (5′-3′) is used to target a component of mitochondrial enzyme complex OGDH:

SEQ ID NO: 293, for targeting mRNA variant X1 of
OGDH complex
CCTTGTTGTTCTGCTGCCTCATGGTTATGATGGCCAAGGTCCTGAGCATT
CCAGTGCCCGCTTGGAGCGCTTCCTTCAGATGAGTGATGATAATCCTTTT
GTCATACCTGAGATGGAACCAACACTTCGCAAGCAGATACAAGAGTGTAA
CTGGCAGGTTGTGAATGTAACAACTCCTGCAAATTATTTTCATTTGTTGC
GTCGGCAGATACATAGGGAGTTCCGGAAGCCACTGATTGTAACAGCTCCT
AAGAACCTGCTTAGGCACAAGGATTGCAAGTCAAATCTTTCAGAGTTTGA

DsRNA and MdsRNA materials with sequence SEQ ID NO: 293 are prepared following procedures outlined in Example 2 of Section 3.

### Example 7. Exogenous Supply of Blends of dsRNA and MdsRNA to Sorghum Plants to Cause Reduced Male Fertility

Sorghum plants are grown in pots using published procedures (Zhao et al. Europ. J. Agronomy 22 (2005) 391-403). Each treatment consists of a test material (or water as control) sprayed once per day one time or two times or three times or four times during the second growth stage, when the reproductive structures of the panicle form and during flowering, at the beginning of the third growth stage (Gerik et al. Texas FARMER Collection (2003)). Thus, spraying is done once or twice or three times or four times, separated in time by intervals of about between 3 and 6 days. Each test material is sprayed at four doses, namely 0.02 mg (at 6 mg of active material/L aqueous solution), 0.2 mg (at 60 or 6 mg/L), 2 mg (at 0.6 or 0.06 g/L), 20 mg (at 6 or 0.6 g/L) per plant. Three test materials are prepared for sequence SEQ ID NO: 293, namely 100% UdsRNA, 100% MdsRNA, and a blend of equal parts by weight of UdsRNA and MdsRNA. A cohort of five plants (5 replicates) is used for each test material and control (water) at each dose and at each stage of plant growth at which the cohort is sprayed.

### Example 8. Evaluation of Sorghum Plants Showing Reduced Male Fertility after Spraying with Blends of UdsRNA and MdsRNA

Pollen viability was used as an indication of male fertility using published procedures (Prasad et al., Agricultural and Forest Meteorology 139 (2006): 237-251). Summarily, pollen viability was tested using 2% tri-phenyl tetrazolium chloride stain. A drop of tetrazolium chloride was added to the dispersed pollen. Tetrazolium chloride stains the live pollen with reddish purple color due to the formation of insoluble red formazan. The numbers of pollen grains stained was recorded 30 min after staining. The percentage of viable pollen was estimated as the percentage of total pollen that was stained. Sorghum plants sprayed with UdsRNA, or MdsRNA or 1:1 blend of UdsRNA and MdsRNA show described phenotypes consistent with lower male fertility than sorghum plants sprayed with water.

SEQUENCES—dsRNA Sequences for Lepidoptera.

SEQ ID NOs: 1-12 are provided above.

Sequence fragments presented herein are identified by the National Center for Biotechnology Information (NCBI) identification number and indicate the nucleotide ranges in the description. The full sequence may be found in the NCBI database.

ID 1. Plutella xylostella V-type proton ATPase subunit E (LOC105389010), mRNA. NCBI Reference Sequence: NM_001305532.1. 51-150

(SEQ ID NO: 13)
GGCAGCAACCATG GCGC TCAGCGATGCAGA
TGTCCAAAAACAGATCAAGCATATGATGGCCTTCATCGAGCAAGAGGCAA
ATGAAAAGGCCGAAGAAATC.

ID 2. Plutella xylostella V-type proton ATPase subunit E (LOC105389010), mRNA 151-250

(SEQ ID NO: 14)
GATGCTAAGGCTGAGGAGGAGTTCAACATCGAGAAGGGGCGTCTGGTGC
AGCAGCAGCGCCTCAAGATCATGGAGTACTACGAGAAGAAGGAGAAGCA
GG.

ID 3. Plutella xylostella V-type proton ATPase subunit E (LOC105389010), mRNA 251-350

(SEQ ID NO: 15)
TGGAACTCCAGAAGAAGATCCAATCCTCCAACATGCTGAACCAGGCCCG
TCTGAAGGTGCTGAAGGTGCGCGAGGACCACGTGGGCCACGTGTTGGAC
GA.

ID 4. Plutella xylostella V-type proton ATPase subunit E (LOC105389010), mRNA 351-450

(SEQ ID NO: 16)
GACGCGCCGCCGCCTCGCCGAGGTGCCCAACGACCAGGGGCTCTACTCC
GACCTGGTGGTCAAGCTCATCGTGCAGGCGCTGTTCCAGCTGGTTGAGC
CA.

ID 5. Plutella xylostella V-type proton ATPase subunit E (LOC105389010), mRNA 451-550

(SEQ ID NO: 17)
ACCGTAACCCTCCGCGTGCGCGAGGCCGACAAGCCGCTGATCGACAGCC
TGCTCGAGCGCGCGCAGGCGCAGTACAAGGAGAAGATCAAGAAGGATGT
GA.

ID 6. Plutella xylostella V-type proton ATPase subunit E (LOC105389010), mRNA 551-650

(SEQ ID NO: 18)
CCTTGAAGGTGGACACGGAGCACTACCTGCCGGTGGGCACCTGCGGCGG
GATTGAGTTGGTCGCCGCTAGGGGCCGCATCAAGATCATCAACACCCTG
GA.

ID 7. Plutella xylostella V-type proton ATPase subunit E (LOC105389010), mRNA 651-750

(SEQ ID NO: 19)
GTCGCGCATGGAGCTGATCGCGCAGCAGCTGCTGCCCGAGATCCGCACG
GCGCTGTTCGGACGGAACCCCAACCGCAAGTTCACCGACTAAACACCAA
CC.

ID 8. Plutella xylostella V-type proton ATPase subunit E (LOC105389010), mRNA 1501-1600

(SEQ ID NO: 20)
AGATTTGACTGGGCGCAGGGCGGTGTCACCCATACATCTTTCAGCTGAA
AAAGACATTCCTCTTTCATGTTTCCTGTCCTGGCAATCAAATGTTTCGG
CT.

ID 9. Plutella xylostella V-type proton ATPase subunit E (LOC105389010), mRNA 1601-1700

(SEQ ID NO: 21)
TGCTTTTAACAGTTCTATCGAAGAGCACCGTAGCTCTATAAATTACATAA
CGAATATAATGTTTAATCCCCATTATGGCACTGTTAAAATTCTTATGTA
A.

ID 10. Plutella xylostella V-type proton ATPase subunit E (LOC105389010), mRNA 1401-1500

(SEQ ID NO: 22)
ATACTGTTTACTATCGTGGACTTCCTGGGAATTATTTGATGCTGTAAGGT
TTATGGGTGACGGCAGCATCCCGCCTTATTCCCACTGAAATCGAAGTGA
A.

ID 11. Plutella xylostella V-type proton ATPase subunit E (LOC105389010), mRNA 51-166

(SEQ ID NO: 23)
GGCAGCAACCATGGCGCTCAGCGATGCAGATGTCCAAAAACAGATCAAGC
ATATGATGGCCTTCATCGAGCAAGAGGCAAATGAAAAGGCCGAAGAAATC
GATGCTAAGGCTGAGG.

ID 12. Plutella xylostella V-type proton ATPase subunit E (LOC105389010), mRNA 1476-1649

(SEQ ID NO: 24)
TTATTCCCACTGAAATCGAAGTGAAAGATTTGACTGGGCGCAGGGCGGTG
TCACCCATACATCTTTCAGCTGAAAAAGACATTCCTCTTTCATGTTTCCT
GTCCTGGCAATCAAATGTTTCGGCTTGCTTTTAACAGTTCTATCGAAGAG
CACCGTAGCTCTATAAATTACATA.

ID 13. Plutella xylostella V-type proton ATPase subunit E (LOC105389010), mRNA sequence

(SEQ ID NO: 25)
GGCAGCAACCATGGCGCTCAGCGATGCAGATGTCCAAAAACAGATCAAGC
ATATGATGGCCTTCATCGAGCAAGAGGCAAATGAAAAGGCCGAAGAAATC
GATGCTAAGGCTGAGGTTATTCCCACTGAAATCGAAGTGAAAGATTTGAC
TGGGCGCAGGGCGGTGTCACCCATACATCTTTCAGCTGAAAAAGACATTC
CTCTTTCATGTTTCCTGTCCTGGCAATCAAATGTTTCGGCTTGCTTTTAA
CAGTTCTATCGAAGAGCACCGTAGCTCTATAAATTACATA.

ID 14. XM_038113977.1|:71-220 PREDICTED: Plutella xylostella V-type proton ATPase catalytic subunit A (LOC105392322), transcript variant X1, mRNA

(SEQ ID NO: 26)
GTGATAAGTTGTGATTCTCCGCCATTCTCGGTCTGTCTTTTCCATCTTTC
TCGTCCAAAAAACAAGGTAATTGTTCAAGATGAGTCACGGGTTGAAGAGG
ATTGCCGATGAGGACAATGAAACCCAGTTCGGTTATGTCTTCGCTGTGT
C.

ID 15. NM_001305532.1|:51-200 Plutella xylostella V-type proton ATPase subunit E (LOC105389010), mRNA

(SEQ ID NO: 27)
GGCAGCAACCATGGCGCTCAGCGATGCAGATGTCCAAAAACAGATCAAGC
ATATGATGGCCTTCATCGAGCAAGAGGCAAATGAAAAGGCCGAAGAAATC
GATGCTAAGGCTGAGGAGGAGTTCAACATCGAGAAGGGGCGTCTGGTGC
A.

ID 16. Plutella xylostella V-type proton ATPase Sequence Cover both submit A and E

(SEQ ID NO: 28)
GTGATAAGTTGTGATTCTCCGCCATTCTCGGTCTGTCTTTTCCATCTTTC
TCGTCCAAAAAACAAGGTAATTGTTCAAGATGAGTCACGGGTTGAAGAGG
ATTGCCGATGAGGACAATGAAACCCAGTTCGGTTATGTCTTCGCTGTGTC
GGCAGCAACCATGGCGCTCAGCGATGCAGATGTCCAAAAACAGATCAAGC
ATATGATGGCCTTCATCGAGCAAGAGGCAAATGAAAAGGCCGAAGAAATC
GATGCTAAGGCTGAGGAGGAGTTCAACATCGAGAAGGGGCGTCTGGTGC
A.

ID 17. 101-200 PREDICTED: Plutella xylostella venom carboxylesterase-6 (LOC105388350), mRNA

(SEQ ID NO: 29)
ACGGAGGGAGATCCTCCTTCTGTGACTATAGCGCAAGGGTCCGTTGTAGG
TACTGCTGTGACCAGCTCTGGATTTACACACTATGAATTCCACGGGATA
C.

ID 18. 201-300 PREDICTED: Plutella xylostella venom carboxylesterase-6 (LOC105388350), mRNA

(SEQ ID NO: 30)
CGTATGCTGACTCTACTTCTGGATCTCACAGGTTCAAGGCGCCACGACCA
GCACCCACATTCACACAGACTTTTGTTGCTGATCGCAAAGGAATCAAAT
G.

ID 19. 301-400 PREDICTED: Plutella xylostella venom carboxylesterase-6 (LOC105388350), mRNA

(SEQ ID NO: 31)
TGTGAAAGCTATAAAGGGGGGATACGAGGGCACCGAAGATTGCTTGGTGG
CCAACGTCTACACACCGGCCATTGATCCAGAAAAGAAATACCCAGTAAT
G.

ID 20. 401-500 PREDICTED: Plutella xylostella venom carboxylesterase-6 (LOC105388350), mRNA

(SEQ ID NO: 32)
GTTTGGATTAAAGGGTCCGAGTTTGAGAAAACTAAGGGACCTGAACTATC
TTTTAGAAATCTTATTGAAAAAGAAGTAATAGTCGTGTCTCTAAACTTC
A.

ID 21. 501-600 PREDICTED: Plutella xylostella venom carboxylesterase-6 (LOC105388350), mRNA

(SEQ ID NO: 33)
GAGAGTCGATTCTCGGGTATCTTTGTCTTGGAACAGAAACTGCGCCTGGT
AACGCTGGATTGAAAGATATAATTGCTGGACTTCAATGGGTGAAAGATA
A.

ID 22. 601-700 PREDICTED: Plutella xylostella venom carboxylesterase-6 (LOC105388350), mRNA

(SEQ ID NO: 34)
CATTGAACAGTTTGGTGGAGACCCTGAGAGTATAACCCTATTTGGGCATG
GTTCTGGAGCTGCGGCGGTAGATTTAGTCACACTCTCTCCAATGTCTAA
G.

ID 23. 801-900 PREDICTED: Plutella xylostella venom carboxylesterase-6 (LOC105388350), mRNA

(SEQ ID NO: 35)
TTGGGCATGAAATTACTGAGGAATTAGATATTGAAAAGCTTTCGGAAGTT
TTTACTAGAACAAGTGTTGCCGCTCTAATGGCAGTTATAGATGAGTTGG
A.

ID 24. 901-1000 PREDICTED: Plutella xylostella venom carboxylesterase-6 (LOC105388350), mRNA

(SEQ ID NO: 36)
TTTAACTGATAACTCATTGGCTTTTGCTCCGTGTATCGAAAATGAGCAT
TTAGATGATGAAAAGTTTCTAGAAAAATCACCTTTTAGTACGCTAACTG
AA.

ID 25. 1001-1100 PREDICTED: Plutella xylostella venom carboxylesterase-6 (LOC105388350), mRNA

(SEQ ID NO: 37)
GGAACTTACACTAAAATACCTATGATCTTCGGATTTGTTGAAAACGAAG
GAACAATACGTTTTGATGAGGCACTAGAAGCTGATTGGCTAACAAAGAT
GG.

ID 25-2. 351-500 PREDICTED: Plutella xylostella venom carboxylesterase-6 (LOC105388350), mRNA

(SEQ ID NO: 38)
CCAACGTCTACACACCGGCCATTGATCCAGAAAAGAAATACCCAGTAAT
GGTTTGGATTAAAGGGTCCGAGTTTGAGAAAACTAAGGGACCTGAACTA
TCTTTTAGAAATCTTATTGAAAAAGAAGTAATAGTCGTGTCTCTAAACT
TCA.

ID 25-3. 951-1100 PREDICTED: Plutella xylostella venom carboxylesterase-6 (LOC105388350), mRNA

(SEQ ID NO: 39)
TAGATGATGAAAAGTTTCTAGAAAAATCACCTTTTAGTACGCTAACTGA
AGGAACTTACACTAAAATACCTATGATCTTCGGATTTGTTGAAAACGAA
GGAACAATACGTTTTGATGAGGCACTAGAAGCTGATTGGCTAACAAAGA
TGG.

ID 25-4. XM_011559245.2|:Plutella xylostella venom carboxylesterase-6 (LOC105388350), mRNA 351-500, 951-1100

(SEQ ID NO: 40)
CCAACGTCTACACACCGGCCATTGATCCAGAAAAGAAATACCCAGTAAT
GGTTTGGATTAAAGGGTCCGAGTTTGAGAAAACTAAGGGACCTGAACTA
TCTTTTAGAAATCTTATTGAAAAAGAAGTAATAGTCGTGTCTCTAAACT
TCATAGATGATGAAAAGTTTCTAGAAAAATCACCTTTTAGTACGCTAAC
TGAAGGAACTTACACTAAAATACCTATGATCTTCGGATTTGTTGAAAAC
GAAGGAACAATACGTTTTGATGAGGCACTAGAAGCTGATTGGCTAACAA
AGATGG.

ID 26. 101-250 Plutella xylostella strain DBM1Ac-S mitogen-activated protein kinase 4 isoform X1 (MAP4K4) mRNA, complete cds, alternatively spliced

(SEQ ID NO: 41)
AATATAAAGTGCGTGATTTTACACATGTCGAATGTCATGAGTGAAAGGA
TCTTTGTAGGTTTCTTTACATTGAAGTGAATTCTCGTGCTTTGTCTTCG
TGTGTGATAATACTCAAAATGGCGCATCAACTGGCTCCGTCTGTGAATT
GCT.

ID 27. 901-1050 Plutella xylostella strain DBM1Ac-S mitogen-activated protein kinase 4 isoform X1 (MAP4K4) mRNA, complete cds, alternatively spliced

(SEQ ID NO: 42)
ATGGCCGAGAGTCAGCCGCCCCTGTGTGACCTTCACCCAATGAGAGCAT
TGTTTCTTATACCAAGAAATCCTCCACCCCGTTTAAAGTCAAAGAAATG
GGCAAAGAAATTTCATAGTTTTATTGAAACTGTTCTTGTGAAAGACTAT
CAC.

ID 28. 51-150 Plutella xylostella prophenoloxidase 1 mRNA, complete cds

(SEQ ID NO: 43)
TTGCTTCATGCAGAAGGGCGACGACAAGACCGTCTTCCAGATCCCGGAC
AACTTCTACCCAGAAAAGTACAAGAAGGTGGGCAACCAGCTGGCCGACC
GG.

ID 29. 301-400 Plutella xylostella prophenoloxidase 1 mRNA, complete cds

(SEQ ID NO: 44)
GGAATGCGTGACGTGGAGGACCTGCAGTCCGTGTGTAGCTACTGCCAGC
TCCGCATCAACCCCTACATGTTCAACTACTGCCTGTCGGTCGCCATGCT
GC.

ID 30. 401-500 Plutella xylostella prophenoloxidase 1 mRNA, complete cds

(SEQ ID NO: 45)
ACAGACCAGACACGAAGGGCCTGTCGCCGCCGACGCTGGCGGAGACGTT
CCCCGACAAGTTCATGGACCCCAAGGTGTTCCGCCGCGCGCGGGAGACC
TC.

ID 31. 501-600 Plutella xylostella prophenoloxidase 1 mRNA, complete cds

(SEQ ID NO: 46)
CACCACCGCGCCTGCTGGGGACAGGATGCCAGTCCTAATCCCGGTCAAC
TACACGGCCTCCGACGCTGAGCCAGAACAACGCATCGCGTACTTCCGCG
AA.

ID 32. 601-700 Plutella xylostella prophenoloxidase 1 mRNA, complete cds

(SEQ ID NO: 47)
GACATCGGCATCAACCTGCACCACTGGCACTGGCACCTGGTGTACCCCTT
CGAGGCGGCCGACCGCGCCGTGGTGGACAAGGACAGGCGCGGCGAGCTGC
GC.

ID 33. 601-700 Plutella xylostella prophenoloxidase 1 mRNA, complete cds

(SEQ ID NO: 48)
TGTACTACATGCACCAGCAGATCATCGCCAGATACAACGCAGAGCGTCT
GTGCAACAACCTGGGCTTCGTGACGCGCTACAACGACTTCCGCGGGCCC
AT.

ID 34. 801-900 Plutella xylostella prophenoloxidase 1 mRNA, complete cds

(SEQ ID NO: 49)
CGCCGAGGGGTACTTCCCCAAGATGGACTCGCAGGTCGCCAGCAGGGCC
TGGCCTCCTAGGTTCTCCGGCACCACGATCCGCGACCTGGACCGCCCCG
TG.

ID 35. 901-1000 Plutella xylostella prophenoloxidase 1 mRNA, complete cds

(SEQ ID NO: 50)
GACCAGATCCGCTCCGACGTGTCTGAGATGGAGACCTGGAGGGACCGCT
TCATCCAGGCCATCGACAGCGGCACTATTGTTCTGCCCAACGGCCGCAC
CC.

ID 36. 1001-1100 Plutella xylostella prophenoloxidase 1 mRNA, complete cds

(SEQ ID NO: 51)
AGCGCCTCGACGAGGAGACCGGCATCGACGTGCTGGCCAACCTCAT
GGAGTCGTCCATCATCAGCCGCAACCGCGCCTACTACGGGGACCTG
CACAACAT

ID 37. 1101-1200 Plutella xylostella prophenoloxidase 1 mRNA, complete cds

(SEQ ID NO: 52)
GGGGCATGTGTTCATCTCCTATGCGCACGACCCCGACCACCGGCAC
TTGGAACAATTCGGCGTGATGGGAGACCCGGCCACGGCCATGAGGG
ACCCGATC.

ID 38. 1201-1300 Plutella xylostella prophenoloxidase 1 mRNA, complete cds

(ID SEQ ID NO: 53)
TTCTACCGCTGGCACGCGTACGTCGACGACATCTTCCAGAGATACA
AGGCCACACTACCAGCCTACACCAGGGAGAGGTTGGACTTCCCAGG
CATCCGCG.

ID 40. 1301-1400 Plutella xylostella prophenoloxidase 1 mRNA, complete cds

(SEQ ID NO: 54)
TCTCCTCCATCGCCATCTCGGGCCGCACTCCGAACCAGTTCTCGAC
GCAGTGGGAGCAGAGTTCAGTGAACCTGGCGCGCGGGCTGGACTTC
ATGCCGCG.

ID 41. 1401-1500 Plutella xylostella prophenoloxidase 1 mRNA, complete cds

(SEQ ID NO: 55)
CGGCGCCGTGCTGGCGCGGTTCACGCATCTGCAGCATGACGAGTTT
GAGTACACCATCGAGTGCGACAACACAACCGGCCAAGCAGCCATGG
GCACCGTC.

ID 42. 1501-1600 Plutella xylostella prophenoloxidase 1 mRNA, complete cds

(SEQ ID NO: 56)
CGCATATTCCTCGCCCCGACCACCGACCAGGCCGGCAACGCACTCA
ACTTCGAGGAGCAGAGGCGACTCATGATCGAGCTGGACAAGTTTAC
TCAGGGCT

ID 43. 1601-1700 Plutella xylostella prophenoloxidase 1 mRNA, complete cds

(SEQ ID NO: 57)
TGCGCCCCGGCAGCAACACCATCCGGCGTCGCAGCATCGACTCCTC
AGTTACCATCCCCTACGAGCGAACATTCCGGGACGAGTCCCAACAC
CCCGGAGA

ID 44. 1701-1800 Plutella xylostella prophenoloxidase 1 mRNA, complete cds

(SEQ ID NO: 58)
CGCTGGCTCAGCTCAGTCAGCCGACTTCGACTTCTGCGGCTGCGGC
TGGCCGCACCACATGCTGATACCGAAGGGGACTCAGCAGGGATGGA
ACTGTGT

ID 45. 1801-1900 Plutella xylostella prophenoloxidase 1 mRNA, complete cds

(SEQ ID NO: 59)
CTCTTCTGCATGATTACCAACTGGAATGAAGATCGGGTGGAGCAAG
ACACAGTAGGAACCTGCAACGACGCAGCCTCCTACTGCGGTATCCG
GGACCGCC

ID 46. 1901-2000 Plutella xylostella prophenoloxidase 1 mRNA, complete cds

(SEQ ID NO: 60)
GCTACCCGGACCGCAAGCCTATGGGATTCCCCTTCGATAGACCAGC
GCCATCTACCGGCAGTTTGGGAGACTTCTTGACCCCCAACATGACT
GTGCAGAA

ID 46-1. 101-200 Plutella xylostella prophenoloxidase 1 mRNA, complete cds

(SEQ ID NO: 61)
TTACAGTGTTCGTGGTATTTTAAGCCCAAACGCTAATCCAAGATGG
CGGACAAAAACAACCTGCTGCTGTTCTTCGACCGCCCCACGGAGCC
CTGCTTCA

ID 46-2. Plutella xylostella prophenoloxidase 1 mRNA, complete cds

(SEQ ID NO: 62)
TCAGGTTTACTGATGCGGTCAGGCAGCGTCAGCAGCGGTAGAGTGT
GGGAGGGAGACATGGTTCATACGTTACGAGAGTTCTAGCTAAACTT
AAACACACAATGAAAGCTGACTGTAACTTTTGTAGATTTTGCCTTC
AAAATGACAATTACTTAGGCACTAATTAGGTTCTAATTTTGTATAA
TTCATTTTTAGCGACA

ID 46-3. Plutella xylostella prophenoloxidase 1 sequence mRNA, complete cds

(SEQ ID NO: 63)
TTACAGTGTTCGTGGTATTTTAAGCCCAAACGCTAATCCAAGATGG
CGGACAAAAACAACCTGCTGCTGTTCTTCGACCGCCCCACGGAGCC
CTGCTTCATCAGGTTTACTGATGCGGTCAGGCAGCGTCAGCAGCGG
TAGAGTGTGGGAGGGAGACATGGTTCATACGTTACGAGAGTTCTAG
CTAAACTTAAACACACAATGAAAGCTGACTGTAACTTTTGTAGATT
TTGCCTTCAAAATGACAATTACTTAGGCACTAATTAGGTTCTAATT
TTGTATAATTCATTTTTAGCGACA

ID 47. 151-250 Plutella xylostella glutathione synthetase (Gss), mRNA

(SEQ ID NO: 64)
CGACCCACGGCTCCAAGTCCGTGCTGACCCCCAACCTCGACGTGCT
GACCCGCTCAGGAGTGTCCCTCCACCGCTACTACACCCACGCTCTC
TGCTCGCC

ID 48. 251-350 Plutella xylostella glutathione synthetase (Gss), mRNA

(SEQ ID NO: 65)
CGCCCGTACCGCTGTGCTCACCGGCAAATACGCCCACACCGTCGGT
ATGCAGGGTATGCCTCTGTCCAACGCTGAGGAGCGTGGTATCCCCC
TAGAGGAG

ID 49. 351-450 Plutella xylostella glutathione synthetase (Gss), mRNA

(SEQ ID NO: 66)
CGCCTGATCTCTCAGTACCTACAGGACGCTGGTTACAGGACCCAGATGGT
CGGAAAGTGGCACGTCGGTCACGCCTTCTTCGAGCAGCTGCCCACTTACA

ID 50. 451-550 Plutella xylostella glutathione synthetase (Gss), mRNA

(SEQ ID NO: 67)
GAGGATTCGAGAACCACTTCGGTGTCCGCGGTGGATTCATCGACTACTAC
GAATACAACGCTCAGGAGCAGCTTGACGGCAGGCCAGTCACTGGACTGTG

ID 51. 551-650 Plutella xylostella glutathione synthetase (Gss), mRNA

(SEQ ID NO: 68)
TCTGTTCGACGACCTGCAGCCCGACTGGACCACCGAGGGATACATCACCG
ACGTCTACACCGAGAAGTCCACCACCATCATTGAGAACCACAACGTCTCC

ID 52. 651-750 Plutella xylostella glutathione synthetase (Gss), mRNA

(SEQ ID NO: 69)
GAGCCCCTGTACCTGCTGCTGACCCACCACGCTCCCCACAACGGCAACGA
AGACGCTTCCCTGCAGGCCCCTCCTGAAGAGGTCCGCGCCCAGAGGCACG

ID 53. 751-850 Plutella xylostella glutathione synthetase (GSS), mRNA

(SEQ ID NO: 70)
TCGAGCTCCACCCCAGACGTATCTTCGCCGCTATGGTTAAGAAACTGGAC
GACAGTATCGGAGAAATCGTCGCTACCCTCGAGAAGAAGGGCATGCTCGA

ID 54. 851-950 Plutella xylostella glutathione synthetase (Gss), mRNA

(SEQ ID NO: 71)
GAACACCATCATCACCTTCTCCACTGACAACGGTGCCCCCACCGTCGGTC
TTGGCGCCAACTCTGGTTCCAACTACCCCCTGAGAGGAGTCAAGAAGTCC

ID 54. 951-1050 Plutella xylostella glutathione synthetase (Gss), mRNA

(SEQ ID NO: 72)
CCCTGGGAGGGAGGTATCCGTGGTAACGCCATGATCTGGGCCGGTCCCGA
GGTCGCCCCCGGAAACGCGTGGCGTGGAAAGGTTTACGACGGCAACATGC

ID 57. 1051-1150 Plutella xylostella glutathione synthetase (Gss), mRNA

(SEQ ID NO: 73)
ACGCCGCTGACTGGGTCCCCACTCTGCTTGAGGCCATCGGTGAGAAGATC
CCCGCCGGTCTGGACGGTATCCCCATGTGGAGCCACATCATCGAGAACAA

ID 58. 1151-1250 Plutella xylostella glutathione synthetase (Gss), mRNA

(SEQ ID NO: 74)
GCCTTCTCCCCGTACCGAGATCTTCGAGATCGACGACTACTTCAACCACT
CCTCTGTCACCCTCGGCCGCCACAAGCTCGTCAAGGGAACCATCGACGAG

ID 59. 1251-1350 Plutella xylostella glutathione synthetase (Gss), mRNA

(SEQ ID NO: 75)
TCTCTCAGCAAGCACTACGGTGAAGACCTCCGTGGCATCATCGGAACTCC
CCCAGACTACAAGCAGAAGCTGCGCGACAGCAAGGCATGGGAGTCTCTGG

ID 60. 1351-1450 Plutella xylostella glutathione synthetase (Gss), mRNA

(SEQ ID NO: 76)
AGACCATCGGCATCCCTCTGGACGCTGACGTCATGGCTGACCGCGATGAG
GCTATCGTCACTTGCGGAAATGTCGTCCCCAAGCCTTGCAGTCCTTCTGC

ID 61. 1451-1550 Plutella xylostella glutathione synthetase (Gss), mRNA

(SEQ ID NO: 77)
CGAGTCTTGGTGCCTGTACGACATCATCGAGGACCCTTGTGAGCTTCGTG
ACCTGTCTGAGGAGCTTCCTCAGCTGGCTCAGATCCTTCTGTACCGTCTG

ID 62. 1551-1650 Plutella xylostella glutathione synthetase (Gss), mRNA

(SEQ ID NO: 78)
GAGCAGGAAGAGGCCAAGATCATCCCCAGGGAGGGCCAGTACGTCGCTGA
CCCCAAGTCTGCCCCCAAGTACTTCAACTACACCTGGGACGCGTACCTGT

ID 63. 1651-1750 Plutella xylostella glutathione synthetase (Gss), mRNA

(SEQ ID NO: 79)
CCGTCGAACCCTACTCCGACTCCGAATAGACGAAGCTCAGCTCAAGCGGC
GCAGTTCGCCGTGAAAGTTGTAAATGTTGATCCTGGCCTTAATTTCAGTA

ID 63-2. Plutella xylostella glutathione synthetase (Gss), mRNA

(SEQ ID NO: 80)
GAGGATTCGAGAACCACTTCGGTGTCCGCGGTGGATTCATCGACTACTAC
GAATACAACGCTCAGGAGCAGCTTGACGGCAGGCCAGTCACTGGACTGTG
TCGAGCTCCACCCCAGACGTATCTTCGCCGCTATGGTTAAGAAACTGGAC
GACAGTATCGGAGAAATCGTCGCTACCCTCGAGAAGAAGGGCATGCTCGA
CCGTCGAACCCTACTCCGACTCCGAATAGACGAAGCTCAGCTCAAGCGGC
GCAGTTCGCCGTGAAAGTTGTAAATGTTGATCCTGGCCTTAATTTCAGTA

ID 64. AY904342.1:160-460 Plutella xylostella pheromone biosynthesis activating neuropeptide (PBAN) mRNA, complete cds

(SEQ ID NO: 81)
ATCCAGCGGGACGCCCGCGACCGCGCCTCAATGTGGTTCGGGCCGCGCCT
CGGGAAGCGAGCCATGCACCTCGCGCCCGACGGTGACGGACAAGCAGTAT
ACAGGATGCTCGAAGCTGCAGACGCGCTCAAGTACTACTACGACCAGCTC
CAGTATTATGGGGCTCAGGCAGACGATCCTGAGACTAAAGTGACAAAGAA
GGTGATCTTCACCCCCAAGTTGGGCCGCAACGCTGATGAAGACCAGCAGC
AGTCGGTGGACTTCACGCCGAGACTAGGGCGCCGCCGGCTCAAGGACTCG
G

ID 65. 151-250 PREDICTED: Plutella xylostella charged multivesicular body protein 4b (LOC105396929), mRNA

(SEQ ID NO: 82)
ACAACCATGAGTTTTCTGGGGAAAATATTCGGTGGTAAGAAGGAGGAGAA
AGGTCCATCAACACACGAAGCTATTCAAAAATTACGCGAGACCGAAGAAT

ID 66. 251-350 PREDICTED: Plutella xylostella charged multivesicular body protein 4b (LOC105396929), mRNA

(SEQ ID NO: 83)
TGCTCATCAAAAAGCAGGACTTCCTGGAGAAGAAAATACAATTAGAAGTA
GACACAGCCAGGAAACATGGCACTAAGAACAAAAGAGCGGCCATCGCTGC

ID 67. 351-450 PREDICTED: Plutella xylostella charged multivesicular body protein 4b (LOC105396929), mRNA

(SEQ ID NO: 84)
ACTTAAACGCAAGAAGCGTTACGAGAAGCAGCTCACACAGATCGACGGCA
CGCTCAGCCAGATAGAGATGCAGAGAGAAGCATTGGAGGGAGCCAACACT

ID 68. 451-550 PREDICTED: Plutella xylostella charged multivesicular body protein 4b (LOC105396929), mRNA

(SEQ ID NO: 85)
AACACTCAAGTACTGAACACGATGCGAGAGGCCGCCGCGGCTATGAAGCT
CGCTCACAAGGATATTGACGTAGACAAAGTGCACGATATCATGGACGACA

ID 69. 551-650 PREDICTED: Plutella xylostella charged multivesicular body protein 4b (LOC105396929), mRNA

(SEQ ID NO: 86)
TCGCTGAACAACATGATGTGGCTCGCGAGATCACGGATGCAATCAGCAAC
AATGTGGCCTTCCCGAGTGACATTGATGATGAGGAGCTGGAGAGAGAGTT

ID 70. 651-751 PREDICTED: Plutella xylostella charged multivesicular body protein 4b (LOC105396929), mRNA

(SEQ ID NO: 87)
GGATGAACTGGAACAGGAGGACCTGGACAAGGAGATGCTGGGCATCAACG
TGCCCACGGACCAGCTGCCCGACGTGCCGTCCGCCGAGCCCGCCGCGCCG
C

ID 71. 751-850 PREDICTED: Plutella xylostella charged multivesicular body protein 4b (LOC105396929), mRNA

(SEQ ID NO: 88)
CGCCCCGCCAAGGCCAAGCCTGCTAGCGAAGACGACGATGATCTGGCTAA
ACTGCAATCGTGGGCGACATAAATGTAAGTGTTGGAAGCGAAACCGAATA

ID 72. 851-950 PREDICTED: Plutella xylostella charged multivesicular body protein 4b (LOC105396929), mRNA

(SEQ ID NO: 89)
CATATGTATAGTCTCCGTTACACATCCAACTACTTAGCTTATTCTAAGGC
TGCGTCCCTGTAGGCAAACAGTATTCTCGTTACTAGTCTATGGTAATTTG

ID 73. 951-1050 PREDICTED: Plutella xylostella charged multivesicular body protein 4b (LOC105396929), mRNA

(SEQ ID NO: 90)
AGTAGAGTGAAAGAACAACTAGCTAATAAAAATAAGGAAGGCCCAAGACA
GAGTGTTGTGGCGCTCCTTGGGATTGGTCTATGTCCAACATTGGACGATA

ID 74. 1051-1150 PREDICTED: Plutella xylostella charged multivesicular body protein 4b (LOC105396929), mRNA

(SEQ ID NO: 91)
ACAGCTTTATGACTTTAATTTATGAGGTACTGGCTATGGCAGATCTATTT
ACGGGCGCATCGCTTTGCGCTTTTAGGAGTTTCCCTCGGGAATTCCGGGA

ID 75. 201-350 PREDICTED: Plutella xylostella charged multivesicular body protein 4b (LOC105396929), mRNA

(SEQ ID NO: 92)
AGGTCCATCAACACACGAAGCTATTCAAAAATTACGCGAGACCGAAGAAT
TGCTCATCAAAAAGCAGGACTTCCTGGAGAAGAAAATACAATTAGAAGTA
GACACAGCCAGGAAACATGGCACTAAGAACAAAAGAGCGGCCATCGCTGC

ID 76. 826-975 PREDICTED: Plutella xylostella charged multivesicular body protein 4b (LOC105396929), mRNA

(SEQ ID NO: 93)
TAAGTGTTGGAAGCGAAACCGAATACATATGTATAGTCTCCGTTACACAT
CCAACTACTTAGCTTATTCTAAGGCTGCGTCCCTGTAGGCAAACAGTATT
CTCGTTACTAGTCTATGGTAATTTGAGTAGAGTGAAAGAACAACTAGCTA

ID 77. 826-975 PREDICTED: Plutella xylostella charged multivesicular body protein 4b (LOC105396929), mRNA

(SEQ ID NO: 94)
TAAGTGTTGGAAGCGAAACCGAATACATATGTATAGTCTCCGTTACACAT
CCAACTACTTAGCTTATTCTAAGGCTGCGTCCCTGTAGGCAAACAGTATT
CTCGTTACTAGTCTATGGTAATTTGAGTAGAGTGAAAGAACAACTAGCTA

ID 78. 201-350, 826-975 PREDICTED: Plutella xylostella charged multivesicular body protein 4b (LOC105396929), purposed sequence mRNA

(SEQ ID NO: 95)
AGGTCCATCAACACACGAAGCTATTCAAAAATTACGCGAGACCGAAGAAT
TGCTCATCAAAAAGCAGGACTTCCTGGAGAAGAAAATACAATTAGAAGTA
GACACAGCCAGGAAACATGGCACTAAGAACAAAAGAGCGGCCATCGCTGC
TAAGTGTTGGAAGCGAAACCGAATACATATGTATAGTCTCCGTTACACAT
CCAACTACTTAGCTTATTCTAAGGCTGCGTCCCTGTAGGCAAACAGTATT
CTCGTTACTAGTCTATGGTAATTTGAGTAGAGTGAAAGAACAACTAGCTA

ID 79. XM_038113977.1|:101-200 PREDICTED: Plutella xylostella V-type proton ATPase catalytic subunit A (LOC105392322), transcript variant X1, mRNA

(SEQ ID NO: 96)
GTCTGTCTTTTCCATCTTTCTCGTCCAAAAAACAAGGTAATTGTTCAAGA
TGAGTCACGGGTTGAAGAGGATTGCCGATGAGGACAATGAAACCCAGTTC

ID 80. 201-300 PREDICTED: Plutella xylostella V-type proton ATPase catalytic subunit A (LOC105392322), transcript variant X1, mRNA

(SEQ ID NO: 97)
GGTTATGTCTTCGCTGTGTCCGGTCCCGTGGTCACAGCGGAGAAGATGTC
CGGCTCGGCCATGTACGAGCTGGTGCGCGTCGGCTACAACGAGCTGGTCG

ID 81. XM_038113977.1|:301-400 PREDICTED: Plutella xylostella V-type proton ATPase catalytic subunit A (LOC105392322), transcript variant X1, mRNA

(SEQ ID NO: 98)
GAGAGATCATCCGTCTGGAGGGGGACATGGCCACCATCCAGGTGTACGAA
GAGACCTCAGGCGTAACCGTCGGCGACCCCGTGCTCCGCACCGGCAAGCC

ID 82. XM_038113977.1|:401-500 PREDICTED: Plutella xylostella V-type proton ATPase catalytic subunit A (LOC105392322), transcript variant X1, mRNA

(SEQ ID NO: 99)
TCTCTCCGTAGAACTGGGACCCGGCATCCTCGGCTCCATCTTCGACGGCA
TCCAGCGCCGCTGAAGGACATCAACGAGCTCACGCAGAGCATCTACATC

ID 83. XM_038113977.1|:501-600 PREDICTED: Plutella xylostella V-type proton ATPase catalytic subunit A (LOC105392322), transcript variant X1, mRNA

(SEQ ID NO: 100)
CCCAAGGGGGTGAACGTGCCCGCGCTGGCCCGCGACACCGAGTGGGAGTT
CCACCGCAGTACATCAAGGTCGGCACCCACATCACCGGCGGGGACTTAT

ID 84. XM_038113977.1|:601-700 PREDICTED: Plutella xylostella V-type proton ATPase catalytic subunit A (LOC105392322), transcript variant X1, mRNA

(SEQ ID NO: 101)
ACGGGATCGTGCACGAAAACACGCTGGTGAAGCACCGCATGCTGGTGCCG
CCCAAGGCCAAGGGCACCGTCACCTACATCGCGCCCGAGGGGAACTACAA

ID 85. XM_038113977.1|:1501-1600 PREDICTED: Plutella xylostella V-type proton ATPase catalytic subunit A (LOC105392322), transcript variant X1, mRNA

(SEQ ID NO: 102)
CTTACAGTAAGTACATGCGCGCCCTGGACGACTTCTACGACAAGAACTAC
CCCGAGTTCGTGCCGCTCAGGACCAAGGTCAAGGAGATCCTGCAAGAGGA

ID 86. XM_038113977.1|:1601-1700 PREDICTED: Plutella xylostella V-type proton ATPase catalytic subunit A (LOC105392322), transcript variant X1, mRNA

(SEQ ID NO: 103)
GGAGGACCTGTCTGAAATCGTGCAGCTGGTCGGAAAGGCGTCCCTCGCCG
AGACCGACAAGATCACCCTCGAGGTGGCCAAGCTGCTGAAGGACGACTTC

ID 87. XM_038113977.1|:1701-1800 PREDICTED: Plutella xylostella V-type proton ATPase catalytic subunit A (LOC105392322), transcript variant X1, mRNA

(SEQ ID NO: 104)
TTGCAGCAGAACAGCTACTCGAACTACGACCGTTTCTGCCCGTTCTACAA
GACGACCGGCATGCTGAAGAACATCATCACGTTCTACGACATGTCGCGAC

ID 88. XM_038113977.1|:1801-1900 PREDICTED: Plutella xylostella V-type proton ATPase catalytic subunit A (LOC105392322), transcript variant X1, mRNA

(SEQ ID NO: 105)
ACGCTGTCGAGTCCACCGCGCAGTCCGACAACAAGGTGACGTGGAACACG
ATCCGTGACGCCATGGGCCCCGTGCTCTACCAGCTGTCCAGCATGAAGTT

ID 89. XM_038113977.1|:1901-2000 PREDICTED: Plutella xylostella V-type proton ATPase catalytic subunit A (LOC105392322), transcript variant X1, mRNA

(SEQ ID NO: 106)
CAAGGACCCCGTGAAAGATGGAGAAGCCAAGATCAAGGCTGACTTCGACC
AGATCGTCGAGGACATGGCCGCTGCCTTCCGTAACCTAGAGGACTAAGTT

ID 90. XM_038113977.1|:2001-2100 PREDICTED: Plutella xylostella V-type proton ATPase catalytic subunit A (LOC105392322), transcript variant X1, mRNA

(SEQ ID NO: 107)
ATCATGCGCAATTATACTCTTTATTCTTGAAGAGGATGTTTGGGTCGGAC
CTCTTGCCGCGCGGTGAAAAAAATAAAACTGTCAACTTAACCATAGAGCT

ID 91. XM_038113977.1|:2101-2200 PREDICTED: Plutella xylostella V-type proton ATPase catalytic subunit A (LOC105392322), transcript variant X1, mRNA

(SEQ ID NO: 108)
GACAGTGTCTATGGTTAGAAATAGCGCGAAAAGATACTGGCTACTGCTAA
GATAGGTTGATGTTTGAGGTCCGATCCAACATTGTTTTGTTAATAAATAT

ID 92. XM_038113977.1|:2301-2400 PREDICTED: Plutella xylostella V-type proton ATPase catalytic subunit A (LOC105392322), transcript variant X1, mRNA

(SEQ ID NO: 109)
AAAGTCGTGTTTCAAATCTTGTTGAATTTCATCTTAGACGTTGAATATAA
GCCGTTGACGGTGTCAATGTATTTTTTATGTAACACGCGGTCACTAGATA

ID 93. XM_038113977.1|:2401-2500 PREDICTED: Plutella xylostella V-type proton ATPase catalytic subunit A (LOC105392322), transcript variant X1, mRNA

(SEQ ID NO: 110)
CTTTCTCGACAATATGATATATACATTTTGTAAATAAAGCCCCCTTTCCA
TTCAAGATTGTGTAATGTTATATAGAGGTACATGGTGCATCGGTCTAGTG

ID 94. XM_038113977.1|:2601-2700 PREDICTED: Plutella xylostella V-type proton ATPase catalytic subunit A (LOC105392322), transcript variant X1, mRNA

(SEQ ID NO: 111)
GCCCAATGATAATTGAATGATTGTTTTCGTCGGTTCATATACCCTGTTGC
ATTCCAGCTTATTTTTAGATAGTTAAAACAACAAATCGGTTATTTTTACT

ID 95. XM_038113977.1|:71-220 PREDICTED: Plutella xylostella V-type proton ATPase catalytic subunit A (LOC105392322), transcript variant X1, mRNA

(SEQ ID NO: 112)
GTGATAAGTTGTGATTCTCCGCCATTCTCGGTCTGTCTTTTCCATCTTTC
TCGTCCAAAAAACAAGGTAATTGTTCAAGATGAGTCACGGGTTGAAGAGG
ATTGCCGATGAGGACAATGAAACCCAGTTCGGTTATGTCTTCGCTGTGTC

ID 96. XM_038113977.1|:1971-2120 PREDICTED: Plutella xylostella V-type proton ATPase catalytic subunit A (LOC105392322), transcript variant X1, mRNA

(SEQ ID NO: 113)
GCTGCCTTCCGTAACCTAGAGGACTAAGTTATCATGCGCAATTATACTCT
TTATTCTTGAAGAGGATGTTTGGGTCGGACCTCTTGCCGCGCGGTGAAAA
AAATAAAACTGTCAACTTAACCATAGAGCTGACAGTGTCTATGGTTAGAA

ID 97. 300 BP Plutella xylostella V-type proton ATPase catalytic subunit A (LOC105392322), transcript variant X1, sequence mRNA

(SEQ ID NO: 114)
GTGATAAGTTGTGATTCTCCGCCATTCTCGGTCTGTCTTTTCCATCTTTC
TCGTCCAAAAAACAAGGTAATTGTTCAAGATGAGTCACGGGTTGAAGAGG
ATTGCCGATGAGGACAATGAAACCCAGTTCGGTTATGTCTTCGCTGTGTC
GCTGCCTTCCGTAACCTAGAGGACTAAGTTATCATGCGCAATTATACTCT
TTATTCTTGAAGAGGATGTTTGGGTCGGACCTCTTGCCGCGCGGTGAAAA
AAATAAAACTGTCAACTTAACCATAGAGCTGACAGTGTCTATGGTTAGAA

ID 98. AY971374.1|:676-975 Plutella xylostella cytochrome P450 (CYP6BF1v1) mRNA, complete cds

(SEQ ID NO: 115)
ACCAATTGCAGGCGTGAAGAATGTCCTCAGATACGGCTACCCTTCCTTCT
TCTACAGCGTGGGATTGGAGCTCTATTCCAGCAAAATTTACCGTTTCTTC
CGATCTGTTATACTTGACGTTATAAACAGTCGTAACGGCGCCAAATCTTC
GAGGAATGACATGGTGGATCTTATTTCCGATTGGAAGAAGAACAAATACA
TAACGGGAGACAGTATTGATAATGGCATAGACGGTGGAAACAAGAAGGTG
CGTATCGAAGTCGACGACGAACTTTTGGTGAGCCAATGTGTGCTGTTCTT

ID 99. 101-200 Plutella xylostella cytochrome P450 (CYP6BF1v1) mRNA, complete cds

(SEQ ID NO: 116)
AACTACTGGAAGAAACAGAACGTCAAGTACTTGACGCCGATCCCTTTCCT
GGGGAACGTGGCTGATGTGATCTTCCAGAGGGACACCTTCGGAGCCGTGA

ID 100. 201-300 Plutella xylostella cytochrome P450 (CYP6BF1v1) mRNA, complete cds

(SEQ ID NO: 117)
CGCAACGGATCTGCCAGCAGTTCCCCGATGAAGCTGTGGTCGGCATGTTC
TACTGCAGCAACCCTGCAGCCCTCGTACAGTGCCCTGACATGCTCAAGAC

ID 101. 301-400 Plutella xylostella cytochrome P450 (CYP6BF1v1) mRNA, complete cds

(SEQ ID NO: 118)
AGTCATGGTCAAGGACTACGCCTACTGCTCCAGTAAGGAGGTCTCCGTCC
ACAGCCACAAGGAACCCATGACCAAGAACATGTTCTTCACCTTCGGAGAC

ID 102. AY971374.1|:401-500 Plutella xylostella cytochrome P450 (CYP6BF1v1) mRNA, complete cds

(SEQ ID NO: 119)
AAGTGGAAGCTCATCCGGCAGAACCTCACGCCGGTCTACACGTCCGCCAA
AATGAAGAACATGTTTCCACTGGTACAGGATTGCTGCAGAATATTCCAGA

ID 103. AY971374.1|:501-600 Plutella xylostella cytochrome P450 (CYP6BF1v1) mRNA, complete cds

(SEQ ID NO: 120)
AGGTTCTCGATGATGAGATAGGAAAGGGCCGGGTGGTGGAAGTGAAGTCT
TTGATAGCTCGGTATACTATGGACTGTATAACTTCGTGTGCATTCGGCGT

ID 104. AY971374.1|:601-700 Plutella xylostella cytochrome P450 (CYP6BF1v1) mRNA, complete cds

(SEQ ID NO: 121)
CGACTCTGGCACGATGTCGAAGGGCGAGGAAGGGAACCCTTTCACAGAAA
CAGGTCACCTTTTATTTGATGAAAGACCAATTGCAGGCGTGAAGAATGTC

ID 105. 701-800 Plutella xylostella cytochrome P450 (CYP6BF1v1) mRNA, complete cds

(SEQ ID NO: 122)
CTCAGATACGGCTACCCTTCCTTCTTCTACAGCGTGGGATTGGAGCTCTA
TTCCAGCAAAATTTACCGTTTCTTCCGATCTGTTATACTTGACGTTATAA

IDs 106. 801-900 Plutella xylostella cytochrome P450 (CYP6BF1v1) mRNA, complete cds

(SEQ ID NO: 123)
ACAGTCGTAACGGCGCCAAATCTTCGAGGAATGACATGGTGGATCTTATT
TCCGATTGGAAGAAGAACAAATACATAACGGGAGACAGTATTGATAATGG

ID 107. 1201-1300 Plutella xylostella cytochrome P450 (CYP6BF1v1) mRNA, complete cds

(SEQ ID NO: 124)
CAAAGACTACACGCTACCGAATGGTGTGCATCTAAAGAAGGGGATGATGA
TACATATTCCTGTTTATCATTTGCATCACAATCCGAAGTATTTCCCGGAG

ID 108. 1301-1400 Plutella xylostella cytochrome P450 (CYP6BF1v1) mRNA, complete cds

(SEQ ID NO: 125)
CCCGAGGTGTTTCGTCCGGAGCGGTTTTCTGAAGAAGGACGGAAAAGTAT
TGTCCCGTATACCTACTTGCCCTTTGGGGACGGGCCGAGGATGTGTATAG

ID 109. AY971374.1|:1401-1500 Plutella xylostella cytochrome P450 (CYP6BF1v1) mRNA, complete cds

(SEQ ID NO: 126)
GCTACCGTTTCGCAAGGCTAGAGATCTTCTCCAGCCTAGCAGTTCTGTTG
AAGAAATACCGAGTGGAGCTGGCCCCCCACATGCCGAGGAAGCTGCAGTT

ID 109-2 AY971374.1|:700-849 Plutella xylostella cytochrome P450 (CYP6BF1v1) mRNA, complete cds

(SEQ ID NO: 127)
CCTCAGATACGGCTACCCTTCCTTCTTCTACAGCGTGGGATTGGAGCTCT
ATTCCAGCAAAATTTACCGTTTCTTCCGATCTGTTATACTTGACGTTATA
AACAGTCGTAACGGCGCCAAATCTTCGAGGAATGACATGGTGGATCTTAT

ID 109-3 AY971374.1|:1151-1300 Plutella xylostella cytochrome P450 (CYP6BF1v1) mRNA, complete cds

(SEQ ID NO: 128)
GAATCCCTCCGCATGTATCCTCCAGTCTCGGTGCTCATGAGAGAGATTTA
CAAAGACTACACGCTACCGAATGGTGTGCATCTAAAGAAGGGGATGATGA
TACATATTCCTGTTTATCATTTGCATCACAATCCGAAGTATTTCCCGGAG

ID 109-3. AY971374.1|:1151-1300 Plutella xylostella cytochrome P450 (CYP6BF1v1) purposed sequence mRNA, complete cds

(SEQ ID NO: 129)
CTCAGATACGGCTACCCTTCCTTCTTCTACAGCGTGGGATTGGAGCTCTA
TTCCAGCAAAATTTACCGTTTCTTCCGATCTGTTATACTTGACGTTATAA
ACAGTCGTAACGGCGCCAAATCTTCGAGGAATGACATGGTGGATCTTATT
GAATCCCTCCGCATGTATCCTCCAGTCTCGGTGCTCATGAGAGAGATTTA
CAAAGACTACACGCTACCGAATGGTGTGCATCTAAAGAAGGGGATGATGA
TACATATTCCTGTTTATCATTTGCATCACAATCCGAAGTATTTCCCGGAG

ID 109.4. AY971374.1|:650-707 Plutella xylostella cytochrome P450 (CYP6BF1v1) mRNA, complete cds

(SEQ ID NO: 130)
ACAGGTCACCTTTTATTTGATGAAAGACCAATTGCAGGCGTGAAGAATGT
CCTCAGAT

ID 109.5. AY971374.1|:765-822 Plutella xylostella cytochrome P450 (CYP6BF1v1) mRNA, complete cds

(SEQ ID NO: 131)
ACCGTTTCTTCCGATCTGTTATACTTGACGTTATAAACAGTCGTAACGGC
GCCAAATC

ID 109.6 AY971374.1|:823-880 Plutella xylostella cytochrome P450 (CYP6BF1v1) mRNA, complete cds

(SEQ ID NO: 132)
TTCGAGGAATGACATGGTGGATCTTATTTCCGATTGGAAGAAGAACAAAT
ACATAACG

ID 109.7 AY971374.1|:1159-1216 Plutella xylostella cytochrome P450 (CYP6BF1v1) mRNA, complete cds

(SEQ ID NO: 133)
CCGCATGTATCCTCCAGTCTCGGTGCTCATGAGAGAGATTTACAAAGACT
ACACGCTA

ID 109.8 AY971374.1|:1217-1274 Plutella xylostella cytochrome P450

(CYP6BF1v1) mRNA, complete cds

(SEQ ID NO: 134)
CCGAATGGTGTGCATCTAAAGAAGGGGATGATGATACATATTCCTGTTTA
TCATTTGC

ID 109.9 AY971374.1|:881-938 Plutella xylostella cytochrome P450 (CYP6BF1v1) mRNA, complete cds

(SEQ ID NO: 135)
GGAGACAGTATTGATAATGGCATAGACGGTGGAAACAAGAAGGTGCGTAT
CGAAGTCG

ID 109-10 939-996 Plutella xylostella cytochrome P450 (CYP6BF1v1) mRNA, complete cds

(SEQ ID NO: 136)
ACGACGAACTTTTGGTGAGCCAATGTGTGCTGTTCTTCCAAGCTGGCTTC
CAGCCAAG

ID 109-11 1101-1158 Plutella xylostella cytochrome P450 (CYP6BF1v1) mRNA, complete cds

(SEQ ID NO: 137)
TGCAGACCGACTGCGTGACCGCCCTGCCTTTCCTCGCCCAGTGCATGGAG
GAATCCCT

ID 109-11 1159-1216 Plutella xylostella cytochrome P450 (CYP6BF1v1) mRNA, complete cds

(SEQ ID NO: 138)
CCGCATGTATCCTCCAGTCTCGGTGCTCATGAGAGAGATTTACAAAGACT
ACACGCTA

ID 109-12 1217-1274 Plutella xylostella cytochrome P450 (CYP6BF1v1) mRNA, complete cds

(SEQ ID NO: 139)
CCGAATGGTGTGCATCTAAAGAAGGGGATGATGATACATATTCCTGTTTA
TCATTTGC

ID 109-13 1275-1333 Plutella xylostella cytochrome P450 (CYP6BF1v1) mRNA, complete cds

(SEQ ID NO: 140)
ATCACAATCCGAAGTATTTCCCGGAGCCCGAGGTGTTTCGTCCGGAGCGG
TTTTCTGAA

ID 109-14 1334-1390 Plutella xylostella cytochrome P450 (CYP6BF1v1) mRNA, complete cds

(SEQ ID NO: 141)
GAAGGACGGAAAAGTATTGTCCCGTATACCTACTTGCCCTTTGGGGACGG
GCCGAGG

ID P1. 101-250 Plutella xylostella cytochrome P450 6k1-like (LOC105392167), mRNA

(SEQ ID NO: 142)
CAGTTTATACTATCGAAATGATTTTAGCAATAATATTGTTATTAGTGATT
ATTATAATAACATTTTCGTTTCTACTGGGCTCCTACAATGAGTCTTACTG
GGCAAAACGAAACGTTAAATACCATGGCGGGAAAAATGCCATAGCAACAT

ID P2. 251-400 Plutella xylostella cytochrome P450 6k1-like (LOC105392167), mRNA

(SEQ ID NO: 143)
TCTCAGAGTTTTTGTTCACTAGCCGTGGCATTTTCGATATATTCGGTAAC
ATTTACAAGCTGTACCCTGAAGAACCTGCAGTGGCCACGCCGTCGCTGCT
TCAACCGGCTTTATTCGTCAAACATCCAGAAAACATACAACATGTGCTGA

ID P3. 401-550 Plutella xylostella cytochrome P450 6k1-like (LOC105392167), mRNA

(SEQ ID NO: 144)
CAGACAATTTTAAAAACTTTTACCACAGAGGTGTTGAAATCGCTAAGAAA
GATAAACTAGCACAAAATGT

Plutella xylostella, mRNA

(SEQ ID NO: 145)
ACCTTTCCTGAACGGCAGTCGGTGGAAACTTATGAGACAAAAAATGACGC
CGCTGTCACTAGTGCGAAGCTGAAGAACA

ID P4. 551-700 Plutella xylostella cytochrome P450 6k1-like (LOC105392167), mRNA

(SEQ ID NO: 146)
TGCACTACATCATAGACAGATGTGCCCAAGACTACATCGGTTATCTGAAA
GAACATGTCAACGATAAAAATGCTAATGCATTCGAAACATTATCTGTGTA
CAGCTGCTCATCGCTACTAGCCCCGATATTCGGAATCCACAGCGGACAGT

ID P5. 701-850 Plutella xylostella cytochrome P450 6k1-like (LOC105392167), mRNA

(SEQ ID NO: 147)
CAACGGTAACTTCACCGCTTCTAAATATGGCGAGAAATGCCACGAAACCG
ACTTTGAAAGCAAATTTAAAATTCATTTTGAACTCTTTGTCGCCGAAAGT
CTTCCAAATGCTGGGACTTAGTTTCTTTGGCGAATATGAGGAACAGTTCA

ID P6. 851-1000 Plutella xylostella cytochrome P450 6k1-like (LOC105392167), mRNA

(SEQ ID NO: 148)
TCGGCGCGATCAGTCAAGTGATAAGACAACGTAAGGAAGAGAATGTGAAA
AAGCACGATTTTGCTGACATTGCTGTGAGTTTGCAGAATGCTGGTACGAT
GAAAGACGAATCCAGCGGCTGTGAAATAGAGCCTACGGATGAAGTTCTAG

ID P7. 751-1050 Plutella xylostella cytochrome P450 6k1-like (LOC105392167), mRNA

(SEQ ID NO: 149)
ACTTTGAAAGCAAATTTAAAATTCATTTTGAACTCTTTGTCGCCGAAAGT
CTTCCAAATGCTGGGACTTAGTTTCTTTGGCGAATATGAGGAACAGTTCA
TCGGCGCGATCAGTCAAGTGATAAGACAACGTAAGGAAGAGAATGTGAAA
AAGCACGATTTTGCTGACATTGCTGTGAGTTTGCAGAATGCTGGTACGAT
GAAAGACGAATCCAGCGGCTGTGAAATAGAGCCTACGGATGAAGTTCTAG
CTGCACAAGCGTTCTTCTTCCTTATAGCAGGAGTAGATCCAGTAACAATG

ID P8. 450-751 Plutella xylostella cytochrome P450 6k1-like (LOC105392167). mRNA

(SEQ ID NO: 150)
AGATAAACTAGCACAAAATGTACCTTTCCTGAACGGCAGTCGGTGGAAAC
TTATGAGACAAAAAATGACGCCGCTGTTCACTAGTGCGAAGCTGAAGAAC
ATGCACTACATCATAGACAGATGTGCCCAAGACTACATCGGTTATCTGAA
AGAACATGTCAACGATAAAAATGCTAATGCATTCGAAACATTATCTGTGT
ACAGCTGCTCATCGCTACTAGCCCCGATATTCGGAATCCACAGCGGACAG
TCAACGGTAACTTCACCGCTTCTAAATATGGCGAGAAATGCCACGAAACC
GA

ID 110. KC789751.1|:72-1432 Spodoptera frugiperda cytochrome P450 CY321A8 mRNA. complete cds

(SEQ ID NO: 151)
TGAGAGCTACTGGAAGAAGCGTGGCGTAAAGTTCTACAGTAAAAACAAAG
TGATTGACCTTACTGGGATTATTTCATAACACAGCGTGCGTTGTTCGAAG
TCTTAACAGACCTGTACAAAAAATATAGACATGAACCAGCTATTGGTATT
GGTCAAGTCCTAACACCTGCGCTCTTTGTCATCGATCCGAAGAATGTTCA
GCAAGTTTTATCAAGTGACTTCCAATCTTTTAACCACAGAGGCATTGAAA
GTATTGAAGGGGATCAACTGACCGATAATATTCTCATGATGAATGGTCCA
AGATGGAAGCTGATGCGACAGAATATGACTCCGTTGTTTACGGCAAACAA
ATTGAAGAACATGTATTACATCATGGACAAAAGTGCTCAAGACTTTGCGA
ACTATTTGAAAAACAACCCCAAGACGCGTGATGGTAACTTATTTGAAACT
CTTATGATGTTCTGTAATGCTGCAGTTTGTGGAGCTATCTTCGGCATTGG
GTCAGAGTCAATCTTTGATTCACCTTTCCTCAAGCTTGCCAAAAACATAT
CGCAATCCAATTTTAAGATGAGAATGAAATTCGTTATTTATAGTCTCAGT
CCAAAGTTGTACAAACTGTTGAGATTACAAGCTTTCAACGAGATCGAAGA
CTTCTTCATTGGTTCAATAAGTCAAGTGATGAAATCAAGAGAACAAGAAA
ATGTAAAGAGACACGACTTTGCTGAAATTGCTGTGGCTATACAGAAAAAT
GGTATTATGAAAGACCGTACAACAGGTTGCGAGATAGAGCCTACTACCGG
CATTTTGTCTGCACAGGCATTTTTCTTCTTTAGTGCTGGTGTAGAGCCTT
GTGCTAATGCAATCTTTTCAACATTATTTCTTTTAAGCAGTCACCCAGAA
ATACTAGAAAGAGTTCACCAAGAAATTGACGAACATTTTGAAAAGCATAG
CAACAACATAAATTACGATGTTATATGTGAAATGAAGTACACAGACAAGG
TGCTGAGTGAAGCGATGAGAATGTTACCTCCAATTGGTCACTTGACAAGA
CAATGTGTTCAAAATACTGTCCTGCCTGTTGGTAATATCCCAGTAGAAAA
GGGGACAAAAATGTTCACTCCAATTTATGCTATTCATCACGACCCAGAAC
TATATCCAGACCCAGAAGTGTTCGACCCGGAGCGATTTGCCAATGATAGA
AAACCGAATGATAACATTTACATGCCATTTGGAATGGGCAACAGGGCATG
CATAGGGGAGAGGTATGCCAAATTACAAGTACAAGCCGGTTTAGTTCACG
TCTTGCGAAACTTCACTGTTAAACCGCAGAAACATGTAAAAGTAACATTT
GCCCAGGAT

ID 111. 301-400 Spodoptera frugiperda cytochrome P450 CY321A8 mRNA, complete cds

(SEQ ID NO: 152)
TTTAACCACAGAGGCATTGAAAGTATTGAAGGGGATCAACTGACCGATAA
TATTCTCATGATGAATGGTCCAAGATGGAAGCTGATGCGACAGAATATGA
AAGCTTTCAACGAGATCGAAGACTTCTTCATTGGTTCAATAAGTCAAGTG
ATGAAATCAAGAGAACAAGAAAATGTAAAGAGACACGACTTTGCTGAAAT
TCCAATTGGTCACTTGACAAGACAATGTGTTCAAAATACTGTCCTGCCTG
TTGGTAATATCCCAGTAGAAAAGGGGACAAAAATGTTCACTCCAATTTAT

ID 112. 701-800 Spodoptera frugiperda cytochrome P450 CY321A8 mRNA, complete cds

(SEQ ID NO: 153)
AAGCTTTCAACGAGATCGAAGACTTCTTCATTGGTTCAATAAGTCAAGTG
ATGAAATCAAGAGAACAAGAAAATGTAAAGAGACACGACTTTGCTGAAAT

ID 113. 1101-1200 Spodoptera frugiperda cytochrome P450 CY321A8 mRNA, complete cds

(SEQ ID NO: 154)
TCCAATTGGTCACTTGACAAGACAATGTGTTCAAAATACTGTCCTGCCTG
TTGGTAATATCCCAGTAGAAAAGGGGACAAAAATGTTCACTCCAATTTAT

ID 114. MN480661.1|:55-1500 Spodoptera frugiperda cytochrome P450 CYP6AE44 mRNA, complete cds

(SEQ ID NO: 155)
CTCGTATCAAAACGAAAATTCAGATACTGGGAACAGAAGAAAGTGCCACA
CCTACCACCAAAACCAATTCTGGGAAATTTCTCCGAATACATTCTCCAGC
AGAAATTCTATGGACGTGTTGAACAGGAGATCTGCAACAAGTTTCCTGAA
GAACCATACGTTGGCTCTTACTTGGGCACGGAACCGACCCTCATCATACA
AGATCCCGAATACATCAAGACCGTCATGACCAAGGACTACTATTTCTTCA
GTGGCCGTGAAGTCTCTGCATACTGTGAAAAGGAACCGTTAACTCAGAAC
CTATTCTTCACTTATGGCGATAAGTGGAAGGTACTGCGTCAGAACCTTAC
GCCTTTGTTCTCATCCGCAAAGATGAAGAACATGTTCCATTTGATCGAGA
AATGTGCCCGTATCTTCGAGAACATGGTCGACCAGGAAGTACAGAAAAGT
AAAGACATTGAAGTGCGAGCCCTGACGGGAAAATTCACCATGGATGCCAT
TGGAAATTTGCTTTTGGAGTTGAGACCCAGACGATGGTAAAGACTGACAA
TAATCCATTCACAAAGTTGGCGATGTTATCTTCGATACTGCCAGAATGAG
AGCATTAAAGGGTGTGCTAAGAAGTATTTGGCCAGCCATGTTTTATAGTT
TTGGAGGTAAAGCAGTCCCTGCTGATGTGATAGCTTTTTCTTCAATTTGA
TGACGAGTATCTTCAAAGGACGCAACTATAATCTGATCAAGGAACGACTT
TGTGGACCTCCTATTGAAGTTCCATAACAACAAAACAGTGACTGGGGATA
GTATGAGAAACTTGAAGGGTGATTCAGGAAAGAAAGTCAGTTTAGAAGTG
GACGATGAGTTTTTGATTGCACAATGTTTCCTGTTCTTTGCTGCTGGGTA
TGAGACGTCGGCGACTACGTTGAGTTACACTTTATATGAGTTGGCAAAGA
ATCCTGAAGCTCAAGAGTTAGCAATTCAAGACGTGGACAACTACTTGCGT
CGCAATGACAATGTGTTGAAGTACGAGTGTGTGACCGAGTTGCCTTATGT
GGAAGCTTGTGTTGATGAGGCTCTTCGTCTATACCCAGTGCTAGGAGTAA
TTACTCGGGAGGTTATTGAGGATTATACATTCCCGACGGGATTGAAATTG
GAGAAAGGACTTCGCGTGCATTTACCGGTGTACCACATGCACACAACCCT
AATTACTTCCCGGAACCAGAGCAATATCGTCCGGAGCGGTTCTTGGGCGA
CGAGAAGAAAAATATTAAGCCGTATACATACTTCCCATTTGGCGAAGGAC
CTCGACTATGTATCGGAATGAGGTTCGCAAAGATGCAGATTACGGCTGGA
ATTATAACATTACTGAAGAAATATCGCGTAGAACTTGCGCCGGGTATGAG
CGAGACAATACAGTTCGAACCTCGATCTGTAATCACCGCC

ID 115. MN480661.1|:701-1001 Spodoptera frugiperda cytochrome P450 CYP6AE44 mRNA. complete cds

(SEQ ID NO: 156)
ATAGTTTTGGAGGTAAAGCAGTCCCTGCTGATGTTGATAGCTTTTTCTTC
AATTTGATGACGAGTATCTTCAAAGGACGCAACTATAATCTGACATCAAG
GAACGACTTTGTGGACCTCCTATTGAAGTTCCATAACAACAAAACAGTGA
CTGGGGATAGTATGAGAAATTGAAGGGTGATTCAGGAAAGAAAGTCAGTT
TAGAAGTGGACGATGAGTTTTTGATTGCACAATGTTTCCTGTTCTTTGCT
GCTGGGTATGAGACGTCGGCGACTACGTTGAGTTACACTTTATATGAGTT

ID 116. Spodoptera frugiperda cytochrome P450 6B2-like (LOC118273915). mRNA NCBI Reference Sequence: XM_035591116.1

(SEQ ID NO: 157)
TATAAATGAT TCATAAGTGT TCGGACCGCG TATTTGGCCA
GTCGCAACCA TGGCGGCCTT ATATTTCCTC GCCGCAGTGC
TAGTGTTAGT GTACGCGTTA TATTATTACT TCACAAGGAC
ATTCAACTAC TGGAAGAGTA GAAATGTGCG AGGACCAAAA
CCAGTTGCAT TATTTGGAAA CATTAAGGAC GCAGCTCTTC
GCAAAGAAAA TTATGGCGTC GTAATGCAAA ATATATACAA
TGCATATCCA AATGAAAAAG TGGTCGGCAT ATTCAGGATG
ACTTCGCCTT GTCTCCTTAT TCGAGACCTG GACATTATCA
AACATATCAT GATCAAAGAC TTCGAAGCCT TCAGTGATCG
TGGAGTGGAA TTCAGCAAAG AAGGATTGGG ACAAAACTTA
TTCCACGCGG ACGGAGATAC ATGGACTGCC TTGAGGAACA
GATTCACTCC CATTTTCACA ACAGGTAAAT TGAAGAACAT
GTTTTACCTA ATAAATGAGG GAGGCGATTC ATTTGTAGAG
TACATCCGTA CAGAATGCCA AAAGAAGGAA GAATTTGATA
TTCAGCCTCT CCTCCAGACG TATACTTTGT CTACGATCTC
CGCCTGTGCA TTCGGAATTA GCTATGACAG TCTTGATGTT
AAAATGGATA CTCTGAAACT TGTGGATAAA ATATTTTCTT
CACCAAGTTT TGCAGTTGAA TTGGATATGA TGTATCCCGG
TCTCCTGAAA TCTCTAAACC TTTCTTTATT CCCTACCGCC
ATAAAAAAGT TCTTTGATAA TCTAGTGAAT AATGTTATAG
AGCAAAGAAA TGGTAAACCA TCGGGTCGAA ATGATTTCAT
GGATCTTATT TTGGCGCTCC GTGAAATGGG AGAGGTCACA
AACTCAAAAT ATGACTCTGC AAAGCCAGTT GAAATAACAC
CTGGTGTGAT AGCAGCGCAA GCTTTTGTGT TTTATGCGGC
TGGTTATGAA ACCAGTGCTA CCACTATGAC GTACATGCTT
TACCAACTAG CAATGAATCC AGACATCCAA AAGAAGTTGA
CTGAAGAAAT TGACGAATCT CTCAAAGCAA ATAATGGACA
AGTTACATAC GAGAGCATTA AGGAAATGAA GTATTTGAAC
AAAGTGTTTG ATGAAACTCT ACGAATGTAC TCGATTGTAG
AACCTCTGCA GAGGAAAGCT GTAAGAGATT ACAAAGTGCC
CGGTACTGAC TTGACGATAG AAAAGAACAC AATTGTGCTG
GTATCTCCGA GAGGTATCCA CTACGACGAG AAATATTACG
ACAACCCTGA ACAGTTCAAC CCTGACAGAT TTGACGCGGA
GGAGGTGGGC AAGCGACATC CGTGCGCTTA CATGCCGTTT
GGAATTGGAC AGAGAAACTG CATCGGAATG AGGTTCGGCA
GACTTCAATC CCAACTGTGC ATAACCAAGT TGCTGTCTAA
GTTCCAAGTG GAGCCATCGA GGAATACTGC AAGGAAGCTG
GAAGTGGAAC CTTGTCGCTT TATCATCGGA CCCAAAGGAG
GGATACGTCT GAATATTGTT CCAAGAAAGC TGAAGGCTTA
ACACATTAAA CGCCATGGGG GGCAAATGTG ACCGGCGTTA
GCAGAGGATT TGCCTTCAAC AGATTTTTGT TGGCAGTCAA
TGTATAACGA CTTTTAAAAC TGTTTATAAA TCCATCCATC
TACCAGGTAA ACTTAGGTAT ACCAATGTGT TATTTTATTT
TTTTCATTGA AAATTCGACT TTGTATGTAA ATAATACGGT
TGAAATCTAA TACAGAAAAC TATCAGATTG CAGTAGCTTA
ACCTGCTGCT CATTGTATAT AGAATATCGT TTTAAATTTG
ATTCAAAATA ATAATTGTAT GTGGCCAGTT TCACCAGTCA
TGTTATTATG GAGTGCGCGG ACTTAGTGGG CTACTCCTTA
GGTGGCGAAC AGGGGTGAGG GGGGGGGAGG CAATTCGCCT
ACGCATATGA GAATAGACAA TGATGATTGA TGACCAATCT
TGAGATATCG GGAATATGAA TGGTTCAATT ATTGAAACAA
AAGACAATTG AACGTATAGA ACTTTTACGC ACGCAACCAA
CTAGTTCCGC GCGCACTATT ATTGAGATAT TATAAGACTC
ATAAAGGGAA GAAACCATCA ACAAACATCG ATTACAAATC
CATTCATTTG CAAATTGTAT ATAATTACTC TTAGCACATT
AATCATGCAT TTAGTTTATA AGTAAGCTAT ATTTAATTAA
ATTATTTAAA AACTA

ID 117. 101-300 PREDICTED: Spodoptera frugiperda cytochrome P450 6B2-like (LOC118273915), mRNA

(SEQ ID NO: 158)
TATTATTACTTCACAAGGACATTCAACTACTGGAAGAGTAGAAATGTGCG
AGGACCAAAACCAGTTGCATTATTTGGAAACATTAAGGACGCAGCTCTTC
GCAAAGAAAATTATGGCGTCGTAATGCAAAATATATACAATGCATATCCA
AATGAAAAAGTGGTCGGCATATTCAGGATGACTTCGCCTTGTCTCCTTAT

ID 118. 1101-1200 PREDICTED: Spodoptera frugiperda cytochrome P450 6B2-like (LOC118273915), mRNA

(SEQ ID NO: 159)
AGGAAATGAAGTATTTGAACAAAGTGTTTGATGAAACTCTACGAATGTAC
TCGATTGTAGAACCTCTGCAGAGGAAAGCTGTAAGAGATTACAAAGTGCC

ID 119. Spodoptera frugiperda cytochrome P450 CYP9A58 mRNA, complete cds GenBank: MN480666.1|:101-250, 1101-1250

(SEQ ID NO: 160)
TCAAGCCTATCCCATTGCTGGGCAATATGGGCACTGTATTGCTTCGAAGA
CAACACTTAGCATATAGTCTTATTGATTTGTATAATGCCTTCCCTGAAGA
AAAATTTGTAGGAAGGTTTGAGTTCATGAACGAGGCAGTGCTGATCAGAG
CAAGTTCGACTTCAACTCGATACAGAGCATGAAGTATATGGATAATGTGG
TGTCAGATTATTACGACGATGGCCTGTAGCTGTAGCTACTGACAGAATTT
GTGAAAAGGACTACAACATGGGTAAACCAAATGAAAGGCTGAGAAGGA

ID 120. 101-200 PREDICTED: Spodoptera frugiperda cytochrome P450 6B2-like (LOC118273915), mRNA

(SEQ ID NO: 161)
TATTATTACTTCACAAGGACATTCAACTACTGGAAGAGTAGAAATGTGCG
AGGACCAAAACCAGTTGCATTATTTGGAAACATTAAGGACGCAGCTCTTC

ID 121. 201-300 PREDICTED: Spodoptera frugiperda cytochrome P450 6B2-like (LOC118273915), mRNA

(SEQ ID NO: 162)
GCAAAGAAAATTATGGCGTCGTAATGCAAAATATATACAATGCATATCCA
AATGAAAAAGTGGTCGGCATATTCAGGATGACTTCGCCTTGTCTCCTTAT

ID 122. 101-300 PREDICTED: Spodoptera frugiperda cytochrome P450 6B2-like (LOC118273915), mRNA

(SEQ ID NO: 163)
TATTATTACTTCACAAGGACATTCAACTACTGGAAGAGTAGAAATGTGCG
AGGACCAAAACCAGTTGCATTATTTGGAAACATTAAGGACGCAGCTCTTC
GCAAAGAAAATTATGGCGTCGTAATGCAAAATATATACAATGCATATCCA
AATGAAAAAGTGGTCGGCATATTCAGGATGACTTCGCCTTGTCTCCTTAT

ID 123. 301-400 PREDICTED: Spodoptera frugiperda cytochrome P450 6B2-like (LOC118273915), mRNA

(SEQ ID NO: 164)
TCGAGACCTGGACATTATCAAACATATCATGATCAAAGACTTCGAAGCCT
TCAGTGATCGTGGAGTGGAATTCAGCAAAGAAGGATTGGGACAAAACTTA

ID 125. 1201-1300 PREDICTED: Spodoptera frugiperda cytochrome P450 6B2-like (LOC118273915), mRNA

(SEQ ID NO: 165)
CGGTACTGACTTGACGATAGAAAAGAACACAATTGTGCTGGTATCTCCGA
GAGGTATCCACTACGACGAGAAATATTACGACAACCCTGAACAGTTCAA

ID 126. 1301-1400 PREDICTED: Spodoptera frugiperda cytochrome P450 6B2-like (LOC118273915), mRNA

(SEQ ID NO: 166)
CCTGACAGATTTGACGCGGAGGAGGTGGGCAAGCGACATCCGTGCGCTTA
CATGCCGTTTGGAATTGGACAGAGAAACTGCATCGGAATGAGGTTCGGCA

ID 127. 101-300 PREDICTED: Spodoptera frugiperda cytochrome P450 6B2-like (LOC118273915), mRNA

(SEQ ID NO: 167)
TATTATTACTTCACAAGGACATTCAACTACTGGAAGAGTAGAAATGTGCG
AGGACCAAAACCAGTTGCATTATTTGGAAACATTAAGGACGCAGCTCTTC
GCAAAGAAAATTATGGCGTCGTAATGCAAAATATATACAATGCATATCCA
AATGAAAAAGTGGTCGGCATATTCAGGATGACTTCGCCTTGTCTCCTTAT

ID 129. PREDICTED: Spodoptera frugiperda cytochrome P450 6B2-like (LOC118273915), mRNA

(SEQ ID NO: 168)
TATTATTACTTCACAAGGACATTCAACTACTGGAAGAGTAGAAATGTGCG
AGGACCAAAACCAGTTGCATTATTTGGAAACATTAAGGACGCAGCTCTTC
GCAAAGAAAATTATGGCGTCGTAATGCAAAATATATACAATGCATATCCA
AATGAAAAAGTGGTCGGCATATTCAGGATGACTTCGCCTTGTCTCCTTAT
AGGAAATGAAGTATTTGAACAAAGTGTTTGATGAAACTCTACGAATGTAC
TCGATTGTAGAACCTCTGCAGAGGAAAGCTGTAAGAGATTACAAAGTGCC

ID 130. 201-300 Spodoptera frugiperda cytochrome P450 CY321A8 mRNA, complete cds

(SEQ ID NO: 169)
ACATGAACCAGCTATTGGTATTGGTCAAGTCCTAACACCTGCGCTCTTTG
TCATCGATCCGAAGAATGTTCAGCAAGTTTTATCAAGTGACTTCCAATCT

ID 133. 801-900 Spodoptera frugiperda cytochrome P450 CY321A8 mRNA, complete cds

(SEQ ID NO: 170)
TGCTGTGGCTATACAGAAAAATGGTATTATGAAAGACCGTACAACAGGTT
GCGAGATAGAGCCTACTACCGGCATTTTGTCTGCACAGGCATTTTTCTTC

ID 135. 1201-1300 Spodoptera frugiperda cytochrome P450 CY321A8 mRNA, complete cds

(SEQ ID NO: 171)
GCTATTCATCACGACCCAGAACTATATCCAGACCCAGAAGTGTTCGACCC
GGAGCGATTTGCCAATGATAGAAAACCGAATGATAACATTTACATGCCAT

ID. 1749-2049 Plutella xylostella strain DBM1Ac-S ABC transporter subfamily H member 1 (ABCH1) mRNA, complete cds Sequence ID: KP260785.1

(SEQ ID NO: 172)
GACTACAACCCGAAGGTGGGCGACATCCCGATCGACTTCAAGGAGCCCAT
CTACGGCGACACCAACCCCTCCTTCACTGACTTCGTCGCTCCCGGTGTTA
TTCTCACTATCGTGTTCTTCCTGGCGGTGGCGCTGACGTCGTCGGCGCTG
ATCGTGGAGCGCATGGAGGGGCTGCTGGACCGCTCGTGGGTGGCCGGCGT
GTCCCCCGGGGAGATCCTGTTCTCGCACGTCGTCACGCAGTTCGTCGTCA
TGTGCGGCCAGACCGCGCTCGTGCTCGTGTTCATGATACTGGTGTTCGGC
G

ID 137. |MN480666.1|:101-250 Spodoptera frugiperda cytochrome P450 CYP9A58 mRNA, complete cds

(SEQ ID NO: 173)
TCAAGCCTATCCCATTGCTGGGCAATATGGGCACTGTATTGCTTCGAAGA
CAACACTTAGCATATAGTCTTATTGATTTGTATAATGCCTTCCCTGAAGA
AAAATTTGTAGGAGGTTTGAGTTCATGAACGAGGCAGTGCTGATCAGAG

ID 138. 1101-1250 Spodoptera frugiperda cytochrome P450 CYP9A58 mRNA, complete cds

(SEQ ID NO: 174)
CAAGTTCGACTTCAACTCGATACAGAGCATGAAGTATATGGATAATGTGG
TGTCAGAATTATTACGACGATGGCCTGTAGCTGTAGCTACTGACAGAATT
TGTGAAAAGGACTCAACATGGGTAAACCAAATGCAAAGGCTGAGAAGGA

ID 139. 151-300 PREDICTED: Spodoptera frugiperda ABC transporter G family member 20-like (LOC118270582), transcript variant X1, mRNA

(SEQ ID NO: 175)
GCGCCAAAATTTTGTGACAGTGACTTTTGTGATTATCTGTGACACTTTAT
TTTTTCTATTTTTACTTCCCTTCGGATTGTTTGAGAGATGGCGCATCCTA
CAGAGTTGCGCTCCGAGAGAGTGAAACAAATGTTTAGTTGGACTACGGG

ID 140. XM_035586211.1|:2801-3000 PREDICTED: Spodoptera frugiperda ABC transporter G family member 20-like (LOC118270582), transcript variant X1, mRNA

(SEQ ID NO: 176)
TTATTTTGACAATTGTTTATATTTGATGCGTAATCTGAACAAAAACATAG
TGGATTACATTTGTAAATGTTTTGGTACGATTGTAAGCTCACGGTAGGAA
GGAATTAGAAGTTCAGGACCACGCCTTATAGTCAGTAAATACTTCTTCAT
TTAAACGGTGAAGGGCGACGAGTCAGTTTGTTGTACACTCTCGAAACTAT

ID 141. 201-300 PREDICTED: Spodoptera frugiperda PBAN-type neuropeptides (LOC118281022), mRNA

(SEQ ID NO: 177)
ATTCTTCAAACTCTTGGAAGCGGCAGACGCATTGAAATATTACTACGATC
GCTTACCTTACGAGATGCAAGCGGACGAACCTGAAACCAGAGTTACCAAA

ID 142. 251-350 PREDICTED: Spodoptera frugiperda PBAN-type neuropeptides (LOC118281022), mRNA

(SEQ ID NO: 178)
GCTTACCTTACGAGATGCAAGCGGACGAACCTGAAACCAGAGTTACCAAA
AAAGTGATATTTACTCCTAAATTGGGAAGGAGTTTGGCTTATGATGATAA

ID 143. 351-450 PREDICTED: Spodoptera frugiperda PBAN-type neuropeptides (LOC118281022), mRNA

(SEQ ID NO: 179)
AGTCTTTGAGAATGTTGAGTTCACACCACGGTTGGGAAGACGGTTGGCTG
ATGATATGCCCGCGACGCCGGCTGATCAGGAACTGTATAGACCAGACCCG

ID 144. 451-550 PREDICTED: Spodoptera frugiperda PBAN-type neuropeptides (LOC118281022), mRNA

(SEQ ID NO: 180)
GATCAGATCGACAGCAGGACGAAGTACTTCTCGCCTAGGCTCGGCAGGAC
CATGATTTTTCACCACGACTCGGCAGGGAATTGTCCTATGAAATGTTAC

ID 145. 551-650 PREDICTED: Spodoptera frugiperda PBAN-type neuropeptides (LOC118281022), mRNA

(SEQ ID NO: 181)
CAAGCAAACTAAGAATGGTGAGGAGTGCCAACAGAACGCAATCGACATAA
ACTTGACATGAGCCCGCCATCAAACATAACCTCAAAATGAGACAAACTAA

ID 146. 551-700 PREDICTED: Spodoptera frugiperda PBAN-type neuropeptides (LOC118281022), mRNA

(SEQ ID NO: 182)
CAAGCAAACTAAGAATGGTGAGGAGTGCCAACAGAACGCAATCGACATAA
ACTTGACATGAGCCCGCCATCAAACATAACCTCAAAATGAGACAAACTAA
TGTACTGTACAATGTATAAATAAATCGCTATTTTGGACGAAATAATGTAC

ID 147. XM_035601452.1|:201-400 PREDICTED: Spodoptera frugiperda PBAN-type neuropeptides (LOC118281022), mRNA

(SEQ ID NO: 183)
ATTCTTCAAACTCTTGGAAGCGGCAGACGCATTGAAATATTACTACGATC
GCTTACCTTACGAGATGCAAGCGGACGAACCTGAAACCAGAGTTACCAAA
AAAGTGATATTTACTCCTAAATTGGGAAGGAGTTTGGCTTATGATGATAA
AGTCTTTGAGAATGTTGAGTTCACACCACGGTTGGGAAGACGGTTGGCTG

ID 149. 151-300 PREDICTED: Spodoptera frugiperda charged multivesicular body protein 4b-like (LOC118279222), mRNA

(SEQ ID NO: 184)
AGTTTTCTGGGGAAGTTATTCGGTGGTAAGAAGGAAGAAAAGGGCCCGAC
AACACATGAAGCTATTCAGAAATTACGCGAGACAGAGGAGCTCTTGCTGA
AAAAACAAGAGTTTCTAGAGAAGAAAATCGATATAGAAATACAGACTGCT

ID 150. 801-950 PREDICTED: Spodoptera frugiperda charged multivesicular body protein 4b-like (LOC118279222), mRNA

(SEQ ID NO: 185)
GTCTTGGGCCACATAATGTATGCACCTCAAGTTGTAATTTTGTGATATAT
TATAGGTATTGTATAGCCATCCGCTTTGTTACGCACTCTTGTGGGCACAG
CACGTCGATGTATACTGGTTTTGTATTGAAAATATGTAGCTTGATATGCT

ID 151. 151-300, and 801-950 Suggested Spodoptera frugiperda charged multivesicular body protein 4b-like (LOC118279222), mRNA

(SEQ ID NO: 186)
AGTTTTCTGGGGAAGTTATTCGGTGGTAAGAAGGAAGAAAAGGGCCCGAC
AACACATGAAGCTATTCAGAAATTACGCGAGACAGAGGAGCTCTTGCTGA
AAAAACAAGAGTTTCTAGAGAAGAAAATCGATATAGAAATACAGACTGCT
GTCTTGGGCCACATAATGTATGCACCTCAAGTTGTAATTTTGTGATATAT
TATAGGTATTGTATAGCCATCCGCTTTGTTACGCACTCTTGTGGGCACAG
CACGTCGATGTATACTGGTTTTGTATTGAAAATATGTAGCTTGATATGCT

ID 152. 301-450 and 1851-2000 Spodoptera frugiperda cytokine receptor mRNA, complete cds

(SEQ ID NO: 187)
TTGTATATTGAACATTTGGACAAACAGGTATACACATTTTACTGTAAGAA
TAATGTGACAAACAAACCTTGCACCACTAGAGTGCTAGTGGACGACTCTC
CCTCCAATGTTACTGACTTTAGCTGCATATCAAAGAATCTTGATGAACTG
AAACATAACTGATCTTAAACCTTATACAACATATCTTTTTACCCTCGCCT
TAAATACTACTTATGGGTTAAAGACAATTGAAAATGCTTCCACTGGAGTG
ACAACTATCGAGGATACTCCCACAAGTCCAAGGAATGTACGCATTACAGA

ID 153. 301-450 Spodoptera frugiperda cytokine receptor mRNA, complete cds

(SEQ ID NO: 188)
TTGTATATTGAACATTTGGACAAACAGGTATACACATTTTACTGTAAGAA
TAATGTGACAAACAAACCTTGCACCACTAGAGTGCTAGTGGACGACTCTC
CCTCCAATGTTACTGACTTTAGCTGCATATCAAAGAATCTTGATGAACTG

ID 154. 1851-2000 Spodoptera frugiperda cytokine receptor mRNA, complete cds

(SEQ ID NO: 189)
AAACATAACTGATCTTAAACCTTATACAACATATCTTTTTACCCTCGCCT
TAAATACTACTTATGGGTTAAAGACAATTGAAAATGCTTCCACTGGAGTG
ACAACTATCGAGGATACTCCCACAAGTCCAAGGAATGTACGCATTACAGA

ID 155. 201-300 Spodoptera frugiperda cytokine receptor mRNA, complete cds

(SEQ ID NO: 190)
GATCTTTTGCGTGGCTGAAAACTATACTGCTGAGAACTTGGAGTTTTACC
ACGGTCGAAAATTTATAGAATCAGAAAAAGTGAATGCAACCACCAGGAGG

ID 156. 301-400 Spodoptera frugiperda cytokine receptor mRNA, complete cds

(SEQ ID NO: 191)
TTGTATATTGAACATTTGGACAAACAGGTATACACATTTTACTGTAAGAA
TAATGTGACAAACAAACCTTGCACCACTAGAGTGCTAGTGGACGACTCTC

ID 157. 401-500 Spodoptera frugiperda cytokine receptor mRNA, complete cds

(SEQ ID NO: 192)
CCTCCAATGTTACTGACTTTAGCTGCATATCAAAGAATCTTGATGAACTG
AACTGTACTTGGACTTCTCCAGAGATTTACAGCATAACTACATACCGGTT

ID 158. 101-200 Spodoptera frugiperda Dredd mRNA, complete cds

(SEQ ID NO: 193)
AAATAATCATTTAAAACGAGAAAATGTTTTCTGTAGACTCAGTTACCACA
ACACCACAAGGAATGGAAGTGGAAAATGTTATTGGAAACAGTGATATTAT

ID 159. 201-300 Spodoptera frugiperda Dredd mRNA, complete cds

(SEQ ID NO: 194)
GAATACTGATATGATGTCAGAAATTGAAAAAGAATTACAAGACAATCCCA
GCGACTTGATATCCCTAGTGTTTCTTCTTTATGAAGTACCGGACACAGCA

ID 160. 301-400 Spodoptera frugiperda Dredd mRNA, complete cds

(SEQ ID NO: 195)
CCACAACGTTTAATTGTTTTTCAACGCGTTTCCAACGATGCCTGTAACTC
TATTAATCTTAACATGCTACACGAGTGGTTTCGATCTACTAAGCACAACC

ID 161. 401-500 Spodoptera frugiperda Dredd mRNA, complete cds

(SEQ ID NO: 196)
CCAACTGGAAACATCAATTTGTGGAAGCTCTCCTTATCTGTCAACTGTAC
AGTATTGTCAGAAAACTTGGATTGAATGTCCCTACAGCACGCAAGTACTA

ID 162. 801-900 Spodoptera frugiperda Dredd mRNA, complete cds

(SEQ ID NO: 197)
GATGAATATGTTGCAACACTCATTGAATGATGAGCACAAGCCAAGTGCCT
CAGCTGCTACCAGTACTCCTATGATGAAACATATGAAAGTGGATGAAACA

ID 163. 901-1000 Spodoptera frugiperda Dredd mRNA, complete cds

(SEQ ID NO: 198)
AATCAAGGGAAAGCTGATGACAATTATTACAATATGGATTTTAATGATGT
GTTTGAAATGCTTGGAGAGTTACAGTTGGATGAAAGAATGCAAGAGTCTC

ID 164. 1001-1100 Spodoptera frugiperda Dredd mRNA, complete cds

(SEQ ID NO: 199)
TGAAGTCTGATAGGAAGCTACTGGACAATGATAGCTATGAAATCAAGAGT
AACAAAAGGGTTGGAGTTTGTGTCATCATAAATCAGGAAACATTTTATCC

ID 165. 1401-1500 Spodoptera frugiperda Dredd mRNA, complete cds

(SEQ ID NO: 200)
TGATGCCAAAAAAATACACAACATGCCTAAACTTTTGATAGTTCAAGCAT
GCCAAGTTGATGAAAATACTCCCCAGATTGTAGTGGCTGACAGCCCAAGA

ID 166. 1451-1550 Spodoptera frugiperda Dredd mRNA, complete cds

(SEQ ID NO: 201)
GCCAAGTTGATGAAAATACTCCCCAGATTGTAGTGGCTGACAGCCCAAGA
GATTACAATTTAAGGAAATCTAACTTCCTTGTTTACTATGCCACTGCACC

ID 167. 1501-1600 Spodoptera frugiperda Dredd mRNA, complete cds

(SEQ ID NO: 202)
GATTACAATTTAAGGAAATCTAACTTCCTTGTTTACTATGCCACTGCACC
TGAACTAGAAGCTTACAGAAATGAAAAAAGAGGATCGATATTCATTCAGG

ID 168. 1601-1700 Spodoptera frugiperda Dredd mRNA, complete cds

(SEQ ID NO: 203)
TGCTATGTAGAACAATTAGAAAGTATGCTAATACTGAACATGTCTATGAT
ATTTTTACTAAGGTCAATAATAATGTGAATTTCATTTGCCAAAAGGTGGG

ID 169. Spodoptera frugiperda Dredd mRNA, complete cds

(SEQ ID NO: 204)
GAATACTGATATGATGTCAGAAATTGAAAAAGAATTACAAGACAATCCCA
GCGACTTGATATCCCTAGTGTTTCTTCTTTATGAAGTACCGGACACAGCA
TGAAGTCTGATAGGAAGCTACTGGACAATGATAGCTATGAAATCAAGAGT
AACAAAAGGGTTGGAGTTTGTGTCATCATAAATCAGGAAACATTTTATCC
TGATGCCAAAAAAATACACAACATGCCTAAACTTTTGATAGTTCAAGCAT
GCCAAGTTGATGAAAATACTCCCCAGATTGTAGTGGCTGACAGCCCAAGA

ID 170. Spodoptera frugiperda Dredd mRNA, complete cds

(SEQ ID NO: 205)
ACACCACAAGGAATGGAAGTGGAAAATGTTATTGGAAACAGTGATATTAT
GAATACTGATATGATGTCAGAAATTGAAAAAGAATTACAAGACAATCCCA
GCGACTTGATATCCCTAGTGTTTCTTCTTTATGAAGTACCGGACACAGCA
CCACAACGTTTAATTGTTTTTCAACGCGTTTCCAACGATGCCTGTAACTC
TATTAATCTTAACATGCTACACGAGTGGTTTCGATCTACTAAGCACAACC
CCAACTGGAAACATCAATTTGTGGAAGCTCTCCTTATCTGTCAACTGTAC

ID 171. 200-300 PREDICTED: Spodoptera frugiperda dual oxidase-like (LOC118269141), transcript variant X1, mRNA

(SEQ ID NO: 206)
ATGTTGGTCTCAACGTTACAAGTTGTGATACCGGTGTTCTGTGATCCAGA
AGTGTACTACGAGAAACAGCGGTATGATGGCTGGTTCAACAACAGAGCTT
A

ID 172. 300-400 PREDICTED: Spodoptera frugiperda dual oxidase-like (LOC118269141), transcript variant X1, mRNA

(SEQ ID NO: 207)
ACCCAGACTGGGGATCCGTTGGCAGTCGTCTCACACGCAAGACTCCAGCA
TCATACGCTGATGGGGTCTACATGATGGCAGGCGTCGATAGACCCGGCGC
C

ID 173. 400-500 PREDICTED: Spodoptera frugiperda dual oxidase-like (LOC118269141), transcript variant X1, mRNA

(SEQ ID NO: 208)
CAGAACATTATCCAAACTCTTCATGAGAGGGCAAGATGGACTGCCTTCTT
TAGCCAACAGGACAGCATTATTGGCGTTCTTTGGCCAGGTCGTAACCGGC
G

ID 174. 500-600 PREDICTED: Spodoptera frugiperda dual oxidase-like (LOC118269141), transcript variant X1, mRNA

(SEQ ID NO: 209)
GAGATTGTAATGGCATCCGAGTCGGGCTGTCCTATCGAGCACCATCGCAT
ACCAGTGGACAAATGTGACCACATGTATGACCCAGAATGTAATGGAGCCA
A

ID 175. PREDICTED: Spodoptera frugiperda dual oxidase-like (LOC118269141), transcript variant X1, mRNA 4100-4200

(SEQ ID NO: 210)
TCACATCAAAGCACAAGGTCCCTGGACGTGGAAACTCAGGAATTACTTCG
ATCCCTGCAACTTTAATACTGAAGATCATCCTAAAATCAGGATCCAAGGT
C

ID 176. 600-700 PREDICTED: Spodoptera frugiperda dual oxidase-like (LOC118269141), transcript variant X1, mRNA

(SEQ ID NO: 211)
AGTACATGCCGTTTCTTAGAGCAGCATATGATCGGAACACGGGGCAGAGT
CCCAACAGTCCTAGAGAACAGATAAACCAAATGACTTCGTGGATCGACGG
C

ID 177. 2600-2700 PREDICTED: Spodoptera frugiperda dual oxidase-like (LOC118269141), transcript variant X1, mRNA

(SEQ ID NO: 212)
CTTGGAATGAAAGCAGATGCTGTATTTGTGAAGAAAATGTTCAACATTGT
CGACAAAGACGGAGATGGCAGGATCTCTTTCCAGGAATTTCTGGACACGG
T

ID 178. 2700-2800 PREDICTED: Spodoptera frugiperda dual oxidase-like (LOC118269141), transcript variant X1, mRNA

(SEQ ID NO: 213)
TAGTCCTATTTTCTCGAGGAGCAACAGAAGACAAACTTCGCATTATCTTC
GACATGTGTGACGATGATCGTAACGGTGTGATCGACAAGGGAGAGCTCAG
T

ID 179. 3800-3900 PREDICTED: Spodoptera frugiperda dual oxidase-like (LOC118269141), transcript variant X1, mRNA

(SEQ ID NO: 214)
CTACGAACTAAGTATCTAGCACTGGATGTTTTGGAAACAGAAATGCTACC
ATCGGATGTGATTAAAATAAAGTTCTATAGACCACCAAATCTAAAATATT
T

ID 180. 3900-4000 PREDICTED: Spodoptera frugiperda dual oxidase-like (LOC118269141), transcript variant X1, mRNA

(SEQ ID NO: 215)
TATCAGGTCAATGGGTACGGTTATCTTGTACGGCGTTCAAGAAAGAAGAG
TTCCACTCGTTCACATTAACCTCAGCTCCTCACGAGAACTTCTTATCGTG
T

ID 181. Spodoptera frugiperda dual oxidase-like (LOC118269141), transcript variant X1, mRNA

(SEQ ID NO: 216)
ATGTTGGTCTCAACGTTACAAGTTGTGATACCGGTGTTCTGTGATCCAGA
AGTGTACTACGAGAAACAGCGGTATGATGGCTGGTTCAACAACAGAGCTT
ACTACGAACTAAGTATCTAGCACTGGATGTTTTGGAAACAGAAATGCTAC
CATCGGATGTGATTAAAATAAAGTTCTATAGACCACCAAATCTAAAATAT
TTCTACGAACTAAGTATCTAGCACTGGATGTTTTGGAAACAGAAATGCTA
CCATCGGATGTGATTAAAATAAAGTTCTATAGACCACCAAATCTAAAATA
TTT

ID 181. Spodoptera frugiperda dual oxidase-like (LOC118269141), transcript variant X1, mRNA

(SEQ ID NO: 217)
CTACGAACTAAGTATCTAGCACTGGATGTTTTGGAAACAGAAATGCTACC
ATCGGATGTGATTAAAATAAAGTTCTATAGACCACCAAATCTAAAATATT
TTATCAGGTCAATGGGTACGGTTATCTTGTACGGCGTTCAAGAAAGAAGA
GTTCCACTCGTTCACATTAACCTCAGCTCCTCACGAGAACTTCTTATCGT
GTTCACATCAAAGCACAAGGTCCCTGGACGTGGAAACTCAGGAATTACTT
CGATCCCTGCAACTTTAATACTGAAGATCATCCTAAAATCAGGATCCAAG
GTC

ID 182. Spodoptera frugiperda dual oxidase-like (LOC118269141), transcript variant X1, mRNA

(SEQ ID NO: 218)
TAAAATGGCGGCCCCGGATACGGTAGGGAGTCGAATATGGCTCCTAGTAA
TGTTGGTCTCAACGTTACAAGTTGTGATACCGGTGTTCTGTGATCCAGAA
GTGTACTACGAGAAACAGCGGTATGATGGCTGGTTCAACAACAGAGCTTA
AGCCAACAGGACAGCATTATTGGCGTTCTTTGGCCAGGTCGTAACCGGCG
AGATTGTAATGGCATCCGAGTCGGGCTGTCCTATCGAGCACCATCGCATA
CCAGTGGACAAATGTGACCACATGTATGACCCAGAATGTAATGGAGCCAA

ID 183. 101-200 PREDICTED: Spodoptera frugiperda glutathione S-transferase 1-like (LOC118261931), mRNA

(SEQ ID NO: 219)
CATGCGAGAAGAGGCTTTGATGGGTGTGAAACTATATGCTGCAGACTTCA
GCCCACCGGTGCGTTCCAGCATGATGGCCCTTGATATCTTCAATGTACCA

ID 184. 201-300 PREDICTED: Spodoptera frugiperda glutathione S-transferase 1-like (LOC118261931), mRNA

(SEQ ID NO: 220)
TTTGAGAAGATAACTCTCAACTTACTGAAACTGGAACACCGCACTCCAGA
ATATTTGGAAAAAAATCCGATTCACTCGATTCCAGTATTGGAAGATGGAG

ID 185. 301-400 PREDICTED: Spodoptera frugiperda glutathione S-transferase 1-like (LOC118261931), mRNA

(SEQ ID NO: 221)
ATTTAATATTACATGATAGCCACGCAATAATGGCCTACCTCGCTGACACC
TACGGAAAGGATGAGTCTTGGTATCCTAAGGACGTGAAAAAACGAGCTTT

ID 186. 401-500 PREDICTED: Spodoptera frugiperda glutathione S-transferase 1-like (LOC118261931), mRNA

(SEQ ID NO: 222)
AGTTAACCAAAAACTGTTTTTCACCACAGGTGTAATTTTCCCTAGACTAC
GAATCATTACATACTATCTTCTAGAAAAAGGAAGAAAAGCTATAGAACAA

ID 187. 501-600 PREDICTED: Spodoptera frugiperda glutathione S-transferase 1-like (LOC118261931), mRNA

(SEQ ID NO: 223)
GAATGGCTAGACAACATTGAAGAAGCCATTGGTTTTGTGGAGCAGTTCTT
GTCTCGGACAAAATTCATCGCCTTGGACCATGTCACCATTGCCGATATTG

ID 188. 601-700 PREDICTED: Spodoptera frugiperda glutathione S-transferase 1-like (LOC118261931), mRNA

(SEQ ID NO: 224)
CAGTGTTAAGTCACCTGTCCTCTGTGGAACCTATTCTACCCATAGATCCG
AAAAAGTACCCAAAGACGGTTGCCTGGTTAGAAATTATGAAAGCGACGCC

ID 189. 651-750 PREDICTED: Spodoptera frugiperda glutathione S-transferase 1-like (LOC118261931), mRNA

(SEQ ID NO: 225)
AAAAAGTACCCAAAGACGGTTGCCTGGTTAGAAATTATGAAAGCGACGCC
ATATTGCAAGAAATATAACGAAGAAGGTGTTAAATCTCTGACTGCTATTA

ID 190. Spodoptera frugiperda glutathione S-transferase 1-like (LOC118261931), mRNA

(SEQ ID NO: 226)
TTTGAGAAGATAACTCTCAACTTACTGAAACTGGAACACCGCACTCCAGA
ATATTTGGAAAAAAATCCGATTCACTCGATTCCAGTATTGGAAGATGGAG
AGTTAACCAAAAACTGTTTTTCACCACAGGTGTAATTTTCCCTAGACTAC
GAATCATTACATACTATCTTCTAGAAAAAGGAAGAAAAGCTATAGAACAA
AAAAAGTACCCAAAGACGGTTGCCTGGTTAGAAATTATGAAAGCGACGCC
ATATTGCAAGAAATATAACGAAGAAGGTGTTAAATCTCTGACTGCTATTA

ID 191. 201-500 PREDICTED: Spodoptera frugiperda glutathione S-transferase 1-like (LOC118261931), mRNA

(SEQ ID NO: 227)
TTTGAGAAGATAACTCTCAACTTACTGAAACTGGAACACCGCACTCCAGA
ATATTTGGAAAAAAATCCGATTCACTCGATTCCAGTATTGGAAGATGGAG
ATTTAATATTACATGATAGCCACGCAATAATGGCCTACCTCGCTGACACC
TACGGAAAGGATGAGTCTTGGTATCCTAAGGACGTGAAAAAACGAGCTTT
AGTTAACCAAAAACTGTTTTTCACCACAGGTGTAATTTTCCCTAGACTAC
GAATCATTACATACTATCTTCTAGAAAAAGGAAGAAAAGCTATAGAACAA

ID 192. 101-200 PREDICTED: Spodoptera frugiperda protein mesh (LOC118271033), transcript variant X1, mRNA

(SEQ ID NO: 228)
ACGGGCACACAATGTTCCAACACTTTAGTCGACCAGTACATTAGCGAGCC
ACCTGTTGTCGTGAAAATTGATAAAAAATAATGTGATGGACATTTTAATA

ID 193. 201-300 PREDICTED: Spodoptera frugiperda protein mesh (LOC118271033), transcript variant X1, mRNA

(SEQ ID NO: 229)
TAAGTGTGAAAATGGGTGTTAAGGTTTTAGCTTTAATAGCACTCTTAGTT
GTTAGTGTACTCGGACAAGACGTTACTGTCAGTGACAACGATGTAACAAA

ID 194. 301-400 PREDICTED: Spodoptera frugiperda protein mesh (LOC118271033), transcript variant X1, mRNA

(SEQ ID NO: 230)
GGAAGTTGTGGTGGACAATTTAGCTAGTGAGGACCCGATAGTATTAGACA
CAGTTACAGAAGCCACAAAAGATGAAGTTGCTGAAGATGCGAACCCAGTG

ID 195. 401-500 PREDICTED: Spodoptera frugiperda protein mesh (LOC118271033), transcript variant X1, mRNA

(SEQ ID NO: 231)
GAAATACTAAGTCCAACGGATGATCTGCAAGTAAGAAGCGGGAAATATCA
GCTCAATGATGGGCTCGTGGGTGAAGAGCCGATGCCACTAGATGCTGTTA

ID 196. 1901-2000 PREDICTED: Spodoptera frugiperda protein mesh (LOC118271033), transcript variant X1, mRNA

(SEQ ID NO: 232)
AAGGAGATTACCATCAGACCCCAGCTTGAATACATAGATATTATCGAAAT
GGGCGTGGCTAACACTGGAGAATATGTGATCAATCCCCAAAACTTTAGGA

ID 197. 2001-2100 PREDICTED: Spodoptera frugiperda protein mesh (LOC118271033), transcript variant X1, mRNA

(SEQ ID NO: 233)
ACCGGGATAACTTCATGCACAATGATATGCAGTTCGGTTTCCTCCAGATT
AATTTAACTACGCCTGAAGTCTTCAAAGGAGTCTCTATCTCGCCTGTACT

ID 198. 201-350 PREDICTED: Spodoptera frugiperda protein mesh (LOC118271033), transcript variant X1, mRNA

(SEQ ID NO: 234)
TAAGTGTGAAAATGGGTGTTAAGGTTTTAGCTTTAATAGCACTCTTAGTT
GTTAGTGTACTCGGACAAGACGTTACTGTCAGTGACAACGATGTAACAAA
GGAAGTTGTGGTGGACAATTTAGCTAGTGAGGACCCGATAGTATTAGACA

ID 199. 1901-2050 PREDICTED: Spodoptera frugiperda protein mesh (LOC118271033), transcript variant X1, mRNA

(SEQ ID NO: 235)
AAGGAGATTACCATCAGACCCCAGCTTGAATACATAGATATTATCGAAAT
GGGCGTGGCTAACACTGGAGAATATGTGATCAATCCCCAAAACTTTAGGA
ACCGGGATAACTTCATGCACAATGATATGCAGTTCGGTTTCCTCCAGATT

ID 200. 201-350, 1901-1050 PREDICTED: Spodoptera frugiperda protein mesh (LOC118271033), transcript variant X1,mRNA

(SEQ ID NO: 236)
TAAGTGTGAAAATGGGTGTTAAGGTTTTAGCTTTAATAGCACTCTTAGTT
GTTAGTGTACTCGGACAAGACGTTACTGTCAGTGACAACGATGTAACAAA
GGAAGTTGTGGTGGACAATTTAGCTAGTGAGGACCCGATAGTATTAGACA
AAGGAGATTACCATCAGACCCCAGCTTGAATACATAGATATTATCGAAAT
GGGCGTGGCTAACACTGGAGAATATGTGATCAATCCCCAAAACTTTAGGA
ACCGGGATAACTTCATGCACAATGATATGCAGTTCGGTTTCCTCCAGATT

ID 202. MT544380.1|:101-400 Spodoptera frugiperda clone Sf_17445 protein mesh mRNA, partial cds

(SEQ ID NO: 237)
TGGACAATTTAGCTAGTGAGGACCCAATAGTATTAGACACAGTTACAGAA
GCCACAAAAGATGAAGTTGCTGAAGATGCGAACCCAGTGGAAATACTAAG
TCCTACGGATGATCTGCAAGTAAGAAGTGGGAAATATCAGCTCAATGATG
GGCTCGTGGGCGAAGAGCCGATACCACTAGATGCTGTTAATTTCGACTCG
AATAATGATGCCGGAGAGAGTGAGAAGCAGTTGCTCTCTCCTGGCACAAC
TCAAGTCACGAACAACGAGTATGCTCATATCGATGGCCGAGTTTTACCGG

ID 203. 301-400 PREDICTED: Spodoptera frugiperda uncharacterized LOC118263801 (LOC118263801), transcript variant X1, mRNA

(SEQ ID NO: 238)
AAGTCGGACGTTATACAAAGAACGATGAGTGTGCTGAGGGGTTTTTAGTG
AGAAATTGTGAAAAATAGAAAGAGTGAAGATGTCGGTAAAAGCGAGCGTT

ID 204. 401-500 PREDICTED: Spodoptera frugiperda uncharacterized LOC118263801 (LOC118263801), transcript variant X1, mRNA

(SEQ ID NO: 239)
GGTTAAACAAAAAGGGTAAAAATGGCCTTCAAAGGATTCTGTGGCGAAGT
GATCGGGTTTTTCCTGGCTGTGGGTTTTTGCATCATATGTCCGGAATATG

ID 205. 701-800 PREDICTED: Spodoptera frugiperda uncharacterized LOC118263801 (LOC118263801), transcript variant X1, mRNA

(SEQ ID NO: 240)
AACTGCCGAGTCAAGAATTTAGGAAATAAAACGTTAAACATGCAAGTATC
GTGGGTACGGCATAGAGACATCCATCTGCTGACAGTCGGCCGGTACACAT

ID 206. 801-900 PREDICTED: Spodoptera frugiperda uncharacterized LOC118263801 (LOC118263801), transcript variant X1, mRNA

(SEQ ID NO: 241)
ACACGAGCGATCAAAGGTTTAGAGCTATTCACTTACCGCACTCCGAGGAC
TGGACTTTACAGATCAAGTATCCGCAACACAGGGATTCGGGAATTTATGA

ID 207. 1351-1450 PREDICTED: Spodoptera frugiperda uncharacterized LOC118263801 (LOC118263801), transcript variant X1, mRNA

(SEQ ID NO: 242)
GTTGGTTGCCTGCCTGTTCATCGCACTCTCTTGACACATACGGATCTTTA
ACTAAATGTAAATACAAGGATTTTATCAACATCAGCTGCTTATATTGACA

ID 211. Spodoptera frugiperda uncharacterized LOC118263801 (LOC118263801), transcript variant X1, mRNA

(SEQ ID NO: 243)
AAGTCGGACGTTATACAAAGAACGATGAGTGTGCTGAGGGGTTTTTAGTG
AGAAATTGTGAAAAATAGAAAGAGTGAAGATGTCGGTAAAAGCGAGCGTT
GGTTAAACAAAAAGGGTAAAAATGGCCTTCAAAGGATTCTGTGGCGAAGT
GATCGGGTTTTTCCTGGCTGTGGGTTTTTGCATCATATGTCCGGAATATG

ID 212. 401-500 Spodoptera frugiperda V-ATPase subunit A mRNA, complete cds

(SEQ ID NO: 244)
AATTCAACCCCTTGAATGTTAAGGTCGGCTCCCACATCACCGGAGGAGAC
TTGTACGGTATCGTACACGAGAACACATTGGTTAAGCACAAGATGTTGAT

ID 213. 1301-1400 Spodoptera frugiperda V-ATPase subunit A mRNA, complete cds

(SEQ ID NO: 245)
TGGACAAGAAGCTCGCGCAGCGCAAGCACTTCCCCGCCATCAACTGGCTC
ATCTCCTACAGCAAGTACATGCGAGCGCTGGATGACTTCTATGAGAAGAA

ID 214. 1501-1600 Spodoptera frugiperda V-ATPase subunit A mRNA, complete cds

(SEQ ID NO: 246)
GCCGAGACTGACAAAATCACCCTTGAGGTTGCCAAGCTGCTCAAAGACGA
CTTCTTGCAACAAAACAGCTACTCGTCATACGATCGTTTCTGTCCGTTCT

ID 218. 100-159, 1-60, Spodoptera frugiperda V-type proton ATPase catalytic subunit A (LOC118267501), transcript variant X1, X2, X3, and X4

(SEQ ID NO: 247)
AATTCAACCCCTTGAATGTTAAGGTCGGCTCCCACATCACCGGAGGAGAC
TTGTACGGTATCGTACACGAGAACACATTGGTTAAGCACAAGATGTTGAT
TGGACAAGAAGCTCGCGCAGCGCAAGCACTTCCCCGCCATCAACTGGCTC
ATCTCCTACAGCAAGTACATGCGAGCGCTGGATGACTTCTATGAGAAGAA
GCCGAGACTGACAAAATCACCCTTGAGGTTGCCAAGCTGCTCAAAGACGA
CTTCTTGCAACAAAACAGCTACTCGTCATACGATCGTTTCTGTCCGTTCT

ID 219. XM_035591116.1|:100-1600 PREDICTED: Spodoptera frugiperda cytochrome P450 6B2-like (LOC118273915), mRNA

(SEQ ID NO: 248)
ATATTATTACTTCACAAGGACATTCAACTACTGGAAGAGTAGAAATGTGC
GAGGACCAAAACCAGTTGCATTATTTGGAAACATTAAGGACGCAGCTCTT
CGCAAAGAAAATTATGGCGTCGTAATGCAAAATATATACAATGCATATCC
AAATGAAAAAGTGGTCGGCATATTCAGGATGACTTCGCCTTGTCTCCTTA
TTCGAGACCTGGACATTATCAAACATATCATGATCAAAGACTTCGAAGCC
TTCAGTGATCGTGGAGTGGAATTCAGCAAAGAAGGATTGGGACAAAACTT
ATTCCACGCGGACGGAGATACATGGACTGCCTTGAGGAACAGATTCACTC
CCATTTTCACAACAGGTAAATTGAAGAACATGTTTTACCTAATAAATGAG
GGAGGCGATTCATTTGTAGAGTACATCCGTACAGAATGCCAAAAGAAGGA
AGAATTTGATATTCAGCCTCTCCTCCAGACGTATACTTTGTCTACGATCT
CCGCCTGTGCATTCGGAATTAGCTATGACAGTCTTGATGTTAAAATGGAT
ACTCTGAAACTTGTGGATAAAATATTTTCTTCACCAAGTTTTGCAGTTGA
ATTGGATATGATGTATCCCGGTCTCCTGAAATCTCTAAACCTTTCTTTAT
TCCCTACCGCCATAAAAAAGTTCTTTGATAATCTAGTGAATAATGTTATA
GAGCAAAGAAATGGTAAACCATCGGGTCGAAATGATTTCATGGATCTTAT
TTTGGCGCTCCGTGAAATGGGAGAGGTCACAAACTCAAAATATGACTCTG
CAAAGCCAGTTGAAATAACACCTGGTGTGATAGCAGCGCAAGCTTTTGTG
TTTTATGCGGCTGGTTATGAAACCAGTGCTACCACTATGACGTACATGCT
TTACCAACTAGCAATGAATCCAGACATCCAAAAGAAGTTGACTGAAGAAA
TTGACGAATCTCTCAAAGCAAATAATGGACAAGTTACATACGAGAGCATT
AAGGAAATGAAGTATTTGAACAAAGTGTTTGATGAAACTCTACGAATGTA
CTCGATTGTAGAACCTCTGCAGAGGAAAGCTGTAAGAGATTACAAAGTGC
CCGGTACTGACTTGACGATAGAAAAGAACACAATTGTGCTGGTATCTCCG
AGAGGTATCCACTACGACGAGAAATATTACGACAACCCTGAACAGTTCAA
CCCTGACAGATTTGACGCGGAGGAGGTGGGCAAGCGACATCCGTGCGCTT
ACATGCCGTTTGGAATTGGACAGAGAAACTGCATCGGAATGAGGTTCGGC
AGACTTCAATCCCAACTGTGCATAACCAAGTTGCTGTCTAAGTTCCAAGT
GGAGCCATCGAGGAATACTGCAAGGAAGCTGGAAGTGGAACCTTGTCGCT
TTATCATCGGACCCAAAGGAGGGATACGTCTGAATATTGTTCCAAGAAAG
CTGAAGGCTTAACACATTAAACGCCATGGGGGGCAAATGTGACCGGCGTT
A

ID 220. 301-400 Spodoptera frugiperda cytochrome P450 CY321A8 mRNA, complete cds

(SEQ ID NO: 249)
TTTAACCACAGAGGCATTGAAAGTATTGAAGGGGATCAACTGACCGATAA
TATTCTCATGATGAATGGTCCAAGATGGAAGCTGATGCGACAGAATATGA

ID 221. 701-800 Spodoptera frugiperda cytochrome P450 CY321A8 mRNA, complete

(SEQ ID NO: 250)
cdsAAGCTTTCAACGAGATCGAAGACTTCTTCATTGGTTCAATAAGTCAA
GTGATGAAATCAAGAGAACAAGAAAATGTAAAGAGACACGACTTTGCTGA
AAT

ID 222. 1101-1200 Spodoptera frugiperda cytochrome P450 CY321A8 mRNA, complete cds

(SEQ ID NO: 251)
TCCAATTGGTCACTTGACAAGACAATGTGTTCAAAATACTGTCCTGCCTG
TTGGTAATATCCCAGTAGAAAAGGGGACAAAAATGTTCACTCCAATTTAT

ID 223. 501-600 PREDICTED: Spodoptera frugiperda uncharacterized LOC118263801 (LOC118263801), transcript variant X1, mRNA

(SEQ ID NO: 252)
TATCATCAGCACAGCGCGAACTCGGCAAGGGTTCGCCGACTAACGCCTCC
GCCACGGCATCGCCGGCCGGTTCTGTGGACAACGCGATCCGGTCCGGAGC

ID. 61-360 Plutella xylostella prophenoloxidase 1 mRNA, complete cds GenBank: KT006134.1

(SEQ ID NO: 253)
cagaagggcg acgacaagac cgtcttccag atcccggaca
acttctaccc agaaaagtac aagaaggtgg gcaaccagct
ggccgaccgg ttcggcacgg acgcgggccg catggtgccg
gtgcgcaaca tcgcgctgcc ggacctcagc ctgccgcagc
agctgccgta ccacaaccag ttctcgctgt ttgtgccgaa
acacaggcgg atggctgcta agctgattga tatatttatg
ggaatgcgtg acgtggagga cctgcagtcc gtgtgtagct
actgccagct ccgcatcaac

ID. BI-P450 Plutella xylostella cytochrome P450 (CYP6BF1v1) mRNA, GenBank: AY971374.1 nt #s 471-520, 556-605, 629-678, 752-802, 856-905, 1317-1366.

(SEQ ID NO: 254)
TGGTACAGGATTGCTGCAGAATATTCCAGAAGGTTCTCGATGATGAGATA
AGCTCGGTATACTATGGACTGTATAACTTCGTGTGCATTCGGCGTCGACT
GAAGGGAACCCTTTCACAGAAACAGGTCACCTTTTATTTGATGAAAGACC
TCCAGCAAAATTTACCGTTTCTTCCGATCTGTTATACTTGACGTTATAAA
TTGGAAGAAGAACAAATACATAACGGGAGACAGTATTGATAATGGCATAG
CGGAGCGGTTTTCTGAAGAAGGACGGAAAAGTATTGTCCCGTATACCTAC

ID. 201-500 Spodoptera frugiperda cytochrome P450 CYP6AE44 mRNA, complete cds Sequence ID: MN480661.1

(SEQ ID NO: 255)
TGAAGAACCATACGTTGGCTCTTACTTGGGCACGGAACCGACCCTCATCA
TACAAGATCCCGAATACATCAAGACCGTCATGACCAAGGACTACTATTTC
TTCAGTGGCCGTGAAGTCTCTGCATACTGTGAAAAGGAACCGTTAACTCA
GAACCTATTCTTCACTTATGGCGATAAGTGGAAGGTACTGCGTCAGAACC
TTACGCCTTTGTTCTCATCCGCAAAGATGAAGAACATGTTCCATTTGATC
GAGAAATGTGCCCGTATCTTCGAGAACATGGTCGACCAGGAAGTACAGAA

ID. 1601-1789 Plutella xylostella strain DBM1Ac-S ABC transporter subfamily H member 1 (ABCH1) mRNA, complete cds Sequence ID: KP260785.1

(SEQ ID NO: 256)
tttggctgacactgcggacgacgagaccatcgagtcgtcagaagtgcagg
tgtggctcgacatgtccaaccagcagatcggcctcatgctcaaccgagac
atacagttctcatacagggacttcgctaagaacttgctgtcgacttgcga
ctacaacccgaaggtgggcgacatcccgatcgacttcaa

ID. 332-632 Plutella xylostella juvenile hormone epoxide hydrolase mRNA, complete cds GenBank: JX297814.2

(SEQ ID NO: 257)
tagattttcggatgagatggtaaaagacttgttatatcgcatcaaccatc
gccgtaaaataagaccctcactgcaagggtcaggaaaccactatgggcca
aactctgcgctggtgaaacaagtattggaccactggagtcaaaaatacga
tttcaaagaacgtgccaagaaactaaaccggcatcctcaatacataacca
atgttaacggcctggacatccactacatacatgttcagccttcagggaat
aaacgcgttgtgccaatcctgctcatccacagcgtagaatccaacagtct
g

ID. M_011553525.1|:161-460 PREDICTED: Plutella xylostella protein mesh-like (LOC105383478), mRNA

(SEQ ID NO: 258)
ACTTATTAGTGTAAGTGTAAAAGGGCAAGAGGACGCTATAATTAATGAGG
ACAGTTTAGTGAATGACGTCACGAGTGTAGTGAGTAATGACTTGGAGGCT
CCTGTAGTTGCTGAAGCGAAAGAGGTGGGAGTGGAAGGGTTTGTGGAGGA
CCCTGCTCCTGTGGAAGTGATAGCCGATTCTGAAGCCGTGGCGGTGAGGA
GTGGGCGGTACCAGGCGCTCAATGATGCCCTGCAGGAGGGGCCGGTGGAC
CTCCAGGCAGTGGACCTGGACGAGCAGCTCGGGAGGCAGCTGTTGAACCC

XM_011553525.1|:3300-3599 PREDICTED: Plutella xylostella protein mesh-like (LOC105383478), mRNA

(SEQ ID NO: 259)
CGGTCCTTTGACGTATCCAGGGCCACCGAACTCTGCCAAGATTCCTACCA
GTGCCGATATGACTACGGAATGACCCTCAACAGAGATATGGCTGAGTTCA
CCAAGAACTATTTGTCTTCTATCACAAACATCAAGGAGAAAAACGCAAGG
AGAGTCATCAGTTGTGGCATCTTGGAGACACCGCGGTTTGGACGGAAGAG
TAACTTCTTCTTTACTCCCGGAACTACGGTGAACTTTGAGTGCAATCAAA
ACTTTATCCTGATTGGTGACAAGCGCCGAGCTTGCGAGTCCGACGGCCGG

ID 25-2. 351-500 PREDICTED: Plutella xylostella venom carboxylesterase-6 (LOC105388350), mRNA

(SEQ ID NO: 260)
CCAACGTCTACACACCGGCCATTGATCCAGAAAAGAAATACCCAGTAATG
GTTTGGATTAAAGGGTCCGAGTTTGAGAAAACTAAGGGACCTGAACTATC
TTTTAGAAATCTTATTGAAAAAGAAGTAATAGTCGTGTCTCTAAACTTCA
TAGATGATGAAAAGTTTCTAGAAAAATCACCTTTTAGTACGCTAACTGAA
GGAACTTACACTAAAATACCTATGATCTTCGGATTTGTTGAAAACGAAGG
AACAATACGTTTTGATGAGGCACTAGAAGCTGATTGGCTAACAAAGATGG.

ID. 4-303 Spodoptera frugiperda V-ATPase subunit E mRNA, complete cds Sequence ID: MT707618.1

(SEQ ID NO: 261)
GCGCTCAGCGATGCAGATGTTCAAAAACAGATCAAGCACATGATGGCCTT
CATCGAGCAAGAAGCCAATGAAAAGGCTGAGGAAATCGATGCAAAGGCCG
AGGAGGAGTTCAACATCGAAAAGGGGCGTCTGGTCCAGCAGCAGAGGTTG
AAGATCATGGAGTACTATGAGAAGAAGGAAAAGCAGGTCGAACTCCAGAA
AAAGATCCAATCCTCAAACATGCTGAACCAGGCTCGTCTCAGGGTGCTCA
AAGTACGTGAAGACCATGTACGCAACCTGTTGGACGAGGCCCGCAAGCGC

ID. Plutella xylostella mRNA for vacuolar ATP syntethase subunit E, GenBank: AB189032.1 191-240, 505-554, 683-812, 875-924, 1091-1140, 1172-1222

(SEQ ID NO: 262)
GTCTGGTGCAGCAGCAGCGCCTCAAGATCATGGAGTACTACGAGAAGAAG
GAGCGCGCGCAGGCGCAGTACAAGGAGAAGATCAAGAAGGATGTGACCTT
GCCCCCCGCGCAAATATTCAACTTTCTTATTATTATTATGTAGAACTAAA
AATGCGTACAACATTATTAACATGTATGAAAGAATCCGTATTAAGTCAGA
CTTAGCTGTAGCTTTTAGGAGACACAGTTAAATTGAAATGTTATACCGAT
AGCGTAAGCATTTTATTGATATAATTCTGGATTGTTGCCATAACAATTAT

ID. 301-400, 401-500, 1351-1450 PREDICTED: Spodoptera frugiperda uncharacterized LOC118263801 (LOC118263801), transcript variant X1, X2, and X3, mRNA

(SEQ ID NO: 263)
AAGTCGGACGTTATACAAAGAACGATGAGTGTGCTGAGGGGTTTTTAGTG
AGAAATTGTGAAAAATAGAAAGAGTGAAGATGTCGGTAAAAGCGAGCGTT
GGTTAAACAAAAAGGGTAAAAATGGCCTTCAAAGGATTCTGTGGCGAAGT
GATCGGGTTTTTCCTGGCTGTGGGTTTTTGCATCATATGTCCGGAATATG
GTTGGTTGCCTGCCTGTTCATCGCACTCTCTTGACACATACGGATCTTTA
ACTAAATGTAAATACAAGGATTTTATCAACATCAGCTGCTTATATTGACA

ID. 252-491 Diabrotica virgifera virgifera charged multivesicular body protein 4b (LOC114337301), mRNA Sequence ID: XM_028287710.1

(SEQ ID NO: 264)
gcaaagaaaaatgcgtcgaaaaataaaagagttgcactccaagccctcaa
aaagaagaaacgattggaaaagacccaactacaaatagatggaaccctta
caactattgaaatgcagagggaagccctcgaaggagctagcacaaatact
gctgtattagattctatgaaaaatgctgcagatgcccttaagaaagctca
taagaatttgaatgtagatgatgttcacgatatcatggat

ID. 87-386 PREDICTED: Plutella xylostella cytokine receptor-like (LOC105380229), mRNA Sequence ID: XM_011549746.3 Domeless:

(SEQ ID NO: 265)
CTTCAAATTATTGTAGTGATGATCATACGGAAGCCGATTTGATAGAATTT
TAATTCACATACGATACTAGCCTATATAACAGTCGATTTGTGCAGTCGGT
CAGTGAGTGTTGTTTAGTGAGTCGTGCAACTCGTACCCTGCATGACTTTG
GACTCCGATGCGCCATTTGGCCTCAGCTATAAAAACAAGGAATGTGTTTA
TCGTCTCTCCCGATTTCCCAAACGACTTTAGCTTGCAGTGATGGTGTGAT
AAGTGAACTTTAGTGATATTCCCCGACATGGCAATTTGTCAGAGCACTCG

ID. 77-376 PREDICTED: Plutella xylostella caspase-8 (LOC105390324), mRNA Sequence ID: XM_011561609.3 Dredd:

(SEQ ID NO: 266)
ATAGGTAGTTAGCTATTTTATTTAGTATTCGGGTGTTACGATAATGTTGC
AACCTGACGCTCTCAAAGGTCATGAGAATGAGGAGTTGAACAGTGTTCAC
AAAAACATACATAATATCACTGTTAATGTGATAGCTGAGGTACAAAGGGA
CTTGACTCCATACGATATCACTTCGCTGGTGTTTCTTCTATACGATATCC
CGGAAACGGCGCTTCAGCGGCTTACTCTGCTGCAGAGAGTGAGCCGGGAC
ATCAGTGGCAAGGATCTGAACTTGCTCTATGACTGGGCGGTGTACGCTCA

ID. 96-395 Plutella xylostella glutathione synthetase (Gss), mRNA Sequence ID: NM_001309054.2 GSS1:

(SEQ ID NO: 267)
ACCAAGCCCCACGTCATCATGATCATGGCTGACGACATGGGATGGGACGA
CACCTCGACCCACGGCTCCAAGTCCGTGCTGACCCCCAACCTCGACGTGC
TGACCCGCTCAGGAGTGTCCCTCCACCGCTACTACACCCACGCTCTCTGC
TCGCCCGCCCGTACCGCTGTGCTCACCGGCAAATACGCCCACACCGTCGG
TATGCAGGGTATGCCTCTGTCCAACGCTGAGGAGCGTGGTATCCCCCTAG
AGGAGCGCCTGATCTCTCAGTACCTACAGGACGCTGGTTACAGGACCCAG

ID. PREDICTED: Plutella xylostella dual oxidase (LOC105389437), mRNA Sequence ID: XM_048622382.1 Duox

(SEQ ID NO: 268)
CCCCAGCTCCTGCGCCCGCGCACGAATCACTTCCGCGATCGAATACCCGC
TTTCGCTCCGTAAACAGTCCGTGTGCAAATCATATTTTCCGTTTTCTCAT
CAACATTGCAAAACGCTAAATTGCACTTCTACACCGGCGGATGTGTTCGG
TGTCAACAAATGTGGTGTTTCTTGGCGCTCGTTGGAATAATGTCTAATTG
GAGTTGAGATTGAGACTATACACGATGGCGCGATGGTCCTCCAGCGAAGT
GTGGACACTCGTAGCGCTCTGTGCCCTCGTCACATCCTGCCTGTCAGACC

ID. MH899215.1|:461-761 Plutella xylostella chitinase 5 mRNA, complete cds

(SEQ ID NO: 269)
ACCTGGACTGGGAGTACCCTGGAGCAGCCGACCGCGGCGGCTCGTTCTCC
GACAAGGACAAGTTCCTGTACCTCGTCCAGGAGTTGAGACGAGCTTTCCT
GAGGGTTGGGAAGGGCTGGGAGCTGACCGCTGCGGTACCGCTGGCCAACT
TCCGACTGATGGAGGGGTACCATGTGCCTGAGTTGTGCCAGGAGCTGGAC
GCGATCCACGTGATGGCGTACGACCTGCGCGGCAACTGGGCCGGGTTCGC
CGACGTGCACTCCCCGCTGTACTCCCGCCCGCACGACCAGTACGCATACT
C

WUTJ02000003.1, 9171857 to 9171915 Spodoptera frugiperda

(SEQ ID NO: 270)
UACCCUGUAGAUCCGAAUUUGUUUGAAGUGAGGCGACAAAUUCGGUUCUA
GAGAGGUUU_

WUTJ02000021.1, 844642 TO 844700 Spodoptera frugiperda

(SEQ ID NO: 271)
CCUUGUCAUUCUUCUUGCCCGUGUGCUUUCAUACUACUGGACGGAGAACU
GAUAAGGGC

WUTJ02000027.1, 10184382 TO 10184433 Spodoptera frugiperda

(SEQ ID NO: 272)
GCUGCAUCCCGUGCGGCCGCAGUGUCACGUGUAGCACCAUGGGAUUCAGC
UC,

WUTJ02000008.1, 5459491 TO 5459550 Spodoptera frugiperda

(SEQ ID NO: 273)
CUGGUUUUCAUAAUGAUUUGACAGAUUGUUCUAAAUUCUGAGAUCAUUGU
GAAAGCUGAU

WUTJ02000027.1, 10183182 TO 10183238 Spodoptera frugiperda

(SEQ ID NO: 274)
CUAGCACUUUGGCUGUGACCUGUGUGUCGUGUCACAUCACAGUCAGAGUU
CUAGCU

Five combined sequences, WUTJ02000003.1, 9171857 to 9171915, WUTJ02000021.1, 844642 TO 844700, WUTJ02000027.1, 10184382 TO 10184433, WUTJ02000008.1, 5459491 TO 5459550, WUTJ02000027.1, 10183182 TO 10183238 Spodoptera frugiperda

(SEQ ID NO: 275)
CTAGCACTTTGGCTGTGACCTGTGTGTCGTAGTCACATCACAGTCAGAGT
TCTAGCTTACCCTGTAGATCCGAATTTGTTTGAAGTGAGGCGACAAATTC
GGTTCTAGAGAGGTTTCCTTGTCATTCTTCTTGCCCGTGTGCTTTCATAC
TACTGGACGGAGAACTGATAAGGGCGCTGCATCCCGTGCGGCCGCAGTGT
CACGTGTAGCACCATGGGATTCAGCTCCTGGTTTTCATAATGATTTGACA
GATTGTTCTAAATTCTGAGATCATTGTGAAAGCTGAT

Spodoptera frugiperda isolate AFR2017 chromosome 1

WUTJ02000001.1, Sequence Range: 7362721 to 7363341,

(SEQ ID NO: 294)
5′-AAATTAAATGTGGTTTTGGCATCAAAGTCGGCTTGTCATAGGTCATA
ACGTAGCTATCACAGCCAGCTTTGATGAGCATGACCTCATTCCTGAACAT
CATTATGCGGACCTTCAGAGGGCTTGTAGTATTAGTGGGTCAATGGTTTC
CCGGGCCTAGCCTGTCAAAGCGGCGGTGAAATGTATCCGTCATCATATCA
CAGCCACTTTGATGTGGTCGGTTTACGGAGAGCACCACGTCCTTACAGCA
CTGCCAGTAGGTAAGCAAGTCCGTGGCCCATTCGTAAAAATGGTTGTGCC
ATGTAGCTGACTCATATCACAGCCATTTTTGACGAGTTGGGCTGGGGGAC
GTTACGACGAGCGGCTCCCTGCTTCCATGACACTTCGAGCAGCACGCGGT
CCGCGAGTGCAGCGGCGCTCACAAAGTGGCTGTAATGTGTGTCCCCGTAC
ATATCACAGCCAGCTTTGATGAGCGCTGAGCATCATTTGCTTTCATCAAT
ATAGTTGAGCGACATCAACAAACCCTCAAGCGCAAGAAGCGCGTCAGGGG
GTCAGCAAAGTGGTTGTGTCTTATGAGATTCATGAACATATCACAGCCAG
CTTTGTTGACTTCTTTGTCGCGTA-3′

Spodoptera frugiperda isolate AFR2017 chromosome 15, WUTJ02000007.1, Sequence Range: 4191113 to 4191206; Spodoptera frugiperda isolate AFR2017 chromosome 1, WUTJ02000001.1, Sequence Range: 7377755 to 7377816; Spodoptera frugiperda isolate AFR2017 chromosome 8, WUTJ02000030.1, Sequence Range: 2113180 to 2113240, Concatenated in a single transcript:

(SEQ ID NO: 295)
5′-auuguacuucaucaggugcucuggugaugaugguuccaggcgcuugu
uggaguacacuuacugaaagacaggaguagugagauguccucgacaucac
aaauucucacuaccuugucuuucaugUCUCAUUCCCGGUUACUCACUCAA
CCUGGGUGUGAUGUGUGCACUAGUUGCUCGGCCCAUCACAACCUCCUUGA
GUGAGCGAUCGUGGAGGAGG-3′

Plutella xylostella strain LV isolate Lockyer Valley chromosome 6, JAHIBW010000006.1, Sequence Range 1:7637161 to 7637259; Plutella xylostella strain LV isolate Lockyer Valley chromosome 26, JAHIBW010000026.1, Sequence Range 1:2561366 to 2561459; Plutella xylostella strain DBM-FZ-S scaffold_156_8, AHIO01005922.1, Sequence Range 1:9249 to 9340; Plutella xylostella strain LV isolate Lockyer Valley chromosome 2, JAHIBW010000002.1, Sequence Range 1:3126454 to 3126533; Plutella xylostella strain LV isolate Lockyer Valley chromosome 19, JAHIBW010000019.1, Sequence Range 1:676463 to 676533, CONCATENATED in a single transcript:

(SEQ ID NO: 296)
5′-tctgttaatgaagagagctatccgtcgacagtattgaccataaaaac
tgtcatggagttgctctctttatgaacggtagcatcaaatattagtgccc
tacatctaccctgtagatccgaatttgtttgaagtgaggcgacaaattcg
gttctagagaggtttgtgtggtgcacgctgggaaggcaagatgtcggcat
agctgattgatgtgatatacggctgtgtcacatcgagccagctcaatgtg
ttagacgaagttggtttgtgaccgtcactaacgggcagtagtctatggtt
tgctcgttttgatgatcgcaaaattaactcgcttgactatcaaggctcca
cgtctgagttggacaggggatctagacagttcgcaacatacttctgccag
atctaactttccagctcacgcgtggagccaac-3′

Plutella xylostella strain LV isolate Lockyer Valley chromosome 6, JAHIBW010000006.1, Sequence Range: 7637161 to 7637259; Plutella xylostella strain LV isolate Lockyer Valley chromosome 26, JAHIBW010000026.1, Sequence Range: 2561366 to 2561459; Plutella xylostella strain DBM-FZ-S scaffold_156_8, AHIO01005922.1, Sequence Range: 9249 to 9340; Plutella xylostella strain LV isolate Lockyer Valley chromosome 2, JAHIBW010000002.1; Sequence Range: 3126454 to 3126533; Plutella xylostella strain LV isolate Lockyer Valley chromosome 19, JAHIBW010000019.1, Sequence Range: 676463 to 676533, CONCATENATED IN A SINGLE TRANSCRIPT:

(SEQ ID NO: 297)
5′-tctgttaatgaagagagctatccgtcgacagtattgaccataaaaac
tgtcatggagttgctctctttatgaacggtagcatcaaatattagtgccc
tacatctaccctgtagatccgaatttgtttgaagtgaggcgacaaattcg
gttctagagaggtttgtgtggtgcacgctgggaaggcaagatgtcggcat
agctgattgatgtgatatacggctgtgtcacatcgagccagctcaatgtg
ttagacgaagttggtttgtgaccgtcactaacgggcagtagtctatggtt
tgctcgttttgatgatcgcaaaattaactcgcttgactatcaaggctcca
cgtctgagttggacaggggatctagacagttcgcaacatacttctgccag
atctaactttccagctcacgcgtggagccaac-3′

Plutella xylostella strain DBM-FZ-S scaffold_70_86, AHIO01024435.1, Sequence Range: 11544 to 11631; Plutella xylostella strain LV isolate Lockyer Valley chromosome 7, JAHIBW010000007.1, Sequence Range: 8613596 to 8613685; Plutella xylostella strain LV isolate Lockyer Valley chromosome 19, JAHIBW010000019.1, Sequence Range: 7047245 to 7047333; Plutella xylostella strain LV isolate Lockyer Valley chromosome 1, JAHIBW010000001.1, Sequence Range: 12328542 to 12328629; Plutella xylostella genome assembly, CABWKK010000094.1, Sequence Range: 6680662 to 6680747, CONCATENATED IN A SINGLE TRANSCRIPT:

(SEQ ID NO: 298)
5′-aaatggccaatttagtggccgttacctctgagcttgtatgtccataa
attcatccaaattcagaggtaacggcacactacattaggctactggttct
tgtctctggcgcggcagtcagaattcgggacaagcccggagcggtggctt
gtcccgaattttgactcggccaaatcaaaacagcggtggtctggctagga
cgtgattcggtgtaacccttgtgtttcccgatcaaattgcaccaatcccg
gcctgccttcggccaccgcccgctctagccatatgaaagacatgggtagt
gagatgtccaagtacatcaaaaattctcactaccttgtctttcatgtgga
ctgagcataatctgacattagtcagtgacatagctgggataagttagtgt
gaaatgaaacctatctcagctatgtcactatctcgtgtcaggaat-3′

SEQ ID NOs: 276 through 290 are presented in the Examples, Section 2.

SEQ ID NOs: 291 through 293 are presented in the Examples, Section 3.

### EMBODIMENTS

1. A composition comprising a post-transcriptionally chemically modified double strand RNA (MdsRNA) wherein the MdsRNA comprises a double strand RNA wherein about 2% to about 30% of all the nucleotides independently comprise Formula (I):

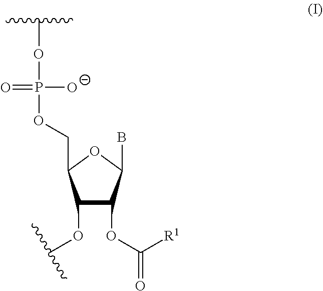

or an acceptable salt thereof, wherein:
B is a nucleobase;
R¹ is selected from:

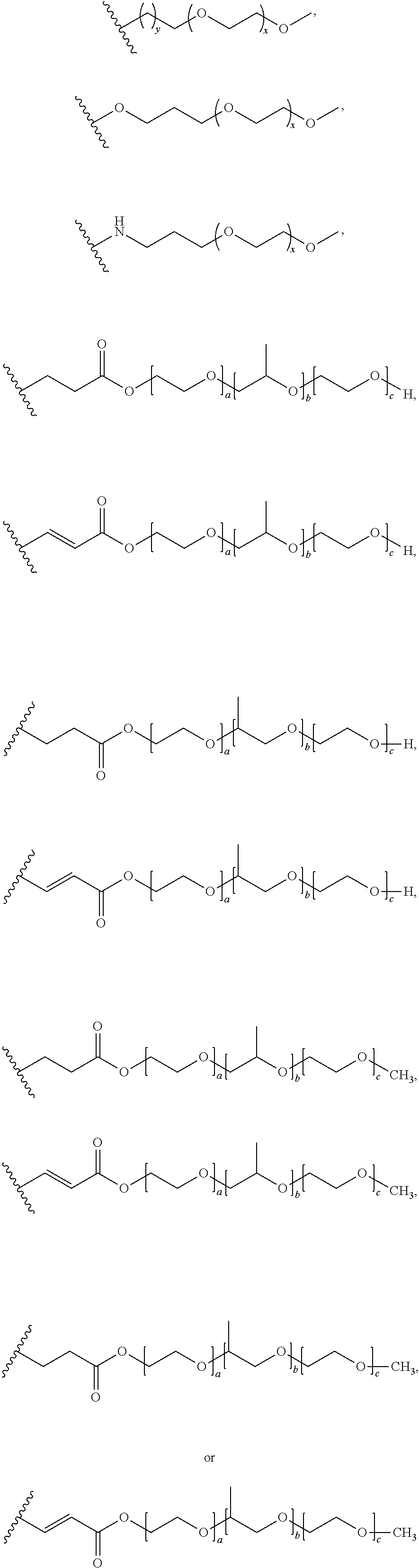

wherein y is an integer from 1-8, x is an integer from 12-1000, a is an integer from 12-1000, b is an integer from 12-1000, and c is an integer from 12-1000; and
optionally wherein between 10% and 60% of all the nucleotides independently comprise LMW nucleotides of Formula (III):

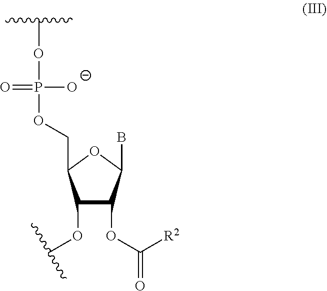

or an acceptable salt thereof, wherein: B is a nucleobase;
R² is selected from C₁-C₂₅ alkyl, C₁-C₂₅ alkenyl, C₁-C₂₅ alkynyl, C₅-C₁₂ aryl or C₅-C₁₂ heteroaryl, wherein R² is optionally substituted with one or more substituents selected from halo, C₁₋₁₂ alkyl, C₁-C₁₂ aminoalkyl, or C₁-C₁₂ alkoxy.

2. A composition comprising a post-transcriptionally chemically modified ssRNA (MRNA) wherein the ssRNA comprises molecules of about 55 nucleotides to about 900 nucleotides, wherein the ssRNA comprises type (A), (B), or (C) RNA, wherein type (A) RNA comprises N or N+1 sense strand sections, Ax, 2N−1 or 2N, respectively, linker sections, Lz, and N antisense strand sections, Ay*, type (B) RNA and type (C) RNA comprises N sense strand sections, By or Cy, respectively, 2N−1 linker sections, Jz or Kz, respectively, and N antisense strand sections, By* or Cy*, respectively, wherein N is an integer from about 2 to about 51, x is an integer from 1 to N+1, y is an integer from 1 to N, and z is an integer from 1 to 2N, type (A) RNA is arranged as follows: 5′-A1-L1-A2-L2- . . . -AN-LN-A1*-LN+1- . . . -L2N−1-AN*-3′ or 5′-A1-L1-A2-L2- . . . -AN-LN-A1*-LN+1- . . . -AN*-L2N-AN+1-3′, type (B) RNA is arranged as follows: 5′-B1-J1-B2-J2- . . . -BN-JN-BN*-JN+1- . . . -B2*-J2N−1-B1*-3′, type (C) RNA is arranged as follows: 5′-C1-K1-C1*-K2-C2-K3-C2*- . . . -CN-K2N−1—CN*-3′, and sense strands A1 through AN or A1 through AN+1, B1 through BN, and C1 through CN, and antisense strands A1* through AN*, B1* through BN*, and C1* through CN* have minimum lengths of 21 nucleotides and maximum lengths of 1000, or 500, or 250, or 125, or 61, or 27 nucleotides, wherein a section of at least one strand or antisense strand comprises a sequence complementary to a nucleic acid in a target host capable of reducing the concentration of one of its expressed RNAs when the composition is administered to the host or one of its cells, wherein from about 10% to about 100% of all the nucleotides in the linker strands L1 through L2N−1 or L1 through L2N, J1 through J2N−1, and K1 through K2N−1 independently comprise Formula (I):

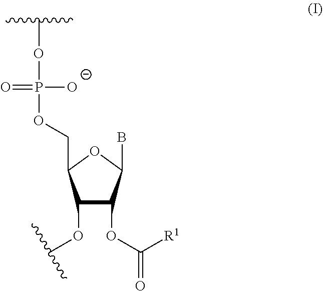

or an acceptable salt thereof, wherein:
B is a nucleobase;
R¹ is selected from:

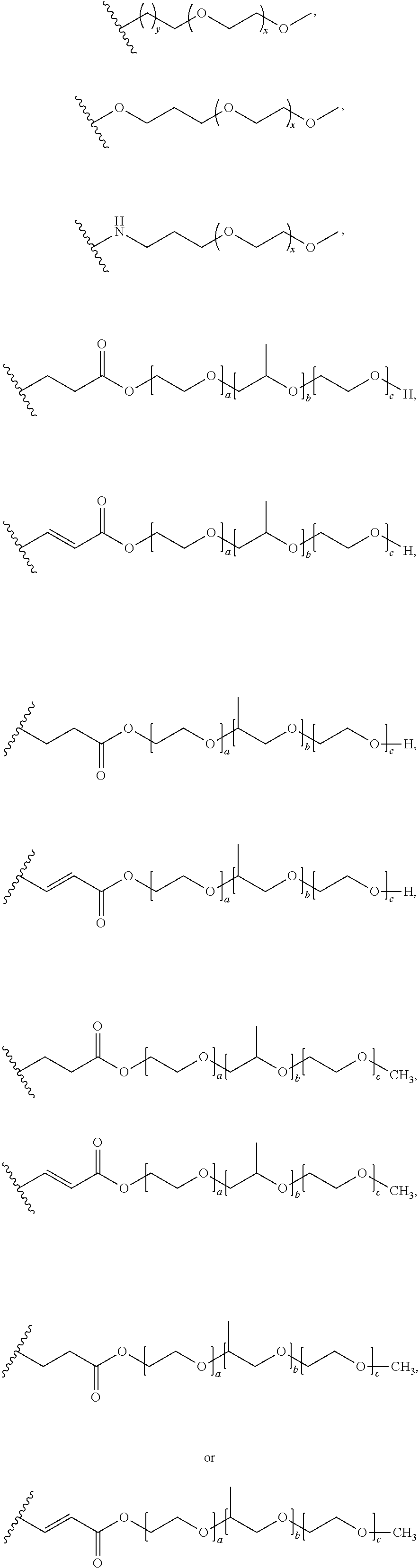

wherein y is an integer from 1-8, x is an integer from 12-1000, a is an integer from 12-1000, b is an integer from 12-1000, and c is an integer from 12-1000; and
optionally wherein at least 20% of all the nucleotides in the linker strands L1 through L2N−1 or L1 through L2N, J1 through J2N−1, and K1 through K2N−1 independently comprise LMW nucleotides of Formula (III):

3. The composition of embodiment 1 or embodiment 2, wherein R¹ is selected from:

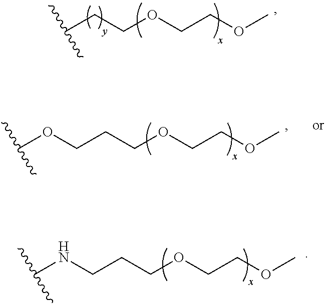

4. The composition of embodiments 1-3, wherein x is an integer from 80-1000.

5. The composition of any one of embodiments 1-4, wherein a is an integer from 80-1000.

6. The composition of any one of embodiments 1-5, wherein b is an integer from 80-1000.

7. The composition of any one of embodiments 1-6, wherein c is an integer from 80-1000.

8. The composition of any one of embodiments 1-7, wherein R¹ has a molecular weight of from about 5,000 Da to about 40,000 Da.

9. The composition of any one of embodiments 1-8, wherein the MdsRNA or the MRNA comprises a sequence complementary to an expressed RNA in a target insect.

10. The composition of any one of embodiments 1-9, wherein the MdsRNA or the MRNA comprises a sequence complementary to a target region in Diamondback moth AChE2, P450, DOMELESS, DOUX, MESH, P450 CYP6BF1v1, Venom or VPASE; Western corn root worm SNF7; Fall armyworm P450, CYP9A58, Cytokine receptor DOMELESS, Dredd, VPASE and Protein MESH.

11. A method of preparing a composition comprising a post-transcriptionally chemically modified double strand RNA (MdsRNA) of embodiment 1 or a post-transcriptionally chemically modified ssRNA (MRNA) of embodiment 2; and
the method comprising:
(a) contacting a compound of Formula (II):

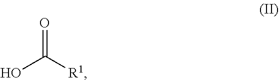

with an activation agent to form a compound of Formula (IIA):

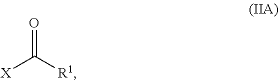

wherein X is a suitable leaving group;
(b) contacting a compound of Formula (IA):

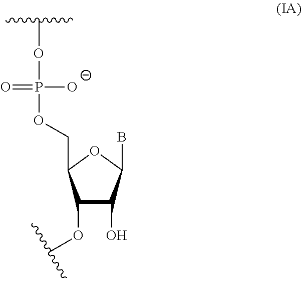

with a compound of Formula (IIA) to form a compound of Formula (I);
(c) optionally contacting a compound of Formula (II):

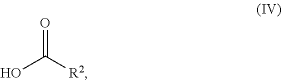

with an activation agent to form a compound of Formula (IVA):

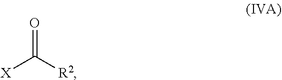

wherein X is a suitable leaving group;
(d) optionally contacting a compound of Formula (IA):

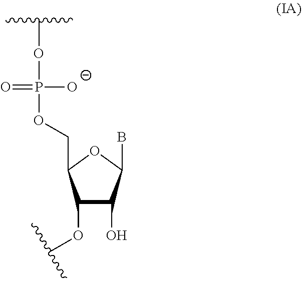

with a compound of Formula (IVA) to form a compound of Formula (III).

12. The method of embodiment 11, wherein (a), (b), (c), or (d) are carried out in a substantially anhydrous solvent.

13. The method of embodiment 12, wherein the anhydrous solvent is selected from DMSO or DCM.

14. The method of any one of embodiments 11-13, wherein (a), (b), (c), and (d) are carried out without intervening purification.

15. The method of any one of embodiments 11-13, wherein there is a purification step between (a), (b), (c), and (d).

16. The method of any one of embodiments 10-14, wherein an ionic solvent is added after (a) or (c).

17. The method of embodiment 15, wherein the ionic solvent is selected from benzyltributyl ammonium chloride or benzyltrimethyl ammonium chloride.

18. The method of any one of embodiments 11-17, wherein the activation agent is carbonyldiimidazole.

19. The method of any one of embodiments 11-18, wherein the suitable leaving group is:

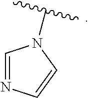 [SMILES: *n1ccnc1]

20. The method of any one of embodiments 11-19, wherein (b) has a ratio of less than ten equivalents of the compound of Formula (IIA) per nucleotides of the dsRNA and (d) has a ratio of between two and fifty equivalents of the compound of Formula (VA) per nucleotide of the dsRNA.

21. The method of any one of embodiments 11-20, wherein (b) has a ratio of less than two equivalents of the compound of Formula (IIA) per nucleotide of the dsRNA and (d) has a ratio of between four and twenty-five equivalents of the compound of Formula (VA) per nucleotide of the dsRNA.

22. The method of any one of embodiments 11-21, wherein R¹ is

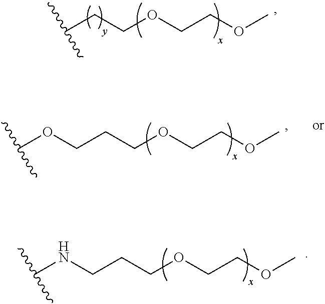

23. The method of any one of embodiments 11-22, wherein x is an integer from 80-1000.

24. The method of any one of embodiments 11-23, wherein a is an integer from 80-1000.

25. The method of any one of embodiments 11-24, wherein b is an integer from 80-1000.

26. The method of any one of embodiments 11-25, wherein c is an integer from 80-1000.

27. The method of any one of embodiments 11-26, wherein R¹ has a molecular weight between 5,000 and 40,000 Da.

28. The method of any one of embodiments 11-27, wherein the MdsRNA comprises a sequence complementary to an expressed RNA in a target insect.

29. The method of any one of embodiments 11-28, wherein the MdsRNA comprises a sequence complementary to a target region in Diamondback moth AChE2, P450, DOMELESS, DOUX, MESH, P450 CYP6BF1v1, Venom or VPASE; Western corn root worm SNF7; Fall armyworm P450, CYP9A58, Cytokine receptor DOMELESS, Dredd, VPASE and Protein MESH.

30. A method of preparing a composition comprising a post-transcriptionally chemically modified double strand RNA (MdsRNA) or a post-transcriptionally chemically modified ssRNA (MRNA) wherein the MdsRNA comprises a double strand RNA or the MRNA comprises single strand RNA wherein at least about 2% of the nucleotides independently comprise Formula (VI):

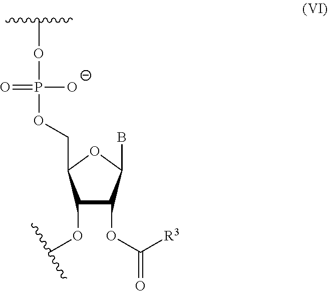

or an acceptable salt thereof, wherein:
B is a nucleobase;
R³ is selected from: amino acids, fatty acids, alkyl; substituted alkyl; alkenyl; substituted alkenyl; alkynyl; substituted alkynyl; aryl; substituted aryl; C1-C10 alkyl, C1-C10 alkenyl, or C1-C10 alkynyl wherein alkyl and alkenyl can be linear, branched or cyclic; hydrogen; methyl; ethyl; propyl; isopropyl; butyl; isobutyl; tert-butyl; pentyl; hexyl; cyclohexyl; heptyl; octyl; nonyl; decyl; vinyl; allyl; ethynyl; benzyl; cinnamyl; C6-C14 aryl; C6-C14 substituted aryl; heterocyclyl; C5-C14 heterocyclyl; phenyl; mono or disubstituted phenyl wherein the substituents are selected from C1-C10 alkyl, C1-C10 alkenyl, C1-C6 alkoxy, halogen, nitro, methylsulfonyl, and trifluoromethyl; 2-nitrophenyl; 4-nitrophenyl; 2;4-dinitrophenyl; 2-trifluoromethylphenyl; 4-trifluoromethylphenyl; styryl; C8-C16 substituted styryl; 2-aminophenyl; mono or disubstituted 2-aminophenyl wherein the substituents are selected from C1-C10 alkyl, C1-C10 alkenyl, C1-C6 alkoxy, halogen, nitro, methylsulfonyl, and trifluoromethyl; N-alkyl-2-aminophenyl or N-aryl-2-aminophenyl wherein alkyl has the formula —CₘH₂ₘ₊₁ (wherein m is an integer less than or equal to 12) and aryl is an aromatic moiety; 2-amino-3-methyl-phenyl; 2-amino-5-chlorophenyl; 2-methyl-5-chlorophenyl; N-methyla-2-minophenyl; N-ethyl-2-aminophenyl; N-propyl-2-aminophenyl; N-butyl2-aminophenyl; N-pentyl-2-aminophenyl; N-methyl-2-amino-4-nitrophenyl; 2-methyl-3-furyl; 2-methylnicotyl or N-trifluoromethyl-2-aminophenyl; silanyl; substituted silanyl; C1-C10 alkylsilanyl; C3-C12 trialkylsilanyl; C2-C12 alkoxyalkyl; C2-C12 alkoxyalkenyl; C2-C12 alkylthioalkyl; alkylsulfonyl; C1-C10 alkylsulfonyl; C1-C10 haloalkyl; C1-C10 haloalkenyl or C1-C10 aminoalkyl; —(CH₂CH₂O)ₚCH₃, —(CH₂CH₂O)ₚH, or —(CH₂CH₂O)ₚCOOR₄ wherein p is an integer from 2 to 8 and R⁴ is H, alkyl, substituted alkyl, aryl, or substituted aryl; —(CH₂CH₂O)₈COOH; —CH₂CH₂OH; —(CH₂CH₂O)₄OH; —(CH₂CH₂O)₆OH; —(CH₂CH₂O)₈OH; —(CH₂CH₂O)₈COOMe; —(CH₂CH₂O)₄OMe; —(CH₂CH₂O)₆OMe; —(CH₂CH₂O)₈OMe; —CH₂OCH₃; —CH₂OCH₂CH₃; or —CH₂OCH₂CH₂OCH₃; and
the method comprising:
(a) contacting a compound of Formula (IV):

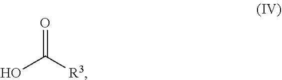

with an activation agent to form a compound of Formula (IVA):

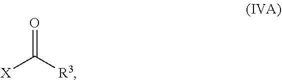

wherein X is a suitable leaving group; and
(b) contacting a compound of Formula (VIA):

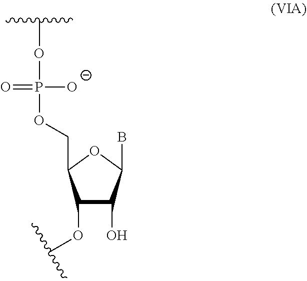

with a compound of Formula (IVA) to form a compound of Formula (VI).

31. The method of embodiment 30, wherein (a) or (b) are carried out in an anhydrous solvent.

32. The method of embodiment 31, wherein the anhydrous solvent is selected from DMSO or DCM.

33. The method of any one of embodiments 30-32, wherein (a) and (b) are carried out without intervening purification.

34. The method of any one of embodiments 30-32, wherein there is a purification step between (a) and (b).

35. The method of any one of embodiments 30-34, wherein an ionic solvent is added after (a).

36. The method of embodiment 35, wherein the ionic solvent is selected from benzyltributyl ammonium chloride or benzyltrimethyl ammonium chloride.

37. The method of any one of embodiments 30-36, wherein the activation agent is carbonyldiimidazole.

38. The method of any one of embodiments 30-37, wherein the suitable leaving group is:

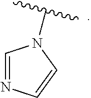 [SMILES: *n1ccnc1]

39. The method of any one of embodiments 30-38, wherein (b) has a ratio of between two and fifty equivalents of the compound of Formula (IVA) per nucleotide of the dsRNA.

40. The method of any one of embodiments 30-39, wherein (b) has a ratio of between four and twenty-five equivalents of the compound of Formula (IVA) per nucleotide of the dsRNA.

41. The method of any one of embodiments 30-40, wherein R³ is N-methyl anthranoyl (NMA), N-benzyl anthranoyl (NBA), dimethyl furoyl, -Tyr, -Trp, -Leu, octanoyl, lauroyl, linoleyl, oleoyl, nicotinoyl or benzoyl.

42. The method of any one of embodiments 30-41, wherein the MdsRNA comprises a sequence complementary to an expressed RNA in a target insect.

43. The method of any one of embodiments 30-42, wherein the MdsRNA comprises a sequence complementary to a target region in Diamondback moth AChE2, P450, DOMELESS, DOUX, MESH, P450 CYP6BF1v1, Venom or VPASE; Western corn root worm SNF7; Fall armyworm P450, Cytokine receptor DOMELESS, Dredd, VPASE and Protein MESH.

44. A method of modifying the expression of a polynucleotide of interest in any of the following: an insect, an acari, a fungus or a weed comprising administering a composition of any one of embodiments 1-10.

45. The method of embodiment 44, wherein the expression is for a target region in Diamondback moth AChE2, P450, DOMELESS, DOUX, MESH, P450 CYP6BF1v1, Venom or VPASE; Western corn root worm SNF7; Fall armyworm P450, VPASE, Cytokine receptor DOMELESS, Dredd, and Protein MESH.

46. The method of embodiment 45, wherein the target region is in P450 CYP6BF1v1, in MESH transcript variant X1 or Venom carboylesterase-6.

47. The method of embodiment 45, wherein the expression is for a target region in Fall armyworm P450 CYP9A58, P450 CYP321A8, P450 CYP6B2-like, Cytokine receptor DOMELESS, Dredd, and Protein MESH transcript variant X1.

48. The method of embodiment 44, wherein the expression is for target region in Western corn root worm SNF7.

49. The method of embodiments 44-48, wherein the modified expression increase mortality or induce growth stunting, or stop instar development or reduces the fertility of the target insect.

50. The method of any one of embodiments 44-49, wherein the modified expression reduces fertility of Lepidopteran insects.

51. The method of embodiment 50, wherein the Lepidopteran insect is Diamondback moth, Gypsy moth, or Fall armyworm.

52. The method of any one of embodiments 44-49, wherein the expression increase mortality or induce growth stunting, or stop instar development or reduces the fertility of Coleopteran insects.

53. The method of embodiment 52, wherein the Coleopteran insect is Colorado potato beetle, Canola flea beetle or Western corn root worm.

54. The method of embodiment 44, wherein the weed is Palmer Amaranth.

55. The method of embodiment 44, wherein the fungus is Fusarium Graminearum or Botrytis.

56. The method of embodiment 44, wherein the acari is Verroa mite.

57. The composition of any one of embodiments 1-9, wherein at least about 2% to about 50% of all the nucleotides independently comprise LMW nucleotides of Formula (III).

58. The composition of any one of embodiments 1-9 and 56, wherein R² is N-methyl anthranoyl (NMA), N-benzyl anthranoyl (NBA), dimethyl furoyl, -Tyr, -Trp, -Leu, octanoyl, lauroyl, linoleyl, oleoyl, nicotinoyl or benzoyl.

59. The method of any one of embodiments 10-55, wherein at least about 2% to about 50% of all the nucleotides independently comprise LMW nucleotides of Formula (III).

60. A method for producing MdsRNA deliverable to pests, comprising: a) transcribing DNA into a ssRNA strand comprising at least one sense section followed by at least one section antisense (e.g., from about 1 section to about 20 sections) to the sense section capable of forming at least one dsRNA stem; b) adding a salt comprising a quaternary ammonium or a quaternary phosphonium cation or a pyrrolidinium or a pyridium or a piperidimium and a neutralizing anion to produce an isolate comprising more than half of the dsRNA produced in step (a); and c) conducting a chemical reaction between suitable reagents and at least about 1% of the 2′OH groups in the RNA resulting from step (b) in a blend comprising at least about 1% of the salt used in step (b), resulting in a post-transcriptionally chemically modified double strand RNA (MdsRNA) or a post-transcriptionally chemically modified RNA (MRNA).

61. The method of embodiment 60, wherein the ssRNA comprises (A) or (B) or both (A) and (B), wherein (A) comprises a sense section followed by a section antisense to the sense section and (B) comprises a sense section i, a sense section j, a sense section k, a section i* antisense to i, a section j* antisense to j, and a section k* antisense to k, wherein such sections are located in order 5′-i-j-k-i*-j*-k*-3′ and wherein, optionally, each section is between 20 and 60 nucleotides long.

62. The method of embodiment 60 or embodiment 61, wherein the transcription of DNA into ssRNA is conducted by a DNA-dependent RNA polymerase in an aqueous phase substantially devoid of microorganisms.

63. The method of embodiment 60 or embodiment 61, wherein the transcription of DNA into ssRNA is conducted by a microorganism chosen from a bacterium and a yeast.

64. The method of embodiment 63, wherein the microorganism also expresses a coat protein substantially identical to that of a levividirae virus.

65. The method of embodiment 64, wherein the levividirae virus is bacteriophage MS2.

66. The method of embodiment 65, wherein the microorganism is E. coli.

67. The method of any one of embodiments 60 through 66, wherein the quaternary ammonium cation is chosen from trimethyloctyl ammonium, trimethyldecyl ammonium, trimethyldodecyl ammonium, trimethyltetradecyl ammonium, trimethylhexadecyl, ammonium, trimethyloctadecyl ammonium, trimethylbenzyl ammonium, tributylbenzyl ammonium, choline, amyltriethylammonium, butyltrimethylammonium, benzylethyldimethylammonium, cyclohexyltrimethylammonium, diethyl(methyl)-propylammonium, diethyl(2-methoxyethyl)-methylammonium, ethyl(2-methoxyethyl)-dimethylammonium, ethyl(3-methoxypropyl)dimethylammonium, ethyl(dimethyl)(2-phenylethyl)-ammonium, methyltri-n-octylammonium, tetrabutylammonium, tetrahexylammonium, tetraamylammonium, tetra-n-octylammonium, tetraheptylammonium, tetraamylammonium, tetrapropylammonium, tributylmethylammonium, trimethylpropylammonium, tributyl(methyl)-ammonium, 1-allyl-3-methylimidazolium, 1-butyl-3-methylimidazolium, 1-butyl-2,3-dimethylimidazolium, 3,3′-(butane-1,4-diyl)-bis(1-vinyl-3-imidazolium), 1,2-dimethyl-3-propylimidazolium, 1-decyl-3-methylimidazolium, 1,3-dimethylimidazolium, 1-dodecyl-3-methylimidazolium, 1-ethyl-3-methylimidazolium, 1-ethyl-2,3-dimethylimidazolium, 1-hexyl-3-methylimidazolium, 1-(2-hydroxyethyl)-3-methylimidazolium, 1-methyl-3-noctylimidazolium, 1-methyl-3-pentylimidazolium, 1-benzyl-3-methylimidazolium, 4-ethyl-4-methylmorpholinium, tributylhexylphosphonium, tributylhexadecylphosphonium, tributylmethylphosphonium, tributyl-noctylphosphonium, tetrabutylphosphonium, tetra-n-octylphosphonium, tributyl(2-methoxyethyl)-phosphonium, tributylmethylphosphonium, trihexyl(tetradecyl)-phosphonium, trihexyl(tetradecyl)-phosphonium, tributyl(ethyl)phosphonium, tributyl(methyl)phosphonium, 1-allyl-1-methylpyrrolidinium, 1-butyl-1-methylpyrrolidinium, 1-ethyl-1-methylpyrrolidinium, 1-methyl-1-propylpyrrolidinium, 1-(2-methoxyethyl)-1-methylpyrrolidinium, 1-methyl-1-noctylpyrrolidinium, 1-methyl-1-pentylpyrrolidinium, tributylsulfonium, triethylsulfonium, and the anion is chosen from chloride, bromide, fluoride, iodide, acetate, propionate, butyrate, hexanoate, octanoate, decanoate, laurate, myristate, palmitate, palmitoleate, stearate, oleate, oxalate, succinate, bis(trifluoromethanesulfonyl)-imide, tetrafluoroborate, hexafluorophosphate, ptoluenesulfonate, trifluoromethanesulfonate, tetrachloroferrate, methanesulfonate, tribromide, hydrogen sulfate, thiocyanate, triflate, hexafluoroantimonate, dimethyl phosphate, methyl sulfate, dicyanamide, nitrate.

### REFERENCES

Andrés et al. (2015). Setup and validation of shake-flask procedures for the determination of partition coefficients (log D) from low drug amounts. European Journal of Pharmaceutical Sciences, 76, 181-191.
Ansell, et al. (2015). Drug resistance in Giardia duodenalis. Biotechnology advances, 33 (6), 888-901.
Barash et al. (2017). Giardia colonizes and encysts in high-density foci in the murine small intestine. mSphere, 2 (3), e00343-16.
Begum et al. (2015). Immune evasion mechanisms of Entamoeba histolytica: progression to disease. Frontiers in microbiology, 6, 1394.
Bendesky et al. (2002). Is metronidazole carcinogenic? Mutation Research/Reviews in Mutation Research, 511 (2), 133-144.
Chan et al. (2017). Comparative Analysis Reveals Furoyl in Vivo Selective Hydroxyl Acylation Analyzed by Primer Extension Reagents Form Stable Ribosyl Ester Adducts. Biochemistry, 56 (13), 1811-1814.
Chen et al. (2004). Poly-2′-DNP-RNAs with enhanced efficacy for inhibiting cancer cell growth. Oligonucleotides, 14 (2), 90-99.
Cosar et al. 1959. The activity of 1-(2-hydroxyethyl)-2-methyl5-nitroimidazole (R. P. 8823) against experimental Trichomonas vaginalis infections. Ann. Inst. Pasteur (Paris) 96:238-24.
Cudmore et al. (2004). Treatment of infections caused by metronidazole-resistant Trichomonas vaginalis. Clinical microbiology reviews, 17 (4), 783-793.
Davis et al. (2010). Evidence of RNAi in humans from systemically administered siRNA via targeted nanoparticles. nature, 464 (7291), 1067.
Diamond et al. (1978). A new medium for the axenic cultivation of Entamoeba histolytica and other Entamoeba. Transactions of the Royal Society of Tropical Medicine and Hygiene, 72 (4), 431-432.
Elliot et al. (2011). Chapter 4: The RNA-Binding Proteins. Molecular Biology of RNA. Oxford University Press.
Fire et al. (1998). Potent and specific genetic interference by double-stranded RNA in Caenorhabditis elegans. Nature, 391 (6669), 806-811.
Friedman et al. (2015). Highly stable aptamers selected from a 2′-fully modified fGmH RNA library for targeting biomaterials. Biomaterials, 36, 110-123.
Gardner et al. (2001). Treatment of giardiasis. Clinical microbiology reviews, 14 (1), 114-128.
Hemingway J et al. “The molecular basis of insecticide resistance in mosquitoes.” Insect Biochemistry and Molecular Biology (2004) 34.7:653-665.
Jin et al. (2014). Application of dsRNA in cancer immunotherapy: current status and future trends. Anti-Cancer Agents in Medicinal Chemistry (Formerly Current Medicinal Chemistry-Anti-Cancer Agents), 14 (2), 241-255.
Khvorova, A., & Watts, J. K. (2017). The chemical evolution of oligonucleotide therapies of clinical utility. Nature Biotechnology, 35 (3), 238-248.
Kujawski et al. (2011). Prediction of log P: ALOGPS application in medicinal chemistry education. Journal of Chemical Education, 89 (1), 64-67.
Leeson et al. (2007). The influence of drug-like concepts on decision-making in medicinal chemistry. Nature Reviews Drug Discovery, 6 (11), 881.
Liao et al. (2005). Biomembrane-permeable and ribonuclease-resistant siRNA with enhanced activity. Oligonucleotides, 15 (3), 196-205.
Lipinski et al. (2004). Lead- and drug-like compounds: the rule-of-five revolution. Drug Discovery Today: Technologies, 1 (4), 337-341.
Liu et al. (2010). Fabrication of stable and RNase-resistant RNA nanoparticles active in gearing the nanomotors for viral DNA packaging. ACS nano, 5 (1), 237-246.
Liu et al. (2015). Digestion of nucleic acids starts in the stomach. Scientific reports, 5, 11936.
López-Rosas et al. (2012). mRNA decay proteins are targeted to poly (A)+RNA and dsRNA-containing cytoplasmic foci that resemble P-bodies in Entamoeba histolytica. PloS one, 7 (9), e45966.
Lorenzi et al. (2010). New assembly, reannotation and analysis of the Entamoeba histolytica genome reveal new genomic features and protein content information. PLoS neglected tropical diseases, 4 (6), e716.
Marcial-Quino et al. (2017). RNAi-Mediated Specific Gene Silencing as a Tool for the Discovery of New Drug Targets in Giardia lamblia; Evaluation Using the NADH Oxidase Gene. Genes, 8 (11), 303.
Merino et al. (2005). RNA structure analysis at single nucleotide resolution by selective 2′-hydroxyl acylation and primer extension (SHAPE). Journal of the American Chemical Society, 127 (12), 4223-4231
Mortimer et al. (2009). Time-resolved RNA SHAPE chemistry: Quantitative RNA structure analysis in one second snapshots and at single nucleotide resolution. Nature protocols, 4 (10), 1413.
Nair et al. (2014). Multivalent N-acetylgalactosamine-conjugated siRNA localizes in hepatocytes and elicits robust RNAimediated gene silencing. Journal of the American Chemical Society, 136 (49), 16958-16961.
Ohnishi et al. (2014). Subjective adverse reactions to metronidazole in patients with amebiasis. Parasitology international, 63 (5), 698-700.
Pompey et al. (2015). A single RNaseIII domain protein from Entamoeba histolytica has dsRNA cleavage activity and can help mediate RNAi gene silencing in a heterologous system. PloS one, 10 (7), e0133740.
Prucca et al. (2008). Antigenic variation in Giardia lamblia is regulated by RNA interference. Nature, 456 (7223), 750.
Ralston et al. (2011). The ways of a killer: how does Entamoeba histolytica elicit host cell death? Essays in biochemistry, 51, 193-210.
Redaèlli et al. (2013). Globalised world, globalised diseases: a case report on an amoebiasis-associated colon perforation. World Journal of Clinical Cases: WJCC, 1 (2), 79.
Samarawickrema et al. (1997). Involvement of superoxide dismutase and pyruvate: ferredoxin oxidoreductase in mechanisms of metronidazole resistance in Entamoeba histolytica. The Journal of antimicrobial chemotherapy, 40 (6), 833-840.
Santi-Rocca et al. (2008). The lysine- and glutamic acid-rich protein KERP1 plays a role in Entamoeba histolytica liver abscess pathogenesis. Cellular microbiology, 10 (1), 202-217.
Savioli et al. 2006. Giardia and Cryptosporidium join the ‘Neglected Diseases Initiative.’ Trends Parasitol. 22:203-208.
Simjee et al. (1985). A comparative trial tinidazole in the treatment amoebic liver abscess. SAMJ, 68, 923.
Solis et al. (2009). Use of bacterially expressed dsRNA to downregulate Entamoeba histolytica gene expression. PLoS One, 4 (12), e8424.
Stanley et al. (2003). Amoebiasis. The Lancet, 361 (9362), 1025-3.
Tiwari S et al. “Insecticide resistance in field populations of Asian citrus psyllid in Florida.” Pest Management Science (2011) 67.10:1258-1268
Ullu et al. (2004). RNA interference in protozoan parasites. Cellular microbiology, 6 (6), 509-519.
Vayssié et al. (2004). Double-stranded RNA mediates homologydependant gene silencing of y-tubulin in the human parasite Entamoeba histolytica. Molecular and biochemical parasitology, 138 (1), 21-28.
Whitt et al. (1988). Influence of indigenous microbiota on activities of alkaline phosphatase, phosphodiesterase I, and thymidine kinase in mouse enterocytes. Applied and environmental microbiology, 54 (10), 2405-2410.
Wilson et al. (2012). Host-Parasite interactions in Entamoeba histolytica and Entamoeba dispar: what have we learned from their genomes?, Parasite immunology, 34 (2-3), 90-99.
Wojcik D P et al. “Red imported fire ants: impact on biodiversity.” American Entomologist-Lanham (2001) 47.1:16-23.
Yoder et al., Centers for Disease Control and Prevention (CDC). MMWR Surveill Summ. 2010 Jun. 11; 59 (6): 15-25.
Zhang et al. (2011). Nucleus-localized antisense small RNAs with 5′polyphosphate termini regulate long term transcriptional gene silencing in Entamoeba histolytica G3 strain. Journal of Biological Chemistry, 286 (52), 44467-44479.
Zhang et al. “Cloning and Expression of Multiple Cytochrome P450 Genes: Induction by Fipronil in Workers of the Red Imported Fire Ant (Solenopsis invicta Buren).” PloS one 11.3 (2016): e0150915.
Ivashuta, et al., “Environmental RNAi in herbivorous insects,” RNA; 21 (5): 850-50 (2015).
Koch, et al., “An RNAi-Based Control of Fusarium graminearum Infections Through Spraying of Long dsRNAs Involves a Plant Passage and Is Controlled by the Fungal Silencing Machinery,” PLoS Pathogens; 12 (10): el005901 (2016).
Baum, et al., “Progress Towards RNAi-Mediated Insect Pest Management,” Advances in Insect Physiology; vol. 47: pp. 249-295 (2014).
Baum, J, “Rnai-mediated insect pest management: challenges and opportunities,” XXVth International Congress of Entomology 2016, Orlando, Florida, U.S.A.
Bolognesi, et al. “Characterizing the Mechanism of Action of Double-Stranded RNA Activity against Western Corn Rootworm (Diabrotica virgifera virgifera LeConte),” PLoS One; 7 (10) (2012).
Gong, et al., “Testing Insecticidal Activity of Novel Chemically Synthesized siRNA against Plutella xylostella under Laboratory and Field Conditions,” PLoS One; 8 (5): e62990 (2013).
Micura, “Small interfering RNAs and their chemical synthesis,” Angewandte Chemie International Ed.; 41 (13): 2265-9 (2002).
Ku, et al., “Chemical and structural modifications of RNAi therapeutics,” Advanced Drug Delivery Reviews; 104:16-28 (2015).
Timmons, “Construction of plasmids for RNA interference and in vitro transcription of double-stranded RNA,” Methods in Molecular Biology; 351:109-17 (2006).

### ADDITIONAL REFERENCES SUPPORTING SEQUENCES

Method for Modifying Insect Resistance of Plants by Utilizing Rnai Technique. Application No: 31293407 Filed: Dec. 4, 2007 Published: Feb. 25, 2010 Earliest Priority: Dec. 4, 2006 Granted: Nov. 25, 2014. Inventors: Chen et al.
Novel Insecticidal Toxin Receptors and Methods of Use. WO 2020/036747 A1 Patent Application Family: 4s/4ex Family Jurisdictions: CN, WO, BR, EP Legal Status: Pending Application No: 2019044771 Filed: Aug. 2, 2019 Published: Feb. 20, 2020 Earliest Priority: Aug. 13, 2018. Applicants: Pioneer Hi Bred Int. Inventors: Bazyk et al.
Compositions and Methods to Control Insect Pests. US US 2017/0253887 A1 Patent Application Family: 4s/4ex Family Jurisdictions: WO, CA, US, AR Legal Status: Discontinued Application No: 201515511196 Filed: Aug. 31, 2015 Published: Sep. 7, 2017 Earliest Priority: Sep. 17, 2014. Applicants: Pioneer Hi-Bred Int Inc, Du Pont. Inventors: Hu et al.
Composition and methods for control of insect infestation in plants. US 2012/0164205 A1 Patent Application. Legal Status: Active Application No: 201113226353 Filed: Sep. 6, 2011 Published: Jun. 28, 2012 Earliest Priority: Apr. 9, 2004 Granted: Jan. 19, 2016. Inventors: Baum et al.
Rnai approach for crop pest protection. WO2018204398A. Earliest Priority: May 1, 2017. Inventor: Bala P. Venkata.
RNA-based control of lepidopteran pests. Application No: 2021032334 Filed: May 13, 2021 Published: Nov. 18, 2021 Earliest Priority: May 13, 2020. Applicants: Greenlight Biosciences Inc. Inventors: Narva et al.
Cui, Gaofeng, et al. “Combined transcriptomic and proteomic analysis of harmine on Spodoptera frugiperda Sf9 cells to reveal the potential resistance mechanism.” Journal of proteomics 211 (2020): 103573.
Fuzita, Felipe Jun, et al. “N-glycosylation in Spodoptera frugiperda (Lepidoptera: Noctuidae) midgut membrane-bound glycoproteins.” Comparative Biochemistry and Physiology Part B: Biochemistry and Molecular Biology 246 (2020): 110464.
Tang, Bowen, et al. “Functional characterization of an a-esterase gene associated with malathion detoxification in Bradysia odoriphaga.” Journal of Agricultural and Food Chemistry 68.22 (2020): 6076-6083.
Webster, Gregory K., and Matthew A. Gragg. “Low level detection of nonionic surfactants of pharmaceutical interest.” Analytical Methods 11.26 (2019): 3314-3323.
Ren, Maozhi, et al. “Fighting against fall armyworm by using multiple genes pyramiding and silencing (MGPS) technology.” Sci China Life Sci 62.12 (2019): 1703-6.
Howard, John D., et al. “Chemically modified dsRNA induces RNAi effects in insects in vitro and in vivo: A potential new tool for improving RNA-based plant protection.” bioRxiv (2022).
Yang, Bing, et al. “Analysis of gene expression in the midgut of Bombyx mori during the larval molting stage.” BMC genomics 17.1 (2016): 1-16.
Gómez, Isabel, et al. “Spodoptera frugiperda (JE Smith) aminopeptidase N1 is a functional receptor of the Bacillus thuringiensis CrylCa toxin.” Applied and environmental microbiology 84.17 (2018): e01089-18.
Altstein, Miriam, and Dick R. Nässel. “Neuropeptide signaling in insects.” Neuropeptide systems as targets for parasite and pest control (2010): 155-165.
Park, Hyun Shin, et al. “Control of RNA with quinone methide reversible acylating reagents.” Organic & Biomolecular Chemistry 19.38 (2021): 8367-8376.
Yang, Zhouning, et al. “SfDredd, a novel initiator caspase possessing activity on effector caspase substrates in Spodoptera frugiperda.” PloS one 11.3 (2016): e0151016.
Hubert, Florence, et al. “Fast fatty acid analysis by core-shell reversed-phase liquid chromatography coupled to evaporative light-scattering detector.” Food and Nutrition Sciences 8.12 (2017): 1051-1062.
Li, Qilin, et al. “RNA-seq analyses of midgut and fat body tissues reveal the molecular mechanism underlying Spodoptera litura resistance to tomatine.” Frontiers in physiology 10 (2019): 8.
Christiaens, Olivier, et al. “Double-stranded RNA technology to control insect pests: Current status and challenges.” Frontiers in plant science 11 (2020): 451.
Giraudo, Maeva, et al. “Cytochrome P450s from the fall armyworm (Spodoptera frugiperda): responses to plant allelochemicals and pesticides.” Insect molecular biology 24.1 (2015): 115-128.
Heckel, David G. “The essential and enigmatic role of ABC transporters in Bt resistance of noctuids and other insect pests of agriculture.” Insects 12.5 (2021): 389.
Hilliou, Frédérique, et al. “Resistance in the genus Spodoptera: Key insect detoxification genes.” Insects 12.6 (2021): 544.
Jonusaite, Sima, Andrew Donini, and Scott P. Kelly. “Salinity alters snakeskin and mesh transcript abundance and permeability in midgut and Malpighian tubules of larval mosquito, Aedes aegypti.” Comparative Biochemistry and Physiology Part A: Molecular & Integrative Physiology 205 (2017): 58-67.
Lin, Junhan, et al. “Immune responses to Bacillus thuringiensis in the midgut of the diamondback moth, Plutella xylostella.” Developmental & Comparative Immunology 107 (2020): 103661.
Guo, Zhaojiang, et al. “MAPK signaling pathway alters expression of midgut ALP and ABCC genes and causes resistance to Bacillus thuringiensis CrylAc toxin in diamondback moth.” PLoS genetics 11.4 (2015): e1005124.
Chen, Xien, and Subba Reddy Palli. “Midgut-specific expression of CYP321A8 P450 gene increases deltamethrin tolerance in the fall armyworm Spodoptera frugiperda.” Journal of Pest Science (2022): 1-13.
Xu, Qiuyun, et al. “Transcriptional profiling of midgut immunity response and degeneration in the wandering silkworm, Bombyx mori.” (2012): e43769.
Parsons, Keith Hampton. Advancement of Interpolyelectrolyte Complexes for the Delivery of Genetic Material. Diss. The University of Southern Mississippi, 2018.
Wang, Rui-Long, et al. “A novel cytochrome P450 CYP6AB14 gene in Spodoptera litura (Lepidoptera: Noctuidae) and its potential role in plant allelochemical detoxification.” Journal of Insect Physiology 75 (2015): 54-62.
Zhu, Bin, et al. “Regulation of GSTu1-mediated insecticide resistance in Plutella xylostella by miRNA and lncRNA.” PLoS Genetics 17.10 (2021): e1009888.
Davis-Vogel, Courtney, et al. “Identification and comparison of key RNA interference machinery from western corn rootworm, fall armyworm, and southern green stink bug.” PLoS One 13.9 (2018): e0203160.
Qi, Weiping, et al. “Characterization and expression profiling of ATP-binding cassette transporter genes in the diamondback moth, Plutella xylostella (L.).” BMC genomics 17.1 (2016): 1-18.
Choi, Man-Yeon, and Robert K. Vander Meer. “Phenotypic effects of PBAN RNAi using oral delivery of dsRNA to corn earworm (Lepidoptera: Noctuidae) and tobacco budworm larvae.” Journal of economic entomology 112.1 (2019): 434-439.
Willow, Jonathan, et al. “RNAi targets in agricultural pest insects: advancements, knowledge gaps, and IPM.” Frontiers in Agronomy (2021): 99.
Xie, Miao, et al. “Molecular characterisation of two α-esterase genes involving chlorpyrifos detoxification in the diamondback moth, Plutella xylostella.” Pest management science 73.6 (2017): 1204-1212.
Zou, Xiaopeng, et al. “Glutathione S-transferase SIGSTE1 in Spodoptera litura may be associated with feeding adaptation of host plants.” Insect biochemistry and molecular biology 70 (2016): 32-43.

The teachings of all patents, published applications and references cited herein are incorporated by reference in their entirety.

What is claimed is:

1. A composition comprising a post-transcriptionally chemically modified double strand RNA (MdsRNA) wherein the MdsRNA comprises a double strand RNA wherein about 2% to about 30% of all the nucleotides independently comprise Formula (I): 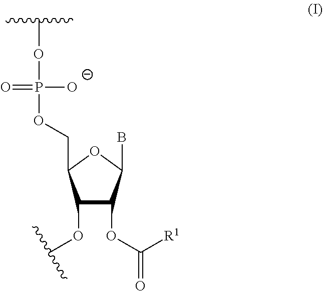
or an acceptable salt thereof, wherein:
B is a nucleobase;
R¹ is selected from: 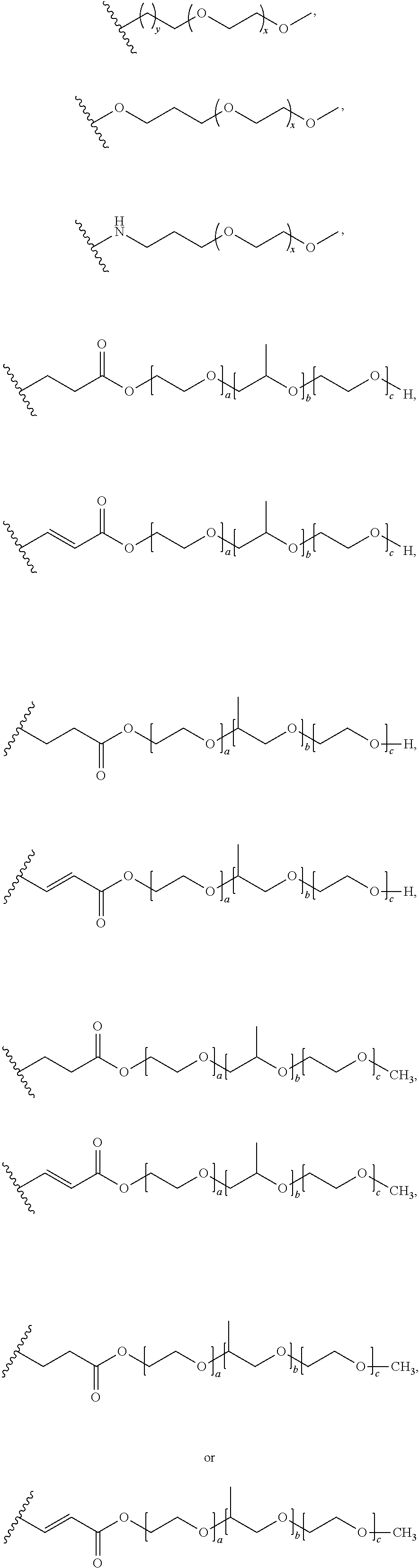
wherein y is an integer from 1-8, x is an integer from 12-1000, a is an integer from 12-1000, b is an integer from 12-1000, and c is an integer from 12-1000; and
optionally wherein from about 2% to about 85% of all the nucleotides independently comprise LMW nucleotides of Formula (III): 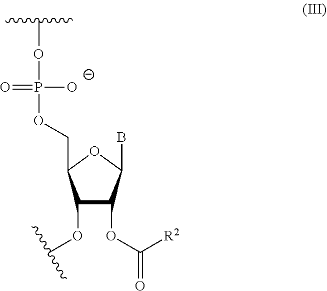
or an acceptable salt thereof, wherein:
B is a nucleobase;
R² is selected from C₁-C₂₅ alkyl, C₁-C₂₅ alkenyl, C₁-C₂₅ alkynyl, C₅-C₁₂ aryl or C₅-C₁₂ heteroaryl, wherein R² is optionally substituted with one or more substituents selected from halo, C₁₋₁₂ alkyl, C₁-C₁₂ aminoalkyl, or C₁-C₁₂ alkoxy.

2. The composition of claim 1, wherein the MdsRNA comprises a sequence complementary to a target region in Diamondback moth AChE2, P450, DOMELESS, DOUX, MESH, P450 CYP6BF1v1, Venom or VPASE; Western corn root worm SNF7; Fall armyworm P450, CYP9A58, Cytokine receptor DOMELESS, Dredd, VPASE and Protein MESH.

3. The composition of claim 1, wherein at least about 2% to about 60% of all the nucleotides independently comprise LMW nucleotides of Formula (III).

4. The composition of claim 1, wherein R² is N-methyl anthranoyl (NMA), N-benzyl anthranoyl (NBA), dimethyl furoyl, -Tyr, -Trp, -Leu, octanoyl, lauroyl, linoleyl, oleoyl, nicotinoyl or benzoyl.

5. A composition comprising a post-transcriptionally chemically modified ssRNA (MRNA) wherein the ssRNA comprises molecules of about 55 nucleotides to about 900 nucleotides, wherein the ssRNA comprises type (A), (B), or (C) RNA, wherein type (A) RNA comprises N or N+1 sense strand sections, Ax, 2N−1 or 2N, respectively, linker sections, Lz, and N antisense strand sections, Ay*, type (B) RNA and type (C) RNA comprises N sense strand sections, By or Cy, respectively, 2N−1 linker sections, Jz or Kz, respectively, and N antisense strand sections, By* or Cy*, respectively, wherein N is an integer from about 2 to about 51, x is an integer from 1 to N+1, y is an integer from 1 to N, and z is an integer from 1 to 2N, type (A) RNA is arranged as follows: 5′-A1-L1-A2-L2- . . . -AN-LN-A1*-LN+1 . . . L2N−1-AN*-3′ or 5′-A1-L1-A2-L2- . . . -AN-LN-A1*-LN+1- . . . -AN*-L2N-AN+1-3′, type (B) RNA is arranged as follows: 5′-B1-J1-B2-J2- . . . -BN-JN-BN*-JN+1- . . . -B2*-J2N−1-B1*-3′, type (C) RNA is arranged as follows: 5′-C1-K1-C1*-K2-C2-K3-C2*- . . . -CN-K2N−1-CN*-3′, and sense strands A1 through AN or A1 through AN+1, B1 through BN, and C1 through CN, and antisense strands A1* through AN*, B1* through BN*, and C1* through CN* have minimum lengths of 21 nucleotides and maximum lengths of 1000, or 500, or 250, or 125, or 61, or 27 nucleotides, wherein a section of at least one strand or antisense strand comprises a sequence complementary to a nucleic acid in a target host capable of reducing the concentration of one of its expressed RNAs when the composition is administered to the host or one of its cells, wherein from about 10% to about 100% of all the nucleotides in the linker strands L1 through L2N−1 or L1 through L2N, J1 through J2N−1, and K1 through K2N−1 independently comprise Formula (I): 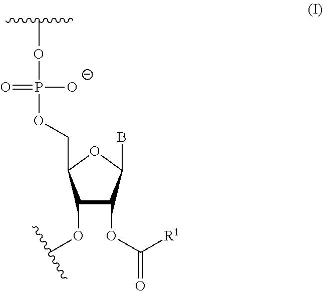
or an acceptable salt thereof, wherein:
B is a nucleobase;
R¹ is selected from: 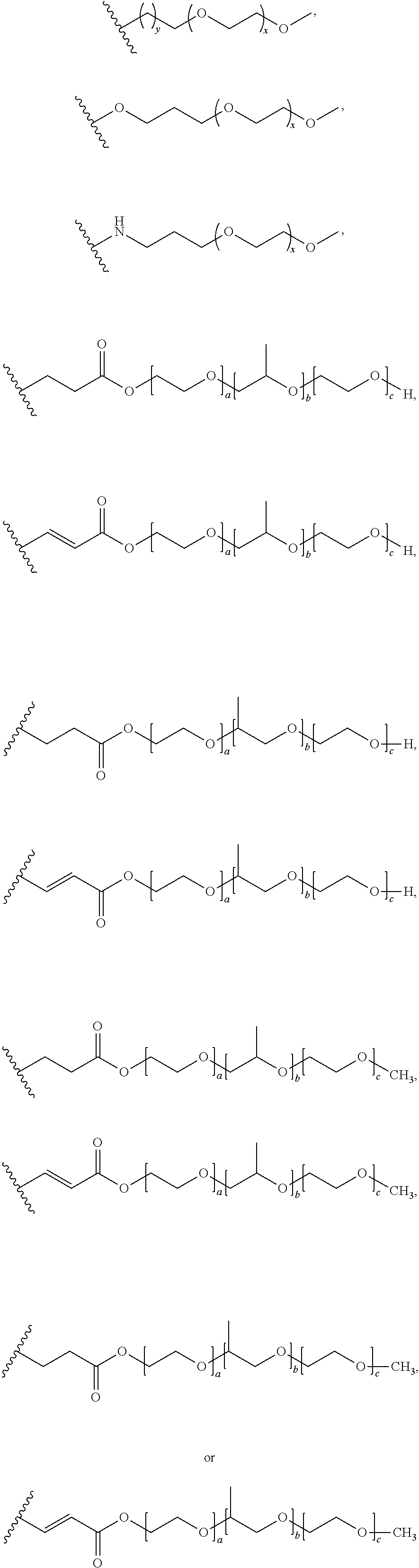
wherein y is an integer from 1-8, x is an integer from 12-1000, a is an integer from 12-1000, b is an integer from 12-1000, and c is an integer from 12-1000; and
optionally wherein at least 20% of all the nucleotides in the linker strands L1 through L2N−1 or L1 through L2N, J1 through J2N−1, and K1 through K2N−1 independently comprise LMW nucleotides of Formula (III): 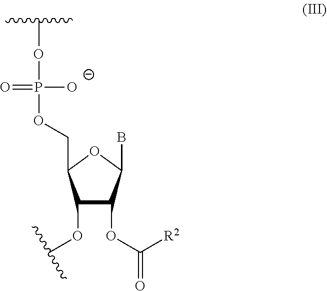
or an acceptable salt thereof, wherein:
B is a nucleobase;
R2 is selected from C1-C25 alkyl, C1-C25 alkenyl, C1-C25 alkynyl, C5-C12 aryl or C5-C12 heteroaryl, wherein R² is optionally substituted with one or more substituents selected from halo, C1-12 alkyl, C1-C12 aminoalkyl, or C1-C12 alkoxy.

6. A method of preparing a composition comprising a post-transcriptionally chemically modified double strand RNA (MdsRNA) of claim 1; and
the method comprising:
(a) contacting a compound of Formula (II): 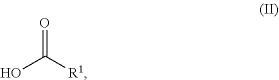
with an activation agent to form a compound of Formula (IIA): 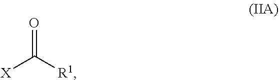
wherein X is a suitable leaving group;
(b) contacting a compound of Formula (IA): 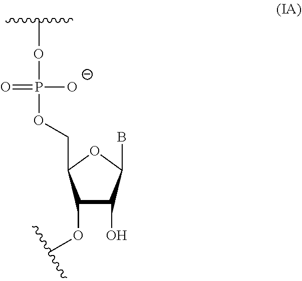
with a compound of Formula (IIA) to form a compound of Formula (I);
(c) optionally contacting a compound of Formula (II): 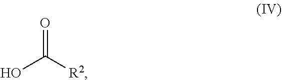
with an activation agent to form a compound of Formula (IVA): 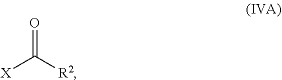
wherein X is a suitable leaving group;
(d) optionally contacting a compound of Formula (IA): 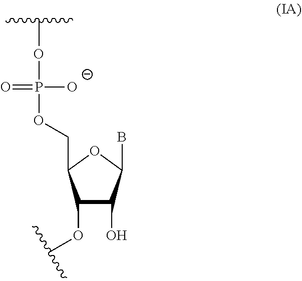
with a compound of Formula (IVA) to form a compound of Formula (III).

7. The method of claim 6, wherein the MdsRNA comprises a sequence complementary to a target region in Diamondback moth AChE2, P450, DOMELESS, DOUX, MESH, P450 CYP6BF1v1, Venom or VPASE; Western corn root worm SNF7; Fall armyworm P450, CYP9A58, Cytokine receptor DOMELESS, Dredd, VPASE and Protein MESH.

8. A method of preparing a composition comprising a post-transcriptionally chemically modified double strand RNA (MdsRNA) wherein the MdsRNA comprises a double strand RNA wherein at least about 2% of the nucleotides independently comprise Formula (VI): 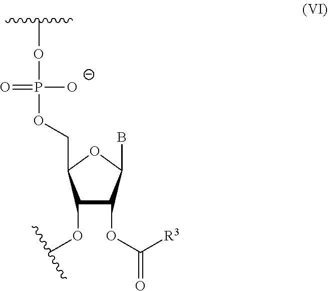
or an acceptable salt thereof, wherein:
B is a nucleobase;
R³ is selected from: amino acids, fatty acids, alkyl; substituted alkyl; alkenyl; substituted alkenyl; alkynyl; substituted alkynyl; aryl; substituted aryl; C1-C10 alkyl, C1-C10 alkenyl, or C1-C10 alkynyl wherein alkyl and alkenyl can be linear, branched or cyclic; hydrogen; methyl; ethyl; propyl; isopropyl; butyl; isobutyl; tert-butyl; pentyl; hexyl; cyclohexyl; heptyl; octyl; nonyl; decyl; vinyl; allyl; ethynyl; benzyl; cinnamyl; C6-C14 aryl; C6-C14 substituted aryl; heterocyclyl; C5-C14 heterocyclyl; phenyl; mono or disubstituted phenyl wherein the substituents are selected from C1-C10 alkyl, C1-C10 alkenyl, C1-C6 alkoxy, halogen, nitro, methylsulfonyl, and trifluoromethyl; 2-nitrophenyl; 4-nitrophenyl; 2;4-dinitrophenyl; 2-trifluoromethylphenyl; 4-trifluoromethylphenyl; styryl; C8-C16 substituted styryl; 2-aminophenyl; mono or disubstituted 2-aminophenyl wherein the substituents are selected from C1-C10 alkyl, C1-C10 alkenyl, C1-C6 alkoxy, halogen, nitro, methylsulfonyl, and trifluoromethyl; N-alkyl-2-aminophenyl or N-aryl-2-aminophenyl wherein alkyl has the formula —CₘH₂ₘ₊₁ (wherein m is an integer less than or equal to 12) and aryl is an aromatic moiety; 2-amino-3-methyl-phenyl; 2-amino-5-chlorophenyl; 2-methyl-5-chlorophenyl; N-methyla-2-minophenyl; N-ethyl-2-aminophenyl; N-propyl-2-aminophenyl; N-butyl2-aminophenyl; N-pentyl-2-aminophenyl; N-methyl-2-amino-4-nitrophenyl; 2-methyl-3-furyl; 2-methylnicotyl or N-trifluoromethyl-2-aminophenyl; silanyl; substituted silanyl; C1-C10 alkylsilanyl; C3-C12 trialkylsilanyl; C2-C12 alkoxyalkyl; C2-C12 alkoxyalkenyl; C2-C12 alkylthioalkyl; alkylsulfonyl; C1-C10 alkylsulfonyl; C1-C10 haloalkyl; C1-C10 haloalkenyl or C1-C10 aminoalkyl; —(CH₂CH₂O)ₚCH₃, —(CH₂CH₂O)ₚH, or —(CH₂CH₂O)ₚCOOR₄ wherein p is an integer from 2 to 8 and R⁴ is H, alkyl, substituted alkyl, aryl, or substituted aryl; —(CH₂CH₂O)₈COOH; —CH₂CH₂OH; —(CH₂CH₂O)₄OH; —(CH₂CH₂O)₆OH; —(CH₂CH₂O)₈OH; —(CH₂CH₂O)₈COOMe; —(CH₂CH₂O)₄OMe; —(CH₂CH₂O)₆OMe; —(CH₂CH₂O)₈OMe; —CH₂OCH₃; —CH₂OCH₂CH₃; or —CH₂OCH₂CH₂OCH₃; and
the method comprising:
(a) contacting a compound of Formula (IV): 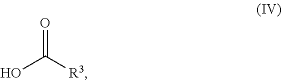
with an activation agent to form a compound of Formula (IVA): 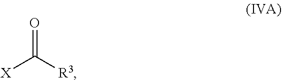
wherein X is a suitable leaving group; and
(b) contacting a compound of Formula (VIA): 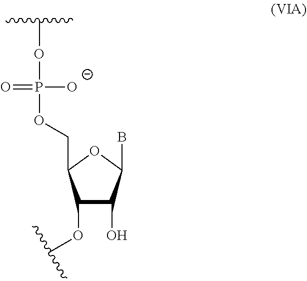
with a compound of Formula (IVA) to form a compound of Formula (VI).

9. The method of claim 8, wherein the MdsRNA comprises a sequence complementary to a target region in Diamondback moth AChE2, P450, DOMELESS, DOUX, MESH, P450 CYP6BF1v1, Venom or VPASE; Western corn root worm SNF7; Fall armyworm P450, Cytokine receptor DOMELESS, Dredd, VPASE and Protein MESH.

10. A method of modifying the expression of a polynucleotide of interest in any of the following: an insect, an acari, a fungus or a weed comprising administering a composition of claim 1.

11. The method of claim 10, wherein the expression is for a target region in Diamondback moth AChE2, P450, DOMELESS, DOUX, MESH, P450 CYP6BF1v1, Venom or VPASE; Western corn root worm SNF7; Fall armyworm P450, VPASE, Cytokine receptor DOMELESS, Dredd, and Protein MESH.

12. The method of claim 11, wherein the target region is in P450 CYP6BF1v1, in MESH transcript variant X1 or Venom carboylesterase-6.

13. The method of claim 11, wherein the expression is for a target region in Fall armyworm P450 CYP9A58, P450 CYP321A8, P450 CYP6B2-like, Cytokine receptor DOMELESS, Dredd, and Protein MESH transcript variant X1.

14. The method of claim 10, wherein the expression is for target region in Western corn root worm SNF7.

15. The method of claim 9, wherein the modified expression increase mortality or induce growth stunting, or stop instar development or reduces the fertility of the target insect.

16. The method of claim 9, wherein the modified expression reduces fertility of Lepidopteran insects.

17. The method of claim 9, wherein the expression increase mortality or induce growth stunting, or stop instar development or reduces the fertility of Coleopteran insects.

18. A method for producing MdsRNA deliverable to pests, comprising:
a) transcribing DNA into a ssRNA strand comprising at least one sense section followed by at least one section antisense to the sense section capable of forming at least one dsRNA stem;
b) adding a salt comprising a quaternary ammonium or a quaternary phosphonium cation or a pyrrolidinium or a pyridium or a piperidimium and a neutralizing anion to produce an isolate comprising more than half of the dsRNA produced in (a); and
c) conducting a chemical reaction between suitable reagents and at least 1% of the 2′OH groups in the RNA resulting from (b) in a blend comprising at least 1% of the salt used in (b), resulting in a post-transcriptionally chemically modified double strand RNA (MdsRNA).

19. The method of claim 18, wherein the ssRNA comprises (A) or (B) or both (A) and (B), wherein (A) comprises a sense section followed by a section antisense to the sense section and (B) comprises a sense section i, a sense section j, a sense section k, a section i* antisense to i, a section j* antisense to j, and a section k* antisense to k, wherein such sections are located in order 5′-i-j-k-i*-j*-k*-3′ and wherein, optionally, each section is between 20 and 60 nucleotides long.

20. The method of claim 18, wherein the quaternary ammonium cation is chosen from trimethyloctyl ammonium, trimethyldecyl ammonium, trimethyldodecyl ammonium, trimethyltetradecyl ammonium, trimethylhexadecyl, ammonium, trimethyloctadecyl ammonium, trimethylbenzyl ammonium, tributylbenzyl ammonium, choline, amyltriethylammonium, butyltrimethylammonium, benzylethyldimethylammonium, cyclohexyltrimethylammonium, diethyl(methyl)-propylammonium, diethyl(2-methoxyethyl)-methylammonium, ethyl(2-methoxyethyl)-dimethylammonium, ethyl(3-methoxypropyl)dimethylammonium, ethyl(dimethyl)(2-phenylethyl)-ammonium, methyltri-n-octylammonium, tetrabutylammonium, tetrahexylammonium, tetraamylammonium, tetra-n-octylammonium, tetraheptylammonium, tetraamylammonium, tetrapropylammonium, tributylmethylammonium, trimethylpropylammonium, tributyl(methyl)-ammonium, 1-allyl-3-methylimidazolium, 1-butyl-3-methylimidazolium, 1-butyl-2,3-dimethylimidazolium, 3,3′-(butane-1,4-diyl)-bis(1-vinyl-3-imidazolium), 1,2-dimethyl-3-propylimidazolium, 1-decyl-3-methylimidazolium, 1,3-dimethylimidazolium, 1-dodecyl-3-methylimidazolium, 1-ethyl-3-methylimidazolium, 1-ethyl-2,3-dimethylimidazolium, 1-hexyl-3-methylimidazolium, 1-(2-hydroxyethyl)-3-methylimidazolium, 1-methyl-3-noctylimidazolium, 1-methyl-3-pentylimidazolium, 1-benzyl-3-methylimidazolium, 4-ethyl-4-methylmorpholinium, tributylhexylphosphonium, tributylhexadecylphosphonium, tributylmethylphosphonium, tributyl-noctylphosphonium, tetrabutylphosphonium, tetra-n-octylphosphonium, tributyl(2-methoxyethyl)-phosphonium, tributylmethylphosphonium, trihexyl(tetradecyl)-phosphonium, trihexyl(tetradecyl)-phosphonium, tributyl(ethyl)phosphonium, tributyl(methyl)phosphonium, 1-allyl-1-methylpyrrolidinium, 1-butyl-1-methylpyrrolidinium, 1-ethyl-1-methylpyrrolidinium, 1-methyl-1-propylpyrrolidinium, 1-(2-methoxyethyl)-1-methylpyrrolidinium, 1-methyl-1-noctylpyrrolidinium, 1-methyl-1-pentylpyrrolidinium, tributylsulfonium, triethylsulfonium, and the anion is chosen from chloride, bromide, fluoride, iodide, acetate, propionate, butyrate, hexanoate, octanoate, decanoate, laurate, myristate, palmitate, palmitoleate, stearate, oleate, oxalate, succinate, bis(trifluoromethanesulfonyl)-imide, tetrafluoroborate, hexafluorophosphate, ptoluenesulfonate, trifluoromethanesulfonate, tetrachloroferrate, methanesulfonate, tribromide, hydrogen sulfate, thiocyanate, triflate, hexafluoroantimonate, dimethyl phosphate, methyl sulfate, dicyanamide, nitrate.


SEQUENCE LISTING (300 sequences)

SEQ ID NO: 1 - RNA, 300 nt, Plutella xylostella
  source 1..300: mol_type = mRNA
catatcggaggattgcctctatttgaacatatgggtgccgcagcacttgcgcgtccgtcaccatcaggacaagccattaaccgagcgaccgaaggttccaatactagtgtggatttacggcgggggttacatgagtggcacggcgacacttgatctatataaagccgacataatggcgtcttcgagtgatgtgatcgtagcctcgatgcagtatagggttggcgcgtttggatttttgtaccttaacaaatatttttcccctggtagcgaggaagcggcaggaaatatgggcttgtggga

SEQ ID NO: 2 - RNA, 190 nt, Plutella xylostella
  source 1..190: mol_type = mRNA
tcacaatgtcacattgtttggagaatcgtccggtgcagtttccgtgtcattacacttactgtctccgctgtcaagaaacatgttttctcaagctattatgcaatctgcagccgcatctgcaccttgggccatcatttccagagaggagagtgtgataaggggcatccgcctggccgaggccgtccactgc

SEQ ID NO: 3 - RNA, 300 nt, Plutella xylostella
  source 1..300: mol_type = mRNA
gctgaggtcggtggaatagacggaaatgcagatgatgattacaccttgaccgaggaggaggtgatcttgcagaactccgccagcgagtccccggaggccgagcaggcgctgcaacaagcggctttgcttctgcgcctgcgcgacggcatgggctcgctcgcgcgcatcctcaagaccatcgacaactacaagggatgcgttcaacacctcgagactcgcccctccaacgccaacgacatccaattcgatgctctcatcaaagtgagcatgtcccgtggcaacctgctccaactcatccga

SEQ ID NO: 4 - RNA, 300 nt, Plutella xylostella
  source 1..300: mol_type = mRNA
tgcaggtcaagccgcagagggtcaagctgcagctgcgcatgaaccagatgcagaaactagacgtcgcctattcccaagcccaagactacccggtggacctgtactacttgatggacctgagtcgttccatgaagaacgacaaggagaagctcagtacattgggcagtctgctgtccagcactatgaggaatatcacgcccaacttccgtcttggcttcggctccttcgtggacaagctcgtcatgccctacgtgtctactgtgcctaagaatttgatatccccttgtgatggctgcgcgg

SEQ ID NO: 5 - RNA, 300 nt, Plutella xylostella
  source 1..300: mol_type = mRNA
ccaggaaacatggcactaagaacaaaagagcggccatcgctgcacttaaacgcaagaagcgttacgagaagcaactcacacagattgacggcacgctcagccagatagagatgcagagagaggcattggagggcgccaacactaacactcaagtactgaacacgatgcgagaggccgccgcggctatgaagctcgctcacaaggatattgacgtagacaaagtgcatgatatcatggacgacatcgctgaacaacatgatgtggctcgcgaaatcacggatgccatcagcaacaatgtgg

SEQ ID NO: 6 - RNA, 239 nt, Plutella xylostella
  source 1..239: mol_type = mRNA
tcagtgttttgttgttgtgctcatgcagggtgtggcgtggtgaccagacagcagtgggatgggctggacccgatacagttggagtacctgccccggcccctggggctggtggtggtccagcacaccgccacccccgcgtgtgacactgacgccgcgtgtgtggagctggtgcagaaatacagaccaatcatatggatgtgctgaagttttgggatattggaccgaacttcctgattggt

SEQ ID NO: 7 - RNA, 300 nt, Plutella xylostella
  source 1..300: mol_type = mRNA
gcgctcagcgatgcagatgtccaaaaacagatcaagcatatgatggccttcatcgagcaagaggcaaatgaaaaggccgaagaaatcgatgctaaggctgaggaggagttcaacatcgagaaggggcgtctggtgcagcagcagcgcctcaagatcatggagtactacgagaagaaggagaagcaggtggaactccagaagaagatccaatcctccaacatgctgaaccaggcccgtctgaaggtgctgaaggtgcgcgaggaccacgtgggccacgtgttggacgagacgcgccgccgc

SEQ ID NO: 8 - RNA, 1434 nt, Diabrotica virgifera
  source 1..1434: mol_type = mRNA
cacaactgacagataacgtcagtagttgtctattttcactggtgactaatttttgagaattagtaattggtttcgtatttttttcttaacaaaagggcaaaatgagcttttttggaaaattgttcggggggaaaaaggaagagatagcccctagtcctggggaggctattcaaaaactcagagagactgaagaaatgttaataaaaaaacaggattttttagaaaagaagatagaagaatttaccatggtagcaaagaaaaatgcgtcgaaaaataaaagagttgcactccaagccctcaaaaagaagaaacgattggaaaagacccaactacaaatagatggaacccttacaactattgaaatgcagagggaagccctcgaaggagctagcacaaatactgctgtattagattctatgaaaaatgctgcagatgcccttaagaaagctcataagaatttgaatgtagatgatgttcacgatatcatggatgacatagccgaacaacacgacatagccaacgaaatcacaaacgctattagcaatcctgtcggattcaccgacgatctggatgacgatgaattagaaaaagaattagaagagctcgaacaagaaggattggaagaagacctgctccaagtgccaggtccaactcaactgccggctgtgcctgctgatgcagttgctactaaaccaatcaaaccagcagctaaaaaagttgaagatgatgacgatatgaaagaattggaagcctgggcctcgtaaaattcctgaaatctgaatatttgtaaacgaaatcacccatctaagatgaaaacattataaatatataggtaataacagctaaaaacgtttcaatgtagaacaagcttttgctgaaaaggcgtcttgcaaaaaatgttgataatttagaattctctatatattatatatttgccctttaaggaaagatttcttttatagtcatagttcaaccaaacgtttcataaattagaatacaggggtgtcagaattctacgtgaatttattgaaaaaaaagaactttccttaaagttggcataatgatatttttgaaattatgtaaataggtatatgtatgctcttactaacggttttaatattgggtttagaagcaccttaattttatttttatacaaggtagtaacattttttcattgtaatttgttaaaaaatattgtataacgcaaaaaatggtgtgataaagcaaaatattattgagtgcttattttcttattttattaaaagatcttatttacgttaaatctaaatatattgggtatctcagaatatgttaacaaaggtttcttgtatcaacagaaaaatacaaacttattcattattgtggttcatttgattatgcttgtgtatatttaatctgccataaacaagttttgataaatgtcactgctctgta

SEQ ID NO: 9 - RNA, 185 nt, Acyrthosiphon pisum
  source 1..185: mol_type = mRNA
ttagccaacactggaataaacgtcaaaataaacattgataaaagtattaaattaccgactcaagaaataggaggcgtcgtggtcacgtccaaagatcgaagggtacatgttgaaaatacgcttgtagtgagattgctctatctcacccaacaagcaataccaataatatgcactggactgtttgg

SEQ ID NO: 10 - RNA, 272 nt, Solenopsis invicta
  source 1..272: mol_type = mRNA
gatctctctccctcgactctaacaccagcgaaagtaacagccaatcaagatgtgtgacgatgatgttgcggcattagtcgtggacaatgggtccggtatgtgcaaggctggattcgcgggggatgatgcaccacgcgctgtgtttcccagcatcgtcggtcgtcctcgtcatcagggtgtgatggtcggtatgggtcaaaaagacagttatgttggcgacgaggcgcaaagtaagagaggtatattgacactaaagtatcctatagaacatg

SEQ ID NO: 11 - RNA, 220 nt, Gibberella zeae
  source 1..220: mol_type = mRNA
cagcaagtttgacgagtccctggccgctctctaccacgacctcgatatgggcttcacccccatcaacttcatgcttcactgggcccctctcccctggaaccgtaagcgcgaccacgcccagcgcactgttgccaagatctacatggacactatcaaggagcgccgcgccaagggcaacaacgaatccgagcatgacatgatgaagcaccttatgaactct

SEQ ID NO: 12 - RNA, 238 nt, Fusarium graminearum
  source 1..238: mol_type = mRNA
attggaagcaccgtacaatatggcatcgacccgtacgcttttttcttcgactgcagagataaatacggcgactgctttacctttattctccttggcaaatcaacgactgtctttcttggtcccaagggcaatgactttatcctcaacggcaaacacgccgatctcaacgccgaggacgtttatgggaaacttaccacgcccgtgtttggtgaggaggttgtttatgactgctccaatg

SEQ ID NO: 13 - RNA, 100 nt, Plutella xylostella
  source 1..100: mol_type = mRNA
ggcagcaaccatggcgctcagcgatgcagatgtccaaaaacagatcaagcatatgatggccttcatcgagcaagaggcaaatgaaaaggccgaagaaatc

SEQ ID NO: 14 - RNA, 100 nt, Plutella xylostella
  source 1..100: mol_type = mRNA
gatgctaaggctgaggaggagttcaacatcgagaaggggcgtctggtgcagcagcagcgcctcaagatcatggagtactacgagaagaaggagaagcagg

SEQ ID NO: 15 - RNA, 100 nt, Plutella xylostella
  source 1..100: mol_type = mRNA
tggaactccagaagaagatccaatcctccaacatgctgaaccaggcccgtctgaaggtgctgaaggtgcgcgaggaccacgtgggccacgtgttggacga

SEQ ID NO: 16 - RNA, 100 nt, Plutella xylostella
  source 1..100: mol_type = mRNA
gacgcgccgccgcctcgccgaggtgcccaacgaccaggggctctactccgacctggtggtcaagctcatcgtgcaggcgctgttccagctggttgagcca

SEQ ID NO: 17 - RNA, 100 nt, Plutella xylostella
  source 1..100: mol_type = mRNA
accgtaaccctccgcgtgcgcgaggccgacaagccgctgatcgacagcctgctcgagcgcgcgcaggcgcagtacaaggagaagatcaagaaggatgtga

SEQ ID NO: 18 - RNA, 100 nt, Plutella xylostella
  source 1..100: mol_type = mRNA
ccttgaaggtggacacggagcactacctgccggtgggcacctgcggcgggattgagttggtcgccgctaggggccgcatcaagatcatcaacaccctgga

SEQ ID NO: 19 - RNA, 100 nt, Plutella xylostella
  source 1..100: mol_type = mRNA
gtcgcgcatggagctgatcgcgcagcagctgctgcccgagatccgcacggcgctgttcggacggaaccccaaccgcaagttcaccgactaaacaccaacc

SEQ ID NO: 20 - RNA, 100 nt, Plutella xylostella
  source 1..100: mol_type = mRNA
agatttgactgggcgcagggcggtgtcacccatacatctttcagctgaaaaagacattcctctttcatgtttcctgtcctggcaatcaaatgtttcggct

SEQ ID NO: 21 - RNA, 100 nt, Plutella xylostella
  source 1..100: mol_type = mRNA
tgcttttaacagttctatcgaagagcaccgtagctctataaattacataacgaatataatgtttaatccccattatggcactgttaaaattcttatgtaa

SEQ ID NO: 22 - RNA, 100 nt, Plutella xylostella
  source 1..100: mol_type = mRNA
atactgtttactatcgtggacttcctgggaattatttgatgctgtaaggtttatgggtgacggcagcatcccgccttattcccactgaaatcgaagtgaa

SEQ ID NO: 23 - RNA, 116 nt, Plutella xylostella
  source 1..116: mol_type = mRNA
ggcagcaaccatggcgctcagcgatgcagatgtccaaaaacagatcaagcatatgatggccttcatcgagcaagaggcaaatgaaaaggccgaagaaatcgatgctaaggctgagg

SEQ ID NO: 24 - RNA, 174 nt, Plutella xylostella
  source 1..174: mol_type = mRNA
ttattcccactgaaatcgaagtgaaagatttgactgggcgcagggcggtgtcacccatacatctttcagctgaaaaagacattcctctttcatgtttcctgtcctggcaatcaaatgtttcggcttgcttttaacagttctatcgaagagcaccgtagctctataaattacata

SEQ ID NO: 25 - RNA, 290 nt, Plutella xylostella
  source 1..290: mol_type = mRNA
ggcagcaaccatggcgctcagcgatgcagatgtccaaaaacagatcaagcatatgatggccttcatcgagcaagaggcaaatgaaaaggccgaagaaatcgatgctaaggctgaggttattcccactgaaatcgaagtgaaagatttgactgggcgcagggcggtgtcacccatacatctttcagctgaaaaagacattcctctttcatgtttcctgtcctggcaatcaaatgtttcggcttgcttttaacagttctatcgaagagcaccgtagctctataaattacata

SEQ ID NO: 26 - RNA, 150 nt, Plutella xylostella
  source 1..150: mol_type = mRNA
gtgataagttgtgattctccgccattctcggtctgtcttttccatctttctcgtccaaaaaacaaggtaattgttcaagatgagtcacgggttgaagaggattgccgatgaggacaatgaaacccagttcggttatgtcttcgctgtgtc

SEQ ID NO: 27 - RNA, 150 nt, Plutella xylostella
  source 1..150: mol_type = mRNA
ggcagcaaccatggcgctcagcgatgcagatgtccaaaaacagatcaagcatatgatggccttcatcgagcaagaggcaaatgaaaaggccgaagaaatcgatgctaaggctgaggaggagttcaacatcgagaaggggcgtctggtgca

SEQ ID NO: 28 - RNA, 300 nt, Plutella xylostella
  source 1..300: mol_type = mRNA
gtgataagttgtgattctccgccattctcggtctgtcttttccatctttctcgtccaaaaaacaaggtaattgttcaagatgagtcacgggttgaagaggattgccgatgaggacaatgaaacccagttcggttatgtcttcgctgtgtcggcagcaaccatggcgctcagcgatgcagatgtccaaaaacagatcaagcatatgatggccttcatcgagcaagaggcaaatgaaaaggccgaagaaatcgatgctaaggctgaggaggagttcaacatcgagaaggggcgtctggtgca

SEQ ID NO: 29 - RNA, 100 nt, Plutella xylostella
  source 1..100: mol_type = mRNA
acggagggagatcctccttctgtgactatagcgcaagggtccgttgtaggtactgctgtgaccagctctggatttacacactatgaattccacgggatac

SEQ ID NO: 30 - RNA, 100 nt, Plutella xylostella
  source 1..100: mol_type = mRNA
cgtatgctgactctacttctggatctcacaggttcaaggcgccacgaccagcacccacattcacacagacttttgttgctgatcgcaaaggaatcaaatg

SEQ ID NO: 31 - RNA, 100 nt, Plutella xylostella
  source 1..100: mol_type = mRNA
tgtgaaagctataaaggggggatacgagggcaccgaagattgcttggtggccaacgtctacacaccggccattgatccagaaaagaaatacccagtaatg

SEQ ID NO: 32 - RNA, 100 nt, Plutella xylostella
  source 1..100: mol_type = mRNA
gtttggattaaagggtccgagtttgagaaaactaagggacctgaactatcttttagaaatcttattgaaaaagaagtaatagtcgtgtctctaaacttca

SEQ ID NO: 33 - RNA, 100 nt, Plutella xylostella
  source 1..100: mol_type = mRNA
gagagtcgattctcgggtatctttgtcttggaacagaaactgcgcctggtaacgctggattgaaagatataattgctggacttcaatgggtgaaagataa

SEQ ID NO: 34 - RNA, 100 nt, Plutella xylostella
  source 1..100: mol_type = mRNA
cattgaacagtttggtggagaccctgagagtataaccctatttgggcatggttctggagctgcggcggtagatttagtcacactctctccaatgtctaag

SEQ ID NO: 35 - RNA, 100 nt, Plutella xylostella
  source 1..100: mol_type = mRNA
ttgggcatgaaattactgaggaattagatattgaaaagctttcggaagtttttactagaacaagtgttgccgctctaatggcagttatagatgagttgga

SEQ ID NO: 36 - RNA, 100 nt, Plutella xylostella
  source 1..100: mol_type = mRNA
tttaactgataactcattggcttttgctccgtgtatcgaaaatgagcatttagatgatgaaaagtttctagaaaaatcaccttttagtacgctaactgaa

SEQ ID NO: 37 - RNA, 100 nt, Plutella xylostella
  source 1..100: mol_type = mRNA
ggaacttacactaaaatacctatgatcttcggatttgttgaaaacgaaggaacaatacgttttgatgaggcactagaagctgattggctaacaaagatgg

SEQ ID NO: 38 - RNA, 150 nt, Plutella xylostella
  source 1..150: mol_type = mRNA
ccaacgtctacacaccggccattgatccagaaaagaaatacccagtaatggtttggattaaagggtccgagtttgagaaaactaagggacctgaactatcttttagaaatcttattgaaaaagaagtaatagtcgtgtctctaaacttca

SEQ ID NO: 39 - RNA, 150 nt, Plutella xylostella
  source 1..150: mol_type = mRNA
tagatgatgaaaagtttctagaaaaatcaccttttagtacgctaactgaaggaacttacactaaaatacctatgatcttcggatttgttgaaaacgaaggaacaatacgttttgatgaggcactagaagctgattggctaacaaagatgg

SEQ ID NO: 40 - RNA, 300 nt, Plutella xylostella
  source 1..300: mol_type = mRNA
ccaacgtctacacaccggccattgatccagaaaagaaatacccagtaatggtttggattaaagggtccgagtttgagaaaactaagggacctgaactatcttttagaaatcttattgaaaaagaagtaatagtcgtgtctctaaacttcatagatgatgaaaagtttctagaaaaatcaccttttagtacgctaactgaaggaacttacactaaaatacctatgatcttcggatttgttgaaaacgaaggaacaatacgttttgatgaggcactagaagctgattggctaacaaagatgg

SEQ ID NO: 41 - RNA, 150 nt, Plutella xylostella
  source 1..150: mol_type = mRNA
aatataaagtgcgtgattttacacatgtcgaatgtcatgagtgaaaggatctttgtaggtttctttacattgaagtgaattctcgtgctttgtcttcgtgtgtgataatactcaaaatggcgcatcaactggctccgtctgtgaattgct

SEQ ID NO: 42 - RNA, 150 nt, Plutella xylostella
  source 1..150: mol_type = mRNA
atggccgagagtcagccgcccctgtgtgaccttcacccaatgagagcattgtttcttataccaagaaatcctccaccccgtttaaagtcaaagaaatgggcaaagaaatttcatagttttattgaaactgttcttgtgaaagactatcac

SEQ ID NO: 43 - RNA, 100 nt, Plutella xylostella
  source 1..100: mol_type = mRNA
ttgcttcatgcagaagggcgacgacaagaccgtcttccagatcccggacaacttctacccagaaaagtacaagaaggtgggcaaccagctggccgaccgg

SEQ ID NO: 44 - RNA, 100 nt, Plutella xylostella
  source 1..100: mol_type = mRNA
ggaatgcgtgacgtggaggacctgcagtccgtgtgtagctactgccagctccgcatcaacccctacatgttcaactactgcctgtcggtcgccatgctgc

SEQ ID NO: 45 - RNA, 100 nt, Plutella xylostella
  source 1..100: mol_type = mRNA
acagaccagacacgaagggcctgtcgccgccgacgctggcggagacgttccccgacaagttcatggaccccaaggtgttccgccgcgcgcgggagacctc

SEQ ID NO: 46 - RNA, 100 nt, Plutella xylostella
  source 1..100: mol_type = mRNA
caccaccgcgcctgctggggacaggatgccagtcctaatcccggtcaactacacggcctccgacgctgagccagaacaacgcatcgcgtacttccgcgaa

SEQ ID NO: 47 - RNA, 102 nt, Plutella xylostella
  source 1..102: mol_type = mRNA
gacatcggcatcaacctgcaccactggcactggcacctggtgtaccccttcgaggcggccgaccgcgccgtggtggacaaggacaggcgcggcgagctgcgc

SEQ ID NO: 48 - RNA, 100 nt, Plutella xylostella
  source 1..100: mol_type = mRNA
tgtactacatgcaccagcagatcatcgccagatacaacgcagagcgtctgtgcaacaacctgggcttcgtgacgcgctacaacgacttccgcgggcccat

SEQ ID NO: 49 - RNA, 100 nt, Plutella xylostella
  source 1..100: mol_type = mRNA
cgccgaggggtacttccccaagatggactcgcaggtcgccagcagggcctggcctcctaggttctccggcaccacgatccgcgacctggaccgccccgtg

SEQ ID NO: 50 - RNA, 100 nt, Plutella xylostella
  source 1..100: mol_type = mRNA
gaccagatccgctccgacgtgtctgagatggagacctggagggaccgcttcatccaggccatcgacagcggcactattgttctgcccaacggccgcaccc

SEQ ID NO: 51 - RNA, 100 nt, Plutella xylostella
  source 1..100: mol_type = mRNA
agcgcctcgacgaggagaccggcatcgacgtgctggccaacctcatggagtcgtccatcatcagccgcaaccgcgcctactacggggacctgcacaacat

SEQ ID NO: 52 - RNA, 100 nt, Plutella xylostella
  source 1..100: mol_type = mRNA
ggggcatgtgttcatctcctatgcgcacgaccccgaccaccggcacttggaacaattcggcgtgatgggagacccggccacggccatgagggacccgatc

SEQ ID NO: 53 - RNA, 100 nt, Plutella xylostella
  source 1..100: mol_type = mRNA
ttctaccgctggcacgcgtacgtcgacgacatcttccagagatacaaggccacactaccagcctacaccagggagaggttggacttcccaggcatccgcg

SEQ ID NO: 54 - RNA, 100 nt, Plutella xylostella
  source 1..100: mol_type = mRNA
tctcctccatcgccatctcgggccgcactccgaaccagttctcgacgcagtgggagcagagttcagtgaacctggcgcgcgggctggacttcatgccgcg

SEQ ID NO: 55 - RNA, 100 nt, Plutella xylostella
  source 1..100: mol_type = mRNA
cggcgccgtgctggcgcggttcacgcatctgcagcatgacgagtttgagtacaccatcgagtgcgacaacacaaccggccaagcagccatgggcaccgtc

SEQ ID NO: 56 - RNA, 100 nt, Plutella xylostella
  source 1..100: mol_type = mRNA
cgcatattcctcgccccgaccaccgaccaggccggcaacgcactcaacttcgaggagcagaggcgactcatgatcgagctggacaagtttactcagggct

SEQ ID NO: 57 - RNA, 100 nt, Plutella xylostella
  source 1..100: mol_type = mRNA
tgcgccccggcagcaacaccatccggcgtcgcagcatcgactcctcagttaccatcccctacgagcgaacattccgggacgagtcccaacaccccggaga

SEQ ID NO: 58 - RNA, 99 nt, Plutella xylostella
  source 1..99: mol_type = mRNA
cgctggctcagctcagtcagccgacttcgacttctgcggctgcggctggccgcaccacatgctgataccgaaggggactcagcagggatggaactgtgt

SEQ ID NO: 59 - RNA, 100 nt, Plutella xylostella
  source 1..100: mol_type = mRNA
ctcttctgcatgattaccaactggaatgaagatcgggtggagcaagacacagtaggaacctgcaacgacgcagcctcctactgcggtatccgggaccgcc

SEQ ID NO: 60 - RNA, 100 nt, Plutella xylostella
  source 1..100: mol_type = mRNA
gctacccggaccgcaagcctatgggattccccttcgatagaccagcgccatctaccggcagtttgggagacttcttgacccccaacatgactgtgcagaa

SEQ ID NO: 61 - RNA, 100 nt, Plutella xylostella
  source 1..100: mol_type = mRNA
ttacagtgttcgtggtattttaagcccaaacgctaatccaagatggcggacaaaaacaacctgctgctgttcttcgaccgccccacggagccctgcttca

SEQ ID NO: 62 - RNA, 200 nt, Plutella xylostella
  source 1..200: mol_type = mRNA
tcaggtttactgatgcggtcaggcagcgtcagcagcggtagagtgtgggagggagacatggttcatacgttacgagagttctagctaaacttaaacacacaatgaaagctgactgtaacttttgtagattttgccttcaaaatgacaattacttaggcactaattaggttctaattttgtataattcatttttagcgaca

SEQ ID NO: 63 - RNA, 300 nt, Plutella xylostella
  source 1..300: mol_type = mRNA
ttacagtgttcgtggtattttaagcccaaacgctaatccaagatggcggacaaaaacaacctgctgctgttcttcgaccgccccacggagccctgcttcatcaggtttactgatgcggtcaggcagcgtcagcagcggtagagtgtgggagggagacatggttcatacgttacgagagttctagctaaacttaaacacacaatgaaagctgactgtaacttttgtagattttgccttcaaaatgacaattacttaggcactaattaggttctaattttgtataattcatttttagcgaca

SEQ ID NO: 64 - RNA, 100 nt, Plutella xylostella
  source 1..100: mol_type = mRNA
cgacccacggctccaagtccgtgctgacccccaacctcgacgtgctgacccgctcaggagtgtccctccaccgctactacacccacgctctctgctcgcc

SEQ ID NO: 65 - RNA, 100 nt, Plutella xylostella
  source 1..100: mol_type = mRNA
cgcccgtaccgctgtgctcaccggcaaatacgcccacaccgtcggtatgcagggtatgcctctgtccaacgctgaggagcgtggtatccccctagaggag

SEQ ID NO: 66 - RNA, 100 nt, Plutella xylostella
  source 1..100: mol_type = mRNA
cgcctgatctctcagtacctacaggacgctggttacaggacccagatggtcggaaagtggcacgtcggtcacgccttcttcgagcagctgcccacttaca

SEQ ID NO: 67 - RNA, 100 nt, Plutella xylostella
  source 1..100: mol_type = mRNA
gaggattcgagaaccacttcggtgtccgcggtggattcatcgactactacgaatacaacgctcaggagcagcttgacggcaggccagtcactggactgtg

SEQ ID NO: 68 - RNA, 100 nt, Plutella xylostella
  source 1..100: mol_type = mRNA
tctgttcgacgacctgcagcccgactggaccaccgagggatacatcaccgacgtctacaccgagaagtccaccaccatcattgagaaccacaacgtctcc

SEQ ID NO: 69 - RNA, 100 nt, Plutella xylostella
  source 1..100: mol_type = mRNA
gagcccctgtacctgctgctgacccaccacgctccccacaacggcaacgaagacgcttccctgcaggcccctcctgaagaggtccgcgcccagaggcacg

SEQ ID NO: 70 - RNA, 100 nt, Plutella xylostella
  source 1..100: mol_type = mRNA
tcgagctccaccccagacgtatcttcgccgctatggttaagaaactggacgacagtatcggagaaatcgtcgctaccctcgagaagaagggcatgctcga

SEQ ID NO: 71 - RNA, 100 nt, Plutella xylostella
  source 1..100: mol_type = mRNA
gaacaccatcatcaccttctccactgacaacggtgcccccaccgtcggtcttggcgccaactctggttccaactaccccctgagaggagtcaagaagtcc

SEQ ID NO: 72 - RNA, 100 nt, Plutella xylostella
  source 1..100: mol_type = mRNA
ccctgggagggaggtatccgtggtaacgccatgatctgggccggtcccgaggtcgcccccggaaacgcgtggcgtggaaaggtttacgacggcaacatgc

SEQ ID NO: 73 - RNA, 100 nt, Plutella xylostella
  source 1..100: mol_type = mRNA
acgccgctgactgggtccccactctgcttgaggccatcggtgagaagatccccgccggtctggacggtatccccatgtggagccacatcatcgagaacaa

SEQ ID NO: 74 - RNA, 100 nt, Plutella xylostella
  source 1..100: mol_type = mRNA
gccttctccccgtaccgagatcttcgagatcgacgactacttcaaccactcctctgtcaccctcggccgccacaagctcgtcaagggaaccatcgacgag

SEQ ID NO: 75 - RNA, 100 nt, Plutella xylostella
  source 1..100: mol_type = mRNA
tctctcagcaagcactacggtgaagacctccgtggcatcatcggaactcccccagactacaagcagaagctgcgcgacagcaaggcatgggagtctctgg

SEQ ID NO: 76 - RNA, 100 nt, Plutella xylostella
  source 1..100: mol_type = mRNA
agaccatcggcatccctctggacgctgacgtcatggctgaccgcgatgaggctatcgtcacttgcggaaatgtcgtccccaagccttgcagtccttctgc

SEQ ID NO: 77 - RNA, 100 nt, Plutella xylostella
  source 1..100: mol_type = mRNA
cgagtcttggtgcctgtacgacatcatcgaggacccttgtgagcttcgtgacctgtctgaggagcttcctcagctggctcagatccttctgtaccgtctg

SEQ ID NO: 78 - RNA, 100 nt, Plutella xylostella
  source 1..100: mol_type = mRNA
gagcaggaagaggccaagatcatccccagggagggccagtacgtcgctgaccccaagtctgcccccaagtacttcaactacacctgggacgcgtacctgt

SEQ ID NO: 79 - RNA, 100 nt, Plutella xylostella
  source 1..100: mol_type = mRNA
ccgtcgaaccctactccgactccgaatagacgaagctcagctcaagcggcgcagttcgccgtgaaagttgtaaatgttgatcctggccttaatttcagta

SEQ ID NO: 80 - RNA, 300 nt, Plutella xylostella
  source 1..300: mol_type = mRNA
gaggattcgagaaccacttcggtgtccgcggtggattcatcgactactacgaatacaacgctcaggagcagcttgacggcaggccagtcactggactgtgtcgagctccaccccagacgtatcttcgccgctatggttaagaaactggacgacagtatcggagaaatcgtcgctaccctcgagaagaagggcatgctcgaccgtcgaaccctactccgactccgaatagacgaagctcagctcaagcggcgcagttcgccgtgaaagttgtaaatgttgatcctggccttaatttcagta

SEQ ID NO: 81 - RNA, 301 nt, Plutella xylostella
  source 1..301: mol_type = mRNA
atccagcgggacgcccgcgaccgcgcctcaatgtggttcgggccgcgcctcgggaagcgagccatgcacctcgcgcccgacggtgacggacaagcagtatacaggatgctcgaagctgcagacgcgctcaagtactactacgaccagctccagtattatggggctcaggcagacgatcctgagactaaagtgacaaagaaggtgatcttcacccccaagttgggccgcaacgctgatgaagaccagcagcagtcggtggacttcacgccgagactagggcgccgccggctcaaggactcgg

SEQ ID NO: 82 - RNA, 100 nt, Plutella xylostella
  source 1..100: mol_type = mRNA
acaaccatgagttttctggggaaaatattcggtggtaagaaggaggagaaaggtccatcaacacacgaagctattcaaaaattacgcgagaccgaagaat

SEQ ID NO: 83 - RNA, 100 nt, Plutella xylostella
  source 1..100: mol_type = mRNA
tgctcatcaaaaagcaggacttcctggagaagaaaatacaattagaagtagacacagccaggaaacatggcactaagaacaaaagagcggccatcgctgc

SEQ ID NO: 84 - RNA, 100 nt, Plutella xylostella
  source 1..100: mol_type = mRNA
acttaaacgcaagaagcgttacgagaagcagctcacacagatcgacggcacgctcagccagatagagatgcagagagaagcattggagggagccaacact

SEQ ID NO: 85 - RNA, 100 nt, Plutella xylostella
  source 1..100: mol_type = mRNA
aacactcaagtactgaacacgatgcgagaggccgccgcggctatgaagctcgctcacaaggatattgacgtagacaaagtgcacgatatcatggacgaca

SEQ ID NO: 86 - RNA, 100 nt, Plutella xylostella
  source 1..100: mol_type = mRNA
tcgctgaacaacatgatgtggctcgcgagatcacggatgcaatcagcaacaatgtggccttcccgagtgacattgatgatgaggagctggagagagagtt

SEQ ID NO: 87 - RNA, 101 nt, Plutella xylostella
  source 1..101: mol_type = mRNA
ggatgaactggaacaggaggacctggacaaggagatgctgggcatcaacgtgcccacggaccagctgcccgacgtgccgtccgccgagcccgccgcgccgc

SEQ ID NO: 88 - RNA, 100 nt, Plutella xylostella
  source 1..100: mol_type = mRNA
cgccccgccaaggccaagcctgctagcgaagacgacgatgatctggctaaactgcaatcgtgggcgacataaatgtaagtgttggaagcgaaaccgaata

SEQ ID NO: 89 - RNA, 100 nt, Plutella xylostella
  source 1..100: mol_type = mRNA
catatgtatagtctccgttacacatccaactacttagcttattctaaggctgcgtccctgtaggcaaacagtattctcgttactagtctatggtaatttg

SEQ ID NO: 90 - RNA, 100 nt, Plutella xylostella
  source 1..100: mol_type = mRNA
agtagagtgaaagaacaactagctaataaaaataaggaaggcccaagacagagtgttgtggcgctccttgggattggtctatgtccaacattggacgata

SEQ ID NO: 91 - RNA, 100 nt, Plutella xylostella
  source 1..100: mol_type = mRNA
acagctttatgactttaatttatgaggtactggctatggcagatctatttacgggcgcatcgctttgcgcttttaggagtttccctcgggaattccggga

SEQ ID NO: 92 - RNA, 150 nt, Plutella xylostella
  source 1..150: mol_type = mRNA
aggtccatcaacacacgaagctattcaaaaattacgcgagaccgaagaattgctcatcaaaaagcaggacttcctggagaagaaaatacaattagaagtagacacagccaggaaacatggcactaagaacaaaagagcggccatcgctgc

SEQ ID NO: 93 - RNA, 150 nt, Plutella xylostella
  source 1..150: mol_type = mRNA
taagtgttggaagcgaaaccgaatacatatgtatagtctccgttacacatccaactacttagcttattctaaggctgcgtccctgtaggcaaacagtattctcgttactagtctatggtaatttgagtagagtgaaagaacaactagcta

SEQ ID NO: 94 - RNA, 150 nt, Plutella xylostella; identical to SEQ ID NO: 93
  source 1..150: mol_type = mRNA

SEQ ID NO: 95 - RNA, 300 nt, Plutella xylostella
  source 1..300: mol_type = mRNA
aggtccatcaacacacgaagctattcaaaaattacgcgagaccgaagaattgctcatcaaaaagcaggacttcctggagaagaaaatacaattagaagtagacacagccaggaaacatggcactaagaacaaaagagcggccatcgctgctaagtgttggaagcgaaaccgaatacatatgtatagtctccgttacacatccaactacttagcttattctaaggctgcgtccctgtaggcaaacagtattctcgttactagtctatggtaatttgagtagagtgaaagaacaactagcta

SEQ ID NO: 96 - RNA, 100 nt, Plutella xylostella
  source 1..100: mol_type = mRNA
gtctgtcttttccatctttctcgtccaaaaaacaaggtaattgttcaagatgagtcacgggttgaagaggattgccgatgaggacaatgaaacccagttc

SEQ ID NO: 97 - RNA, 100 nt, Plutella xylostella
  source 1..100: mol_type = mRNA
ggttatgtcttcgctgtgtccggtcccgtggtcacagcggagaagatgtccggctcggccatgtacgagctggtgcgcgtcggctacaacgagctggtcg

SEQ ID NO: 98 - RNA, 100 nt, Plutella xylostella
  source 1..100: mol_type = mRNA
gagagatcatccgtctggagggggacatggccaccatccaggtgtacgaagagacctcaggcgtaaccgtcggcgaccccgtgctccgcaccggcaagcc

SEQ ID NO: 99 - RNA, 99 nt, Plutella xylostella
  source 1..99: mol_type = mRNA
tctctccgtagaactgggacccggcatcctcggctccatcttcgacggcatccagcgccgctgaaggacatcaacgagctcacgcagagcatctacatc

SEQ ID NO: 100 - RNA, 99 nt, Plutella xylostella
  source 1..99: mol_type = mRNA
cccaagggggtgaacgtgcccgcgctggcccgcgacaccgagtgggagttccaccgcagtacatcaaggtcggcacccacatcaccggcggggacttat

SEQ ID NO: 101 - RNA, 100 nt, Plutella xylostella
  source 1..100: mol_type = mRNA
acgggatcgtgcacgaaaacacgctggtgaagcaccgcatgctggtgccgcccaaggccaagggcaccgtcacctacatcgcgcccgaggggaactacaa

SEQ ID NO: 102 - RNA, 100 nt, Plutella xylostella
  source 1..100: mol_type = mRNA
cttacagtaagtacatgcgcgccctggacgacttctacgacaagaactaccccgagttcgtgccgctcaggaccaaggtcaaggagatcctgcaagagga

SEQ ID NO: 103 - RNA, 100 nt, Plutella xylostella
  source 1..100: mol_type = mRNA
ggaggacctgtctgaaatcgtgcagctggtcggaaaggcgtccctcgccgagaccgacaagatcaccctcgaggtggccaagctgctgaaggacgacttc

SEQ ID NO: 104 - RNA, 100 nt, Plutella xylostella
  source 1..100: mol_type = mRNA
ttgcagcagaacagctactcgaactacgaccgtttctgcccgttctacaagacgaccggcatgctgaagaacatcatcacgttctacgacatgtcgcgac

SEQ ID NO: 105 - RNA, 100 nt, Plutella xylostella
  source 1..100: mol_type = mRNA
acgctgtcgagtccaccgcgcagtccgacaacaaggtgacgtggaacacgatccgtgacgccatgggccccgtgctctaccagctgtccagcatgaagtt

SEQ ID NO: 106 - RNA, 100 nt, Plutella xylostella
  source 1..100: mol_type = mRNA
caaggaccccgtgaaagatggagaagccaagatcaaggctgacttcgaccagatcgtcgaggacatggccgctgccttccgtaacctagaggactaagtt

SEQ ID NO: 107 - RNA, 100 nt, Plutella xylostella
  source 1..100: mol_type = mRNA
atcatgcgcaattatactctttattcttgaagaggatgtttgggtcggacctcttgccgcgcggtgaaaaaaataaaactgtcaacttaaccatagagct

SEQ ID NO: 108 - RNA, 100 nt, Plutella xylostella
  source 1..100: mol_type = mRNA
gacagtgtctatggttagaaatagcgcgaaaagatactggctactgctaagataggttgatgtttgaggtccgatccaacattgttttgttaataaatat

SEQ ID NO: 109 - RNA, 100 nt, Plutella xylostella
  source 1..100: mol_type = mRNA
aaagtcgtgtttcaaatcttgttgaatttcatcttagacgttgaatataagccgttgacggtgtcaatgtattttttatgtaacacgcggtcactagata

SEQ ID NO: 110 - RNA, 100 nt, Plutella xylostella
  source 1..100: mol_type = mRNA
ctttctcgacaatatgatatatacattttgtaaataaagccccctttccattcaagattgtgtaatgttatatagaggtacatggtgcatcggtctagtg

SEQ ID NO: 111 - RNA, 100 nt, Plutella xylostella
  source 1..100: mol_type = mRNA
gcccaatgataattgaatgattgttttcgtcggttcatataccctgttgcattccagcttatttttagatagttaaaacaacaaatcggttatttttact

SEQ ID NO: 112 - RNA, 150 nt, Plutella xylostella; identical to SEQ ID NO: 26
  source 1..150: mol_type = mRNA

SEQ ID NO: 113 - RNA, 150 nt, Plutella xylostella
  source 1..150: mol_type = mRNA
gctgccttccgtaacctagaggactaagttatcatgcgcaattatactctttattcttgaagaggatgtttgggtcggacctcttgccgcgcggtgaaaaaaataaaactgtcaacttaaccatagagctgacagtgtctatggttagaa

SEQ ID NO: 114 - RNA, 300 nt, Plutella xylostella
  source 1..300: mol_type = mRNA
gtgataagttgtgattctccgccattctcggtctgtcttttccatctttctcgtccaaaaaacaaggtaattgttcaagatgagtcacgggttgaagaggattgccgatgaggacaatgaaacccagttcggttatgtcttcgctgtgtcgctgccttccgtaacctagaggactaagttatcatgcgcaattatactctttattcttgaagaggatgtttgggtcggacctcttgccgcgcggtgaaaaaaataaaactgtcaacttaaccatagagctgacagtgtctatggttagaa

SEQ ID NO: 115 - RNA, 300 nt, Plutella xylostella
  source 1..300: mol_type = mRNA
accaattgcaggcgtgaagaatgtcctcagatacggctacccttccttcttctacagcgtgggattggagctctattccagcaaaatttaccgtttcttccgatctgttatacttgacgttataaacagtcgtaacggcgccaaatcttcgaggaatgacatggtggatcttatttccgattggaagaagaacaaatacataacgggagacagtattgataatggcatagacggtggaaacaagaaggtgcgtatcgaagtcgacgacgaacttttggtgagccaatgtgtgctgttctt

SEQ ID NO: 116 - RNA, 100 nt, Plutella xylostella
  source 1..100: mol_type = mRNA
aactactggaagaaacagaacgtcaagtacttgacgccgatccctttcctggggaacgtggctgatgtgatcttccagagggacaccttcggagccgtga

SEQ ID NO: 117 - RNA, 100 nt, Plutella xylostella
  source 1..100: mol_type = mRNA
cgcaacggatctgccagcagttccccgatgaagctgtggtcggcatgttctactgcagcaaccctgcagccctcgtacagtgccctgacatgctcaagac

SEQ ID NO: 118 - RNA, 100 nt, Plutella xylostella
  source 1..100: mol_type = mRNA
agtcatggtcaaggactacgcctactgctccagtaaggaggtctccgtccacagccacaaggaacccatgaccaagaacatgttcttcaccttcggagac

SEQ ID NO: 119 - RNA, 100 nt, Plutella xylostella
  source 1..100: mol_type = mRNA
aagtggaagctcatccggcagaacctcacgccggtctacacgtccgccaaaatgaagaacatgtttccactggtacaggattgctgcagaatattccaga

SEQ ID NO: 120 - RNA, 100 nt, Plutella xylostella
  source 1..100: mol_type = mRNA
aggttctcgatgatgagataggaaagggccgggtggtggaagtgaagtctttgatagctcggtatactatggactgtataacttcgtgtgcattcggcgt

SEQ ID NO: 121 - RNA, 100 nt, Plutella xylostella
  source 1..100: mol_type = mRNA
cgactctggcacgatgtcgaagggcgaggaagggaaccctttcacagaaacaggtcaccttttatttgatgaaagaccaattgcaggcgtgaagaatgtc

SEQ ID NO: 122 - RNA, 100 nt, Plutella xylostella
  source 1..100: mol_type = mRNA
ctcagatacggctacccttccttcttctacagcgtgggattggagctctattccagcaaaatttaccgtttcttccgatctgttatacttgacgttataa

SEQ ID NO: 123 - RNA, 100 nt, Plutella xylostella
  source 1..100: mol_type = mRNA
acagtcgtaacggcgccaaatcttcgaggaatgacatggtggatcttatttccgattggaagaagaacaaatacataacgggagacagtattgataatgg

SEQ ID NO: 124 - RNA, 100 nt, Plutella xylostella
  source 1..100: mol_type = mRNA
caaagactacacgctaccgaatggtgtgcatctaaagaaggggatgatgatacatattcctgtttatcatttgcatcacaatccgaagtatttcccggag

SEQ ID NO: 125 - RNA, 100 nt, Plutella xylostella
  source 1..100: mol_type = mRNA
cccgaggtgtttcgtccggagcggttttctgaagaaggacggaaaagtattgtcccgtatacctacttgccctttggggacgggccgaggatgtgtatag

SEQ ID NO: 126 - RNA, 100 nt, Plutella xylostella
  source 1..100: mol_type = mRNA
gctaccgtttcgcaaggctagagatcttctccagcctagcagttctgttgaagaaataccgagtggagctggccccccacatgccgaggaagctgcagtt

SEQ ID NO: 127 - RNA, 150 nt, Plutella xylostella
  source 1..150: mol_type = mRNA
cctcagatacggctacccttccttcttctacagcgtgggattggagctctattccagcaaaatttaccgtttcttccgatctgttatacttgacgttataaacagtcgtaacggcgccaaatcttcgaggaatgacatggtggatcttat

SEQ ID NO: 128 - RNA, 150 nt, Plutella xylostella
  source 1..150: mol_type = mRNA
gaatccctccgcatgtatcctccagtctcggtgctcatgagagagatttacaaagactacacgctaccgaatggtgtgcatctaaagaaggggatgatgatacatattcctgtttatcatttgcatcacaatccgaagtatttcccggag

SEQ ID NO: 129 - RNA, 300 nt, Plutella xylostella
  source 1..300: mol_type = mRNA
ctcagatacggctacccttccttcttctacagcgtgggattggagctctattccagcaaaatttaccgtttcttccgatctgttatacttgacgttataaacagtcgtaacggcgccaaatcttcgaggaatgacatggtggatcttattgaatccctccgcatgtatcctccagtctcggtgctcatgagagagatttacaaagactacacgctaccgaatggtgtgcatctaaagaaggggatgatgatacatattcctgtttatcatttgcatcacaatccgaagtatttcccggag

SEQ ID NO: 130 - RNA, 58 nt, Plutella xylostella
  source 1..58: mol_type = mRNA
acaggtcaccttttatttgatgaaagaccaattgcaggcgtgaagaatgtcctcagat

SEQ ID NO: 131 - RNA, 58 nt, Plutella xylostella
  source 1..58: mol_type = mRNA
accgtttcttccgatctgttatacttgacgttataaacagtcgtaacggcgccaaatc

SEQ ID NO: 132 - RNA, 58 nt, Plutella xylostella
  source 1..58: mol_type = mRNA
ttcgaggaatgacatggtggatcttatttccgattggaagaagaacaaatacataacg

SEQ ID NO: 133 - RNA, 58 nt, Plutella xylostella
  source 1..58: mol_type = mRNA
ccgcatgtatcctccagtctcggtgctcatgagagagatttacaaagactacacgcta

SEQ ID NO: 134 - RNA, 58 nt, Plutella xylostella
  source 1..58: mol_type = mRNA
ccgaatggtgtgcatctaaagaaggggatgatgatacatattcctgtttatcatttgc

SEQ ID NO: 135 - RNA, 58 nt, Plutella xylostella
  source 1..58: mol_type = mRNA
ggagacagtattgataatggcatagacggtggaaacaagaaggtgcgtatcgaagtcg

SEQ ID NO: 136 - RNA, 58 nt, Plutella xylostella
  source 1..58: mol_type = mRNA
acgacgaacttttggtgagccaatgtgtgctgttcttccaagctggcttccagccaag

SEQ ID NO: 137 - RNA, 58 nt, Plutella xylostella
  source 1..58: mol_type = mRNA
tgcagaccgactgcgtgaccgccctgcctttcctcgcccagtgcatggaggaatccct

SEQ ID NO: 138 - RNA, 58 nt, Plutella xylostella; identical to SEQ ID NO: 133
  source 1..58: mol_type = mRNA

SEQ ID NO: 139 - RNA, 58 nt, Plutella xylostella; identical to SEQ ID NO: 134
  source 1..58: mol_type = mRNA

SEQ ID NO: 140 - RNA, 59 nt, Plutella xylostella
  source 1..59: mol_type = mRNA
atcacaatccgaagtatttcccggagcccgaggtgtttcgtccggagcggttttctgaa

SEQ ID NO: 141 - RNA, 57 nt, Plutella xylostella
  source 1..57: mol_type = mRNA
gaaggacggaaaagtattgtcccgtatacctacttgccctttggggacgggccgagg

SEQ ID NO: 142 - RNA, 150 nt, Plutella xylostella
  source 1..150: mol_type = mRNA
cagtttatactatcgaaatgattttagcaataatattgttattagtgattattataataacattttcgtttctactgggctcctacaatgagtcttactgggcaaaacgaaacgttaaataccatggcgggaaaaatgccatagcaacat

SEQ ID NO: 143 - RNA, 150 nt, Plutella xylostella
  source 1..150: mol_type = mRNA
tctcagagtttttgttcactagccgtggcattttcgatatattcggtaacatttacaagctgtaccctgaagaacctgcagtggccacgccgtcgctgcttcaaccggctttattcgtcaaacatccagaaaacatacaacatgtgctga

SEQ ID NO: 144 - RNA, 70 nt, Plutella xylostella
  source 1..70: mol_type = mRNA
cagacaattttaaaaacttttaccacagaggtgttgaaatcgctaagaaagataaactagcacaaaatgt

SEQ ID NO: 145 - RNA, 79 nt, Plutella xylostella
  source 1..79: mol_type = mRNA
acctttcctgaacggcagtcggtggaaacttatgagacaaaaaatgacgccgctgtcactagtgcgaagctgaagaaca

SEQ ID NO: 146 - RNA, 150 nt, Plutella xylostella
  source 1..150: mol_type = mRNA
tgcactacatcatagacagatgtgcccaagactacatcggttatctgaaagaacatgtcaacgataaaaatgctaatgcattcgaaacattatctgtgtacagctgctcatcgctactagccccgatattcggaatccacagcggacagt

SEQ ID NO: 147 - RNA, 150 nt, Plutella xylostella
  source 1..150: mol_type = mRNA
caacggtaacttcaccgcttctaaatatggcgagaaatgccacgaaaccgactttgaaagcaaatttaaaattcattttgaactctttgtcgccgaaagtcttccaaatgctgggacttagtttctttggcgaatatgaggaacagttca

SEQ ID NO: 148 - RNA, 150 nt, Plutella xylostella
  source 1..150: mol_type = mRNA
tcggcgcgatcagtcaagtgataagacaacgtaaggaagagaatgtgaaaaagcacgattttgctgacattgctgtgagtttgcagaatgctggtacgatgaaagacgaatccagcggctgtgaaatagagcctacggatgaagttctag

SEQ ID NO: 149 - RNA, 300 nt, Plutella xylostella
  source 1..300: mol_type = mRNA
actttgaaagcaaatttaaaattcattttgaactctttgtcgccgaaagtcttccaaatgctgggacttagtttctttggcgaatatgaggaacagttcatcggcgcgatcagtcaagtgataagacaacgtaaggaagagaatgtgaaaaagcacgattttgctgacattgctgtgagtttgcagaatgctggtacgatgaaagacgaatccagcggctgtgaaatagagcctacggatgaagttctagctgcacaagcgttcttcttccttatagcaggagtagatccagtaacaatg

SEQ ID NO: 150 - RNA, 302 nt, Plutella xylostella
  source 1..302: mol_type = mRNA
agataaactagcacaaaatgtacctttcctgaacggcagtcggtggaaacttatgagacaaaaaatgacgccgctgttcactagtgcgaagctgaagaacatgcactacatcatagacagatgtgcccaagactacatcggttatctgaaagaacatgtcaacgataaaaatgctaatgcattcgaaacattatctgtgtacagctgctcatcgctactagccccgatattcggaatccacagcggacagtcaacggtaacttcaccgcttctaaatatggcgagaaatgccacgaaaccga

SEQ ID NO: 151 - RNA, 1359 nt, Spodoptera frugiperda
  source 1..1359: mol_type = mRNA
tgagagctactggaagaagcgtggcgtaaagttctacagtaaaaacaaagtgattgaccttactgggattatttcataacacagcgtgcgttgttcgaagtcttaacagacctgtacaaaaaatatagacatgaaccagctattggtattggtcaagtcctaacacctgcgctctttgtcatcgatccgaagaatgttcagcaagttttatcaagtgacttccaatcttttaaccacagaggcattgaaagtattgaaggggatcaactgaccgataatattctcatgatgaatggtccaagatggaagctgatgcgacagaatatgactccgttgtttacggcaaacaaattgaagaacatgtattacatcatggacaaaagtgctcaagactttgcgaactatttgaaaaacaaccccaagacgcgtgatggtaacttatttgaaactcttatgatgttctgtaatgctgcagtttgtggagctatcttcggcattgggtcagagtcaatctttgattcacctttcctcaagcttgccaaaaacatatcgcaatccaattttaagatgagaatgaaattcgttatttatagtctcagtccaaagttgtacaaactgttgagattacaagctttcaacgagatcgaagacttcttcattggttcaataagtcaagtgatgaaatcaagagaacaagaaaatgtaaagagacacgactttgctgaaattgctgtggctatacagaaaaatggtattatgaaagaccgtacaacaggttgcgagatagagcctactaccggcattttgtctgcacaggcatttttcttctttagtgctggtgtagagccttgtgctaatgcaatcttttcaacattatttcttttaagcagtcacccagaaatactagaaagagttcaccaagaaattgacgaacattttgaaaagcatagcaacaacataaattacgatgttatatgtgaaatgaagtacacagacaaggtgctgagtgaagcgatgagaatgttacctccaattggtcacttgacaagacaatgtgttcaaaatactgtcctgcctgttggtaatatcccagtagaaaaggggacaaaaatgttcactccaatttatgctattcatcacgacccagaactatatccagacccagaagtgttcgacccggagcgatttgccaatgatagaaaaccgaatgataacatttacatgccatttggaatgggcaacagggcatgcataggggagaggtatgccaaattacaagtacaagccggtttagttcacgtcttgcgaaacttcactgttaaaccgcagaaacatgtaaaagtaacatttgcccaggat

SEQ ID NO: 152 - RNA, 300 nt, Spodoptera frugiperda
  source 1..300: mol_type = mRNA
tttaaccacagaggcattgaaagtattgaaggggatcaactgaccgataatattctcatgatgaatggtccaagatggaagctgatgcgacagaatatgaaagctttcaacgagatcgaagacttcttcattggttcaataagtcaagtgatgaaatcaagagaacaagaaaatgtaaagagacacgactttgctgaaattccaattggtcacttgacaagacaatgtgttcaaaatactgtcctgcctgttggtaatatcccagtagaaaaggggacaaaaatgttcactccaatttat

SEQ ID NO: 153 - RNA, 100 nt, Spodoptera frugiperda
  source 1..100: mol_type = mRNA
aagctttcaacgagatcgaagacttcttcattggttcaataagtcaagtgatgaaatcaagagaacaagaaaatgtaaagagacacgactttgctgaaat

SEQ ID NO: 154 - RNA, 100 nt, Spodoptera frugiperda
  source 1..100: mol_type = mRNA
tccaattggtcacttgacaagacaatgtgttcaaaatactgtcctgcctgttggtaatatcccagtagaaaaggggacaaaaatgttcactccaatttat

SEQ ID NO: 155 - RNA, 1440 nt, Spodoptera frugiperda
  source 1..1440: mol_type = mRNA
ctcgtatcaaaacgaaaattcagatactgggaacagaagaaagtgccacacctaccaccaaaaccaattctgggaaatttctccgaatacattctccagcagaaattctatggacgtgttgaacaggagatctgcaacaagtttcctgaagaaccatacgttggctcttacttgggcacggaaccgaccctcatcatacaagatcccgaatacatcaagaccgtcatgaccaaggactactatttcttcagtggccgtgaagtctctgcatactgtgaaaaggaaccgttaactcagaacctattcttcacttatggcgataagtggaaggtactgcgtcagaaccttacgcctttgttctcatccgcaaagatgaagaacatgttccatttgatcgagaaatgtgcccgtatcttcgagaacatggtcgaccaggaagtacagaaaagtaaagacattgaagtgcgagccctgacgggaaaattcaccatggatgccattggaaatttgcttttggagttgagacccagacgatggtaaagactgacaataatccattcacaaagttggcgatgttatcttcgatactgccagaatgagagcattaaagggtgtgctaagaagtatttggccagccatgttttatagttttggaggtaaagcagtccctgctgatgtgatagctttttcttcaatttgatgacgagtatcttcaaaggacgcaactataatctgatcaaggaacgactttgtggacctcctattgaagttccataacaacaaaacagtgactggggatagtatgagaaacttgaagggtgattcaggaaagaaagtcagtttagaagtggacgatgagtttttgattgcacaatgtttcctgttctttgctgctgggtatgagacgtcggcgactacgttgagttacactttatatgagttggcaaagaatcctgaagctcaagagttagcaattcaagacgtggacaactacttgcgtcgcaatgacaatgtgttgaagtacgagtgtgtgaccgagttgccttatgtggaagcttgtgttgatgaggctcttcgtctatacccagtgctaggagtaattactcgggaggttattgaggattatacattcccgacgggattgaaattggagaaaggacttcgcgtgcatttaccggtgtaccacatgcacacaaccctaattacttcccggaaccagagcaatatcgtccggagcggttcttgggcgacgagaagaaaaatattaagccgtatacatacttcccatttggcgaaggacctcgactatgtatcggaatgaggttcgcaaagatgcagattacggctggaattataacattactgaagaaatatcgcgtagaacttgcgccgggtatgagcgagacaatacagttcgaacctcgatctgtaatcaccgcc

SEQ ID NO: 156 - RNA, 300 nt, Spodoptera frugiperda
  source 1..300: mol_type = mRNA
atagttttggaggtaaagcagtccctgctgatgttgatagctttttcttcaatttgatgacgagtatcttcaaaggacgcaactataatctgacatcaaggaacgactttgtggacctcctattgaagttccataacaacaaaacagtgactggggatagtatgagaaattgaagggtgattcaggaaagaaagtcagtttagaagtggacgatgagtttttgattgcacaatgtttcctgttctttgctgctgggtatgagacgtcggcgactacgttgagttacactttatatgagtt

SEQ ID NO: 157 - RNA, 2255 nt, Spodoptera frugiperda
  source 1..2255: mol_type = mRNA
tataaatgattcataagtgttcggaccgcgtatttggccagtcgcaaccatggcggccttatatttcctcgccgcagtgctagtgttagtgtacgcgttatattattacttcacaaggacattcaactactggaagagtagaaatgtgcgaggaccaaaaccagttgcattatttggaaacattaaggacgcagctcttcgcaaagaaaattatggcgtcgtaatgcaaaatatatacaatgcatatccaaatgaaaaagtggtcggcatattcaggatgacttcgccttgtctccttattcgagacctggacattatcaaacatatcatgatcaaagacttcgaagccttcagtgatcgtggagtggaattcagcaaagaaggattgggacaaaacttattccacgcggacggagatacatggactgccttgaggaacagattcactcccattttcacaacaggtaaattgaagaacatgttttacctaataaatgagggaggcgattcatttgtagagtacatccgtacagaatgccaaaagaaggaagaatttgatattcagcctctcctccagacgtatactttgtctacgatctccgcctgtgcattcggaattagctatgacagtcttgatgttaaaatggatactctgaaacttgtggataaaatattttcttcaccaagttttgcagttgaattggatatgatgtatcccggtctcctgaaatctctaaacctttctttattccctaccgccataaaaaagttctttgataatctagtgaataatgttatagagcaaagaaatggtaaaccatcgggtcgaaatgatttcatggatcttattttggcgctccgtgaaatgggagaggtcacaaactcaaaatatgactctgcaaagccagttgaaataacacctggtgtgatagcagcgcaagcttttgtgttttatgcggctggttatgaaaccagtgctaccactatgacgtacatgctttaccaactagcaatgaatccagacatccaaaagaagttgactgaagaaattgacgaatctctcaaagcaaataatggacaagttacatacgagagcattaaggaaatgaagtatttgaacaaagtgtttgatgaaactctacgaatgtactcgattgtagaacctctgcagaggaaagctgtaagagattacaaagtgcccggtactgacttgacgatagaaaagaacacaattgtgctggtatctccgagaggtatccactacgacgagaaatattacgacaaccctgaacagttcaaccctgacagatttgacgcggaggaggtgggcaagcgacatccgtgcgcttacatgccgtttggaattggacagagaaactgcatcggaatgaggttcggcagacttcaatcccaactgtgcataaccaagttgctgtctaagttccaagtggagccatcgaggaatactgcaaggaagctggaagtggaaccttgtcgctttatcatcggacccaaaggagggatacgtctgaatattgttccaagaaagctgaaggcttaacacattaaacgccatggggggcaaatgtgaccggcgttagcagaggatttgccttcaacagatttttgttggcagtcaatgtataacgacttttaaaactgtttataaatccatccatctaccaggtaaacttaggtataccaatgtgttattttatttttttcattgaaaattcgactttgtatgtaaataatacggttgaaatctaatacagaaaactatcagattgcagtagcttaacctgctgctcattgtatatagaatatcgttttaaatttgattcaaaataataattgtatgtggccagtttcaccagtcatgttattatggagtgcgcggacttagtgggctactccttaggtggcgaacaggggtgagggggggggaggcaattcgcctacgcatatgagaatagacaatgatgattgatgaccaatcttgagatatcgggaatatgaatggttcaattattgaaacaaaagacaattgaacgtatagaacttttacgcacgcaaccaactagttccgcgcgcactattattgagatattataagactcataaagggaagaaaccatcaacaaacatcgattacaaatccattcatttgcaaattgtatataattactcttagcacattaatcatgcatttagtttataagtaagctatatttaattaaattatttaaaaacta

SEQ ID NO: 158 - RNA, 200 nt, Spodoptera frugiperda
  source 1..200: mol_type = mRNA
tattattacttcacaaggacattcaactactggaagagtagaaatgtgcgaggaccaaaaccagttgcattatttggaaacattaaggacgcagctcttcgcaaagaaaattatggcgtcgtaatgcaaaatatatacaatgcatatccaaatgaaaaagtggtcggcatattcaggatgacttcgccttgtctccttat

SEQ ID NO: 159 - RNA, 100 nt, Spodoptera frugiperda
  source 1..100: mol_type = mRNA
aggaaatgaagtatttgaacaaagtgtttgatgaaactctacgaatgtactcgattgtagaacctctgcagaggaaagctgtaagagattacaaagtgcc

SEQ ID NO: 160 - RNA, 298 nt, Spodoptera frugiperda
  source 1..298: mol_type = mRNA
tcaagcctatcccattgctgggcaatatgggcactgtattgcttcgaagacaacacttagcatatagtcttattgatttgtataatgccttccctgaagaaaaatttgtaggaaggtttgagttcatgaacgaggcagtgctgatcagagcaagttcgacttcaactcgatacagagcatgaagtatatggataatgtggtgtcagattattacgacgatggcctgtagctgtagctactgacagaatttgtgaaaaggactacaacatgggtaaaccaaatgaaaggctgagaagga

SEQ ID NO: 161 - RNA, 100 nt, Spodoptera frugiperda
  source 1..100: mol_type = mRNA
tattattacttcacaaggacattcaactactggaagagtagaaatgtgcgaggaccaaaaccagttgcattatttggaaacattaaggacgcagctcttc

SEQ ID NO: 162 - RNA, 100 nt, Spodoptera frugiperda
  source 1..100: mol_type = mRNA
gcaaagaaaattatggcgtcgtaatgcaaaatatatacaatgcatatccaaatgaaaaagtggtcggcatattcaggatgacttcgccttgtctccttat

SEQ ID NO: 163 - RNA, 200 nt, Spodoptera frugiperda; identical to SEQ ID NO: 158
  source 1..200: mol_type = mRNA

SEQ ID NO: 164 - RNA, 100 nt, Spodoptera frugiperda
  source 1..100: mol_type = mRNA
tcgagacctggacattatcaaacatatcatgatcaaagacttcgaagccttcagtgatcgtggagtggaattcagcaaagaaggattgggacaaaactta

SEQ ID NO: 165 - RNA, 99 nt, Spodoptera frugiperda
  source 1..99: mol_type = mRNA
cggtactgacttgacgatagaaaagaacacaattgtgctggtatctccgagaggtatccactacgacgagaaatattacgacaaccctgaacagttcaa

SEQ ID NO: 166 - RNA, 100 nt, Spodoptera frugiperda
  source 1..100: mol_type = mRNA
cctgacagatttgacgcggaggaggtgggcaagcgacatccgtgcgcttacatgccgtttggaattggacagagaaactgcatcggaatgaggttcggca

SEQ ID NO: 167 - RNA, 200 nt, Spodoptera frugiperda; identical to SEQ ID NO: 158
  source 1..200: mol_type = mRNA

SEQ ID NO: 168 - RNA, 300 nt, Spodoptera frugiperda
  source 1..300: mol_type = mRNA
tattattacttcacaaggacattcaactactggaagagtagaaatgtgcgaggaccaaaaccagttgcattatttggaaacattaaggacgcagctcttcgcaaagaaaattatggcgtcgtaatgcaaaatatatacaatgcatatccaaatgaaaaagtggtcggcatattcaggatgacttcgccttgtctccttataggaaatgaagtatttgaacaaagtgtttgatgaaactctacgaatgtactcgattgtagaacctctgcagaggaaagctgtaagagattacaaagtgcc

SEQ ID NO: 169 - RNA, 100 nt, Spodoptera frugiperda
  source 1..100: mol_type = mRNA
acatgaaccagctattggtattggtcaagtcctaacacctgcgctctttgtcatcgatccgaagaatgttcagcaagttttatcaagtgacttccaatct

SEQ ID NO: 170 - RNA, 100 nt, Spodoptera frugiperda
  source 1..100: mol_type = mRNA
tgctgtggctatacagaaaaatggtattatgaaagaccgtacaacaggttgcgagatagagcctactaccggcattttgtctgcacaggcatttttcttc

SEQ ID NO: 171 - RNA, 100 nt, Spodoptera frugiperda
  source 1..100: mol_type = mRNA
gctattcatcacgacccagaactatatccagacccagaagtgttcgacccggagcgatttgccaatgatagaaaaccgaatgataacatttacatgccat

SEQ ID NO: 172 - RNA, 301 nt, Plutella xylostella
  source 1..301: mol_type = mRNA
gactacaacccgaaggtgggcgacatcccgatcgacttcaaggagcccatctacggcgacaccaacccctccttcactgacttcgtcgctcccggtgttattctcactatcgtgttcttcctggcggtggcgctgacgtcgtcggcgctgatcgtggagcgcatggaggggctgctggaccgctcgtgggtggccggcgtgtcccccggggagatcctgttctcgcacgtcgtcacgcagttcgtcgtcatgtgcggccagaccgcgctcgtgctcgtgttcatgatactggtgttcggcg

SEQ ID NO: 173 - RNA, 149 nt, Spodoptera frugiperda
  source 1..149: mol_type = mRNA
tcaagcctatcccattgctgggcaatatgggcactgtattgcttcgaagacaacacttagcatatagtcttattgatttgtataatgccttccctgaagaaaaatttgtaggaggtttgagttcatgaacgaggcagtgctgatcagag

SEQ ID NO: 174 - RNA, 149 nt, Spodoptera frugiperda
  source 1..149: mol_type = mRNA
caagttcgacttcaactcgatacagagcatgaagtatatggataatgtggtgtcagaattattacgacgatggcctgtagctgtagctactgacagaatttgtgaaaaggactcaacatgggtaaaccaaatgcaaaggctgagaagga

SEQ ID NO: 175 - RNA, 149 nt, Spodoptera frugiperda
  source 1..149: mol_type = mRNA
gcgccaaaattttgtgacagtgacttttgtgattatctgtgacactttattttttctatttttacttcccttcggattgtttgagagatggcgcatcctacagagttgcgctccgagagagtgaaacaaatgtttagttggactacggg

SEQ ID NO: 176 - RNA, 200 nt, Spodoptera frugiperda
  source 1..200: mol_type = mRNA
ttattttgacaattgtttatatttgatgcgtaatctgaacaaaaacatagtggattacatttgtaaatgttttggtacgattgtaagctcacggtaggaaggaattagaagttcaggaccacgccttatagtcagtaaatacttcttcatttaaacggtgaagggcgacgagtcagtttgttgtacactctcgaaactat

SEQ ID NO: 177 - RNA, 100 nt, Spodoptera frugiperda
  source 1..100: mol_type = mRNA
attcttcaaactcttggaagcggcagacgcattgaaatattactacgatcgcttaccttacgagatgcaagcggacgaacctgaaaccagagttaccaaa

SEQ ID NO: 178 - RNA, 100 nt, Spodoptera frugiperda
  source 1..100: mol_type = mRNA
gcttaccttacgagatgcaagcggacgaacctgaaaccagagttaccaaaaaagtgatatttactcctaaattgggaaggagtttggcttatgatgataa

SEQ ID NO: 179 - RNA, 100 nt, Spodoptera frugiperda
  source 1..100: mol_type = mRNA
agtctttgagaatgttgagttcacaccacggttgggaagacggttggctgatgatatgcccgcgacgccggctgatcaggaactgtatagaccagacccg

SEQ ID NO: 180 - RNA, 99 nt, Spodoptera frugiperda
  source 1..99: mol_type = mRNA
gatcagatcgacagcaggacgaagtacttctcgcctaggctcggcaggaccatgatttttcaccacgactcggcagggaattgtcctatgaaatgttac

SEQ ID NO: 181 - RNA, 100 nt, Spodoptera frugiperda
  source 1..100: mol_type = mRNA
caagcaaactaagaatggtgaggagtgccaacagaacgcaatcgacataaacttgacatgagcccgccatcaaacataacctcaaaatgagacaaactaa

SEQ ID NO: 182 - RNA, 150 nt, Spodoptera frugiperda
  source 1..150: mol_type = mRNA
caagcaaactaagaatggtgaggagtgccaacagaacgcaatcgacataaacttgacatgagcccgccatcaaacataacctcaaaatgagacaaactaatgtactgtacaatgtataaataaatcgctattttggacgaaataatgtac

SEQ ID NO: 183 - RNA, 200 nt, Spodoptera frugiperda
  source 1..200: mol_type = mRNA
attcttcaaactcttggaagcggcagacgcattgaaatattactacgatcgcttaccttacgagatgcaagcggacgaacctgaaaccagagttaccaaaaaagtgatatttactcctaaattgggaaggagtttggcttatgatgataaagtctttgagaatgttgagttcacaccacggttgggaagacggttggctg

SEQ ID NO: 184 - RNA, 150 nt, Spodoptera frugiperda
  source 1..150: mol_type = mRNA
agttttctggggaagttattcggtggtaagaaggaagaaaagggcccgacaacacatgaagctattcagaaattacgcgagacagaggagctcttgctgaaaaaacaagagtttctagagaagaaaatcgatatagaaatacagactgct

SEQ ID NO: 185 - RNA, 150 nt, Spodoptera frugiperda
  source 1..150: mol_type = mRNA
gtcttgggccacataatgtatgcacctcaagttgtaattttgtgatatattataggtattgtatagccatccgctttgttacgcactcttgtgggcacagcacgtcgatgtatactggttttgtattgaaaatatgtagcttgatatgct

SEQ ID NO: 186 - RNA, 300 nt, Spodoptera frugiperda
  source 1..300: mol_type = mRNA
agttttctggggaagttattcggtggtaagaaggaagaaaagggcccgacaacacatgaagctattcagaaattacgcgagacagaggagctcttgctgaaaaaacaagagtttctagagaagaaaatcgatatagaaatacagactgctgtcttgggccacataatgtatgcacctcaagttgtaattttgtgatatattataggtattgtatagccatccgctttgttacgcactcttgtgggcacagcacgtcgatgtatactggttttgtattgaaaatatgtagcttgatatgct

SEQ ID NO: 187 - RNA, 300 nt, Spodoptera frugiperda
  source 1..300: mol_type = mRNA
ttgtatattgaacatttggacaaacaggtatacacattttactgtaagaataatgtgacaaacaaaccttgcaccactagagtgctagtggacgactctccctccaatgttactgactttagctgcatatcaaagaatcttgatgaactgaaacataactgatcttaaaccttatacaacatatctttttaccctcgccttaaatactacttatgggttaaagacaattgaaaatgcttccactggagtgacaactatcgaggatactcccacaagtccaaggaatgtacgcattacaga

SEQ ID NO: 188 - RNA, 150 nt, Spodoptera frugiperda
  source 1..150: mol_type = mRNA
ttgtatattgaacatttggacaaacaggtatacacattttactgtaagaataatgtgacaaacaaaccttgcaccactagagtgctagtggacgactctccctccaatgttactgactttagctgcatatcaaagaatcttgatgaactg

SEQ ID NO: 189 - RNA, 150 nt, Spodoptera frugiperda
  source 1..150: mol_type = mRNA
aaacataactgatcttaaaccttatacaacatatctttttaccctcgccttaaatactacttatgggttaaagacaattgaaaatgcttccactggagtgacaactatcgaggatactcccacaagtccaaggaatgtacgcattacaga

SEQ ID NO: 190 - RNA, 100 nt, Spodoptera frugiperda
  source 1..100: mol_type = mRNA
gatcttttgcgtggctgaaaactatactgctgagaacttggagttttaccacggtcgaaaatttatagaatcagaaaaagtgaatgcaaccaccaggagg

SEQ ID NO: 191 - RNA, 100 nt, Spodoptera frugiperda
  source 1..100: mol_type = mRNA
ttgtatattgaacatttggacaaacaggtatacacattttactgtaagaataatgtgacaaacaaaccttgcaccactagagtgctagtggacgactctc

SEQ ID NO: 192 - RNA, 100 nt, Spodoptera frugiperda
  source 1..100: mol_type = mRNA
cctccaatgttactgactttagctgcatatcaaagaatcttgatgaactgaactgtacttggacttctccagagatttacagcataactacataccggtt

SEQ ID NO: 193 - RNA, 100 nt, Spodoptera frugiperda
  source 1..100: mol_type = mRNA
aaataatcatttaaaacgagaaaatgttttctgtagactcagttaccacaacaccacaaggaatggaagtggaaaatgttattggaaacagtgatattat

SEQ ID NO: 194 - RNA, 100 nt, Spodoptera frugiperda
  source 1..100: mol_type = mRNA
gaatactgatatgatgtcagaaattgaaaaagaattacaagacaatcccagcgacttgatatccctagtgtttcttctttatgaagtaccggacacagca

SEQ ID NO: 195 - RNA, 100 nt, Spodoptera frugiperda
  source 1..100: mol_type = mRNA
ccacaacgtttaattgtttttcaacgcgtttccaacgatgcctgtaactctattaatcttaacatgctacacgagtggtttcgatctactaagcacaacc

SEQ ID NO: 196 - RNA, 100 nt, Spodoptera frugiperda
  source 1..100: mol_type = mRNA
ccaactggaaacatcaatttgtggaagctctccttatctgtcaactgtacagtattgtcagaaaacttggattgaatgtccctacagcacgcaagtacta

SEQ ID NO: 197 - RNA, 100 nt, Spodoptera frugiperda
  source 1..100: mol_type = mRNA
gatgaatatgttgcaacactcattgaatgatgagcacaagccaagtgcctcagctgctaccagtactcctatgatgaaacatatgaaagtggatgaaaca

SEQ ID NO: 198 - RNA, 100 nt, Spodoptera frugiperda
  source 1..100: mol_type = mRNA
aatcaagggaaagctgatgacaattattacaatatggattttaatgatgtgtttgaaatgcttggagagttacagttggatgaaagaatgcaagagtctc

SEQ ID NO: 199 - RNA, 100 nt, Spodoptera frugiperda
  source 1..100: mol_type = mRNA
tgaagtctgataggaagctactggacaatgatagctatgaaatcaagagtaacaaaagggttggagtttgtgtcatcataaatcaggaaacattttatcc

SEQ ID NO: 200 - RNA, 100 nt, Spodoptera frugiperda
  source 1..100: mol_type = mRNA
tgatgccaaaaaaatacacaacatgcctaaacttttgatagttcaagcatgccaagttgatgaaaatactccccagattgtagtggctgacagcccaaga

SEQ ID NO: 201 - RNA, 100 nt, Spodoptera frugiperda
  source 1..100: mol_type = mRNA
gccaagttgatgaaaatactccccagattgtagtggctgacagcccaagagattacaatttaaggaaatctaacttccttgtttactatgccactgcacc

SEQ ID NO: 202 - RNA, 100 nt, Spodoptera frugiperda
  source 1..100: mol_type = mRNA
gattacaatttaaggaaatctaacttccttgtttactatgccactgcacctgaactagaagcttacagaaatgaaaaaagaggatcgatattcattcagg

SEQ ID NO: 203 - RNA, 100 nt, Spodoptera frugiperda
  source 1..100: mol_type = mRNA
tgctatgtagaacaattagaaagtatgctaatactgaacatgtctatgatatttttactaaggtcaataataatgtgaatttcatttgccaaaaggtggg

SEQ ID NO: 204 - RNA, 300 nt, Spodoptera frugiperda
  source 1..300: mol_type = mRNA
gaatactgatatgatgtcagaaattgaaaaagaattacaagacaatcccagcgacttgatatccctagtgtttcttctttatgaagtaccggacacagcatgaagtctgataggaagctactggacaatgatagctatgaaatcaagagtaacaaaagggttggagtttgtgtcatcataaatcaggaaacattttatcctgatgccaaaaaaatacacaacatgcctaaacttttgatagttcaagcatgccaagttgatgaaaatactccccagattgtagtggctgacagcccaaga

SEQ ID NO: 205 - RNA, 300 nt, Spodoptera frugiperda
  source 1..300: mol_type = mRNA
acaccacaaggaatggaagtggaaaatgttattggaaacagtgatattatgaatactgatatgatgtcagaaattgaaaaagaattacaagacaatcccagcgacttgatatccctagtgtttcttctttatgaagtaccggacacagcaccacaacgtttaattgtttttcaacgcgtttccaacgatgcctgtaactctattaatcttaacatgctacacgagtggtttcgatctactaagcacaaccccaactggaaacatcaatttgtggaagctctccttatctgtcaactgtac

SEQ ID NO: 206 - RNA, 101 nt, Spodoptera frugiperda
  source 1..101: mol_type = mRNA
atgttggtctcaacgttacaagttgtgataccggtgttctgtgatccagaagtgtactacgagaaacagcggtatgatggctggttcaacaacagagctta

SEQ ID NO: 207 - RNA, 101 nt, Spodoptera frugiperda
  source 1..101: mol_type = mRNA
acccagactggggatccgttggcagtcgtctcacacgcaagactccagcatcatacgctgatggggtctacatgatggcaggcgtcgatagacccggcgcc

SEQ ID NO: 208 - RNA, 101 nt, Spodoptera frugiperda
  source 1..101: mol_type = mRNA
cagaacattatccaaactcttcatgagagggcaagatggactgccttctttagccaacaggacagcattattggcgttctttggccaggtcgtaaccggcg

SEQ ID NO: 209 - RNA, 101 nt, Spodoptera frugiperda
  source 1..101: mol_type = mRNA
gagattgtaatggcatccgagtcgggctgtcctatcgagcaccatcgcataccagtggacaaatgtgaccacatgtatgacccagaatgtaatggagccaa

SEQ ID NO: 210 - RNA, 101 nt, Spodoptera frugiperda
  source 1..101: mol_type = mRNA
tcacatcaaagcacaaggtccctggacgtggaaactcaggaattacttcgatccctgcaactttaatactgaagatcatcctaaaatcaggatccaaggtc

SEQ ID NO: 211 - RNA, 101 nt, Spodoptera frugiperda
  source 1..101: mol_type = mRNA
agtacatgccgtttcttagagcagcatatgatcggaacacggggcagagtcccaacagtcctagagaacagataaaccaaatgacttcgtggatcgacggc

SEQ ID NO: 212 - RNA, 101 nt, Spodoptera frugiperda
  source 1..101: mol_type = mRNA
cttggaatgaaagcagatgctgtatttgtgaagaaaatgttcaacattgtcgacaaagacggagatggcaggatctctttccaggaatttctggacacggt

SEQ ID NO: 213 - RNA, 101 nt, Spodoptera frugiperda
  source 1..101: mol_type = mRNA
tagtcctattttctcgaggagcaacagaagacaaacttcgcattatcttcgacatgtgtgacgatgatcgtaacggtgtgatcgacaagggagagctcagt

SEQ ID NO: 214 - RNA, 101 nt, Spodoptera frugiperda
  source 1..101: mol_type = mRNA
ctacgaactaagtatctagcactggatgttttggaaacagaaatgctaccatcggatgtgattaaaataaagttctatagaccaccaaatctaaaatattt

SEQ ID NO: 215 - RNA, 101 nt, Spodoptera frugiperda
  source 1..101: mol_type = mRNA
tatcaggtcaatgggtacggttatcttgtacggcgttcaagaaagaagagttccactcgttcacattaacctcagctcctcacgagaacttcttatcgtgt

SEQ ID NO: 216 - RNA, 303 nt, Spodoptera frugiperda
  source 1..303: mol_type = mRNA
atgttggtctcaacgttacaagttgtgataccggtgttctgtgatccagaagtgtactacgagaaacagcggtatgatggctggttcaacaacagagcttactacgaactaagtatctagcactggatgttttggaaacagaaatgctaccatcggatgtgattaaaataaagttctatagaccaccaaatctaaaatatttctacgaactaagtatctagcactggatgttttggaaacagaaatgctaccatcggatgtgattaaaataaagttctatagaccaccaaatctaaaatattt

SEQ ID NO: 217 - RNA, 303 nt, Spodoptera frugiperda
  source 1..303: mol_type = mRNA
ctacgaactaagtatctagcactggatgttttggaaacagaaatgctaccatcggatgtgattaaaataaagttctatagaccaccaaatctaaaatattttatcaggtcaatgggtacggttatcttgtacggcgttcaagaaagaagagttccactcgttcacattaacctcagctcctcacgagaacttcttatcgtgttcacatcaaagcacaaggtccctggacgtggaaactcaggaattacttcgatccctgcaactttaatactgaagatcatcctaaaatcaggatccaaggtc

SEQ ID NO: 218 - RNA, 300 nt, Spodoptera frugiperda
  source 1..300: mol_type = mRNA
taaaatggcggccccggatacggtagggagtcgaatatggctcctagtaatgttggtctcaacgttacaagttgtgataccggtgttctgtgatccagaagtgtactacgagaaacagcggtatgatggctggttcaacaacagagcttaagccaacaggacagcattattggcgttctttggccaggtcgtaaccggcgagattgtaatggcatccgagtcgggctgtcctatcgagcaccatcgcataccagtggacaaatgtgaccacatgtatgacccagaatgtaatggagccaa

SEQ ID NO: 219 - RNA, 100 nt, Spodoptera frugiperda
  source 1..100: mol_type = mRNA
catgcgagaagaggctttgatgggtgtgaaactatatgctgcagacttcagcccaccggtgcgttccagcatgatggcccttgatatcttcaatgtacca

SEQ ID NO: 220 - RNA, 100 nt, Spodoptera frugiperda
  source 1..100: mol_type = mRNA
tttgagaagataactctcaacttactgaaactggaacaccgcactccagaatatttggaaaaaaatccgattcactcgattccagtattggaagatggag

SEQ ID NO: 221 - RNA, 100 nt, Spodoptera frugiperda
  source 1..100: mol_type = mRNA
atttaatattacatgatagccacgcaataatggcctacctcgctgacacctacggaaaggatgagtcttggtatcctaaggacgtgaaaaaacgagcttt

SEQ ID NO: 222 - RNA, 100 nt, Spodoptera frugiperda
  source 1..100: mol_type = mRNA
agttaaccaaaaactgtttttcaccacaggtgtaattttccctagactacgaatcattacatactatcttctagaaaaaggaagaaaagctatagaacaa

SEQ ID NO: 223 - RNA, 100 nt, Spodoptera frugiperda
  source 1..100: mol_type = mRNA
gaatggctagacaacattgaagaagccattggttttgtggagcagttcttgtctcggacaaaattcatcgccttggaccatgtcaccattgccgatattg

SEQ ID NO: 224 - RNA, 100 nt, Spodoptera frugiperda
  source 1..100: mol_type = mRNA
cagtgttaagtcacctgtcctctgtggaacctattctacccatagatccgaaaaagtacccaaagacggttgcctggttagaaattatgaaagcgacgcc

SEQ ID NO: 225 - RNA, 100 nt, Spodoptera frugiperda
  source 1..100: mol_type = mRNA
aaaaagtacccaaagacggttgcctggttagaaattatgaaagcgacgccatattgcaagaaatataacgaagaaggtgttaaatctctgactgctatta

SEQ ID NO: 226 - RNA, 300 nt, Spodoptera frugiperda
  source 1..300: mol_type = mRNA
tttgagaagataactctcaacttactgaaactggaacaccgcactccagaatatttggaaaaaaatccgattcactcgattccagtattggaagatggagagttaaccaaaaactgtttttcaccacaggtgtaattttccctagactacgaatcattacatactatcttctagaaaaaggaagaaaagctatagaacaaaaaaagtacccaaagacggttgcctggttagaaattatgaaagcgacgccatattgcaagaaatataacgaagaaggtgttaaatctctgactgctatta

SEQ ID NO: 227 - RNA, 300 nt, Spodoptera frugiperda
  source 1..300: mol_type = mRNA
tttgagaagataactctcaacttactgaaactggaacaccgcactccagaatatttggaaaaaaatccgattcactcgattccagtattggaagatggagatttaatattacatgatagccacgcaataatggcctacctcgctgacacctacggaaaggatgagtcttggtatcctaaggacgtgaaaaaacgagctttagttaaccaaaaactgtttttcaccacaggtgtaattttccctagactacgaatcattacatactatcttctagaaaaaggaagaaaagctatagaacaa

SEQ ID NO: 228 - RNA, 100 nt, Spodoptera frugiperda
  source 1..100: mol_type = mRNA
acgggcacacaatgttccaacactttagtcgaccagtacattagcgagccacctgttgtcgtgaaaattgataaaaaataatgtgatggacattttaata

SEQ ID NO: 229 - RNA, 100 nt, Spodoptera frugiperda
  source 1..100: mol_type = mRNA
taagtgtgaaaatgggtgttaaggttttagctttaatagcactcttagttgttagtgtactcggacaagacgttactgtcagtgacaacgatgtaacaaa

SEQ ID NO: 230 - RNA, 100 nt, Spodoptera frugiperda
  source 1..100: mol_type = mRNA
ggaagttgtggtggacaatttagctagtgaggacccgatagtattagacacagttacagaagccacaaaagatgaagttgctgaagatgcgaacccagtg

SEQ ID NO: 231 - RNA, 100 nt, Spodoptera frugiperda
  source 1..100: mol_type = mRNA
gaaatactaagtccaacggatgatctgcaagtaagaagcgggaaatatcagctcaatgatgggctcgtgggtgaagagccgatgccactagatgctgtta

SEQ ID NO: 232 - RNA, 100 nt, Spodoptera frugiperda
  source 1..100: mol_type = mRNA
aaggagattaccatcagaccccagcttgaatacatagatattatcgaaatgggcgtggctaacactggagaatatgtgatcaatccccaaaactttagga

SEQ ID NO: 233 - RNA, 100 nt, Spodoptera frugiperda
  source 1..100: mol_type = mRNA
accgggataacttcatgcacaatgatatgcagttcggtttcctccagattaatttaactacgcctgaagtcttcaaaggagtctctatctcgcctgtact

SEQ ID NO: 234 - RNA, 150 nt, Spodoptera frugiperda
  source 1..150: mol_type = mRNA
taagtgtgaaaatgggtgttaaggttttagctttaatagcactcttagttgttagtgtactcggacaagacgttactgtcagtgacaacgatgtaacaaaggaagttgtggtggacaatttagctagtgaggacccgatagtattagaca

SEQ ID NO: 235 - RNA, 150 nt, Spodoptera frugiperda
  source 1..150: mol_type = mRNA
aaggagattaccatcagaccccagcttgaatacatagatattatcgaaatgggcgtggctaacactggagaatatgtgatcaatccccaaaactttaggaaccgggataacttcatgcacaatgatatgcagttcggtttcctccagatt

SEQ ID NO: 236 - RNA, 300 nt, Spodoptera frugiperda
  source 1..300: mol_type = mRNA
taagtgtgaaaatgggtgttaaggttttagctttaatagcactcttagttgttagtgtactcggacaagacgttactgtcagtgacaacgatgtaacaaaggaagttgtggtggacaatttagctagtgaggacccgatagtattagacaaaggagattaccatcagaccccagcttgaatacatagatattatcgaaatgggcgtggctaacactggagaatatgtgatcaatccccaaaactttaggaaccgggataacttcatgcacaatgatatgcagttcggtttcctccagatt

SEQ ID NO: 237 - RNA, 300 nt, Spodoptera frugiperda
  source 1..300: mol_type = mRNA
tggacaatttagctagtgaggacccaatagtattagacacagttacagaagccacaaaagatgaagttgctgaagatgcgaacccagtggaaatactaagtcctacggatgatctgcaagtaagaagtgggaaatatcagctcaatgatgggctcgtgggcgaagagccgataccactagatgctgttaatttcgactcgaataatgatgccggagagagtgagaagcagttgctctctcctggcacaactcaagtcacgaacaacgagtatgctcatatcgatggccgagttttaccgg

SEQ ID NO: 238 - RNA, 100 nt, Spodoptera frugiperda
  source 1..100: mol_type = mRNA
aagtcggacgttatacaaagaacgatgagtgtgctgaggggtttttagtgagaaattgtgaaaaatagaaagagtgaagatgtcggtaaaagcgagcgtt

SEQ ID NO: 239 - RNA, 100 nt, Spodoptera frugiperda
  source 1..100: mol_type = mRNA
ggttaaacaaaaagggtaaaaatggccttcaaaggattctgtggcgaagtgatcgggtttttcctggctgtgggtttttgcatcatatgtccggaatatg

SEQ ID NO: 240 - RNA, 100 nt, Spodoptera frugiperda
  source 1..100: mol_type = mRNA
aactgccgagtcaagaatttaggaaataaaacgttaaacatgcaagtatcgtgggtacggcatagagacatccatctgctgacagtcggccggtacacat

SEQ ID NO: 241 - RNA, 100 nt, Spodoptera frugiperda
  source 1..100: mol_type = mRNA
acacgagcgatcaaaggtttagagctattcacttaccgcactccgaggactggactttacagatcaagtatccgcaacacagggattcgggaatttatga

SEQ ID NO: 242 - RNA, 100 nt, Spodoptera frugiperda
  source 1..100: mol_type = mRNA
gttggttgcctgcctgttcatcgcactctcttgacacatacggatctttaactaaatgtaaatacaaggattttatcaacatcagctgcttatattgaca

SEQ ID NO: 243 - RNA, 200 nt, Spodoptera frugiperda
  source 1..200: mol_type = mRNA
aagtcggacgttatacaaagaacgatgagtgtgctgaggggtttttagtgagaaattgtgaaaaatagaaagagtgaagatgtcggtaaaagcgagcgttggttaaacaaaaagggtaaaaatggccttcaaaggattctgtggcgaagtgatcgggtttttcctggctgtgggtttttgcatcatatgtccggaatatg

SEQ ID NO: 244 - RNA, 100 nt, Spodoptera frugiperda
  source 1..100: mol_type = mRNA
aattcaaccccttgaatgttaaggtcggctcccacatcaccggaggagacttgtacggtatcgtacacgagaacacattggttaagcacaagatgttgat

SEQ ID NO: 245 - RNA, 100 nt, Spodoptera frugiperda
  source 1..100: mol_type = mRNA
tggacaagaagctcgcgcagcgcaagcacttccccgccatcaactggctcatctcctacagcaagtacatgcgagcgctggatgacttctatgagaagaa

SEQ ID NO: 246 - RNA, 100 nt, Spodoptera frugiperda
  source 1..100: mol_type = mRNA
gccgagactgacaaaatcacccttgaggttgccaagctgctcaaagacgacttcttgcaacaaaacagctactcgtcatacgatcgtttctgtccgttct

SEQ ID NO: 247 - RNA, 300 nt, Spodoptera frugiperda
  source 1..300: mol_type = mRNA
aattcaaccccttgaatgttaaggtcggctcccacatcaccggaggagacttgtacggtatcgtacacgagaacacattggttaagcacaagatgttgattggacaagaagctcgcgcagcgcaagcacttccccgccatcaactggctcatctcctacagcaagtacatgcgagcgctggatgacttctatgagaagaagccgagactgacaaaatcacccttgaggttgccaagctgctcaaagacgacttcttgcaacaaaacagctactcgtcatacgatcgtttctgtccgttct

SEQ ID NO: 248 - RNA, 1501 nt, Spodoptera frugiperda
  source 1..1501: mol_type = mRNA
atattattacttcacaaggacattcaactactggaagagtagaaatgtgcgaggaccaaaaccagttgcattatttggaaacattaaggacgcagctcttcgcaaagaaaattatggcgtcgtaatgcaaaatatatacaatgcatatccaaatgaaaaagtggtcggcatattcaggatgacttcgccttgtctccttattcgagacctggacattatcaaacatatcatgatcaaagacttcgaagccttcagtgatcgtggagtggaattcagcaaagaaggattgggacaaaacttattccacgcggacggagatacatggactgccttgaggaacagattcactcccattttcacaacaggtaaattgaagaacatgttttacctaataaatgagggaggcgattcatttgtagagtacatccgtacagaatgccaaaagaaggaagaatttgatattcagcctctcctccagacgtatactttgtctacgatctccgcctgtgcattcggaattagctatgacagtcttgatgttaaaatggatactctgaaacttgtggataaaatattttcttcaccaagttttgcagttgaattggatatgatgtatcccggtctcctgaaatctctaaacctttctttattccctaccgccataaaaaagttctttgataatctagtgaataatgttatagagcaaagaaatggtaaaccatcgggtcgaaatgatttcatggatcttattttggcgctccgtgaaatgggagaggtcacaaactcaaaatatgactctgcaaagccagttgaaataacacctggtgtgatagcagcgcaagcttttgtgttttatgcggctggttatgaaaccagtgctaccactatgacgtacatgctttaccaactagcaatgaatccagacatccaaaagaagttgactgaagaaattgacgaatctctcaaagcaaataatggacaagttacatacgagagcattaaggaaatgaagtatttgaacaaagtgtttgatgaaactctacgaatgtactcgattgtagaacctctgcagaggaaagctgtaagagattacaaagtgcccggtactgacttgacgatagaaaagaacacaattgtgctggtatctccgagaggtatccactacgacgagaaatattacgacaaccctgaacagttcaaccctgacagatttgacgcggaggaggtgggcaagcgacatccgtgcgcttacatgccgtttggaattggacagagaaactgcatcggaatgaggttcggcagacttcaatcccaactgtgcataaccaagttgctgtctaagttccaagtggagccatcgaggaatactgcaaggaagctggaagtggaaccttgtcgctttatcatcggacccaaaggagggatacgtctgaatattgttccaagaaagctgaaggcttaacacattaaacgccatggggggcaaatgtgaccggcgtta

SEQ ID NO: 249 - RNA, 100 nt, Spodoptera frugiperda
  source 1..100: mol_type = mRNA
tttaaccacagaggcattgaaagtattgaaggggatcaactgaccgataatattctcatgatgaatggtccaagatggaagctgatgcgacagaatatga

SEQ ID NO: 250 - RNA, 100 nt, Spodoptera frugiperda; identical to SEQ ID NO: 153
  source 1..100: mol_type = mRNA

SEQ ID NO: 251 - RNA, 100 nt, Spodoptera frugiperda; identical to SEQ ID NO: 154
  source 1..100: mol_type = mRNA

SEQ ID NO: 252 - RNA, 100 nt, Spodoptera frugiperda
  source 1..100: mol_type = mRNA
tatcatcagcacagcgcgaactcggcaagggttcgccgactaacgcctccgccacggcatcgccggccggttctgtggacaacgcgatccggtccggagc

SEQ ID NO: 253 - RNA, 300 nt, Plutella xylostella
  source 1..300: mol_type = mRNA
cagaagggcgacgacaagaccgtcttccagatcccggacaacttctacccagaaaagtacaagaaggtgggcaaccagctggccgaccggttcggcacggacgcgggccgcatggtgccggtgcgcaacatcgcgctgccggacctcagcctgccgcagcagctgccgtaccacaaccagttctcgctgtttgtgccgaaacacaggcggatggctgctaagctgattgatatatttatgggaatgcgtgacgtggaggacctgcagtccgtgtgtagctactgccagctccgcatcaac

SEQ ID NO: 254 - RNA, 300 nt, Plutella xylostella
  source 1..300: mol_type = mRNA
tggtacaggattgctgcagaatattccagaaggttctcgatgatgagataagctcggtatactatggactgtataacttcgtgtgcattcggcgtcgactgaagggaaccctttcacagaaacaggtcaccttttatttgatgaaagacctccagcaaaatttaccgtttcttccgatctgttatacttgacgttataaattggaagaagaacaaatacataacgggagacagtattgataatggcatagcggagcggttttctgaagaaggacggaaaagtattgtcccgtatacctac

SEQ ID NO: 255 - RNA, 300 nt, Spodoptera frugiperda
  source 1..300: mol_type = mRNA
tgaagaaccatacgttggctcttacttgggcacggaaccgaccctcatcatacaagatcccgaatacatcaagaccgtcatgaccaaggactactatttcttcagtggccgtgaagtctctgcatactgtgaaaaggaaccgttaactcagaacctattcttcacttatggcgataagtggaaggtactgcgtcagaaccttacgcctttgttctcatccgcaaagatgaagaacatgttccatttgatcgagaaatgtgcccgtatcttcgagaacatggtcgaccaggaagtacagaa

SEQ ID NO: 256 - RNA, 189 nt, Plutella xylostella
  source 1..189: mol_type = mRNA
tttggctgacactgcggacgacgagaccatcgagtcgtcagaagtgcaggtgtggctcgacatgtccaaccagcagatcggcctcatgctcaaccgagacatacagttctcatacagggacttcgctaagaacttgctgtcgacttgcgactacaacccgaaggtgggcgacatcccgatcgacttcaa

SEQ ID NO: 257 - RNA, 301 nt, Plutella xylostella
  source 1..301: mol_type = mRNA
tagattttcggatgagatggtaaaagacttgttatatcgcatcaaccatcgccgtaaaataagaccctcactgcaagggtcaggaaaccactatgggccaaactctgcgctggtgaaacaagtattggaccactggagtcaaaaatacgatttcaaagaacgtgccaagaaactaaaccggcatcctcaatacataaccaatgttaacggcctggacatccactacatacatgttcagccttcagggaataaacgcgttgtgccaatcctgctcatccacagcgtagaatccaacagtctg

SEQ ID NO: 258 - RNA, 300 nt, Plutella xylostella
  source 1..300: mol_type = mRNA
acttattagtgtaagtgtaaaagggcaagaggacgctataattaatgaggacagtttagtgaatgacgtcacgagtgtagtgagtaatgacttggaggctcctgtagttgctgaagcgaaagaggtgggagtggaagggtttgtggaggaccctgctcctgtggaagtgatagccgattctgaagccgtggcggtgaggagtgggcggtaccaggcgctcaatgatgccctgcaggaggggccggtggacctccaggcagtggacctggacgagcagctcgggaggcagctgttgaaccc

SEQ ID NO: 259 - RNA, 300 nt, Plutella xylostella
  source 1..300: mol_type = mRNA
cggtcctttgacgtatccagggccaccgaactctgccaagattcctaccagtgccgatatgactacggaatgaccctcaacagagatatggctgagttcaccaagaactatttgtcttctatcacaaacatcaaggagaaaaacgcaaggagagtcatcagttgtggcatcttggagacaccgcggtttggacggaagagtaacttcttctttactcccggaactacggtgaactttgagtgcaatcaaaactttatcctgattggtgacaagcgccgagcttgcgagtccgacggccgg

SEQ ID NO: 260 - RNA, 300 nt, Plutella xylostella; identical to SEQ ID NO: 40
  source 1..300: mol_type = mRNA

SEQ ID NO: 261 - RNA, 300 nt, Spodoptera frugiperda
  source 1..300: mol_type = mRNA
gcgctcagcgatgcagatgttcaaaaacagatcaagcacatgatggccttcatcgagcaagaagccaatgaaaaggctgaggaaatcgatgcaaaggccgaggaggagttcaacatcgaaaaggggcgtctggtccagcagcagaggttgaagatcatggagtactatgagaagaaggaaaagcaggtcgaactccagaaaaagatccaatcctcaaacatgctgaaccaggctcgtctcagggtgctcaaagtacgtgaagaccatgtacgcaacctgttggacgaggcccgcaagcgc

SEQ ID NO: 262 - RNA, 300 nt, Plutella xylostella
  source 1..300: mol_type = mRNA
gtctggtgcagcagcagcgcctcaagatcatggagtactacgagaagaaggagcgcgcgcaggcgcagtacaaggagaagatcaagaaggatgtgaccttgccccccgcgcaaatattcaactttcttattattattatgtagaactaaaaatgcgtacaacattattaacatgtatgaaagaatccgtattaagtcagacttagctgtagcttttaggagacacagttaaattgaaatgttataccgatagcgtaagcattttattgatataattctggattgttgccataacaattat

SEQ ID NO: 263 - RNA, 300 nt, Spodoptera frugiperda
  source 1..300: mol_type = mRNA
aagtcggacgttatacaaagaacgatgagtgtgctgaggggtttttagtgagaaattgtgaaaaatagaaagagtgaagatgtcggtaaaagcgagcgttggttaaacaaaaagggtaaaaatggccttcaaaggattctgtggcgaagtgatcgggtttttcctggctgtgggtttttgcatcatatgtccggaatatggttggttgcctgcctgttcatcgcactctcttgacacatacggatctttaactaaatgtaaatacaaggattttatcaacatcagctgcttatattgaca

SEQ ID NO: 264 - RNA, 240 nt, Diabrotica virgifera
  source 1..240: mol_type = mRNA
gcaaagaaaaatgcgtcgaaaaataaaagagttgcactccaagccctcaaaaagaagaaacgattggaaaagacccaactacaaatagatggaacccttacaactattgaaatgcagagggaagccctcgaaggagctagcacaaatactgctgtattagattctatgaaaaatgctgcagatgcccttaagaaagctcataagaatttgaatgtagatgatgttcacgatatcatggat

SEQ ID NO: 265 - RNA, 300 nt, Plutella xylostella
  source 1..300: mol_type = mRNA
cttcaaattattgtagtgatgatcatacggaagccgatttgatagaattttaattcacatacgatactagcctatataacagtcgatttgtgcagtcggtcagtgagtgttgtttagtgagtcgtgcaactcgtaccctgcatgactttggactccgatgcgccatttggcctcagctataaaaacaaggaatgtgtttatcgtctctcccgatttcccaaacgactttagcttgcagtgatggtgtgataagtgaactttagtgatattccccgacatggcaatttgtcagagcactcg

SEQ ID NO: 266 - RNA, 300 nt, Plutella xylostella
  source 1..300: mol_type = mRNA
ataggtagttagctattttatttagtattcgggtgttacgataatgttgcaacctgacgctctcaaaggtcatgagaatgaggagttgaacagtgttcacaaaaacatacataatatcactgttaatgtgatagctgaggtacaaagggacttgactccatacgatatcacttcgctggtgtttcttctatacgatatcccggaaacggcgcttcagcggcttactctgctgcagagagtgagccgggacatcagtggcaaggatctgaacttgctctatgactgggcggtgtacgctca

SEQ ID NO: 267 - RNA, 300 nt, Plutella xylostella
  source 1..300: mol_type = mRNA
accaagccccacgtcatcatgatcatggctgacgacatgggatgggacgacacctcgacccacggctccaagtccgtgctgacccccaacctcgacgtgctgacccgctcaggagtgtccctccaccgctactacacccacgctctctgctcgcccgcccgtaccgctgtgctcaccggcaaatacgcccacaccgtcggtatgcagggtatgcctctgtccaacgctgaggagcgtggtatccccctagaggagcgcctgatctctcagtacctacaggacgctggttacaggacccag

SEQ ID NO: 268 - RNA, 300 nt, Plutella xylostella
  source 1..300: mol_type = mRNA
ccccagctcctgcgcccgcgcacgaatcacttccgcgatcgaatacccgctttcgctccgtaaacagtccgtgtgcaaatcatattttccgttttctcatcaacattgcaaaacgctaaattgcacttctacaccggcggatgtgttcggtgtcaacaaatgtggtgtttcttggcgctcgttggaataatgtctaattggagttgagattgagactatacacgatggcgcgatggtcctccagcgaagtgtggacactcgtagcgctctgtgccctcgtcacatcctgcctgtcagacc

SEQ ID NO: 269 - RNA, 301 nt, Plutella xylostella
  source 1..301: mol_type = mRNA
acctggactgggagtaccctggagcagccgaccgcggcggctcgttctccgacaaggacaagttcctgtacctcgtccaggagttgagacgagctttcctgagggttgggaagggctgggagctgaccgctgcggtaccgctggccaacttccgactgatggaggggtaccatgtgcctgagttgtgccaggagctggacgcgatccacgtgatggcgtacgacctgcgcggcaactgggccgggttcgccgacgtgcactccccgctgtactcccgcccgcacgaccagtacgcatactc

SEQ ID NO: 270 - RNA, 59 nt, Spodoptera frugiperda
  source 1..59: mol_type = genomic RNA
taccctgtagatccgaatttgtttgaagtgaggcgacaaattcggttctagagaggttt

SEQ ID NO: 271 - RNA, 59 nt, Spodoptera frugiperda
  source 1..59: mol_type = mRNA
ccttgtcattcttcttgcccgtgtgctttcatactactggacggagaactgataagggc

SEQ ID NO: 272 - RNA, 52 nt, Spodoptera frugiperda
  source 1..52: mol_type = mRNA
gctgcatcccgtgcggccgcagtgtcacgtgtagcaccatgggattcagctc

SEQ ID NO: 273 - RNA, 60 nt, Spodoptera frugiperda
  source 1..60: mol_type = mRNA
ctggttttcataatgatttgacagattgttctaaattctgagatcattgtgaaagctgat

SEQ ID NO: 274 - RNA, 56 nt, Spodoptera frugiperda
  source 1..56: mol_type = mRNA
ctagcactttggctgtgacctgtgtgtcgtgtcacatcacagtcagagttctagct

SEQ ID NO: 275 - RNA, 287 nt, Spodoptera frugiperda
  source 1..287: mol_type = mRNA
ctagcactttggctgtgacctgtgtgtcgtagtcacatcacagtcagagttctagcttaccctgtagatccgaatttgtttgaagtgaggcgacaaattcggttctagagaggtttccttgtcattcttcttgcccgtgtgctttcatactactggacggagaactgataagggcgctgcatcccgtgcggccgcagtgtcacgtgtagcaccatgggattcagctcctggttttcataatgatttgacagattgttctaaattctgagatcattgtgaaagctgat

SEQ ID NO: 276 - protein, 615 aa, Coat protein
GCATGCAGATCTCGATCCCGCGAAATTAATACGACTCACTATAGGGAGACCACAACGGTTTCCCTCTAGATCCCTACATTAGAGAAGAGAGCTAATAAAGAAGGAGATATACATATGGCGTCTAACTTTACCCAATTCGTTCTGGTTGATAACGGCGGTACGGGTGACGTTACCGTAGCTCCGTCCAACTTCGCCAACGGTGTTGCGGAATGGATTAGCTCTAACAGCCGCTCTCAGGCCTACAAAGTCACGTGCTCCGTTCGTCAGTCTAGCGCGCAGAATCGCAAATACACCATCAAAGTTGAAGTACCGAAAGTCGCAACGCAGACCGTAGGCGGCGTAGAACTCCCAGTTGCGGCCTGGCGCTCTTACCTCAACATGGAACTGACTATTCCGATTTTTGCGACGAACTCCGACTGCGAACTGATTGTTAAGGCAATGCAGGGCCTGCTGAAAGACGGTAATCCGATCCCATCTGCAATCGCTGCTAACTCTGGCATTTACTAATAAGCGGACGCGCTGCCACCGCTGAGCAATAACTAGCATAACCCCTTGGGGCCTCTAAACGGGTCTTGAGGGGTTTTTTGCTGAAAGGAGGAACTATATCCGTCGCGA

SEQ ID NO: 277 - protein, 1111 aa, Plutella xylostella cytochrome P450
AAGCTTAGATCTCGATCCCGCGAAATTAATACGACTCACTATAGGGAGACCACAACGGTTTCCCTCTAGATCACAAGTTTGTACAAAAAAGCAGGCTAAGAAGGAGATATACATACGCCGGCCATTCAAACATGAGGATTACCCATGTATTTAAATACCCATGTCCAGGCGCGCTCCGCGATCGCGGCAATAAAGCGGTTACAAGCCCGCAAAAATAGCAGAGTAATGTCGCGATAGCGCGGCATTAATGCAGCTTTATTGATTCCTAGTCCTCAGATACGGCTACCCTTCCTTCTTCTACAGCGTGGGATTGGAGCTCTATTCCAATTCCTAGCAAGTAGGTATACGGGACAATACTTTTCCGTCCTTCTTCAGAAAACCGCTCCGGACGATCCCTACATGAGGATTACCCATGTATCCCTCAATAAAGCTGCATTTTACCGTTTCTTCCGATCTGTTATACTTGACGTTATAAACAGTATTCCTATGGAATAGAGCTCCAATCCCACGCTGTAGAAGAAGGAAGGGTAGCCGTATCTGAGGACATTCCTACGTCCGGAGCGGTTTTCTGAAGAAGGACGGAAAAGTATTGTCCCGTATACCTACTTGCATCCCTACATGAGGATCACCCATGTATCCCTACTGTTTATAACGTCAAGTATAACAGATCGGAAGAAACGGTAAGGCCGGCATGGTCCCAGCCTCCTCGCTGGCGCCGGCTGGGCAACATTCCGAGGGGACCGTCCCCTCGGTAATGGCGAATGGGACCCCGGACCGAATACCCGGTCTGAACGAGGGCGGCCGCGGTACCCAAGAAGTACTTAGAGTTAATTAAGGAGTTCAAACATGAGGATCACCCATGTCGAAGCTCCCACACCCTAGCATAACCCCTTGGGGCCTCTAAACGGGTCTTGAGGGGTTTTTTGCTGAAAGGAGGAACTATATCCGGAATTCCGCAGGACTGGGCGGCGGGCTAGCATCGTCCATTCCGACAGCATCGCCAGTCACTATGGCGTGCTGCTAGCGCTATATGCGTTGATGCAATTTCTATGCGCACCCGTTCTCGGAGCACTGTCCGACCGCTTTGGCCGCCGCCCAGTCCTGCGCATGC

SEQ ID NO: 278 - protein, 1371 aa, Plutella xylostella
AAGCTTAGATCTCGATCCCGCGAAATTAATACGACTCACTATAGGGAGACCACAACGGTTTCCCTCTAGATCACAAGTTTGTACAAAAAAGCAGGCTAAGAAGGAGATATACATACGCCGGCCATTCAAACATGAGGATTACCCATGTATTTAAATACCCATGTCCAGGCGCGCTCCGCGATCGCGGCAATAAAGCGGTTACAAGCCCGCAAAAATAGCAGAGTAATGTCGCGATAGCGCGGCATTAATGCAGCTTTATTGATTCCTAGTCCTCAGATACGGCTACCCTTCCTTCTTCTACAGCGTGGGATTGGAGCTCTATTCCAATTCCTAACTGTTTATAACGTCAAGTATAACAGATCGGAAGAAACGGTAAATTTTGCTGGAATAGATTCCTACCGTCTATGCCATTATCAATACTGTCTCCCGTTATGTATTTGTTCTTCTTCCAATCGGATTCCTAGCAAGTAGGTATACGGGACAATACTTTTCCGTCCTTCTTCAGAAAACCGCTCCGGACGATCCCTACATGAGGATTACCCATGTATCCCTCAATAAAGCTGCATTCTATGGACTGTATAACTTCGTGTGCATTCGGCGTCGACTCTGGATTCCTATGGAATAGAGCTCCAATCCCACGCTGTAGAAGAAGGAAGGGTAGCCGTATCTGAGGACATTCCTACTATTCCAGCAAAATTTACCGTTTCTTCCGATCTGTTATACTTGACGTTATAAACAGTATTCCTACCGATTGGAAGAAGAACAAATACATAACGGGAGACAGTATTGATAATGGCATAGACGGATTCCTACGTCCGGAGCGGTTTTCTGAAGAAGGACGGAAAAGTATTGTCCCGTATACCTACTTGCATCCCTACATGAGGATCACCCATGTATCCCTCCAGAGTCGACGCCGAATGCACACGAAGTTATACAGTCCATAGGGCCGGCATGGTCCCAGCCTCCTCGCTGGCGCCGGCTGGGCAACATTCCGAGGGGACCGTCCCCTCGGTAATGGCGAATGGGACCCCGGACCGAATACCCGGTCTGAACGAGGGCGGCCGCGGTACCCAAGAAGTACTTAGAGTTAATTAAGGAGTTCAAACATGAGGATCACCCATGTCGAAGCTCCCACACCCTAGCATAACCCCTTGGGGCCTCTAAACGGGTCTTGAGGGGTTTTTTGCTGAAAGGAGGAACTATATCCGGAATTCCGCAGGACTGGGCGGCGGGCTAGCATCGTCCATTCCGACAGCATCGCCAGTCACTATGGCGTGCTGCTAGCGCTATATGCGTTGATGCAATTTCTATGCGCACCCGTTCTCGGAGCACTGTCCGACCGCTTTGGCCGCCGCCCAGTCCTGCGCATGC

SEQ ID NO: 279 - protein, 1111 aa, Plutella xylostella
AAGCTTAGATCTCGATCCCGCGAAATTAATACGACTCACTATAGGGAGACCACAACGGTTTCCCTCTAGATCACAAGTTTGTACAAAAAAGCAGGCTAAGAAGGAGATATACATACGCCGGCCATTCAAACATGAGGATTACCCATGTATTTAAATACCCATGTCCAGGCGCGCTCCGCGATCGCGGCAATAAAGCGGTTACAAGCCCGCAAAAATAGCAGAGTAATGTCGCGATAGCGCGGCATTAATGCAGCTTTATTGATTCCTAACGCGCCCCCCGCGCAAATATTCAACTTTCTTATTATTATTATGTAGAACTAAAATGTATTCCTAATTAAATGCGTACAACATTATTAACATGTATGAAAGAATCCGTATTAAGTCAGAAAATATCCCTACATGAGGATTACCCATGTATCCCTCAATAAAGCTGCATTGTTCTTGATCTTCTCCTTGTACTGCGCCTGCGCGCGCTCGAGCATTCCTAACATTTTAGTTCTACATAATAATAATAAGAAAGTTGAATATTTGCGCGGGGGGCGCGTATTCCTAATTTTCTGACTTAATACGGATTCTTTCATACATGTTAATAATGTTGTACGCATTTAATATCCCTACATGAGGATCACCCATGTATCCCTGCTCGAGCGCGCGCAGGCGCAGTACAAGGAGAAGATCAAGAACGGCCGGCATGGTCCCAGCCTCCTCGCTGGCGCCGGCTGGGCAACATTCCGAGGGGACCGTCCCCTCGGTAATGGCGAATGGGACCCCGGACCGAATACCCGGTCTGAACGAGGGCGGCCGCGGTACCCAAGAAGTACTTAGAGTTAATTAAGGAGTTCAAACATGAGGATCACCCATGTCGAAGCTCCCACACCCTAGCATAACCCCTTGGGGCCTCTAAACGGGTCTTGAGGGGTTTTTTGCTGAAAGGAGGAACTATATCCGGAATTCCGCAGGACTGGGCGGCGGGCTAGCATCGTCCATTCCGACAGCATCGCCAGTCACTATGGCGTGCTGCTAGCGCTATATGCGTTGATGCAATTTCTATGCGCACCCGTTCTCGGAGCACTGTCCGACCGCTTTGGCCGCCGCCCAGTCCTGCGCATGC

SEQ ID NO: 280 - protein, 1371 aa, Plutella xylostella
AAGCTTAGATCTCGATCCCGCGAAATTAATACGACTCACTATAGGGAGACCACAACGGTTTCCCTCTAGATCACAAGTTTGTACAAAAAAGCAGGCTAAGAAGGAGATATACATACGCCGGCCATTCAAACATGAGGATTACCCATGTATTTAAATACCCATGTCCAGGCGCGCTCCGCGATCGCGGCAATAAAGCGGTTACAAGCCCGCAAAAATAGCAGAGTAATGTCGCGATAGCGCGGCATTAATGCAGCTTTATTGATTCCTAACGCGCCCCCCGCGCAAATATTCAACTTTCTTATTATTATTATGTAGAACTAAAATGTATTCCTAATTAAATGCGTACAACATTATTAACATGTATGAAAGAATCCGTATTAAGTCAGAAAATATTCCTATTAGCTTAGCTGTAGCTTTTAGGAGACACAGTTAAATTGAAATGTTATACCGATGCTTATTCCTATGTAAGCGTAAGCATTTTATTGATATAATTCTGGATTGTTGCCATAACAATTATTACAATCCCTACATGAGGATTACCCATGTATCCCTCAATAAAGCTGCATTGTTCTTGATCTTCTCCTTGTACTGCGCCTGCGCGCGCTCGAGCATTCCTAACATTTTAGTTCTACATAATAATAATAAGAAAGTTGAATATTTGCGCGGGGGGCGCGTATTCCTAATTTTCTGACTTAATACGGATTCTTTCATACATGTTAATAATGTTGTACGCATTTAATATTCCTAAAGCATCGGTATAACATTTCAATTTAACTGTGTCTCCTAAAAGCTACAGCTAAGCTAAATTCCTATGTAATAATTGTTATGGCAACAATCCAGAATTATATCAATAAAATGCTTACGCTTACAATCCCTACATGAGGATCACCCATGTATCCCTGCTCGAGCGCGCGCAGGCGCAGTACAAGGAGAAGATCAAGAACGGCCGGCATGGTCCCAGCCTCCTCGCTGGCGCCGGCTGGGCAACATTCCGAGGGGACCGTCCCCTCGGTAATGGCGAATGGGACCCCGGACCGAATACCCGGTCTGAACGAGGGCGGCCGCGGTACCCAAGAAGTACTTAGAGTTAATTAAGGAGTTCAAACATGAGGATCACCCATGTCGAAGCTCCCACACCCTAGCATAACCCCTTGGGGCCTCTAAACGGGTCTTGAGGGGTTTTTTGCTGAAAGGAGGAACTATATCCGGAATTCCGCAGGACTGGGCGGCGGGCTAGCATCGTCCATTCCGACAGCATCGCCAGTCACTATGGCGTGCTGCTAGCGCTATATGCGTTGATGCAATTTCTATGCGCACCCGTTCTCGGAGCACTGTCCGACCGCTTTGGCCGCCGCCCAGTCCTGCGCATGC

SEQ ID NO: 281 - protein, 1023 aa, Plutella xylostella
AAGCTTAGATCTCGATCCCGCGAAATTAATACGACTCACTATAGGGAGACCACAACGGTTTCCCTCTAGATCACAAGTTTGTACAAAAAAGCAGGCTAAGAAGGAGATATACATACGCCGGCCATTCAAATTTAAATACCCATGTCCAGGCGCGCTCCGCGATCGCGGCAATAAAGCGGTTACAAGCCCGCAAAAATAGCAGAGTAATGTCGCGATAGCGCGGCATTAATGCAGCTTTATTGATTCCTAGTCCTCAGATACGGCTACCCTTCCTTCTTCTACAGCGTGGGATTGGAGCTCTATTCCAATTCCTAGCAAGTAGGTATACGGGACAATACTTTTCCGTCCTTCTTCAGAAAACCGCTCCGGACGATCCCTCAATAAAGCTGCATTTTACCGTTTCTTCCGATCTGTTATACTTGACGTTATAAACAGTATTCCTATGGAATAGAGCTCCAATCCCACGCTGTAGAAGAAGGAAGGGTAGCCGTATCTGAGGACATTCCTACGTCCGGAGCGGTTTTCTGAAGAAGGACGGAAAAGTATTGTCCCGTATACCTACTTGCATCCCTACTGTTTATAACGTCAAGTATAACAGATCGGAAGAAACGGTAAGGCCGGCATGGTCCCAGCCTCCTCGCTGGCGCCGGCTGGGCAACATTCCGAGGGGACCGTCCCCTCGGTAATGGCGAATGGGACCCCGGACCGAATACCCGGTCTGAACGAGGGCGGCCGCGGTACCCAAGAAGTACTTAGAGTTAATTAAGGAGTTCAACGAAGCTCCCACACCCTAGCATAACCCCTTGGGGCCTCTAAACGGGTCTTGAGGGGTTTTTTGCTGAAAGGAGGAACTATATCCGGAATTCCGCAGGACTGGGCGGCGGGCTAGCATCGTCCATTCCGACAGCATCGCCAGTCACTATGGCGTGCTGCTAGCGCTATATGCGTTGATGCAATTTCTATGCGCACCCGTTCTCGGAGCACTGTCCGACCGCTTTGGCCGCCGCCCAGTCCTGCTCGCGA

SEQ ID NO: 282 - protein, 1953 aa, Plutella xylostella
NOPACAAGCTTAGATCTCGATCCCGCGAAATTAATACGACTCACTATAGGGAGACCACAACGGTTTCCCTCTAGATCACAAGTTTGTACAAAAAAGCAGGCTAAGAAGGAGATATACATACGCCGGCCATTCAAATTTAAATACCCATGTCCAGGCGCGCTCCGCGATCGCACCAATTGCAGGCGTGAAGAATGTCCTCAGATACGGCTACCCTTCCTTCTTCTACAGCGTGGGATTGGAGCTCTATTCCAGCAAAATTTACCGTTTCTTCCGATCTGTTATACTTGACGTTATAAACAGTCGTAACGGCGCCAAATCTTCGAGGAATGACATGGTGGATCTTATTTCCGATTGGAAGAAGAACAAATACATAACGGGAGACAGTATTGATAATGGCATAGACGGTGGAAACAAGAAGGTGCGTATCGAAGTCGACGACGAACTTTTGGTGAGCCAATGTGTGCTGTTCTTGTTTAAACCCTCTAGCTGCTTTACAAAGTACTGGTTCCCTTTCCAGCGGGATGCTTTATCTAAACGCAATGAGAGAGGTATTCCTCAGGCCACATCGCTTCCTAGTTCCGCTGGGATCCATCGTTGGCGGCCGAAGCCGCCATTCCATAGTGAGTTCTGGCGCGCCAAGAACAGCACACATTGGCTCACCAAAAGTTCGTCGTCGACTTCGATACGCACCTTCTTGTTTCCACCGTCTATGCCATTATCAATACTGTCTCCCGTTATGTATTTGTTCTTCTTCCAATCGGAAATAAGATCCACCATGTCATTCCTCGAAGATTTGGCGCCGTTACGACTGTTTATAACGTCAAGTATAACAGATCGGAAGAAACGGTAAATTTTGCTGGAATAGAGCTCCAATCCCACGCTGTAGAAGAAGGAAGGGTAGCCGTATCTGAGGACATTCTTCACGCCTGCAATTGGTCGGACCGAATACCCGGTCTGAACGAGGGCGGCCGCGGTACCCAAGAAGTACTTAGAGTTAATTAAGGAGTTCAACGAAGCTCCCACACCCTAGCATAACCCCTTGGGGCCTCTAAACGGGTCTTGAGGGGTTTTTTGCTGAAAGGAGGAACTATATCCGGAATTCCGCAGGACTGGGCGGCGGGCTAGCATCGTCCATTCCGACAGCATCGCCAGTCACTATGGCGTGCTGCTAGCGCTATATGCGTTGATGCAATTTCTATGCGCACCCGTTCTCGGAGCACTGTCCGACCGCTTTGGCCGCCGCCCAGTCCTGCGGCAATAAAGCGGTTACAAGCCCGCAAAAATAGCAGAGTAATGTCGCGATAGCGCGGCATTAATGCAGCTTTATTGATTCCTAACGCGCCCCCCGCGCAAATATTCAACTTTCTTATTATTATTATGTAGAACTAAAATGTATTCCTAATTAAATGCGTACAACATTATTAACATGTATGAAAGAATCCGTATTAAGTCAGAAAATATCCCTCAATAAAGCTGCATTGTTCTTGATCTTCTCCTTGTACTGCGCCTGCGCGCGCTCGAGCATTCCTAACATTTTAGTTCTACATAATAATAATAAGAAAGTTGAATATTTGCGCGGGGGGCGCGTATTCCTAATTTTCTGACTTAATACGGATTCTTTCATACATGTTAATAATGTTGTACGCATTTAATATCCCTGCTCGAGCGCGCGCAGGCGCAGTACAAGGAGAAGATCAAGAACGGCCGGCATGGTCCCAGCCTCCTCGCTGGCGCCGGCTGGGCAACATTCCGAGGGGACCGTCCCCTCGGTAATGGCGAATGGGACCCCGGACCGAATACCCGGTCTGAACGAGGGCGGCCGCGGTACCCAAGAAGTACTTAGAGTTAATTAAGGAGTTCAACGAAGCTCCCACACCCTAGCATAACCCCTTGGGGCCTCTAAACGGGTCTTGAGGGGTTTTTTGCTGAAAGGAGGAACTATATCCGTCGCGA

SEQ ID NO: 283 - protein, 915 aa, Plutella xylostella
AAGCTTAGATCTCGATCCCGCGAAATTAATACGACTCACTATAGGGACCAATTGCAGGCGTGAAGAATGTCCTCAGATACGGCTACCCTTCCTTCTTCTACAGCGTGGGATTGGAGCTCTATTCCAGCAAAATTTACCGTTTCTTCCGATCTGTTATACTTGACGTTATAAACAGTCGTAACGGCGCCAAATCTTCGAGGAATGACATGGTGGATCTTATTTCCGATTGGAAGAAGAACAAATACATAACGGGAGACAGTATTGATAATGGCATAGACGGTGGAAACAAGAAGGTGCGTATCGAAGTCGACGACGAACTTTTGGTGAGCCAATGTGTGCTGTTCTTAAGTACTGCGATCGCGTTAACGCTTTATCACGATACCTTCTACCACATATCACTAACAACATCAACACTCATCACTCTCGACGACATCCACTCGATCACTACTCTCACACGACCGATTAACTCCTCATCCACGCGGCCGCCTGCAGGAGCAAGAACAGCACACATTGGCTCACCAAAAGTTCGTCGTCGACTTCGATACGCACCTTCTTGTTTCCACCGTCTATGCCATTATCAATACTGTCTCCCGTTATGTATTTGTTCTTCTTCCAATCGGAAATAAGATCCACCATGTCATTCCTCGAAGATTTGGCGCCGTTACGACTGTTTATAACGTCAAGTATAACAGATCGGAAGAAACGGTAAATTTTGCTGGAATAGAGCTCCAATCCCACGCTGTAGAAGAAGGAAGGGTAGCCGTATCTGAGGACATTCTTCACGCCTGCAATTGGTAAGCTTGCCATCTGTTTTCTTGCAAGATCAGCTGAGCAATAACTAGCATAGATAACAGATACTTCGGTATCTGTTATCTGTTTTTTTTGCTGAAAGGAGGAACTATATCCGGATCGCGA

SEQ ID NO: 284 - protein, 954 aa, Plutella xylostella
AAGCTTAGATCTCGATCCCGCGAAATTAATACGACTCACTATAGGGACCAATTGCAGGCGTGAAGAATGTCCTCAGATACGGCTACCCTTCCTTCTTCTACAGCGTGGGATTGGAGCTCTATTCCAGCAAAATTTACCGTTTCTTCCGATCTGTTATACTTGACGTTATAAACAGTCGTAACGGCGCCAAATCTTCGAGGAATGACATGGTGGATCTTATTTCCGATTGGAAGAAGAACAAATACATAACGGGAGACAGTATTGATAATGGCATAGACGGTGGAAACAAGAAGGTGCGTATCGAAGTCGACGACGAACTTTTGGTGAGCCAATGTGTGCTGTTCTTAAGTACTGCGATCGCGTTAACGCTTTATCACGATACCTTCTACCACATATCACTAACAACATCAACACTCATCACTCTCGACGACATCCACTCGATCACTACTCTCACACGACCGATTAACTCCTCATCCACGCGGCCGCCTGCAGGAGCAAGAACAGCACACATTGGCTCACCAAAAGTTCGTCGTCGACTTCGATACGCACCTTCTTGTTTCCACCGTCTATGCCATTATCAATACTGTCTCCCGTTATGTATTTGTTCTTCTTCCAATCGGAAATAAGATCCACCATGTCATTCCTCGAAGATTTGGCGCCGTTACGACTGTTTATAACGTCAAGTATAACAGATCGGAAGAAACGGTAAATTTTGCTGGAATAGAGCTCCAATCCCACGCTGTAGAAGAAGGAAGGGTAGCCGTATCTGAGGACATTCTTCACGCCTGCAATTGGTAAGCTTGCCATCTGTTTTCTTGCAAGATCAGCTGAGCAATAACTAGCATAGATAACAGATACTTCGGTATCTGTTATCTGTTTTTTTTCAACAGATAGCCGCGTTCGCGCGGCTATCTGTTTTTTTTGCTGAAAGGAGGAACTATATCCGGATCGCGA

SEQ ID NO: 285 - DNA, 2767 bp, Kanamycin-resistant pUC vector
  source 1..2767: mol_type = genomic DNA
ttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttattcgactctatagtgaagttcctattctctagaaagtataggaacttctgaagtggggaagcttaaattttcgcgaaaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgctt

SEQ ID NO: 286 - DNA, 4469 bp, Plutella xylostella
  source 1..4469: mol_type = genomic DNA
ttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttattcgactctatagtgaagttcctattctctagaaagtataggaacttctgaagtggggaagcttagatctcgatcccgcgaaattaatacgactcactatagggagaccacaacggtttccctctagatcacaagtttgtacaaaaaagcaggctaagaaggagatatacatacgccggccattcaaacatgaggattacccatgtatttaaatacccatgtccaggcgcgctccgcgatcgcggcaataaagcggttacaagcccgcaaaaatagcagagtaatgtcgcgatagcgcggcattaatgcagctttattgattcctagtcctcagatacggctacccttccttcttctacagcgtgggattggagctctattccaattcctagcaagtaggtatacgggacaatacttttccgtccttcttcagaaaaccgctccggacgatccctacatgaggattacccatgtatccctcaataaagctgcattttaccgtttcttccgatctgttatacttgacgttataaacagtattcctatggaatagagctccaatcccacgctgtagaagaaggaagggtagccgtatctgaggacattcctacgtccggagcggttttctgaagaaggacggaaaagtattgtcccgtatacctacttgcatccctacatgaggatcacccatgtatccctactgtttataacgtcaagtataacagatcggaagaaacggtaaggccggcatggtcccagcctcctcgctggcgccggctgggcaacattccgaggggaccgtcccctcggtaatggcgaatgggaccccggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccggaattccgcaggactgggcggcgggctagcatcgtccattccgacagcatcgccagtcactatggcgtgctgctagcgctatatgcgttgatgcaatttctatgcgcacccgttctcggagcactgtccgaccgctttggccgccgcccagtcctgcgcatgcagatctcgatcccgcgaaattaatacgactcactatagggagaccacaacggtttccctctagatccctacattagagaagagagctaataaagaaggagatatacatatggcgtctaactttacccaattcgttctggttgataacggcggtacgggtgacgttaccgtagctccgtccaacttcgccaacggtgttgcggaatggattagctctaacagccgctctcaggcctacaaagtcacgtgctccgttcgtcagtctagcgcgcagaatcgcaaatacaccatcaaagttgaagtaccgaaagtcgcaacgcagaccgtaggcggcgtagaactcccagttgcggcctggcgctcttacctcaacatggaactgactattccgatttttgcgacgaactccgactgcgaactgattgttaaggcaatgcagggcctgctgaaagacggtaatccgatcccatctgcaatcgctgctaactctggcatttactaataagcggacgcgctgccaccgctgagcaataactagcataaccccttggggcctctaaacgggtcttgaggggttttttgctgaaaggaggaactatatccgtcgcgaaaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgctt

SEQ ID NO: 287 - DNA, 4729 bp, Plutella xylostella
  source 1..4729: mol_type = genomic DNA
ttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttattcgactctatagtgaagttcctattctctagaaagtataggaacttctgaagtggggaagcttagatctcgatcccgcgaaattaatacgactcactatagggagaccacaacggtttccctctagatcacaagtttgtacaaaaaagcaggctaagaaggagatatacatacgccggccattcaaacatgaggattacccatgtatttaaatacccatgtccaggcgcgctccgcgatcgcggcaataaagcggttacaagcccgcaaaaatagcagagtaatgtcgcgatagcgcggcattaatgcagctttattgattcctagtcctcagatacggctacccttccttcttctacagcgtgggattggagctctattccaattcctaactgtttataacgtcaagtataacagatcggaagaaacggtaaattttgctggaatagattcctaccgtctatgccattatcaatactgtctcccgttatgtatttgttcttcttccaatcggattcctagcaagtaggtatacgggacaatacttttccgtccttcttcagaaaaccgctccggacgatccctacatgaggattacccatgtatccctcaataaagctgcattctatggactgtataacttcgtgtgcattcggcgtcgactctggattcctatggaatagagctccaatcccacgctgtagaagaaggaagggtagccgtatctgaggacattcctactattccagcaaaatttaccgtttcttccgatctgttatacttgacgttataaacagtattcctaccgattggaagaagaacaaatacataacgggagacagtattgataatggcatagacggattcctacgtccggagcggttttctgaagaaggacggaaaagtattgtcccgtatacctacttgcatccctacatgaggatcacccatgtatccctccagagtcgacgccgaatgcacacgaagttatacagtccatagggccggcatggtcccagcctcctcgctggcgccggctgggcaacattccgaggggaccgtcccctcggtaatggcgaatgggaccccggaccgaatacccggtctgaacgagggcggccgcggtacccaagaagtacttagagttaattaaggagttcaaacatgaggatcacccatgtcgaagctcccacaccctagcataaccccttggggcctctaaacgggtcttgaggggttttttgctgaaaggaggaactatatccggaattccgcaggactgggcggcgggctagcatcgtccattccgacagcatcgccagtcactatggcgtgctgctagcgctatatgcgttgatgcaatttctatgcgcacccgttctcggagcactgtccgaccgctttggccgccgcccagtcctgcgcatgcagatctcgatcccgcgaaattaatacgactcactatagggagaccacaacggtttccctctagatccctacattagagaagagagctaataaagaaggagatatacatatggcgtctaactttacccaattcgttctggttgataacggcggtacgggtgacgttaccgtagctccgtccaacttcgccaacggtgttgcggaatggattagctctaacagccgctctcaggcctacaaagtcacgtgctccgttcgtcagtctagcgcgcagaatcgcaaatacaccatcaaagttgaagtaccgaaagtcgcaacgcagaccgtaggcggcgtagaactcccagttgcggcctggcgctcttacctcaacatggaactgactattccgatttttgcgacgaactccgactgcgaactgattgttaaggcaatgcagggcctgctgaaagacggtaatccgatcccatctgcaatcgctgctaactctggcatttactaataagcggacgcgctgccaccgctgagcaataactagcataaccccttggggcctctaaacgggtcttgaggggttttttgctgaaaggaggaactatatccgtcgcgaaaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgctt

SEQ ID NO: 288 - DNA, 3772 bp, Plutella xylostella
  source 1..3772: mol_type = genomic DNA
ttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttattcgactctatagtgaagttcctattctctagaaagtataggaacttctgaagtggggaagcttagatctcgatcccgcgaaattaatacgactcactatagggagaccacaacggtttccctctagatcacaagtttgtacaaaaaagcaggctaagaaggagatatacatacgccggccattcaaatttaaatacccatgtccaggcgcgctccgcgatcgcggcaataaagcggttacaagcccgcaaaaatagcagagtaatgtcgcgatagcgcggcattaatgcagctttattgattcctagtcctcagatacggctacccttccttcttctacagcgtgggattggagctctattccaattcctagcaagtaggtatacgggacaatacttttccgtccttcttcagaaaaccgctccggacgatccctcaataaagctgcattttaccgtttcttccgatctgttatacttgacgttataaacagtattcctatggaatagagctccaatcccacgctgtagaagaaggaagggtagccgtatctgaggacattcctacgtccggagcggttttctgaagaaggacggaaaagtattgtcccgtatacctacttgcatccctactgtttataacgtcaagtataacagatcggaagaaacggtaaggccggcatggtcccagcctcctcgctggcgccggctgggcaacattccgaggggaccgtcccctcggtaatggcgaatgggaccccggaccgaatacccggtctgaacgagggcggccgcggtacccaagaagtacttagagttaattaaggagttcaacgaagctcccacaccctagcataaccccttggggcctctaaacgggtcttgaggggttttttgctgaaaggaggaactatatccggaattccgcaggactgggcggcgggctagcatcgtccattccgacagcatcgccagtcactatggcgtgctgctagcgctatatgcgttgatgcaatttctatgcgcacccgttctcggagcactgtccgaccgctttggccgccgcccagtcctgctcgcgaaaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgctt

SEQ ID NO: 289 - DNA, 4697 bp, Plutella xylostella
  source 1..4697: mol_type = genomic DNA
ttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttattcgactctatagtgaagttcctattctctagaaagtataggaacttctgaagtggggaagcttagatctcgatcccgcgaaattaatacgactcactatagggagaccacaacggtttccctctagatcacaagtttgtacaaaaaagcaggctaagaaggagatatacatacgccggccattcaaatttaaatacccatgtccaggcgcgctccgcgatcgcaccaattgcaggcgtgaagaatgtcctcagatacggctacccttccttcttctacagcgtgggattggagctctattccagcaaaatttaccgtttcttccgatctgttatacttgacgttataaacagtcgtaacggcgccaaatcttcgaggaatgacatggtggatcttatttccgattggaagaagaacaaatacataacgggagacagtattgataatggcatagacggtggaaacaagaaggtgcgtatcgaagtcgacgacgaacttttggtgagccaatgtgtgctgttcttgtttaaaccctctagctgctttacaaagtactggttccctttccagcgggatgctttatctaaacgcaatgagagaggtattcctcaggccacatcgcttcctagttccgctgggatccatcgttggcggccgaagccgccattccatagtgagttctggcgcgccaagaacagcacacattggctcaccaaaagttcgtcgtcgacttcgatacgcaccttcttgtttccaccgtctatgccattatcaatactgtctcccgttatgtatttgttcttcttccaatcggaaataagatccaccatgtcattcctcgaagatttggcgccgttacgactgtttataacgtcaagtataacagatcggaagaaacggtaaattttgctggaatagagctccaatcccacgctgtagaagaaggaagggtagccgtatctgaggacattcttcacgcctgcaattggtcggaccgaatacccggtctgaacgagggcggccgcggtacccaagaagtacttagagttaattaaggagttcaacgaagctcccacaccctagcataaccccttggggcctctaaacgggtcttgaggggttttttgctgaaaggaggaactatatccggaattccgcaggactgggcggcgggctagcatcgtccattccgacagcatcgccagtcactatggcgtgctgctagcgctatatgcgttgatgcaatttctatgcgcacccgttctcggagcactgtccgaccgctttggccgccgcccagtcctgcggcaataaagcggttacaagcccgcaaaaatagcagagtaatgtcgcgatagcgcggcattaatgcagctttattgattcctaacgcgccccccgcgcaaatattcaactttcttattattattatgtagaactaaaatgtattcctaattaaatgcgtacaacattattaacatgtatgaaagaatccgtattaagtcagaaaatatccctcaataaagctgcattgttcttgatcttctccttgtactgcgcctgcgcgcgctcgagcattcctaacattttagttctacataataataataagaaagttgaatatttgcgcggggggcgcgtattcctaattttctgacttaatacggattctttcatacatgttaataatgttgtacgcatttaatatccctgctcgagcgcgcgcaggcgcagtacaaggagaagatcaagaacggccggcatggtcccagcctcctcgctggcgccggctgggcaacattccgaggggaccgtcccctcggtaatggcgaatgggaccccggaccgaatacccggtctgaacgagggcggccgcggtacccaagaagtacttagagttaattaaggagttcaacgaagctcccacaccctagcataaccccttggggcctctaaacgggtcttgaggggttttttgctgaaaggaggaactatatccgtcgcgaaaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgctt

SEQ ID NO: 290 - DNA, 58 bp, Plutella xylostella
  source 1..58: mol_type = genomic DNA
gtcctcagatacggctacccttccttcttctacagcgtgggattggagctctattcca

SEQ ID NO: 291 - RNA, 300 nt, Zea mays
  source 1..300: mol_type = mRNA
ccttgttgttctgttgcctcatggttatgatggccaaggtcctgagcattctagtgcccgcttggagcgcttccttcagatgagtgatgataatccttttgtcatacctgagatggaaccaacacttcgcaagcagatacaagagtgtaactggcaagttgtgaatgtgacaactcctgcaaattattttcatctgttgcgtcggcagatacatagggagttccggaagccgctgattgtaacagctcctaagaacctgcttaggcacaaggattgcaagtcaaatctttcagagtttga

SEQ ID NO: 292 - RNA, 300 nt, Zea mays
  source 1..300: mol_type = mRNA
catcaggttgtgagaggtgttgtagaaagtttgaaaattattacccgccaagcaagtttgagagtggcagaatatgcttttcattatgccaaggccaatggccgggaaagggtctctgcgattcacaaggccaatattatgaggaagacagatggtcttttcctcaagtgttgccgtgaagtggctgagacgtaccctgaaattcaatacgaggaggtcatcattgacaattgttgtatgacgcttgtgaagaatcctggtctttttgatgtattagtgatgccaaacctctatggtgac

SEQ ID NO: 293 - RNA, 300 nt, Sorghum bicolor
  source 1..300: mol_type = mRNA
ccttgttgttctgctgcctcatggttatgatggccaaggtcctgagcattccagtgcccgcttggagcgcttccttcagatgagtgatgataatccttttgtcatacctgagatggaaccaacacttcgcaagcagatacaagagtgtaactggcaggttgtgaatgtaacaactcctgcaaattattttcatttgttgcgtcggcagatacatagggagttccggaagccactgattgtaacagctcctaagaacctgcttaggcacaaggattgcaagtcaaatctttcagagtttga

SEQ ID NO: 294 - RNA, 621 nt, Spodoptera frugiperda
  source 1..621: mol_type = mRNA
aaattaaatgtggttttggcatcaaagtcggcttgtcataggtcataacgtagctatcacagccagctttgatgagcatgacctcattcctgaacatcattatgcggaccttcagagggcttgtagtattagtgggtcaatggtttcccgggcctagcctgtcaaagcggcggtgaaatgtatccgtcatcatatcacagccactttgatgtggtcggtttacggagagcaccacgtccttacagcactgccagtaggtaagcaagtccgtggcccattcgtaaaaatggttgtgccatgtagctgactcatatcacagccatttttgacgagttgggctgggggacgttacgacgagcggctccctgcttccatgacacttcgagcagcacgcggtccgcgagtgcagcggcgctcacaaagtggctgtaatgtgtgtccccgtacatatcacagccagctttgatgagcgctgagcatcatttgctttcatcaatatagttgagcgacatcaacaaaccctcaagcgcaagaagcgcgtcagggggtcagcaaagtggttgtgtcttatgagattcatgaacatatcacagccagctttgttgacttctttgtcgcgta

SEQ ID NO: 295 - RNA, 217 nt, Spodoptera frugiperda
  source 1..217: mol_type = mRNA
attgtacttcatcaggtgctctggtgatgatggttccaggcgcttgttggagtacacttactgaaagacaggagtagtgagatgtcctcgacatcacaaattctcactaccttgtctttcatgtctcattcccggttactcactcaacctgggtgtgatgtgtgcactagttgctcggcccatcacaacctccttgagtgagcgatcgtggaggagg

SEQ ID NO: 296 - RNA, 429 nt, Plutella xylostella
  source 1..429: mol_type = mRNA
tctgttaatgaagagagctatccgtcgacagtattgaccataaaaactgtcatggagttgctctctttatgaacggtagcatcaaatattagtgccctacatctaccctgtagatccgaatttgtttgaagtgaggcgacaaattcggttctagagaggtttgtgtggtgcacgctgggaaggcaagatgtcggcatagctgattgatgtgatatacggctgtgtcacatcgagccagctcaatgtgttagacgaagttggtttgtgaccgtcactaacgggcagtagtctatggtttgctcgttttgatgatcgcaaaattaactcgcttgactatcaaggctccacgtctgagttggacaggggatctagacagttcgcaacatacttctgccagatctaactttccagctcacgcgtggagccaac

SEQ ID NO: 297 - RNA, 429 nt, Plutella xylostella; identical to SEQ ID NO: 296
  source 1..429: mol_type = mRNA

SEQ ID NO: 298 - RNA, 442 nt, Plutella xylostella
  source 1..442: mol_type = mRNA
aaatggccaatttagtggccgttacctctgagcttgtatgtccataaattcatccaaattcagaggtaacggcacactacattaggctactggttcttgtctctggcgcggcagtcagaattcgggacaagcccggagcggtggcttgtcccgaattttgactcggccaaatcaaaacagcggtggtctggctaggacgtgattcggtgtaacccttgtgtttcccgatcaaattgcaccaatcccggcctgccttcggccaccgcccgctctagccatatgaaagacatgggtagtgagatgtccaagtacatcaaaaattctcactaccttgtctttcatgtggactgagcataatctgacattagtcagtgacatagctgggataagttagtgtgaaatgaaacctatctcagctatgtcactatctcgtgtcaggaat

SEQ ID NO: 299 - RNA, 19 nt, Plutella xylostella
  source 1..19: mol_type = mRNA
acatgaggattacccatgt

SEQ ID NO: 300 - RNA, 19 nt, Plutella xylostella
  source 1..19: mol_type = mRNA
acatgaggatcacccatgt